(19) United States
(12) Patent Application Publication
(10) Pub. No.: US 2023/0190871 A1
(43) Pub. Date: Jun. 22, 2023

(54) METHODS AND COMPOSITIONS FOR TREATMENT OF VIRAL INFECTIONS

(71) Applicant: Sana Biotechnology, Inc., Seattle, WA (US)

(72) Inventors: Richard C. MULLIGAN, Seattle, WA (US); Michael J. VOLLES, Seattle, WA (US)

(73) Assignee: Sana Biotechnology, Inc., Seattle, WA (US)

(21) Appl. No.: 17/926,564

(22) PCT Filed: May 19, 2021
(86) PCT No.: PCT/US2021/033273

§ 371 Date: Nov. 18, 2022

Related U.S. Application Data
(60) Provisional application No. 63/072,041, filed on Aug. 28, 2020.
(60) Provisional application No. 63/027,904, filed on May 20, 2020.

(51) Int. Cl. A61K 38/17; A61K 48/00; A61K 47/64; A61P 31/14

(52) U.S. Cl. CPC A61K 38/1709; A61K 47/645; A61K 48/0058; A61K 48/0066; A61P 31/14

(57) ABSTRACT

Provided herein are methods of treating or reducing the likelihood of a virus infection, such as a coronavirus infection, by delivering to a subject in need a Chromosome 19 Open Reading Frame 66 (C19orf66) or a regulatory factor that increases expression of the gene encoding C19ord66 in a cell in a subject. The C19orf66 or regulatory factor may be delivered as a polynucleotide (e.g. mRNA or DNA) or as a protein, and may be contained in a vehicle for delivery, such as a viral or non-viral vector. Also provided are polynucleotides, proteins, and vehicles (e.g. viral and non-viral vector) and composition thereof, including for use in the methods.

### CROSS-REFERENCE TO RELATED APPLICATIONS

This application claims priority to U.S. Provisional Application 63/027,904 entitled “Methods and Compositions for Treatment of Viral Infections”, filed May 20, 2020 and to U.S. Provisional Application 63/072,041 entitled “Methods and Compositions for Treatment of Viral Infections”, filed Aug. 28, 2020, the contents of each of which are incoperated by reference in their entirety for all purposes.

### INCORPORATION BY REFERENCE OF SEQUENCE LISTING

The present application is being filed along with a Sequence Listing in electronic format. The Sequence Listing is provided as a file entiled 18615_2003940_SEQ created May 17, 2021 which is 35,739,205 bytes in size. The information in the electronic format of the Sequence Listing is incorporated by reference in its entirety.

### FIELD

The present disclosure relates to methods of treating or reducing the likelihood of a virus infection, such as a coronavirus infection, by delivering to a subject in need a Chromosome 19 Open Reading Frame 66 (C19orf66) or a regulatory factor that increases expression of the gene encoding C19ord66 in a cell in a subject. The C19orf66 or regulatory factor may be delivered as a polynucleotide (e.g. DNA or mRNA) or as a protein, and may be contained in a vehicle for delivery, such as a viral or non-viral vector. The present disclosure also relates to the polynucleotides, proteins, and vehicles (e.g. viral and non-viral vector) and composition thereof, including for use in the methods.

### BACKGROUND

Coronaviruses (CoVs) constitute a group of phylogenetically diverse enveloped viruses that encode the large plus strand RNA genomes and replicate efficiently in most mammals. Human CoV infections typically result in mild to severe upper and lower respiratory tract symptoms and disease. Severe Acute Respiratory Syndrome Coronavirus (SARS-CoV, also known as SARS-CoV-1) emerged in 2002-2003 causing acute respiratory distress syndrome (ARDS) with 10% mortality overall and up to 50% mortality in aged individuals. Middle Eastern Respiratory Syndrome Coronavirus (MERS-CoV) emerged in the Middle East in April of 2012, manifesting as severe pneumonia, acute respiratory distress syndrome (ARDS) and acute renal failure. More recently, SARS-CoV-2, which is the strain of coronavirus that causes coronavirus disease 2019 (COVID-19), has emerged as an infectious strain in humans.

There are limited therapies for the treatment or prevention of coronavirus infection, including SARS-CoV-2 infection. Many of the drugs currently under investigation were originally designed for other pathogens and were promptly repurposed for the current COVID-19 trial, including remdesivir, hydroxychloroquine, and favipiravir. In some aspects, there is insufficient evidence that any existing anti-viral drugs can efficiently treat coronavirus infection. There remains a need for improved therapies for treating virus infection, including coronavirus infections, including those associated with SARS-CoV-2.

### SUMMARY

Provided herein are polynucleotides and proteins, vehicles for delivery thereof, and compositions containing the same, for use in methods for delivering Chromosome 19 Open Reading Frame 66 (C19orf66) or for regulating the expression of C19orf66 in a cell in a subject that is known or suspected of having a virus infection or that is at risk of being infected with a virus. The methods and compositions provided herein provide several advantages. C19orf66 is one of numerous interferon stimulated genes (ISGs). However, many viruses prevent the expression of ISGs, including C19orf66, either by interfering with interferon action or by affecting other mechanisms which lead to ISG expression. The methods and compositions disclosed herein enable the expression of C19orf66 in infected cells by bypassing any mechanism that viruses may use to prevent the expression of C19orf66. In addition, the methods and compositions disclosed herein enable the selective expression of C 19orf66, rather than have the whole spectrum of ISGs expressed. This can enable expression of specific combinations of C19orf66 and other specific ISGs. Further, the methods and compositions disclosed herein can, in some aspects, restrict the expression of C19orf66 to infected cells or the specific cell types susceptible to virus infection. This selectivity can avoid potentially deleterious effects of expression of C19orf66 in uninfected or non-susceptible cells.

Provided herein is a method of treating a coronavirus infection in a subject, the method comprising administering to a subject known or suspected of having a coronavirus infection a composition comprising an agent for delivery of a Chromosome 19 Open Reading Frame 66 (C19orf66) protein to the subject or an agent for delivery of a regulatory factor that increases expression of the gene encoding C19orf66 in a cell in the subject.

Also provided herein is a method of reducing the likelihood of a coronavirus infection in a subject, the method comprising administering to a subject known or suspected of being exposed to a coronavirus a composition comrpising an agent for delivery of a Chromosome 19 Open Reading Frame 66 (C19orf66) protein to the subject or an agent for delivery of a regulatory factor that increases expression of the gene encoding C 19orf66 in a cell in the subject.

In some of any embodiments, the agent for delivery is heterologous to the subject. In some of any embodiments, the cell in the subject is infected with the coronavirus.

In some of any embodiments, the subject is administered an agent for delivery of a C19orf66 protein to the subject, and the agent is a nucleotide sequence encoding the C19orf66 protein. In some of any embodiments, the subject is administered an agent for delivery of a regulatory factor that increases expression of the gene encoding C 19orf66, and the agent is a nucleotide sequence encoding the regulatory factor.

In some of any embodiments, nucleotide sequence is operably linked to a promoter to control expression.

In some of any embodiments, the promoter is an inducible promoter. In some embodiments, expression of the nucleotide sequence, e.g. encoding C19orf66, is controlled by an inducible expression system. In some embodiments, the inducible expression system comprises a first nucleic acid sequence comprising the nucleotide sequence operably linked to a drug response element and a second nucleic acid sequence comprising a drug-controlled transactivator operably linked to a promoter. In some embodiments, the drug response element is a tetracycline response element or a modified form thereof, optionally wherein the modified form is Tet-On 3G, and the drug-controlled transactivator is a reverse Tet transactivator (rtTA). In some of any such embodiments, the method further comprises administering to the subject an effective amount of a drug for inducing expression of the nucleotide sequence by the inducible expression system, optionally wherein the drug is doxycycline. In some embodiments, the effective amount is an amount to control or regulate expression of the nucleotide sequence, e.g. encoding C 19orf66, as desired.

In some of any embodiments, the promoter is a constitutive promoter. In some of any ambodiments, the promoter is a human Ubiquitin C (UbC) promoter, a human elongation factor 1α (EF1α) promoter, an SV40 promoter, a Cytomegalovirus (CMV) promoter, or a PGK-1 promoter. In some of any emdboiments, the nucleotide sequence is operably linked to a promoter to control expression in the lung. In some of any embodiments, the promoter is a human surfactant A promoter , a human surfactant B promoter, a human surfactant C promoter, a human surfactant D promoter, human ROBO4 promoter, or a human CDH1 gene. In some of any embodiments, the promoter is the human surfactant B promoter set forth in SEQ ID NO: 10.

In some of any embodiments, the subject is administered an agent for delivery of a C19orf66 protein to the subject, and the agent is the C19orf66 protein. In some of any embodiments, the C19orf66 protein is a recombinant protein. In some of any embodiments, the C19orf66 protein is linked to a cell penetrating peptide. In some of any embodiments, the protein is linked indirectly to the cell penetrating peptide via a peptide linker.

In some of any embodiments, the subject is administered an agent for delivery of a regulatory factor that increases expression of the gene encoding C 19orf66, and the agent is a regulatory factor protein or a protein complex. In some of any embodiments, the regulatory factor protein or protein complex is linked to a cell penetrating peptide. In some of any embodiments, the protein or protein complex is linked indirectly to the cell penetrating peptide via a peptide linker.

In some of any emdboiments, the cell penetrating peptide is a peptide that facilitates delivery to the interior of a cell. In some of any embodiments, the cell penetrating peptide is selected from the group consisting of: TAT (SEQ ID NO: 13), Penetratin (SEQ ID NO: 14), Transporant (SEQ ID NO: 15), Pept 1 (SEQ ID NO: 16), Pept 2 (SEQ ID NO: 17), Transportan (SEQ ID NO: 18), IgV (SEQ ID NO: 19 ).

In some of any embodiments, the administration of the agent inhibits or prevents viral replication of the coronavirus in the subject. In some of any embodiments, the administration of the agent inhibits or prevents ribosomal frameshifting in the subject. In some of any embodiments, the administration of the agent inhibits or prevents viral RNA processing in the subject.

In some of any emdboiments, the C19orf66 protein is or comprises the sequence of amino acids set forth in SEQ ID NO: 1, or a sequence of amino acids that has at least 90%, at least 92%, at least 95%, or at least 98% sequence identity to the sequence of amino acids set forth in SEQ ID NO:1. In some of any embodiments, C19orf66 protein is or comprises the sequence set forth in SEQ ID NO:1. In some of any embodiments, the C19orf66 protein is encoded by the nucleotide sequence set forth in SEQ ID NO: 2, or a nucleotide sequence that has at least 90%, at least 92%, at least 95%, or at least 98% sequence identity to SEQ ID NO:2. In some of any embodiments, the C19orf66 protein is encoded by the nucleotide sequence set forth in SEQ ID NO:2. In some of any embodiments, the C19orf66 protein is or comprises the sequence of amino acids set forth in SEQ ID NO: 3, or a sequence of amino acids that has at least 90%, at least 92%, at least 95%, or at least 98% sequence identity to the sequence of amino acids set forth in SEQ ID NO:3. In some of any embodiments, the C19orf66 protein is or comprises the sequence set forth in SEQ ID NO:3. In some of any embodiments, the C19orf66 protein is encoded by the nucleotide sequence set forth in SEQ ID NO: 4, or a nucleotide sequence that has at least 90%, at least 92%, at least 95%, or at least 98% sequence identity to the sequence set forth in SEQ ID NO:4. In some of any embodiments, the C19orf66 protein is encoded by the sequence set forth in SEQ ID NO:4. In some of any embodiments, the C19orf66 protein is or comprises the sequence of amino acids set forth in SEQ ID NO: 5, or a sequence of amino acids that has at least 90%, at least 92%, at least 95%, or at least 98% sequence identity to the sequence of amino acids set forth in SEQ ID NO:5. In some of any embodiments, the C19orf66 protein is or comprises the sequence set forth in SEQ ID NO:5. In some of any embodiments, the C19orf66 is encoded by the nucleotide sequence set forth in SEQ ID NO: 6, or a nucleotide sequence that has at least 90%, at least 92%, at least 95%, or at least 98% sequence identity to the sequence set forth in SEQ ID NO:6. In some of any embodiments, the C19orf66 protein is encoded by the sequence set forth in SEQ ID NO:6. In some of any embodiments, the C19orf66 protein is or comprises the sequence of amino acids set forth in SEQ ID NO: 7, or a sequence of amino acids that has at least 90%, at least 92%, at least 95%, or at least 98% sequence identity to the sequence of amino acids set forth in SEQ ID NO:7. In some of any embodiments, the C19orf66 protein is or comprises the sequence set forth in SEQ ID NO:7. In some of any embodiments, the C19orf66 protein is encoded by the nucleotide sequence set forth in SEQ ID NO: 8, or a sequence of nucleic acids that has at least 90%, at least 92%, at least 95%, or at least 98% sequence identity to the sequence set forth in SEQ ID NO: 8. In some of any embodiments, the C19orf66 protein is encoded by the nucleotide sequence set forth in SEQ ID NO:.8

In some of any embodiments, the C19orf66 protein comprises a nuclear localization signal. In some of any embodiments, the nuclear localization signal is selected from the group consisting of: KKRXKR (SEQ ID NO: 81), KRPAATKKAGQAKKKK (SEQ ID NO: 82) PAAKRBKLD (SEQ ID NO: 83), PKKKRKVEDP (SEQ ID NO: 84), and RRVPQRKEVSRCRKCRK (SEQ ID: NO 86). In some of any embodiments, the nuclear localization signal has the sequence of nucleic acids set forth in SEQ ID NO: 86.

In some of any embodiments, the C19orf66 protein comprises a nuclear export signal. In some of any embodiments, the nuclear export signal is selected from LXXXLXXLXL (SEQ ID NO:87) or LEDLDNLIL (SEQ ID: NO 85). In some of any embodiments, the nuclear export signal has the sequence of nucleic acids set forth in SEQ ID NO: 87.

In some of any embodiments, the regulatory factor controls targeted transcriptional activation of the gene encoding C19orf66. In some of any embodiments, the regulatory factor is a fusion protein comprising a site-specific binding domain specific for the C19orf66 gene, and a transcriptional activator. In some of any embodiments, the site-specific binding domain is selected from the group consisting of: zinc fingers, transcription activation like (TAL) effectors, meganucleases, and CRISPR/Cas system, or a modified form thereof. In some of any embodiments, the regulatory factor is a zinc finger transcription factor (ZF-TF). In some of any embodiments, the CRISPR/Cas system comprises a modified Cas nuclease that lacks nuclease activity and a guide RNA (gRNA). In some of any embodiments, the modified nuclease is a catalytically dead Cas9 (dCas9).

In some of any embodiments, the transcriptional activator is selected from Herpes simplex-derived transactivation domain, Dnmt3a methyltransferase domain, p65, VP16, and VP64. In some of any embodiments, the transcriptional activator is the tripartite activator VP64-p65-Rta (VPR).

In some of any embodiments, the agent is comprised in a vehicle that is a lipid particle or a non-lipid particle. In some of any embodiments, the vehicle is a lipid particle that is a viral vector or a viral-like particle.

In some of any embodiments, the viral vector or viral-like particle is derived from an Adeno-associated virus (AAV). In some of any embodiments, the AAV is of serotype 1, 2, 5, 6. In some of any embodiments, the AAV is of serotype 5. In some of any embodiments, the AAV is of serotype 6.

In some of any embodiments, the viral vector or viral-like particle is derived from a lentivirus. In some of any embodiments, the lentivirus is Human Immunodeficiency Virus-1 (HIV-1).

In some of any embodiments, the viral vector or viral-like particle is a virus-like particle. In some of any embodiments, the virus-like particle is replication defective.

In some of any embodiments, the viral vector or viral-like particle comprises a fusogen. In some of any embodiments, the vehicle is a lipid particle, wherein the lipid particle comprises (i) a lipid bilayer enclosing a lumen, and (ii) a fusogen, wherein the fusogen is embedded in the lipid bilayer. In some of any embodiments, the lipid bilayer is derived from a membrane of a host cell used for producing a virus or virus-like particle. In some of any embodiments, the lipid bilayer is derived from a membrane of a host cell used for producing a virus-like particle, wherein the virus-like particle is replication defective.

In some of any embodiments, the virus or virus-like particle is a retrovirus. In some of any embodiments, the retrovirus is a lentivirus. In some of any embodiments, the virus or virus-like particle is an adenovirus.

In some of any embodiments, the fusogen is a viral fusogen selected from a Class I viral membrane fusion protein, a Class II viral membrane protein, a Class II viral membrane fusion protein, a viral membrane glycoprotein, or a viral envelope protein. In some of any embodiments, the fusogen is a vesicular stomatitis virus envelope glycoprotein (VSV-G). In some of any embodiments, the fusogen is a syncytin.

In some of any embodiments, the fusogen is from a coronavirus. In some of any embodiments, the fusogen is a Severe Acute Respiratory Syndrome (SARS) coronavirus 1 (SARS CoV-1) spike glycoprotein. In some of any embodiments, the fusogen is a Severe Acute Respiratory Syndrome (SARS) coronavirus 2 (SARS CoV-2) spike glycoprotein. In some of any embodiments, the fusogen is an alpha coronavirus CD13 protein.

In some of any embodiments, the fusogen comprises an F protein molecule or a biologically active portion thereof from a Paramyxovirus and/or a glycoprotein G (G protein) or a biologically active portion thereof from a Paramyxovirus. In some of any embodiments, the fusogen is derived from an F protein molecule or a biologically active portion thereof from a Paramyxovirus and/or a glycoprotein G (G protein) or a biologically active portion thereof from a Paramyxovirus. In some of any embodiments, the Paramyxovirus is a henipavirus. In some of any embodiments, the Paramyxovirus is Nipah virus. In some of any embodiments, the Paramyxovirus is Hendra virus.

In some of any embodiments, the fusogen is a re-targeted fusogen comprising a targeting moiety that binds to a molecule on a target cell. In some of any embodiments, the targeting moiety is a Design ankyrin repeat proteins (DARPin), a single domain antibody (sdAb), a single chain variable fragment (scFv), or an antigen-binding fibronectin type III (Fn3) scaffold.

In some of any embodiments, the target cell is known or suspected of being infected by a coronavirus. In some of any embodiments, the targeting moiety binds a receptor of a coronavirus. In some of any embodiments, he targeting moiety binds angiotensin-converting enzyme 2 (ACE2). In some of any embodiments, the targeting moiety binds to transmembrane proteinase, serine 2 (TMPRSS2). In some of any embodiments, wherein the targeting moiety binds to dipeptidyl peptidase 4 (DPP4).

In some of any embodiments, the fusogen is modified to reduce its native binding tropism. In some of any embodiments, the G protein or the biologically active portion thereof is a mutant NiV-G protein that exhibits reduced binding to Ephrin B2 or Ephrin B3. In some of any embodiments, the mutant NiV-G protein comprises one or more amino acid substitutions corresponding to amino acid substitutions selected from the group consisting of E501A, W504A, Q530A and E533A with reference to numbering set forth in SEQ ID NO:26. In some of any embodiments, the mutant NiV-G protein has the amino acid sequence set forth in SEQ ID NO: 69 or an amino acid sequence having at or about 80%, at least at or about 81%, at least at or about 82%, at least at or about 83%, at or about 84%, at least at or about 85%, at least at or about 86%, or at least at or about 87%, at least at or about 88%, or at least at or about 89%, at least at or about 90%, at least at or about 91%, at least at or about 92%, at least at or about 93%, at least at or about 94%, at least at or about 95%, at or about 96%, at least at or about 97%, at least at or about 98%, or at least at or about 99% sequence identity to SEQ ID NO:69. In some of any embodiments, the mutant NiV-G protein has the amino acid sequence set forth in SEQ ID NO: 70 or an amino acid sequence having at or about 80%, at least at or about 81%, at least at or about 82%, at least at or about 83%, at or about 84%, at least at or about 85%, at least at or about 86%, or at least at or about 87%, at least at or about 88%, or at least at or about 89%, at least at or about 90%, at least at or about 91%, at least at or about 92%, at least at or about 93%, at least at or about 94%, at least at or about 95%, at or about 96%, at least at or about 97%, at least at or about 98%, or at least at or about 99% sequence identity to SEQ ID NO:70.

In some of any embodiments, the NiV-F protein is a biologically active portion thereof that has a 20 amino acid truncation at or near the C-terminus of the wild-type NiV-F protein (SEQ ID NO:37). In some of any embodiments, the NiV-F protein has an amino acid sequence set forth in SEQ ID NO:76 or an amino acid sequence having at or about 80%, at least at or about 81%, at least at or about 82%, at least at or about 83%, at or about 84%, at least at or about 85%, at least at or about 86%, or at least at or about 87%, at least at or about 88%, or at least at or about 89%, at least at or about 90%, at least at or about 91%, at least at or about 92%, at least at or about 93%, at least at or about 94%, at least at or about 95%, at or about 96%, at least at or about 97%, at least at or about 98%, or at least at or about 99% sequence identity to SEQ ID NO: 76. In some of any embodiments, the NiV-F protein has an amino acid sequence set forth in SEQ ID NO:91 or an amino acid sequence having at or about 80%, at least at or about 81%, at least at or about 82%, at least at or about 83%, at or about 84%, at least at or about 85%, at least at or about 86%, or at least at or about 87%, at least at or about 88%, or at least at or about 89%, at least at or about 90%, at least at or about 91%, at least at or about 92%, at least at or about 93%, at least at or about 94%, at least at or about 95%, at or about 96%, at least at or about 97%, at least at or about 98%, or at least at or about 99% sequence identity to SEQ ID NO: 91. In some of any embodiments, the NiV-F protein is a biologically active portion thereof that has a 22 amino acid truncation at or near the C-terminus of the wild-type NiV-F protein (SEQ ID NO:37). In some of any embodiments, the NiV-F protein has an amino acid sequence set forth in SEQ ID NO:75 or a sequence having at or about 80%, at least at or about 81%, at least at or about 82%, at least at or about 83%, at or about 84%, at least at or about 85%, at least at or about 86%, or at least at or about 87%, at least at or about 88%, or at least at or about 89%, at least at or about 90%, at least at or about 91%, at least at or about 92%, at least at or about 93%, at least at or about 94%, at least at or about 95%, at or about 96%, at least at or about 97%, at least at or about 98%, or at least at or about 99% sequence identity to SEQ ID NO: 75. In some of any embodiments, the NiV-F protein has an amino acid sequence set forth in SEQ ID NO:80 or a sequence having at or about 80%, at least at or about 81%, at least at or about 82%, at least at or about 83%, at or about 84%, at least at or about 85%, at least at or about 86%, or at least at or about 87%, at least at or about 88%, or at least at or about 89%, at least at or about 90%, at least at or about 91%, at least at or about 92%, at least at or about 93%, at least at or about 94%, at least at or about 95%, at or about 96%, at least at or about 97%, at least at or about 98%, or at least at or about 99% sequence identity to SEQ ID NO: 80.

In some of any embodiments, the NiV-F protein comprises a point mutation on an N-linked glycosylation site. In some of any embodiments, the NiV-F protein is a biologically active portion thereof that comprises: i) a 20 amino acid truncation at or near the C-terminus of the wild-type NiV-F protein (SEQ ID NO:37); and ii) a point mutation on an N-linked glycosylation site.

In some of any embodiments, the NiV-F protein has an amino acid sequence set forth in SEQ ID NO:74 or an amino acid sequence having at or about 80%, at least at or about 81%, at least at or about 82%, at least at or about 83%, at or about 84%, at least at or about 85%, at least at or about 86%, or at least at or about 87%, at least at or about 88%, or at least at or about 89%, at least at or about 90%, at least at or about 91%, at least at or about 92%, at least at or about 93%, at least at or about 94%, at least at or about 95%, at or about 96%, at least at or about 97%, at least at or about 98%, or at least at or about 99% sequence identity to SEQ ID NO: 74.

In some of any embodiments, the agent is comprised in a non-viral vector. In some of any embodiments, the non-viral particle is a liposome, a microparticle, a nanoparticle, a nanogel, a dendrimer or a dendrisome. In some of any embodiments, the non-viral vector is a nanoparticle. In some of any embodiments, the non-viral vector is a liposome. In some of any embodiments, the non-viral vector is a plasmid.

In some of any embodiments, the non-viral vector further comprises a vector-surface targeting moiety that binds to a molecule on a target cell, optionally where the target cell is a lung cell. In some of any embodiments, the target cell is known or suspected of being infected by a coronavirus. In some of any embodiments, the targeting moiety binds a receptor of a coronavirus. In some of any embodiments, the targeting moiety binds angiotensin-converting enzyme 2 (ACE2). In some of any embodiments, the targeting moiety binds to transmembrane proteinase, serine 2 (TMPRSS2). In some of any embodiments, the targeting moiety binds to dipeptidyl peptidase 4 (DPP4). In some of any embodiments, the vector-surface targeting moiety is a peptide or a polypeptide. In some of any embodiments, the polypeptide is an antibody or antigen-binding fragment.

In some of any embodiments, the non-viral vector is freeze dried. In some of any embodiments, the non-viral vector is subject to freeze and thaw prior to its administration.

In some of any embodiments, the agent is administered as a naked nucleic acid. In some of any embodiments, the agent is administered as an mRNA. In some of any embodiments, the agent is freeze dried. In some of any embodiments, the agent is subject to freeze and thaw prior to its administration.

Provided herein is a polynucleotide comprising a nucleotide sequence encoding Chromosome 19 Open Reading Frame 66 (C19orf66) or a nucleotide sequence encoding a regulatory factor that increases expression of the gene encoding C19orf66 in a cell , wherein the nucleotide sequence is operably linked to a promoter to control expression in the lung.

In some of any embodiments, the nucleotide sequence encodes C19orf66. In some of any embodiments, the nucleotide sequence encodes a regulatory factor that that increases expression of the gene encoding C19orf66 when the polynucleotide is administered to a cell in a subject.

In some of any embodiments, the encoded C19orf66 inhibits or prevents viral replication, optionally wherein the encoded C19orf66 inhibits or prevents viral replication of a Coronavirus. In some of any embodiments, the encoded C19orf66 inhibits or prevents ribosomal frameshifting. In some of any embodiments, the encoded C19orf66 inhibits or prevents viral RNA processing.

In some of any embodiments, the encoded C19orf66 is or comprises the sequence of amino acids set forth in SEQ ID NO: 1, or a sequence of amino acids that has at least 90%, at least 92%, at least 95%, or at least 98% sequence identity to the sequence of amino acids set forth in SEQ ID NO:1. In some of any embodiments, the encoded C19orf66 is or comprises the sequence set forth in SEQ ID NO:1. In some of any embodiments, the nucleotide sequence encoding C19orf66 is or comprises the sequence set forth in SEQ ID NO: 2, or a sequence that has at least 90%, at least 92%, at least 95%, or at least 98% sequence identity to the sequence set forth in SEQ ID NO:2. In some of any embodiments, the nucleotide sequence is or comprises the sequence set forth in SEQ ID NO:2. In some of any embodiments, the encoded C19orf66 is or comprises the sequence of amino acids set forth in SEQ ID NO: 3, or a sequence of amino acids that has at least 90%, at least 92%, at least 95%, or at least 98% sequence identity to the sequence of amino acids set forth in SEQ ID NO:3. In some of any embodiments, the encoded C19orf66 has the sequence set forth in SEQ ID NO:3. In some of any embodiments, the nucleotide sequence is or comprises the sequence set forth in SEQ ID NO: 4, or a sequence that has at least 90%, at least 92%, at least 95%, or at least 98% sequence identity to the sequence set forth in SEQ ID NO:4. In some of any embodiments, the nucleotide sequence is or comprises the sequence set forth in SEQ ID NO:4. In some of any embodiments, the encoded C19orf66 is or comprises the sequence of amino acids set forth in SEQ ID NO: 5, or a sequence of amino acids that has at least 90%, at least 92%, at least 95%, or at least 98% sequence identity to the sequence of amino acids set forth in SEQ ID NO:5. In some of any embodiments, the encoded C19orf66 is or comprises the sequence set forth in SEQ ID NO:5. In some of any embodiments, the nucleotide sequence is or comprises the sequence set forth in SEQ ID NO: 6, or a sequence that has at least 90%, at least 92%, at least 95%, or at least 98% sequence identity to the sequence set forth in SEQ ID NO:6. In some of any embodiments, the nucleotide sequence is or comprises the sequence set forth in SEQ ID NO:6. In some of any embodiments, the encoded C19orf66 is or comprises the sequence of amino acids set forth in SEQ ID NO: 7, or a sequence of amino acids that has at least 90%, at least 92%, at least 95%, or at least 98% sequence identity to the sequence of amino acids set forth in SEQ ID NO:7. In some of any embodiments, the encoded C19orf66 is or comprises the sequence set forth in SEQ ID NO:7. In some of any embodiments, the nucleotide sequence is or comprises the sequence set forth in SEQ ID NO: 8, or a sequence that has at least 90%, at least 92%, at least 95%, or at least 98% sequence identity to the sequence set forth in SEQ ID NO:.8. In some of any embodiments, the nucleotide sequence is or comprises the sequence set forth in SEQ ID NO:.8.

In some of any embodiments, the encoded C19orf66 comprises a nuclear localization signal. In some of any embodiments, the nuclear localization signal is selected from the group consisting of: KKRXKR (SEQ ID NO: 81), KRPAATKKAGQAKKKK (SEQ ID NO: 82), PAAKRBKLD (SEQ ID NO: 83), PKKKRKVEDP (SEQ ID NO: 84), and RVPQRKEVSRCRKCRK (SEQ ID: NO 86). In some of any embodiments, the nuclear localization signal has the sequence of nucleic acids set forth in SEQ ID NO: 86.

In some of any embodiments, the encoded C19orf66 comprises a nuclear export signal. In some of any embodiments, the nuclear export signal is selected from LXXXLXXLXL (SEQ ID NO:87) or LEDLDNLIL (SEQ ID: NO 85). In some of any embodiments, the nuclear export signal has the sequence of nucleic acids set forth in SEQ ID NO: 87. In some of any embodiments, the nuclear export signal has the sequence of nucleic acids set forth in SEQ ID NO: 85.

In some of any embodiments, the encoded regulatory factor controls targeted transcriptional activation of the gene encoding C19orf66. In some of any embodiments, the encoded regulatory factor is a fusion protein comprising a site-specific binding domain specific for the C19orf66 gene and a transcriptional activator. In some of any embodiments, the site-specific binding domain is selected from the group consisting of: zinc fingers, transcription activation like (TAL) effectors, meganucleases, and CRISPR/Cas9 system components, or a modified form thereof. In some of any embodiments, the encoded regulatory factor is a zinc finger transcription factor (ZF-TF). In some of any embodiments, the site-specific binding domain is a CRISPR/Cas system, wherein the CRISPR/Cas system comprises a modified Cas nuclease that lacks nuclease activity and a guide RNA (gRNA). In some of any embodiments, the modified nuclease is a catalytically dead Cas9 (dCas9).

In some of any embodiments, the transcriptional activator is selected from Herpes simplex-derived transactivation domain, Dnmt3a methyltransferase domain, p65, VP16, and VP64. In some of any embodiments, the transcriptional activator is the tripartite activator VP64-p65-Rta (VPR).

In some of any embodiments, the operably connected promoter is an inducible promoter. In some embodiments, the inducible promoter is part of a drug response element further comprising a drug operator sequence that is responsive to binding of a drug-controlled transactivator. In some embodiments, the nucleotide sequence is a first nucleic acid and the polynucleotide further comprises a nucleic acid encoding the drug-controlled transactivator operably linked to a promoter, optionally wherein the nucleic acid encoding the drug-controlled transactivator is in the forward orientation and the nucleotide sequence is in the reverse orientation. In some embodiments, the drug response element is a tetracycline response element (TRE), and wherein the drug-controlled transactivator is a reverse Tet transactivator (rtTA). Provided herein is a polynucleotide, comprising a first nucleic acid comprising a nucleotide sequence encoding Chromosome 19 Open Reading Frame 66 (C19orf66) operably linked to a tetracycline response element (TRE) and a second nucleic acid a comprising a reverse tetracycline transactivator (rtTA) operably linked to a promoter, optionally wherein the second nucleic acid is in the forward orientation and the first nucleic acid is in the reverse orientation. In some embodiments, the nucleotide sequence encoding C19orf66 is or comprises the sequence set forth in SEQ ID NO: 2, 4, 6 or 8, or a sequence that has at least 90%, at least 92%, at least 95%, or at least 98% sequence identity to the sequence set forth in SEQ ID NO:2, 4, 6 or 8. In some embodiments, the rtTA is controlled by doxycycline.

In some of any embodiments, the promoter (e.g. of the promoter operably connected to the nucleotide sequence encoding the C19orf66 or a nucleotide sequence encoding a regulatory factor that increases expression of the gene encoding C19orf66 in a cell) is a constitutive promoter. In some of any embodiments, the promoter (e.g. the promer operably connected to the drug-controlled transactivator, e.g. rtTa, is a constitutive promoter. In some emobdiments, the promoter is a ubiquitous promoter. In some embodiments, the promoter is a human Ubiquitin C (UbC) promoter, a human elongation factor 1α (EF1α) promoter, an SV40 promoter, a Cytomegalovirus (CMV) promoter, or a PGK-1 promoter. In some embodiments, the promoter is a tissue- or cell-specific promoter. In some embodiments, the promoter controls expression in the lung. In some of any embodiments, the promoter is a human surfactant A promoter , a human surfactant B promoter, a human surfactant C promoter, a human surfactant D promoter, human ROBO4 promoter, or a human CDH1 gene. In some of any embodiments, the promoter is t the human surfactant B promoter set forth in SEQ ID NO: 10.

In some of any embodiments, the nucleotide sequence is an mRNA. In some of any embodiments, the polynucleotide of any one of the provided emdbodiments is freeze-dried.

Provided here is a vehicle comprising any one of the polynuclepotides provided herein.

Provided herein is a fusion protein, comprising (1) a Chromosome 19 Open Reading Frame 66 (C19orf66) protein or a regulatory factor that increases expression of the gene encoding C19orf66 in a cell; and (2) a cell penetrating peptide.

In some of any embodiments, the fusion protein comprises (1) a C19Orf66 protein; and (2) a cell penetrating peptide. In some of any embodiments, the C19orf66 protein is linked indirectly to the cell penetrating peptide via a peptide linker. In some of any embodiments, the fusion protein comprises (1) a regulatory factor that increases expression of the gene encoding C19orf66 in a cell; and (2) a cell penetrating peptide.

In some of any embodiments, the C19orf66 protein or the regulatory factor is linked indirectly to the cell penetrating peptide via a peptide linker.

In some of any embodiments, the fusion protein inhibits or prevents viral replication. Optionally, wherein the encoded C19orf66 inhibits or prevents viral replication of a Coronavirus. In some of any embodiments, the fusion protein inhibits or prevents ribosomal frameshifting. In some of any embodiments, the fusion protein inhibits or prevents viral RNA processing.

In some of any embodiments, the C19orf66 protein is or comprises the sequence of amino acids set forth in SEQ ID NO: 1, or a sequence of amino acids that has at least 90%, at least 92%, at least 95%, or at least 98% sequence identity to the sequence of amino acids set forth in SEQ ID NO:1. In some of any embodiments, the C19orf66 protein is or comprises the sequence set forth in SEQ ID NO:1. In some of any embodiments, the C19orf66 protein is encoded by the nucleotide sequence set forth in SEQ ID NO: 2, or a nucleotide sequence that has at least 90%, at least 92%, at least 95%, or at least 98% sequence identity to SEQ ID NO:2. In some of any embodiments, the C19orf66 protein is encoded by the nucleotide sequence set forth in SEQ ID NO:2. In some of any embodiments, the C19orf66 protein is or comprises the sequence of amino acids set forth in SEQ ID NO: 3, or a sequence of amino acids that has at least 90%, at least 92%, at least 95%, or at least 98% sequence identity to the sequence of amino acids set forth in SEQ ID NO:3. In some of any embodiments, the C19orf66 protein is or comprises the sequence set forth in SEQ ID NO:3. In some of any embodiments, the C19orf66 protein is encoded by the nucleotide sequence set forth in SEQ ID NO: 4, or a nucleotide sequence that has at least 90%, at least 92%, at least 95%, or at least 98% sequence identity to the sequence set forth in SEQ ID NO:4. In some of any embodiments, the C19orf66 protein is encoded by the sequence set forth in SEQ ID NO:4. In some of any embodiments, the C19orf66 protein is or comprises the sequence of amino acids set forth in SEQ ID NO: 5, or a sequence of amino acids that has at least 90%, at least 92%, at least 95%, or at least 98% sequence identity to the sequence of amino acids set forth in SEQ ID NO:5. In some of any embodiments, the C19orf66 protein is or comprises the sequence set forth in SEQ ID NO:5. In some of any embodiments, the C19orf66 is encoded by the nucleotide sequence set forth in SEQ ID NO: 6, or a nucleotide sequence that has at least 90%, at least 92%, at least 95%, or at least 98% sequence identity to the sequence set forth in SEQ ID NO:6. In some of any embodiments, the C19orf66 protein is encoded by the sequence set forth in SEQ ID NO:6. In some of any embodiments, the C19orf66 protein is or comprises the sequence of amino acids set forth in SEQ ID NO: 7, or a sequence of amino acids that has at least 90%, at least 92%, at least 95%, or at least 98% sequence identity to the sequence of amino acids set forth in SEQ ID NO:7. In some of any embodiments, the fusion protein of any of claims 164-171 and 184, wherein the C19orf66 protein is or comprises the sequence set forth in SEQ ID NO:7. In some of any embodiments, the C19orf66 protein is encoded by the nucleotide sequence set forth in SEQ ID NO: 8, or a sequence of nucleic acids that has at least 90%, at least 92%, at least 95%, or at least 98% sequence identity to the sequence set forth in SEQ ID NO:.8 . In some of any embodiments, the C19orf66 protein is encoded by the nucleotide sequence set forth in SEQ ID NO:.8

In some of any embodiments, the C19orf66 protein comprises a nuclear localization signal. In some of any embodiments, the nuclear localization signal is selected from the group consisting of: KKRXKR (SEQ ID NO: 81), KRPAATKKAGQAKKKK (SEQ ID NO: 82), PAAKRBKLD (SEQ ID NO: 83), PKKKRKVEDP (SEQ ID NO: 84), and RRVPQRKEVSRCRKCRK (SEQ ID: NO 86). In some of any embodiments, the nuclear localization signal has the sequence of nucleic acids set forth in SEQ ID NO: 86.

In some of any embodiments, the C19orf66 protein comprises a nuclear export signal. In some of any embodiments, the nuclear export signal is selected from LXXXLXXLXL (SEQ ID NO:85) or LEDLDNLIL (SEQ ID: NO 87). In some of any embodiments, the nuclear export signal has the sequence of nucleic acids set forth in SEQ ID NO: 87.

In some of any embodiments, the regulatory factor controls targeted transcriptional activation of the gene encoding C19orf66. In some of any embodiments, the regulatory factor is a fusion protein comprising a site-specific binding domain specific for the C19orf66 gene, and a transcriptional activator. In some of any embodiments, the site-specific binding domain is selected from the group consisting of: zinc fingers, transcription activation like (TAL) effectors, meganucleases, and CRISPR/Cas system, or a modified form thereof. Insome of any embodiments, the regulatory factor is a zinc finger transcription factor (ZF-TF). In some of any embodiments, the site-specific binding domain is a CRISPR/Cas system, wherein the CRISPR/Cas system comprises a modified Cas nuclease that lacks nuclease activity and a guide RNA (gRNA). In some of any embodiments, the modified nuclease is a catalytically dead Cas9 (dCas9). In some of any embodiments, the transcriptional activator is selected from Herpes simplex-derived transactivation domain, Dnmt3a methyltransferase domain, p65, VP16, and VP64, optionally wherein the transcriptional activator is the tripartite activator VP64-p65-Rta (VPR).

In some of any embodiments, the cell penetrating peptide is a peptide that facilitates delivery to the interior of a cell. In some of any embodiments, the cell penetrating peptide is selected from the group consisting of: TAT (SEQ ID NO: 13), Penetratin (SEQ ID NO: 14), Transporant (SEQ ID NO: 15), Pept 1 (SEQ ID NO: 16), Pept 2 (SEQ ID NO: 17), Transportan (SEQ ID NO: 18) , IgV (SEQ ID NO: 19.

Provided herein is a vehicle, comprising any one of the fusion proteins provided herein. Provided herein is a vehicle comprising a nucleotide sequence encoding Chromosome 19 Open Reading Frame 66 (C19orf66). Also provided herein is a vehicle comprising a Chromosome 19 Open Reading Frame 66 (C19orf66) protein.

In some of any embodiments, the C19orf66 is a recombinant protein.

Provided herein is a vehicle comprising a nucleotide sequence encoding a regulatory factor capable of increasing expression of the gene encoding C19orf66. Also provided herein is a vehicle comprising a regulatory factor protein capable of increasing expression of a gene encoding C19orf66. In some of any embodiments, the regulatory factor is a recombinant fusion protein or is a protein complex.

In some of any embodiments, the C19orf66 protein is or comprises the sequence of amino acids set forth in SEQ ID NO: 1, or a sequence of amino acids that has at least 90%, at least 92%, at least 95%, or at least 98% sequence identity to the sequence of amino acids set forth in SEQ ID NO:1. in some of any embodiments, the C19orf66 protein is or comprises the sequence set forth in SEQ ID NO:1. In some of any embodiments, the C19orf66 protein is encoded by the nucleotide sequence set forth in SEQ ID NO: 2, or a nucleotide sequence that has at least 90%, at least 92%, at least 95%, or at least 98% sequence identity to SEQ ID NO:2. In some of any embodiments, the C19orf66 protein is encoded by the nucleotide sequence set forth in SEQ ID NO:2. In some of any embodiments, the C19orf66 protein is or comprises the sequence of amino acids set forth in SEQ ID NO: 3, or a sequence of amino acids that has at least 90%, at least 92%, at least 95%, or at least 98% sequence identity to the sequence of amino acids set forth in SEQ ID NO:3. In some of any embodiments, the C19orf66 protein is or comprises the sequence set forth in SEQ ID NO:3. In some of any embodiments, the C19orf66 protein is encoded by the nucleotide sequence set forth in SEQ ID NO: 4, or a nucleotide sequence that has at least 90%, at least 92%, at least 95%, or at least 98% sequence identity to the sequence set forth in SEQ ID NO:4. In some of any embodiments, the C19orf66 protein is encoded by the sequence set forth in SEQ ID NO:4. In some of any embodiments, the C19orf66 protein is or comprises the sequence of amino acids set forth in SEQ ID NO: 5, or a sequence of amino acids that has at least 90%, at least 92%, at least 95%, or at least 98% sequence identity to the sequence of amino acids set forth in SEQ ID NO:5. In some of any embodiments, the C19orf66 protein is or comprises the sequence set forth in SEQ ID NO:5. In some of any embodiments, the C19orf66 is encoded by the nucleotide sequence set forth in SEQ ID NO: 6, or a nucleotide sequence that has at least 90%, at least 92%, at least 95%, or at least 98% sequence identity to the sequence set forth in SEQ ID NO:6. In some of any embodiments, the C19orf66 protein is encoded by the sequence set forth in SEQ ID NO:6. In some of any embodiments, the C19orf66 protein is or comprises the sequence of amino acids set forth in SEQ ID NO: 7, or a sequence of amino acids that has at least 90%, at least 92%, at least 95%, or at least 98% sequence identity to the sequence of amino acids set forth in SEQ ID NO:7. In some of any embodiments, the C19orf66 protein is or comprises the sequence set forth in SEQ ID NO:7. In some of any embodiments, the C19orf66 protein is encoded by the nucleotide sequence set forth in SEQ ID NO: 8, or a sequence of nucleic acids that has at least 90%, at least 92%, at least 95%, or at least 98% sequence identity to the sequence set forth in SEQ ID NO:.8. In some of any embodiments, the C19orf66 protein is encoded by the nucleotide sequence set forth in SEQ ID NO:.8

In some of any embodiments, the C19orf66 protein comprises a nuclear localization signal. In some of any embodiments, the nuclear localization signal is selected from the group consisting of: KKRXKR (SEQ ID NO: 81), KRPAATKKAGQAKKKK (SEQ ID NO: 82), PAAKRBKLD (SEQ ID NO: 83), PKKKRKVEDP (SEQ ID NO: 84), and RRVPQRKEVSRCRKCRK (SEQ ID: NO 86). In some of any embodiments, the nuclear localization signal has the sequence of nucleic acids set forth in SEQ ID NO: 86.

In some of any embodiments, the C19orf66 protein comprises a nuclear export signal. In some of any embodiments, the nuclear export signal is selected from LXXXLXXLXL (SEQ ID NO:85) or LEDLDNLIL (SEQ ID: NO 87). In some of any embodiments, the nuclear export signal has the sequence of nucleic acids set forth in SEQ ID NO: 87.

In some of any embodiments, the regulatory factor controls targeted transcriptional activation of the gene encoding C19orf66. In some of any embodiments, the regulatory factor is a fusion protein comprising a site-specific binding domain specific for the C19orf66 gene, and a transcriptional activator. In some of any embodiments, the site-specific binding domain is selected from the group consisting of: zinc fingers, transcription activation like (TAL) effectors, meganucleases, and CRISPR/Cas system, or a modified form thereof. In some of any embodiments, the regulatory factor is a zinc finger transcription factor (ZF-TF). In some of any embodiments, the site-specific binding domain is a CRISPR/Cas system, wherein the CRISPR/Cas system comprises a modified Cas nuclease that lacks nuclease activity and a guide RNA (gRNA). In some of any embodiments, the modified nuclease is a catalytically dead Cas9 (dCas9). In some of any embodiments, the transcriptional activator is selected from Herpes simplex-derived transactivation domain, Dnmt3a methyltransferase domain, p65, VP16, and VP64. In some of any embodiments, the transcriptional activator is the tripartite activator VP64-p65-Rta (VPR).

In some of any embodiments, the vehicle is a lipid particle or a non-lipid particle. In some of any embodiments, the vehicle is a lipid particle that is a viral vector or a viral-like particle.

In some of any embodiments, the viral vector or viral-like particle is derived from an Adeno-associated virus (AAV) vector particle. In some of any embodiments, the AAV is of serotype 1, 2, 5, 6. In some of any embodiments, the AAV is of serotype 5. In some of any embodiments, the AAV is of serotype 6. In some of any embodiments, the viral vector or viral-like particle is derived from a lentivirus. In some of any embodiments, the lentivirus is Human Immunodeficiency Virus-1 (HIV-1).

In some of any embodiments, the viral vector or viral-like particle is a virus-like particle. In some of any embodiments, the viral vector or viral-like particle comprises a fusogen. In some of any embodiments, the vehicle is a lipid particle, wherein the lipid particle comprises (i) a lipid bilayer enclosing a lumen, and (ii) a fusogen, wherein the fusogen is embedded in the lipid bilayer. In some of any embodiments, the lipid bilayer is derived from a membrane of a host cell used for producing a virus or virus-like particle. In some of any embodiments, the lipid bilayer is derived from a membrane of a host cell used for producing a virus-like particle, wherein the virus-like particle is replication defective. In some of any embodiments, the virus or virus-like particle is a retrovirus. In some of any embodiments, the retrovirus is a lentivirus. In some of any embodiments, the virus or virus-like particle is an adenovirus.

In some of any embodiments, the viral vector or viral-like particle comprises a fusogen glycoprotein derived from a Paramyxovirus. In some of any embodiments, the fusogen is a viral fusogen selected from a Class I viral membrane fusion protein, a Class II viral membrane protein, a Class II viral membrane fusion protein, a viral membrane glycoprotein, or a viral envelope protein. In some of any embodiments, the fusogen is a vesicular stomatitis virus envelope glycoprotein (VSV-G). In some of any embodiments, the fusogen is a syncytin.

In some of any embodiments, the fusogen is from a coronavirus. In some of any embodiments, the the fusogen is a Severe Acute Respiratory Syndrome (SARS) coronavirus 1 (SARS CoV-1) spike glycoprotein. In some of any embodiments, the fusogen is a Severe Acute Respiratory Syndrome (SARS) coronavirus 2 (SARS CoV-2) spike glycoprotein. In some of any embodiments, the fusogen is an alpha coronavirus CD13 protein.

In some of any embodiments, the fusogen comprises an F protein molecule or a biologically active portion thereof from a Paramyxovirus and/or a glycoprotein G (G protein) or a biologically active portion thereof from a Paramyxovirus. In some of any embodiments, the fusogen is derived from an F protein molecule or a biologically active portion thereof from a Paramyxovirus and/or a glycoprotein G (G protein) or a biologically active portion thereof from a Paramyxovirus. In some of any embodiments, the Paramyxovirus is a henipavirus. In some of any embodiments, the Paramyxovirus is Nipah virus. In some of any embodiments, the Paramyxovirus is Hendra virus.

In some of any embodiments, the fusogen is a re-targeted fusogen comprising a targeting moiety that binds to a molecule on a target cell. In some of any embodiments, the targeting moiety is a Design ankyrin repeat proteins (DARPin), a single domain antibody (sdAb), a single chain variable fragment (scFv), or an antigen-binding fibronectin type III (Fn3) scaffold.

In some of any embodiments, the target cell is known or suspected of being infected by a coronavirus. In some of any embodiments, the targeting moiety binds a receptor of a coronavirus. In some of any embodiments, the targeting moiety binds angiotensin-converting enzyme 2 (ACE2). In some of any embodiments, the targeting moiety binds to transmembrane proteinase, serine 2 (TMPRSS2). In some of any embodiments, the targeting moiety binds to dipeptidyl peptidase 4 (DPP4).

In some of any embodiments, the fusogen is modified to reduce its native binding tropism. In some of any embodiments, the G protein or the biologically active portion thereof is a mutant NiV-G protein that exhibits reduced binding to Ephrin B2 or Ephrin B3. In some of any embodiments, the mutant NiV-G protein comprises one or more amino acid substitutions corresponding to amino acid substitutions selected from the group consisting of E501A, W504A, Q530A and E533A with reference to numbering set forth in SEQ ID NO:26.

In some of any embodiments, the mutant NiV-G protein has the amino acid sequence set forth in SEQ ID NO: 69 or an amino acid sequence having at or about 80%, at least at or about 81%, at least at or about 82%, at least at or about 83%, at or about 84%, at least at or about 85%, at least at or about 86%, or at least at or about 87%, at least at or about 88%, or at least at or about 89%, at least at or about 90%, at least at or about 91%, at least at or about 92%, at least at or about 93%, at least at or about 94%, at least at or about 95%, at or about 96%, at least at or about 97%, at least at or about 98%, or at least at or about 99% sequence identity to SEQ ID NO:69.

In some of any embodiments, the mutant NiV-G protein has the amino acid sequence set forth in SEQ ID NO: 70 or an amino acid sequence having at or about 80%, at least at or about 81%, at least at or about 82%, at least at or about 83%, at or about 84%, at least at or about 85%, at least at or about 86%, or at least at or about 87%, at least at or about 88%, or at least at or about 89%, at least at or about 90%, at least at or about 91%, at least at or about 92%, at least at or about 93%, at least at or about 94%, at least at or about 95%, at or about 96%, at least at or about 97%, at least at or about 98%, or at least at or about 99% sequence identity to SEQ ID NO:70. In some of any embodiments, the NiV-F protein is a biologically active portion thereof that has a 20 amino acid truncation at or near the C-terminus of the wild-type NiV-F protein (SEQ ID NO:37).

In some of any embodiments, the NiV-F protein has an amino acid sequence set forth in SEQ ID NO:76 or a sequence having at or about 80%, at least at or about 81%, at least at or about 82%, at least at or about 83%, at or about 84%, at least at or about 85%, at least at or about 86%, or at least at or about 87%, at least at or about 88%, or at least at or about 89%, at least at or about 90%, at least at or about 91%, at least at or about 92%, at least at or about 93%, at least at or about 94%, at least at or about 95%, at or about 96%, at least at or about 97%, at least at or about 98%, or at least at or about 99% sequence identity to SEQ ID NO: 76. In some of any embodiments, the NiV-F protein is a biologically active portion thereof that has a 22 amino acid truncation at or near the C-terminus of the wild-type NiV-F protein (SEQ ID NO:37).

In some of any embodiments, the NiV-F protein has an amino acid sequence set forth in SEQ ID NO:75 or a sequence having at or about 80%, at least at or about 81%, at least at or about 82%, at least at or about 83%, at or about 84%, at least at or about 85%, at least at or about 86%, or at least at or about 87%, at least at or about 88%, or at least at or about 89%, at least at or about 90%, at least at or about 91%, at least at or about 92%, at least at or about 93%, at least at or about 94%, at least at or about 95%, at or about 96%, at least at or about 97%, at least at or about 98%, or at least at or about 99% sequence identity to SEQ ID NO: 75.

In some of any embodiments, the NiV-F protein has an amino acid sequence set forth in SEQ ID NO:80 or a sequence having at or about 80%, at least at or about 81%, at least at or about 82%, at least at or about 83%, at or about 84%, at least at or about 85%, at least at or about 86%, or at least at or about 87%, at least at or about 88%, or at least at or about 89%, at least at or about 90%, at least at or about 91%, at least at or about 92%, at least at or about 93%, at least at or about 94%, at least at or about 95%, at or about 96%, at least at or about 97%, at least at or about 98%, or at least at or about 99% sequence identity to SEQ ID NO: 80. In some of any embodiments, the NiV-F protein comprises a point mutation on an N-linked glycosylation site.

In some of any embodiments, the NiV-F protein is a biologically active portion thereof that comprises: i) a 20 amino acid truncation at or near the C-terminus of the wild-type NiV-F protein (SEQ ID NO:37); and ii) a point mutation on an N-linked glycosylation site. In some of any embodiments, the NiV-F protein has an amino acid sequence set forth in SEQ ID NO:74 or a sequence having at or about 80%, at least at or about 81%, at least at or about 82%, at least at or about 83%, at or about 84%, at least at or about 85%, at least at or about 86%, or at least at or about 87%, at least at or about 88%, or at least at or about 89%, at least at or about 90%, at least at or about 91%, at least at or about 92%, at least at or about 93%, at least at or about 94%, at least at or about 95%, at or about 96%, at least at or about 97%, at least at or about 98%, or at least at or about 99% sequence identity to SEQ ID NO: 74.

In some of any embodiments, the vehicle is a non-viral vector. In some of any embodiments, the non-viral particle is a liposome, a microparticle, a nanoparticle, a nanogel, a dendrimer or a dendrisome. In some of any embodiments, the non-viral vector is a nanoparticle. In some of any embodiments, the non-viral vector is a lipsosome. In some of any embodiments, the non-viral vector is a plasmid.

In some of any embodiments, the vehicle is freeze dried. In some of any embodiments, the non-viral vector further comprises a vector-surface targeting moiety that binds to a molecule on a target cell. In some of any embodiments, the vector-surface targeting moiety is a peptide or a polypeptide. In some of any embodiments, the polypeptide is an antibody or antigen-binding fragment.

In some of any embodiments, the target cell is known or suspected of being infected by a coronavirus. In some of any embodiments, the target cell is a lung cell.

In some of any embodiments, the targeting moiety binds a receptor of a coronavirus. In some of any embodiments, the targeting moiety binds angiotensin-converting enzyme 2 (ACE2). In some of any embodiments, the targeting moiety binds to transmembrane proteinase, serine 2 (TMPRSS2). In some of any embodiments, the targeting moiety binds to dipeptidyl peptidase 4 (DPP4).

Provided herein is a composition comprising the polynucleotides, fusion proteins, or the vehicles, provided herein. In some of any embodiments, the composition further comprises a pharmaceutically acceptable carrier. In some of any embodiments, the composition is a pharmaceutical composition. In some of any embodiments, the composition is sterile.

Provided herein is a method of treating a viral infection, comprising administering to a subject known or suspected of having a virus infection any of the compositions provided herein. Also provided herein is a method of reducing the likelihood a viral infection, comprising administering to a subject known or suspected of having a virus infection any of the compositions provided herein.

Also provided is any of the provided compositions for use in a method of treating a viral infection in a subject known or suspected of having a virus infection. Also provided is any of the provided compositions for use in a method of reducing the likelihood of a viral infection in a subject known or suspected of being exposed to a virus.

Also provided is use of any of the provided compositions for manufacture of a medicament for use in a method of treating a viral infection in a subject known or suspected of having a virus infection. Also provided is use of any of the provided compositions for manufacture of a medicament for use in a method of reducing the likelihood of a viral infection in a subject known or suspected of being exposed to a virus.

In some of any embodiments, the virus relies on frameshifting. In some of any embodiments, the viral infection is caused by a coronavirus. In some of any embodiments, the viral infection is caused by SARS CoV-2.

In some of any embodiments, the composition is administered to the lung tissue of a subject. In some of any embodiments, the lung tissue is the bronchial or tracheal epithelium.

In some of any embodiments, the composition is administered by nebulization. In some of any embodiments, the composition is administered by inhalation. In some of any embodiments, the composition is administered by topical instillation. In some of any embodiments, the composition is administered by oral tablet. In some of any embodiments, the composition is administered parenterally, optionally subcutaneously or intravenously. In some of any embodiments, the composition is administered by injection. In some of any embodiments, the composition is administered by infusion.

In some of any embodiments, the subject is known, suspected, or predicted to have been exposed to a SARS coronavirus. In some of any embodiments, the subject is known, suspected, or predicted to have been exposed to a SARS CoV-1 virus. In some of any embodiments, the subject is known, suspected, or predicted to have been exposed to a SARS CoV-2 virus. In some of any embodiments, the subject is known or suspected of having Stage 1 Coronavirus disease 2019 (COVID-19). In some of any embodiments, the subject is known or suspected of having Stage 2 Coronavirus disease 2019 (COVID-19). In some of any embodiments, the subject is known or suspected of having Stage 2 Coronavirus disease 2019 (COVID-19). In some of any embodiments, the subject is known or suspected of having Severe Acute Respiratory Syndrome (SARS).

### BRIEF DESCRIPTION OF THE DRAWINGS

FIG. 1A shows the results of doxycycline induced c19orf66 expression in exemplary bulk sorted inducible cell lines, while corresponding reporter molecule expression (EGFP) is shown as a flow cytometry plot in FIG. 1B. These data are also shown for cells first isolated as single clones in FIG. 2, both as a western blot in FIG. 2A and as assayed for expression of reporter EGFP in FIG. 2B.

FIGS. 3A-3D depict induced expression of EGFP as meansured by flow cytometery in the presence of various concentrations of doxycycline. FIG. 3A depicts flow cytometry plots for EGFP expression for each cell population at 0 ng/mL doxycycline induction, and following treatment with various concentrations of doxycycline in the bulk sorted cell population shown in FIG. 3B, exemplary Clone 9 cells shown in FIG. 3C, and exemplary Clone 21 cells shown in FIG. 3D. FIG. 3E depticts a western blot probing for c19orf66 protein.

FIGS. 4A-D depict effects of induced expression of c19orf66 on infection of Vero cells with SARS-CoV2. FIG. 4A shows the percent infected cells as a result of infection with live SARS CoV-2 virus in Naive Vero cells as depicted in FIG. 4A, a sorted population of Vero cells in FIG. 4B, as well as exemplary individual clones 9 and 21 shown in FIG. 4C and FIG. 4D, respectively.

### DETAILED DESCRIPTION

Provided herein are methods of treating a viral infection, such as a coronavirus infection, the method comprising administering an agent for delivering a Chromosome 19 Open Reading Frame 66 (C19orf66) to a subject known or suspected of having a viral infection, such as a coronavirus infection. Also provided herein are methods of treating a viral infection, such as a coronavirus infection, the method comprising administering a regulatory factor controlling, such as increasing, expression of Chromosome 19 Open Reading Frame 66 (C19orf66) to a subject known or suspected of having a viral infection, such as a coronavirus infection. In certain embodiments, the regulator factor increases C19orf66 gene expression in a cell in a subject. In embodiments of the provided methods, C 19orf66 or regulatory factor that is administered is heterologous to the subject being treated. In some embodiments, the agent is a protein or a polynucleotide. For example, C19orf66 or the regulatory factor is administered as a polynucleotide. In some embodiments, C19orf66 or the regulatory factor thereof is administered as a protein or protein complex. For example, a C19orf66 recobinant protein may be administered to the subject. The polynucleotide or protein also may be contained in a vehicle, just as a viral or non-viral vector, for delivery. In particular embodiments, the provided methods are for use in treating a virus infection. For example, the provided embodiments include methods and uses for treating a Coronavirus infection, such as caused or due to SARS-CoV-2.

Provided herein are methods of reducing the likelihood of a viral infection, such as a coronavirus infection, the method comprising administering an agent for delivering a heterologous Chromosome 19 Open Reading Frame 66 (C19orf66) to a subject known or suspected of being exposed to a virus that may cause an infection, such as to a coronavirus. Also provided herein are methods of reducing the likelihood of a viral infection, such as a coronavirus infection, the method comprising administering a regulatory factor controlling, such as increasing, expression of Chromosome 19 Open Reading Frame 66 (C19orf66) to a subject known or suspected of being exposed to a virus, such as a coronavirus. In certain embodiments, the regulator factor increases C19orf66 gene expression in a cell in a subject. In embodiments of the provided methods, C 19orf66 or regulatory factor that is administered is heterologous to the subject being treated. In some embodiments, the agent is a protein or a polynucleotide. For example, C19orf66 or the regulatory factor thereof is administered as a polynucleotide. In some embodiments, C19orf66 or the regulatory factor thereof is administered as a protein or protein complex. For example, a C19orf66 recobinant protein may be administered to the subject. The polynucleotide or protein also may be contained in a vehicle, just as a viral or non-viral vector, for delivery. In particular embodiments, the provided methods are for use in preventing or reducing the likelihood of a Coronavirus infection, such as caused or due to SARS-CoV-2.

C19orf66, also known as Shiftless Inhibitor of Ribosomal Frameshifting (SHFL), RyDen, IRAV, SVA-1, and UPF0515, is encoded in 10 exons on human chromosome 19 encoding a primary isoform of 291 amino acids in length (SEQ ID NO:1). The encoded C19orf66 gene can further comprise an alternative in-frame splice site within the 3′ coding region. In some embodiments, SHFL is encoded in 10 exons on human chromosome 19 encoding a secondary isoform of 240 amino acids in length (SEQ ID NO:3).

In some embodiments, C19orf66 has a structure which contains alpha helices and beta pleated sheets. In some embodiments, C19orf66 has a structure which contains 8 alpha helices and 7 beta pleated sheets. In some embodiments, the activity of C19orf66 also has been shown to involve a nuclear localization signal (residues 121-137 of SEQ ID NO:1). In some embodiments, the activity of C19orf66 has been shown to involve a nuclear export signal (residues 261-269 of SEQ ID NO:1). In some aspects, C19orf66 contains a nucleic acid interaction domain. In some aspects, C19orf66 contains a PABPC1 interaction domain (residues 102-150 of SEQ ID NO: 1). In some aspects, the nucleic acid interaction domain can bind any single-stranded nucleic acid species. In some aspects, the nucleic acid interaction domain can bind any double-stranded nucleic acid species. In some embodiments, the nucleic-acid interaction domain displays higher affinity for single stranded nucleic acids. C19orf66 also features a coiled-coil motif and a glu-rich region in its C-terminal half.

In some aspects, C19orf66 is an interferon-stimulated gene (ISG) whose expression has been shown to be increased in response to signaling by type I interferons of the innate immune system.

In aspects of the provided embodiments, C19orf66 inhibits viral replication. In some embodiments, the virus is a member of any one of seven Baltimore classifications. In some embodiments, the virus comprises a DNA genome. In some embodiments, the virus comprises an RNA genome. In some embodiments, the viral genome is a single stranded nucleic acid species. In some embodiments, the viral genome is a double stranded nucleic acid species. In some embodiments, the virus is a member of any one of seven Baltimore classifications. Developed and proposed by David Baltimore, the Baltimore classifications divide viruses into major groups based on their genomic structure and replication strategy. In some embodiments, C19orf66 inhibits the replication of Baltimore Group I viruses. In some aspects, group I viruses comprise a double-stranded DNA genome, such as viruses belonging to Herpesviridae (e.g., Herpes simplex virus type 1), Adenoviridae (e.g., human Adenovirus), and Papovaviridae. In some embodiments, C19orf66 inhibits the replication of Baltimore Group II viruses. In some aspects, group II viruses comprise a single-stranded DNA genome, such as viruses belonging to Parvoviridae. In some embodiments, C19orf66 inhibits the replication of Baltimore Group III viruses. In some aspects, group III viruses comprise a double-stranded RNA genome, such as viruses belonging to Reoviridae and Birnaviridae. In some embodiments, C19orf66 inhibits the replication of Baltimore Group IV viruses. In some aspects, group IV viruses comprise a single-stranded RNA genome of the positive sense, such as viruses belonging to Coronaviridae, Flavivirdae (e.g., Dengue virus, West Nile virus, and Hepatitis C virus), Togaviridae (e..g, Chikungunya virus), and Picornaviridae. In some embodiments, C19orf66 inhibits the replication of Baltimore Group V viruses. In some aspects, group V viruses comprise a single-stranded RNA genome of the negative sense, such as viruses belonging to Orthomyxoviridae (e.g., Influenza viruses), Paramyxoviridae, Filoviridae, and Rhabdoviridae. In some embodiments, C19orf66 inhibits the replication of Baltimore Group VI viruses, such as retroviruses (e.g., lentiviruses such as HIV-1 and HIV-2). In some aspects, group VII viruses comprise a double-stranded RNA genome that requires and RNA intermediate, such as pararetroviruses (E.g., Hepatitis B). In some embodiments, the virus is within Baltimore Group IV. In some embodiments, the virus is a member of the family Coronaviridae. In some embodiments, the virus is a coronavirus.

The provided embodiments relate to the ability of C19orf66 to inhibit or prevent viral replication. In some embodiments, the C19orf66 inhibits or prevents viral RNA processing. In some embodiments, the encoded C19orf66 interacts with endogenous RNA binding proteins. In some aspects, the mechanism of inhibition is inhibition of ribosomal frameshifting.

Most known ribosomal frameshifting occurrences involve shifting -1 position, -1 mRNA nucleotide toward the 5′ end. A majority of these translational events involve dissociation of P-site tRNA anticodon:codon pairing and realignment at a new and overlapping codon. Frameshifting involving re-pairing at a non-overlapping new codon, also known as “hopping” or “bypassing”, is observed less frequently and is less understood.

In some aspects, many viruses utilize frameshifting in the generation of viral proteins. Frameshifting allows for high efficiency genomic organization wherein coding capacity and versatility is increased relative to genomic size. For example, a -1 frameshift is involved in the synthesis of the GagPol precursor protein of HIV-1. The HIV protease required for precursor cleavage is encoded from the 5′ end of the pol gene, and so is in-frame with the reverse transcriptase encoding sequence. This facilitates packaging of the inactive form of the pol precursor with the gag capsid components. In some aspects, Coronaviruses have been demonstrated to use frameshifting for the production of a viral polymerase. For example, Human coronavirus 229E has been observed as utilizing high level -1 frameshifting (Siddell, Nucleic Acids Res. 21(25):5838-42, 1993). A list of exemplary viruses with known and predicted occurrences of ribosomal frameshifting is shown in Table 1. In some embodiments, the provided methods relate to treating or reducing the likelihood of a viral infection of any virus of a taxon set forth in Table 1.

TABLE 1

Exemplary viruses with known and predicted occurrences of ribosomal frameshifting

| | |
|---|---|
| Virus Taxon | Exemplary Translational Product |
| Coronaviridae | Replicase |
| Retroviridae | Gag-Pol |
| Flaviviridae | Non-structural proteins |
| Orthomyxoviridae | PA-X |
| Herpesviridae | ARF |
| Togaviridae | Various structural proteins |
| Adenoviridae | L1 protein products |

Provided herein are methods for treating or reducing the likelihood of a viral infection. In some embodiments, the virus is of the order Nidovirales. In some embodiments, the virus is of sub-order Cornidovirineae. In some embodiments, the virus is of family Coronaviridae. In some embodiments, the virus is of sub-family Orthocoronavirinae. In some aspects, the virus is of genera Alphacoronavirus. In some aspects, the virus is of genera Betacoronavirus.

In provided embodiments, the methods relate to treating or reducing the likelihood of a coronavirus infection. Coronaviridae is a family of related enveloped viruses. Coronaviruses feature positive sense single stranded RNA genomes within a helical nucelocapsid and icosahedral protein coat. The genome of a coronavirus can vary between 26 to roughly 32 kilobases, some of the largest viral genomes recorded. The first human coronavirus were discovered in the 1960′s, including strains B814, 229E, IBV and OC43. More recently discovered human coronaviruses include NL63 and HKU1, identified in 2003 and 2004 respectively. Many human coronaviruses circulate in the population and cause seasonal epidemics and/or sporadic disease associated with the common cold.

In some embodiments, the virus is a coronavirus. In some embodiments, the coronavirus is of any of coronavirus subgroups: 1a, 1b, 2a, 2b, 2c, 2d, or 3.

In some embodiments, the virus is a subgroup 1a coronavirus. Nonlimiting examples of a subgroup 1a coronavirus of this invention include FCov.FIPV.79.1 146.VR.2202 (GenBank Accession No. NV_007025), transmissible gastroenteritis vims (TGEV) (GenBank Accession No. NC J302306; GenBank Accession No. Q81 1789.2; GenBank Accession No. DQ81 1786.2; GenBank Accession No. DQ811788.1 ; GenBank Accession No. DQ811785.1 ; GenBank Accession No. X52157.1 ; GenBank Accession No. AJ01 1482.1 ; GenBank Accession No. KC962433.1 ; GenBank Accession No. AJ271965.2; GenBank Accession No. JQ693060.1 ; GenBank Accession No. C609371.1 ; GenBank Accession No. JQ693060.1 ; GenBank Accession No. JQ693059.1 ; GenBank Accession No. JQ693058.1 ; GenBank Accession No. JQ693057.1 ; GenBank Accession No. JQ693052.1 ; GenBank Accession No. JQ693051.1 ; GenBank Accession No. JQ693050.1), porcine reproductive and respiratory syndrome virus (PRRSV) (GenBank Accession No. NC_0019 1.1 ; GenBank Accession No. DQ81 1787), as well as any other subgroup 1a coronavirus now known (e.g., as can be found in the GenBank® Database) or later identified, and any combination thereof.

In some embodiments, the virus is a subgroup 1b coronavirus. Nonlimiting examples of a subgroup 1b coronavirus of this invention include BtCoV. 1A.AFCD62 (GenBank Accession No. NC_010437), BtCoV. 1B.AFCD307 (GenBank Accession No. NCJH0436), BtCov.H U8.AFCD77 (GenBank Accession No. NC_010438), BtCoV.512.2005 (GenBank Accession No. DQ648858), porcine epidemic diarrhea virus PEDV.CV777 (GenBank Accession No. NCJ)034365 GenBank Accession No. DQ355224.1, GenBank Accession No. DQ355223.1, GenBank Accession No. DQ355221.1, GenBank Accession No. JN601062.1 , GenBank Accession No. JN601061.1, GenBank Accession No. JN601060.1, GenBank Accession No. J601059.1 , GenBank Accession No. JN601058.1, GenBank Accession N0.JN601057.1, GenBank Accession No, JN601056.1 , GenBank Accession N0.JN6OI 055, 1 , GenBank Accession No. JN601054.1 , GenBank Accession No. JN601053.1 , GenBank Accession No. JN601052.1 , GenBank Accession No. JN400902.1, GenBank Accession No.JN547395.1 , GenBank Accession No. FJ687473.1 , GenBank Accession No.FJ687472.1, GenBank Accession No. FJ687471.1 , GenBank Accession No. FJ687470.1 , GenBank Accession No. FJ687469.1 , GenBank Accession No.FJ687468.1, GenBank Accession No. FJ687467.1 , GenBank Accession No. FJ687466.1, GenBank Accession No. FJ687465.1, GenBank Accession No. FJ687464.1 , GenBank Accession No. FJ687463.1 , GenBank Accession No.FJ687462, 1 , GenBank Accession No. FJ68746U, GenBank Accession No. FJ687460.1 , GenBank Accession No. FJ687459.1, GenBank Accession No. FJ687458.1 , GenBank Accession No. FJ687457.1 , GenBank Accession No. FJ687456.1 , GenBank Accession No. FJ687455.1 , GenBank Accession No. FJ687454.1 , GenBank Accession No. FJ687453 GenBank Accession No. FJ687452.1 , GenBank Accession No. FJ687451.1, GenBank Accession No. FJ687450.1, GenBank Accession No. FJ687449.1 , GenBank Accession No. AF500215.1, GenBank Accession No. KF476061.1, GenBank Accession No. KF476060.1, GenBank Accession No. F476059.1, GenBank Accession No. KF476058.1, GenBank Accession No. KF476057.1 , GenBank Accession No. F476056.1 , GenBank Accession No. KF476055.1 , GenBank Accession No, KF476054.1, GenBank Accession No. KF476053.1 , GenBank Accession No. KF476052.1 , GenBank Accession No. KF476051.1 , GenBank Accession No. KF476050.1 , GenBank Accession No. F476049.1, GenBank Accession No. KF476048.1, GenBank Accession No. KF 177258.1 , GenBank Accession No. KF177257.1, GenBank Accession No. KF177256.1, GenBank Accession No. KF177255.1), HCoV.229E (GenBank Accession No. NCJ)02645), HCoV.NL63. Amsterdam.! (GenBank Accession No. NC_005831), BtCoV.H U2.HK.298.2006 (GenBank Accession No. EF203066), BtCoV.HKU2.HK.33.2006 (GenBank Accession No. EF203067), BtCoV.HKU2.HK.46.2006 (GenBank Accession No. EF203065), BtCoV.HKU2.GD.430.2006 (GenBank Accession No. EF203064), as well as any other subgroup 1b coronavirus now known (e.g., as can be found in the GenBank® Database) or later identified, and any combination thereof.

In some embodiments, the virus is a subgroup 2a coronavirus. Nonlimiting examples of a subgroup 2a coronavirus of this invention include HCoV.HKU1 .CN5 (GenBank Accession No. DQ339101), MHV.A59 (GenBank Accession No. NC_001846), PHEV.VW572 (GenBank Accession No. NC_007732), HCoV.OC43.ATCC.VR.759 (GenBank Accession No. NC_005147), bovine enteric coronavirus (BCoV.ENT) (GenBank Accession No. NC_003045), as well as any other subgroup 2a coronavirus now known (e.g., as can be found in the GenBank® Database) or later identified, and any combination thereof.

In some embodiments, the virus is a subgroup 2b coronavirus. Nonlimiting examples of a subgroup 2b coronavirus of this invention include BtSARS.HK1D.1 (GenBank Accession No, DQ022305), BtSARS.HKU3.2 (GenBank Accession No. DQ084199), BtSARS.HKU3.3 (GenBank Accession No. DQ084200), BtSARS.Rml (GenBank Accession No. DQ412043), BtCoV.279.2005 (GenBank Accession No. DQ648857), BtSARS.Rfl (GenBank Accession No. DQ412042), BtCoV.273.2005 (GenBank Accession No. DQ648856), BtSARS.Rp3 (GenBank Accession No. DQ071615), SARS CoV.A022 (GenBank Accession No. AY686863), SARSCoV.CUHK-Wl (GenBank Accession No. AY278554), SARSCoV.GDO1 (GenBank Accession No. AY278489), SARSCoV.HC.SZ.61.03 (GenBank Accession No. AY515512), SARSCoV.SZ 16 (GenBank Accession No. AY304488), SARSCoV.Urbani (GenBank Accession No. AY278741), SARSCoV.civetOlO (GenBank Accession No. AY572035), SARSCoV.MA.15 (GenBank Accession No. DQ497008), SARSCoV_Wuhan-HU-1 (GenBank Accession No. NC045512), SARSCoV_Unknown-UQ-581 (GenBank Accession No. MT412243), as well as any other subgroup 2b coronavirus now known (e.g., as can be found in the GenBank® Database) or later identified, and any combination thereof.

In some embodiments, the virus is a subgroup 2c coronavirus. Nonlimiting examples of a subgroup 2c coronavirus of this invention include Middle East respiratory syndrome coronavirus isolate Riyadh_2_2012 (GenBank Accession No. KF600652.1), Middle East respiratory syndrome coronavirus isolate Al-HasaJ 8J 013 (GenBank Accession No. F600651.1), Middle East respiratory syndrome coronavirus isolate Al-Hasa_17_2013 (GenBank Accession No. F600647.1), Middle East respiratory syndrome coronavirus isolate Al- Hasa_15_2013 (GenBank Accession No. F600645.1), Middle East respiratory syndrome coronavirus isolate Al-Hasa_16_2013 (GenBank Accession No. KF600644.1), Middle East respiratory syndrome coronavirus isolate Al-Hasa_21_2013 (GenBank Accession No. KF600634), Middle East respiratory syndrome coronavirus isolate Al-Hasa_19_ 2013 (GenBank Accession No. KF600632.), Middle East respiratory syndrome coronavirus isolate Buraidah_1_2013 (GenBank Accession No. KF600630.1), Middle East respiratory syndrome coronavirus isolate Ffafr-Al-Batin_1_2013 (GenBank Accession No. F600628.1), Middle East respiratory syndrome coronavirus isolate Al-Hasa_12_2013 (GenBank Accession No. KF600627.1), Middle East respiratory syndrome coronavirus isolate Bisha_1_2012 (GenBank Accession No. KF600620.1), Middle East respiratory syndrome coronavirus isolate Riyadh_3_2013 (GenBank Accession No. KF600613.1), Middle East respiratory syndrome coronavirus isolate RiyadhJ_2012 (GenBank Accession No. KF600612.1), Middle East respiratory syndrome coronavirus isolate AI-Hasa_3_2013 (GenBank Accession No. KF 186565.1), Middle East respiratory syndrome coronavirus isolate Al-Hasa_1_2013 (GenBank Accession No. KF186567.1), Middle East respiratory syndrome coronavirus isolate Al- Hasa_2_2013 (GenBank Accession No. F186566.1), Middle East respiratory syndrome coronavirus isolate Al-Hasa_4_2013 (GenBank Accession No. KF186564.1), Middle East respiratory syndrome coronavirus (GenBank Accession No. KF192507.1), Betacoronavirus England 1-N1 (GenBank Accession No. NC_019843), MERS-CoV_SA-Nl (GenBank Accession No. KC667074), following isolates of Middle East Respiratory Syndrome Coronavirus (GenBank Accession No: KF600656.1, GenBank Accession No; KF600655.1 , GenBank Accession No: KF600654.1, GenBank Accession No: KF600649.1 , GenBank Accession No: KF600648.1 , GenBank Accession No: KF600646.1 , GenBank Accession No: KF600643.1, GenBank Accession No: KF600642.1, GenBank Accession No: KF600640.1 , GenBank Accession No: KF600639.1 , GenBank Accession No: KF600638.1, GenBank Accession No: KF600637.1 , GenBank Accession No: KF600636.1 , GenBank Accession No: KF600635.1 , GenBank Accession No: KF600631.1 , GenBank Accession No: KF600626.1 , GenBank Accession No: KF600625.1 , GenBank Accession No: KF600624.1 , GenBank Accession No: KF600623.1 , GenBank Accession No: KF600622.1, GenBank Accession No: KF600621.1 , GenBank Accession No: KF600619.1 , GenBank Accession No: KF600618.1 , GenBank Accession No: KF600616.1, GenBank Accession No: KF600615.1 , GenBank Accession No: KF600614.1, GenBank Accession No: KF 600641.1, GenBank Accession No: KF600633.1 , GenBank Accession No: KF600629.1, GenBank Accession No: KF600617.1), Coronavirus Neoromicia/PML PHE1/RSA/201 1 GenBank Accession: KC869678.2, Bat Coronavirus Taper/CII_KSA_287/Bisha/Saudi ArabialGenBank Accession No: KF493885.1 ,Bat coronavirus Rhhar/CII_KSAJ)03/Bisha Saudi Arabia/2013 GenBank^ Accession No : KF493888.1 , Bat coronavirus Pikuh/CII^KSA_001 /Riyadh/Saudi Arabia/2013 GenBank_Accession No :KF493887.1 , Bat coronavirus Rhhar/CII KSA_002/Bisha/Saudi Arabia/2013 GenBank Accession No: KF493886.1, Bat Coronavirus Rhhar/CIIJiSA_ 004/Bisha/Saudi Arabia 2013 GenBank Accession No : KF493884.1 , BtCoV.HKU4.2 (GenBank Accession No. EF065506), BtCoV.HKU4.1 (GenBank Accession No. NC_009019), BtCoV.HKU4.3 (GenBank Accession No. EF065507), BtCoV.HKU4.4 (GenBank Accession No. EF065508), BtCoV133.2005 (GenBank Accession No. NCJ)08315), BtCoV.HKU5.5 (GenBank Accession No. EF065512); BtCoV.HKU5.1 (GenBank Accession No. NCJ)09020), BtCoV.HKU5.2 (GenBank Accession No. EF0655 I0), BtCoV.HKU5.3 (GenBank Accession No. EF06551 1), human betacoronavirus 2c Jordan-N3/2012 (GenBank Accession No. C776174.1 ; human betacoronavirus 2c EMC/2012, (GenBank Accession No. JX869059.2), Pipistrellus bat coronavirus HKU5 isolates (GenBank Accession No: KC522089.1, GenBank Accession No: KC522088.1, GenBank Accession No: KC522087.1, GenBank Accession No: C522086.1, GenBank Accession No: KC522085.1 , GenBank Accession No: C522084.1, GenBank Accession No:KC522083.1 , GenBank Accession No: KC522082.1 , GenBank Accession No: KC522081 , 1 , GenBank Accession No: KC522080.1 , GenBank Accession No: KC522079.1, GenBank Accession No: KC522078.1 , GenBank Accession No: C522077.1, GenBank Accession No: KC522076.1, GenBank Accession No: KC522075.1 , GenBank Accession No: KC522104.1, GenBank Accession No: C522104.1 , GenBank Accession No: KC522103.1 , GenBank Accession No: KC522102.1 , GenBank Accession No: C522101.1 , GenBank Accession No: KC522100.1, GenBank Accession No: KC522099.1 , GenBank Accession No: C522098.1, GenBank Accession No: KC522097.1 , GenBank Accession No: KC522096.1 , GenBank Accession No: KC522095.1, GenBank Accession No: KC522094.1 , GenBank Accession No: KC522093.1 , GenBank Accession No: KC522092.1 , GenBank Accession No: KC522091.1, GenBank Accession No: KC522090.1 , GenBank Accession No: KC5221 19.1 GenBank Accession No: C5221 18.1 GenBank Accession No: C5221 17.1 GenBank Accession No: C5221 16.1 GenBank Accession No: C5221 15.1 GenBank Accession No: C5221 14.1 GenBank Accession No; KC5221 13,1 GenBank Accession No: C5221 12.1 GenBank Accession No: KC 522 1 1.1 GenBank Accession No: KC5221 10.1 GenBank Accession No: KC522109.1 GenBank Accession No: KC522108.1 , GenBank Accession No: KC522107.1, GenBank Accession No: KC522106.1, GenBank Accession No: KC522105.1) Pipistrellus bat coronavirus HKU4 isolates(GenBank Accession No: KC522048.1 , GenBank Accession No: KC522047.1, GenBank Accession No:KC522046, 1, GenBank Accession No:KC522045.1 , GenBank Accession No: KC522044.1 , GenBank Accession No: KC522043. I , GenBank Accession No: KC522042.1 , GenBank Accession No: KC522041.1, GenBank Accession No:KC522040.1 GenBank Accession No:KC522039.1 , GenBank Accession No: C522038.1, GenBank Accession No: C522037.1 , GenBank Accession No:KC522036.1, GenBank Accession No: C522048.1 GenBank Accession No:KC522047.1 GenBank Accession No :KC522046.1 GenBank Accession No:KC522045.1 GenBank Accession No: C522044,1 GenBank Accession No:KC522043.1 GenBank Accession No:KC522042.1 GenBank Accession No:KC522041 .1 GenBank Accession No:KC522040, 1 , GenBank Accession No:KC522039.1 GenBank Accession No:KC522038.1 GenBank Accession No:KC522037.1 GenBank Accession No:KC522036.1, GenBank Accession No:KC52206U GenBank Accession No:KC522060.1 GenBank Accession No:KC522059.1 GenBank Accession No:KC522058.1 GenBank Accession No:KC522057.1 GenBank Accession No: C522056.1 GenBank Accession No:KC522055, 1 GenBank Accession No: C522054.1 GenBank Accession No:KC522053, 1 GenBank Accession No:KC522052,1 GenBank Accession No:KC522051.1 GenBank Accession No:KC522050.1 GenBank Accession No:KC522049.1 GenBank Accession No:KC522074.1 , GenBank Accession No:KC522073.1 GenBank Accession No:KC522072.1 GenBank Accession No:KC522071.1 GenBank Accession No: C522070.1 GenBank Accession No:KC522069.1 GenBank Accession No:KC522068.1 GenBank Accession No:KC522067.1 , GenBank Accession No :KC522066.1 GenBank Accession No:KC522065.1 GenBank Accession No:KC522064,1, GenBank Accession No: C522063.1 , GenBank Accession No:KC522062.1), as well as any other subgroup 2c coronavirus now known (e.g., as can be found in the GenBank® Database) or later identified, and any combination thereof.

In some embodiments, the virus is a subgroup 2d coronavirus. Nonlimiting examples of a subgroup 2d coronavirus of this invention include BtCoV.HKU9.2 (GenBank Accession No. EF065514), BtCoV.HKU9.1 (GenBank Accession No. NCJ)09021), BtCoV.HkU9.3 (GenBank Accession No. EF065515), BtCoV.HKU9.4 (GenBank Accession No. EF065516), as well as any other subgroup 2d coronavirus now known (e.g., as can be found in the GenBank® Database) or later identified, and any combination thereof.

In some embodiments, the virus is a subgroup 3 coronavirus. Nonlimiting examples of a subgroup 3 coronavirus of this invention include IBV.BeaudetteIBV.p65 (GenBank Accession No. DQ001339), as well as any other subgroup 3 coronavirus now known (e.g., as can be found in the GenBank® Database) or later identified, and any combination thereof.

The coronaviruses in the respective subgroups 1a, 1b, 2a, 2b, 2c, 2d and 3 can be included in the methods and compositions of this invention in any combination, as would be well understood to one of ordinary skill in the art.

In some embodiments, the virus is a subgroup 2b coronavirus. In some embodiments, the virus is SARS CoV-2. The SARS-CoV-2 virus has been identified as the causative agent of the coronavirus disease first reported in China in 2019 (COVID-19) and was declared to be a global pandemic in March of 2020. This novel human coronavirus is a respiratory pathogen, known to be communicable via respiratory droplets and fomite transmission especially to the mucosa of the face and lungs. The latent period following exposure can be as along as 14-21 days. While many confirmed cases of SARS-CoV-2 infection result in asymptomatic presentation, death can result from severe disease. The related SARS-CoV-1 virus was identified as the causative agent for the first major SARS outbreak reported in East Asia in 2003. The Middle Eastern Respiratory Virus (MERS) was first reported as infected humans in 2012. It is thought that each of SARS CoV-1, SARS CoV-2, and MERS, are resultant from zoonotic transmission events via bats and possibly other livestock animals like swine and camelids. In some embodiments, the virus is SARS CoV-1. In some embodiments, the virus is MERS.

There are currently no FDA approved treatments, vaccines, or other prophylaxis for SARS-CoV-1, SARS-CoV-2 or MERS. There is an immense unmet need to provide methods of preventing and treating coronavirus infection.

The provided embodiments relate to methods for delivering a C19orf66 to a subject. The C19orf66 can be delivered as a protein or a nucleic acid agent. In provided methods, the administered C19orf66 is heterologous to the subject. In some embodiments, a C19orf66 protein (or polypeptide), such as a recombinant protein is administered to the subject. In some embodiments, a nucleic acid encoding C19orf66 is administered to the subject. In a particular embodiment, the agent, e.g. the protein or encoding polynucleotide, for delivering C19orf66 to a subject can be contained in any vehicle (e.g. viral and non-viral vectors) that can be engineered to contain C19orf66 or a nucleic acid encoding C19orf66. The vehicles (e.g. non-viral and viral vectors) can additionally be engineered to express a targeting moiety, such as a fusogen or polypeptide with specificity for a ligand expressed in the lung. For example, in some cases the vehicles are targeting for delivery to a cell expressing a virus entry receptor, such as ACE2, TMPRSS2, or DPP4 . In some embodiments, the agent for delivering C19orf66 is a naked nucleic acid, such as mRNA. In other embodiments, the agent for delivering C19orf66 is a protein, such as a recombinant protein. In any of such embodiments, the agents can further be complexed or fused to a cell penetrating peptide to increase targeting to a cell.

The provided embodiments further relate to methods for delivering a regulatory factor for controlling C19orf66 in a cell in a subject. The regulatory factor can be a single molecule or can be a complex of one or more molecules. In particular embodiments, the regulatory factor includes an activator protein, such as a transcriptional activator, for site-specific targeting increased C19orf66 gene expression. For example, the regulatory factor can include a site-specific binding domain and a transcriptional activator. In some cases, The regulatory factor can be delivered as a protein or as nucleic acid agent. In some cases, the regulatory factor can be delivered as a protein complex, including a complex of a protein and a nucleic acid, such as including a ribonucleoprotein complex (e.g. dCas-gRNA complex). In provided methods, the administered regulatory factor is heterologous to the subject. In some embodiments, the regulatory factor that is administered is a protein or a protein complex. In some embodiments, a nucleic acid encoding the regulatory factor is administered to the subject. In a particular embodiment, the agent, e.g. the protein or encoding polynucleotide, for delivering the regulatory factor to a subject can be contained in any vehicle (e.g. viral and non-viral vectors) that can be engineered to contain the regulatory factor protein or a complex of the protein with a site-directed targeting agent (e.g. gRNA), or a nucleic acid encoding the regulatory factor. The vehicles (e.g. non-viral and viral vectors) can additionally be engineered to express a targeting moiety, such as a fusogen or polypeptide with specificity for a ligand expressed in the lung. For example, in some cases the vehicles are targeting for delivery to a cell expressing a virus entry receptor, such as ACE2, TMPRSS2, or DPP4.

Also provided herein are methods and uses of the polynucleotides and vehicles provided herein, such in prophylactic and therapeutic methods. Also provided are polynucleotides, compositions containing the vehicles and polynucleotides, and kits for using and administering the particles.

All publications, including patent documents, scientific articles and databases, referred to in this application are incorporated by reference in their entirety for all purposes to the same extent as if each individual publication were individually incorporated by reference. If a definition set forth herein is contrary to or otherwise inconsistent with a definition set forth in the patents, applications, published applications and other publications that are herein incorporated by reference, the definition set forth herein prevails over the definition that is incorporated herein by reference.

The section headings used herein are for organizational purposes only and are not to be construed as limiting the subject matter described.

### I. Chromosome 19 Open Reading Frame (c19orf66) Proteins and Encoding Polynucleotides

Provided herein are polynucleotides (nucleic acid molecules) encoding C19orf66. Also provided here are polypeptides (proteins) encoding C19orf66. The polynucleotides or polypeptides can be administered to subjects for treating a viral infection, or reducing a viral infection, according to the provided embodiments and methods.

In some aspects, the C19orf66 inhibits viral replication. For example, in some instances, the encoded C19orf66 inhibits ribosomal frameshifting and /or inhibits viral RNA processing. In some embodiments, the encoded C19orf66 interacts with endogenous RNA binding proteins.

C19orf66, also known as Shiftless Inhibitor of Ribosomal Frameshifting (SHFL) or Shiftless, RyDen, IRAV, SVA-1, and UPF0515, is encoded in 10 exons on human chromosome 19 encoding a primary isoform of 291 amino acids in length (SEQ ID NO: 1). The encoded C19orf66 gene can further comprise an alternative in-frame splice site within the 3′ coding region. In some embodiments, SHFL is encoded in 10 exons on human chromosome 19 encoding a secondary isoform of 240 amino acids in length (SEQ ID NO:3).

In some embodiments, the encoded C19orf66 is of a primary isoform of 291 amino acids in length (UniProt Q9NUL5-1). In some embodiments, the encoded C19orf66 has the sequence of amino acids set forth in SEQ ID NO: 1, or a sequence of amino acids that has at least 90%, at least 92%, at least 95%, or at least 98% sequence identity to the sequence of amino acids set forth in SEQ ID NO:1. In some embodiments, the encoded C19orf66 has the sequence set forth in SEQ ID NO:1. In some embodiments, the encoded C19orf66 has the sequence of nucleic acids set forth in SEQ ID NO: 2, or a sequence of nucleic acids that has at least 90%, at least 92%, at least 95%, or at least 98% sequence identity to the sequence of nucleic acids set forth in SEQ ID NO:2. In some embodiments, the encoded C19orf66 has the sequence set forth in SEQ ID NO:2.

In some embodiments, the administered C19orf66, or the C19orf66 encoded by an administered polynucleotide, lacks the N-terminal methionine (Met). The removal of N-terminal Met residue is catalyzed endogenously in eukaryotes by methionine aminopeptidase (MetAP). In some instances, removal of the initiator methionine may improve the function and stability of proteins. Thus, in some embodiments, the encoded initial initiator methionine is removed or is not present in the C19orf66 polypeptide. In some embodiments, the C19orf66 has the sequence of amino acids set forth in SEQ ID NO: 5, or a sequence of amino acids that has at least 90%, at least 92%, at least 95%, or at least 98% sequence identity to the sequence of amino acids set forth in SEQ ID NO:5. In some embodiments, the C19orf66 has the sequence set forth in SEQ ID NO:5. In some embodiments, the C19orf66 has the sequence of nucleic acids set forth in SEQ ID NO: 6, or a sequence of nucleic acids that has at least 90%, at least 92%, at least 95%, or at least 98% sequence identity to the sequence of nucleic acids set forth in SEQ ID NO:6.

In some embodiments, the encoded C19orf66 is of a secondary isoform of 240 amino acids in length (UniProt Q9NUL5-4). In some embodiments, the encoded C19orf66 has the sequence of amino acids set forth in SEQ ID NO: 3, or a sequence of amino acids that has at least 90%, at least 92%, at least 95%, or at least 98% sequence identity to the sequence of amino acids set forth in SEQ ID NO:3. In some embodiments, the encoded C19orf66 has the sequence set forth in SEQ ID NO:3. In some embodiments, the encoded C19orf66 is of a second isoform. In some embodiments, the encoded C19orf66 has the sequence of nucleic acids set forth in SEQ ID NO: 4, or a sequence of nucleic acids that has at least 90%, at least 92%, at least 95%, or at least 98% sequence identity to the sequence of nucleic acids set forth in SEQ ID NO:4. In some embodiments, the encoded C19orf66 has the sequence set forth in SEQ ID NO:4.

In some embodiments, the administered C19orf66, or the C19orf66 encoded by an administered polynucleotide, lacks the N-terminal methionine (Met). The removal of N-terminal Met residue is catalyzed endogenously in eukaryotes by methionine aminopeptidase (MetAP). In some instances, removal of the initiator methionine may improve the function and stability of proteins. Thus, in some embodiments, the encoded initial initiator methionine is removed from or is not present in the C19orf66 polypeptide. In some embodiments, the encoded C19orf66 has the sequence set forth in SEQ ID NO:6. In some embodiments, the encoded C19orf66 has the sequence of amino acids set forth in SEQ ID NO: 7, or a sequence of amino acids that has at least 90%, at least 92%, at least 95%, or at least 98% sequence identity to the sequence of amino acids set forth in SEQ ID NO:7. In some embodiments, the encoded C19orf66 has the sequence set forth in SEQ ID NO:7. In some embodiments, the encoded C19orf66 has the sequence of nucleic acids set forth in SEQ ID NO: 8, or a sequence of nucleic acids that has at least 90%, at least 92%, at least 95%, or at least 98% sequence identity to the sequence of nucleic acids set forth in SEQ ID NO:8. In some embodiments, the encoded C19orf66 has the sequence set forth in SEQ ID NO:8.

In some aspects, the encoded C19orf66 comprises a nuclear localization signal. The nuclear localization signal of C19orf66 has been implicated in viral inhibition. In some embodiments, the nuclear localization signal encodes a nuclear localization signal with an amino acid sequence selected from the group consisting of SEQ ID NOS: 81, 82, 83, 84, and 86. Thus, in some embodiments, the encoded nuclear localization signal has the sequence set forth in SEQ ID NO: 86.

In some aspects, the encoded C19orf66 comprises a nuclear export signal. The nuclear export signal of C19orf66 has been implicated in viral inhibition. In some embodiments, the nuclear export signal is selected from LXXXLXXLXL (SEQ ID NO:9) or LEDLDNLIL (SEQ ID: NO 85). Thus, in some embodiments, the encoded nuclear export signal has the sequence set forth in SEQ NO: 85.

### A. Delivery of Polynucleotides

In provided embodiments, the methods for treating a viral infection (e.g. a coronavirus infection), or for reducing the likelihood of a viral infection (e.g. coronavirus), include delivering an agent containing a polynucleotide encoding C19orf66. The polynucleotides can be administered as a naked nucleic acid (e.g. mRNA) or can be delivered in a carrier or vehicle for delivery to the subject. In provided embodiments, the polynucleotide encodes any C19orf66 polypeptide as described above.

In some embodiments, the polynucleotide has the sequence of nucleic acids set forth in SEQ ID NO: 2, or a sequence of nucleic acids that has at least 90%, at least 92%, at least 95%, or at least 98% sequence identity to the sequence of nucleic acids set forth in SEQ ID NO:2. In some embodiments, the encoded C19orf66 has the sequence set forth in SEQ ID NO:2. In some embodiments, the encoded C19orf66 is of a second isoform. In some embodiments, the encoded C19orf66 has the sequence of nucleic acids set forth in SEQ ID NO: 4, or a sequence of nucleic acids that has at least 90%, at least 92%, at least 95%, or at least 98% sequence identity to the sequence of nucleic acids set forth in SEQ ID NO:4. In some embodiments, the encoded C19orf66 has the sequence set forth in SEQ ID NO:4. In some embodiments, the encoded C19orf66 has the sequence of nucleic acids set forth in SEQ ID NO: 6, or a sequence of nucleic acids that has at least 90%, at least 92%, at least 95%, or at least 98% sequence identity to the sequence of nucleic acids set forth in SEQ ID NO:6. In some embodiments, the encoded C19orf66 has the sequence set forth in SEQ ID NO:6. In some embodiments, the encoded C19orf66 has the sequence of nucleic acids set forth in SEQ ID NO: 8, or a sequence of nucleic acids that has at least 90%, at least 92%, at least 95%, or at least 98% sequence identity to the sequence of nucleic acids set forth in SEQ ID NO:8. In some embodiments, the encoded C19orf66 has the sequence set forth in SEQ ID NO:8.

In some embodiments, a polynucleotide encoding C19orf66 is contained in a vehicle, such as viral-particles, viral-like particles, or non-viral particles, for delivering the polynucleotides to the subject. Exemplary vehicles for delivery are described in Section III. In some embodiments, the polynucleotide is delivered as a naked nucleic acid. In some embodiments, the polynucleotide is administered as an mRNA.

Polynucleotides encoding C19orf66 of the present invention may be delivered to a cell naked. As used herein in, “naked” refers to delivering C19orf66 free from agents which promote internalization. For example, the polynucleotide delivered to the cell may contain no modifications. The naked polynucleotides may be delivered to the cell using routes of administration known in the art and described herein.

In some aspects, mRNAs may be delivered as packaged particles (e.g., encapsulated in a delivery vehicle) or unpackaged (i.e., naked). In some aspects, mRNA may be transcribed within host cells. Exogenous mRNA delivery was first investigated in 1990, wherein Wolff and colleagues observed protein expression in mice following injection of mRNA encoding a reporter gene (Wolff et al., Science (247) 1465, 1990). Once exogenous mRNA has been transmitted to the cytosol, in some aspects, host cellular machinery can produce a mature polypeptide. In some embodiments, the polypeptide can be subject to post-translational modifications. In some aspects, proteins produced from exogenous mRNA delivery are degraded by normal physiological processes. In some embodiments, mRNA delivery reduces risk of metabolite toxicity (Pardi et al., Nat Rev Drug Discov (17) 4, 2018).

According to the provided embodiments, polynucleotides administered mRNA may have a capping region. The capping region may comprise a single cap or a series of nucleotides forming the cap. In this embodiment the capping region may be from 1 to 10, e.g. 2-9, 3-8, 4-7, 1-5, 5-10, or at least 2, or 10 or fewer nucleotides in length. In some embodiments, the cap is absent.

Wild type untranslated regions (UTRs) of a gene are transcribed but not translated. In mRNA, the 5′UTR starts at the transcription start site and continues to the start codon but does not include the start codon; whereas, the 3′UTR starts immediately following the stop codon and continues until the transcriptional termination signal. There is growing body of evidence about the regulatory roles played by the UTRs in terms of stability of the nucleic acid molecule and translation. The regulatory features of a UTR can be incorporated into the polynucleotides of the present invention to, among other things, enhance the stability of the molecule. In some aspects, the in vivo half-life of mRNA can be regulated via modifications to the 3′ poly-adenosine tail. The specific features can also be incorporated to ensure controlled down-regulation of the transcript in case they are misdirected to undesired organs sites.

In some embodiments, the polynucleotide encoding C19orf66 is operably linked to a promoter to control expression.

In some embodiments, promoter elements regulate the frequency of transcriptional initiation. A promoter may be one naturally associated with a gene or polynucleotide sequence, as may be obtained by isolating the 5′ non-coding sequences located upstream of the coding segment and/or exon. Such a promoter can be referred to as “endogenous.” Alternatively, certain advantages will be gained by positioning the coding polynucleotide segment under the control of a recombinant or heterologous promoter, which refers to a promoter that is not normally associated with a polynucleotide sequence in its natural environment. Such promoters may include promoters of other genes, and promoters or enhancers isolated from any other prokaryotic, viral, or eukaryotic cell, and promoters or enhancers not “naturally occurring,” i.e., containing different elements of different transcriptional regulatory regions, and/or mutations that alter expression. In addition to producing nucleic acid sequences of promoters and enhancers synthetically, sequences may be produced using recombinant cloning and/or nucleic acid amplification technology, including PCR, in connection with the compositions disclosed herein (U.S. Pat. Nos. 4,683,202 and 5,928,906).

In some embodiments, a suitable promoter is the immediate early cytomegalovirus (CMV) promoter sequence or a shortened Cytomegalovirus immediate early (CMVie) promoter (Ostedgaard et al., Proc. Natl. Acad. Sci. USA 2005, 102, 2952-2957).. In some embodiments, the promoter sequence is a strong constitutive promoter sequence capable of driving high levels of expression of any polynucleotide sequence operatively linked thereto. In some embodiments, a suitable promoter is Elongation Growth Factor- 1a (EF-1 a). In some embodiments, other constitutive promoter sequences may also be used, including, but not limited to the simian virus 40 (SV40) early promoter, silencing-prone spleen focus forming virus (SFFV) promoter, silencing-prone spleen focus forming virus (SFFV) promoter, mouse mammary tumor virus (MMTV), human immunodeficiency virus (HIV) long terminal repeat (LTR) promoter, MoMuLV promoter, an avian leukemia virus promoter, a CAG promoter (Halbert et al.,Hum. Gene Ther. 2007, 18, 344-354), an Epstein-Barr virus immediate early promoter, a Rous sarcoma virus promoter, a F5tg83 promoter (Yan et al., 2015, Hum. Gene Ther., 26:334-346), as well as human gene promoters such as, but not limited to, the actin promoter, the myosin promoter, the hemoglobin promoter, and the creatine kinase promoter, and hybrid promoters, such as a hybrid promoter having a human cytomegalovirus (CMV) enhancer and the elongation factor 1a promoter (hCEF).

In some embodiments, the nucleotide sequence is operably linked to a promoter to control expression in the lung. Some promoters confer specificity to the genes they regulate. For example, proteins expressed exclusively in one organ are often under the control of organ specific promoters. In a particular embodiment, it may be desirable to use a tissue-specific promoter to achieve cell type specific, lineage specific, or tissue-specific expression of the polynucleotides provided herein (e.g., polynucleotides encoding C19orf66).

According to certain embodiments, the cell type specific promoter is specific for cell types found in the lung (e.g., pulmonary epithelial cells), brain (e.g., neurons, glial cells), liver (e.g., hepatocytes), pancreas, skeletal muscle (e.g., myocytes), immune system (e.g., T cells, B cells, macrophages), heart (e.g., cardiac myocytes), retina, skin (e.g., keratinocytes), bone (e.g., osteoblasts or osteoclasts), etc. In some embodiments, the polynucleic acid sequence provided herein is operably linked to a promoter to control expression in the lung. In some embodiments, the promoter is a human surfactant A promoter , a human surfactant B promoter, a human surfactant C promoter, a human surfactant D promoter, human ROBO4 promoter. In some embodiments, the promoter is a human CDH1. In some embodiments, the promoter is the human surfactant B promoter set forth in SEQ ID NO: 10.

In some embodiments, the promoter is a constitutive promoter. In some aspects, a constitutive promoters may be a ubiquitous promoter that allows expression in a wide variety of cell and tissue types. In some embodiments, the promoter is a human Ubiquitin C (UbC) promoter, a human elongation factor 1α (EF1α) promoter, an SV40 promoter, a Cytomegalovirus (CMV) promoter, or a PGK-1 promoter.

In some embodiments, the promoter is an inducible promoter. In some embodiments, the inducible promoter is a chemically inducible promoter that responsive to a chemical agent. Ins ome embodiments, the inducible promoter is responsive to tetracycline or an analog thereof (e.g. doxycycline). In some embodiments, the inducible promoter is a tetracycline minimal promoter. In some embodiments, the inducible promoter is part of an inducible expression system.

In some embodiments, expression of the polynucleotide is controlled by an inducible gene expression system. For instance, a delivery vehicle (e.g. viral vector) may be engineered to include a sequence encoding c19orf16 operably linked to a Tet Response Element (TRE) and a sequence containing a reverse tetracycline-controlled transactivator (rtTA), such that, in the presence of doxycycline or other tetracycline analog, rtTA binds the TRE and c19orf16 is expressed. The tetracycline (Tet)-On system is an inducible gene expression system for mammalian cells, in which the reverse Tet transactivator (rtTA) fusion protein, which is composed of the doxycycline-binding Tet- repressor mutant protein and the C terminal activator domain from the herpes simplex virus VP 16 protein, is engineered to control gene expression with doxycycline (Dox). In the presence of Dox, rtTA activates the minimal promoters that are fused downstream of an array of seven repeated Tet-operator sequences (Loew et al. BMC Biotechnol 10, 81 (2010). A one-vector system has recently been developed (Heinz et al.(2011) Human gene therapy 22, 166-176), which has enabled easy transduction of a gene of interest into primary immune cells (Sakemura et al. (2016) Cancer Immunol Res 4, 658-668). Using this inducible gene expression system, c19orf66 is expressed only when the inducer (Dox) is present.

As shown herein, using this system it was demonstrated that the viral vectors engineered with the inducible expression system could be delivered to infected cells, and doxycycline is able to induce c 19orf16 expression and reduce the percentage of cells infected with SARs-CoV-2. These results support that a diversity of control systems, including drug-controllable systems may also be alternatively employed. Exemplary drug-controllable systems include, but are not limited to, the cumate operator (CuO) and its repressor (CymR), which is responsive to cumate. Other regulation systems include those based on the use of ligands like RU486 (Wang, K.E., et al. (1994) PNAS, 91:8180-8184), ecdysone (No, D. et al. (1996) PNAS 93:3346-3351), and rapamycin (Spencer, D.M. et al. (1993) Science 262:1019-1024). In some embodiments, is a promoter induced by an inductor. An example is given by the TRE that includes a tetracycline promoter, and promoters regulated by the presence of other inductors like ecdysone, rapamycin, RU486, dexamethasone or heavy metal like Zn or Cd, are suitable as well. Such a promoter can be operatively linked to regulatory elements like tetracycline-responsive transactivators (rtTA), thereby enabling the tight regulation of the transcription unit.

In some embodiments, expression of the C19orf66 is regulated by a Tet- on system, which utilizes a transactivator rtTA (reverse tetracycline-controlled transactivator). In some embodiments, the transcriptional activator is rtTA. In some embodiments, the rtTA comprises a Tet-R repressor fused with VP16.

In some embodiments of an inducible system, the transactivator protein is capable of binding and activating an inducible promoter only when the transactivator protein is bound by an induction agent. In some embodiments, the transactivator protein is selected from a modified TetR, a rTetR, a rtTA and a Tet-On 3G transactivator protein, the inducible promoter comprises a Tet operator sequence, and the induction agent is tetracycline or a derivative thereof. In such embodiments, the modified TetR, rTetR, rtTA, or Tet-On 3G transactivator protein is only capable of binding and activating the inducible promoter when the transactivator protein is bound by tetracycline or a derivative thereof. Derivatives of tetracycline are known in the art, and includes doxcycline (Dox).

In certain embodiments, the transactivator protein is a reverse tetracycline- controlled transactivator protein (rtTA). In certain embodiments, the transactivator protein is a Tet-On 3G transactivator protein. In other embodiments, the inducible promoter comprises a Tet operator sequence. In other embodiments, the inducible promoter comprises one or more repeats of the Tet operator sequence. In other embodiments, the inducible promoter is a TRE3GS promoter.

In some of any embodiments, the transactivator (e.g. rtTA) is expressed in the forward direction from an operably connected promoter, and the nucleic acid encoding c19orf66 is expressed from an operably connected tetracycline promoter containing Tet operator sequences (e.g. TRE) in the reverse orientation. In some of any embodiments, the transactivator may be under the control of a constitutive promoter (e.g. CMV). In some of any embodiments, the transactivator may be under the control ofa cell- or tissue-specific promoter (e.g. lung-specific), such as any described herein.

In some embodiments, continuous exposure of the inducible cd19orf66 expression system to tetracycline or a derivative thereof, results in continuous expression of c19orf66. In some embodiments, expression of c19orf66 can be reduced or halted upon withdrawal of the induction agent, e.g., doxycycline. In some embodiments, expression of c19orf66 can be fine-tuned depending on the amount of the induction agent, e.g., doxycycline, that is exposed to the inducible system. For example, a higher dose of doxycycline can induce a higher level of expression of c19orf66. As such, an inducible c19orf66 expression system provided herein is a tunable expression system, and the level of expression of c19orf66 is dose-dependent with respect to the dose of induction agent the inducible expression system is exposed to. In some embodiments, after withdrawal of the induction agent (e.g., doxycycline), c19orf66 is no longer expressed. In some embodiments, re-introduction of the induction agent (e.g., doxycycline) re-induces expression of c19orf66.

In some aspects, a derivative of tetracycline is the preffered drug inducer for expression. Without wishing to be bound by theory, doxycycline has been observed to have high affinity for Tet-R, rtTA, and other regulatory factors associated with tetracycline binding. In some aspects, doxycycline is associated with lower toxicity, a known half-life of 24 hours, and a more favorable tissue distribution in comparison to tetracycline.

In some embodiments, there is provided a nucleic acid comprising a sequence encoding c 19orf16 operably linked to a drug respone element and a nucleic acid comprising a sequence containing a drug-controlled transactivator. For instance, the system may include any of the inducible expression systems described above. In some embodiments, the drug response element contains a response element with Tet operator sequences and the drug-controlled transactivtor is a transactivator controlled by tetracycline or a derivative thereof (e.g. doxycycline). In some embodiments, there is provided a nucleic acid comprising a sequence encoding c19orf66 operably linked to a response element that contains a Tet operator sequence, e.g. such as a TRE, and nucleic acid comprising a sequence containing a transactivator, such as rtTA. In some embodiments, each nucleic acid sequence may be provided by two separate vectors. In some embodiments, the nucleic acid sequence may be provided as a single polynucleotide in an all-in-one (AIO) vector system. For instance, provided herein is a viral vector or virus-like particle (e.g. lentiviral vector) containing a polynucleotide that expresses a sequence encoding c19orf16 operably linked to a response element that contains a Tet operator sequence, e.g. such as a TRE, and a nucleic acid comprising a sequence containing a transactivator, such as rtTA. In some embodiments, the transactivator is provided in the forward orientation, and the nucleic acid expression cassette encoding c19orf66 is provided in the reverse orientation. In some embodiments, the viral vector or virus-like particle (e.g. lentiviral vector) includes a targeting moiety, such as any described in Section III.E, to target the viral vector for delivery to a cell that is or likely to be infected by SARS-CoV2. In some embodiments, when doxycycline (Dox) is administered to a cell introduced with the vector, the gene expression system of c19orf66 is induced and the encoded protein is expressed.

In some embodiments, any of the provided polynucleotides encoding C19orf66 can be modified to remove CpG motifs and/or to optimize codons for translation in a particular species, such as human, canine, feline, equine, ovine, bovine, etc. species. In some embodiments, the polynucleotides are optimized for human codon usage (i.e., human codon-optimized). In some embodiments, the polynucleotides are modified to remove CpG motifs. In other embodiments, the provided polynucleotides are modified to remove CpG motifs and are codon-optimized, such as human codon-optimized. Methods of codon optimization and CpG motif detection and modification are well-known. Typically, polynucleotide optimization enhances transgene expression, increases transgene stability and preserves the amino acid sequence of the encoded polypeptide.

### B. Delivery of Polypeptides

In provided embodiments, the methods for treating a viral infection (e.g. a coronavirus infection), or for reducing the likelihood of a viral infection (e.g. coronavirus), include delivering C19orf66 protein to a subject. In some embodiments, the protein can be administered as a recombinant or purified protein. In some embodiments, the protein can be delivered in a carrier or vehicle for delivery to the subject. In provided embodiments, the C19orf66 protein can have the amino acid sequence of any of C19orf66 polypeptide as described above.

In some embodiments, the polypeptide has the sequence of nucleic acids set forth in SEQ ID NO: 1, or a sequence of nucleic acids that has at least 90%, at least 92%, at least 95%, or at least 98% sequence identity to the sequence of nucleic acids set forth in SEQ ID NO:1. In some embodiments, the encoded C19orf66 has the sequence set forth in SEQ ID NO:1. In some embodiments, the encoded C19orf66 is of a second isoform. In some embodiments, the encoded C19orf66 has the sequence of nucleic acids set forth in SEQ ID NO: 3, or a sequence of nucleic acids that has at least 90%, at least 92%, at least 95%, or at least 98% sequence identity to the sequence of nucleic acids set forth in SEQ ID NO:3. In some embodiments, the encoded C19orf66 has the sequence set forth in SEQ ID NO:3. In some embodiments, the encoded C19orf66 has the sequence of nucleic acids set forth in SEQ ID NO: 5, or a sequence of nucleic acids that has at least 90%, at least 92%, at least 95%, or at least 98% sequence identity to the sequence of nucleic acids set forth in SEQ ID NO:5. In some embodiments, the encoded C19orf66 has the sequence set forth in SEQ ID NO:5. In some embodiments, the encoded C19orf66 has the sequence of nucleic acids set forth in SEQ ID NO: 7, or a sequence of nucleic acids that has at least 90%, at least 92%, at least 95%, or at least 98% sequence identity to the sequence of nucleic acids set forth in SEQ ID NO:7. In some embodiments, the encoded C19orf66 has the sequence set forth in SEQ ID NO:7.

In some embodiments, the protein is produced using recombinant DNA techniques. In some embodiments, a nucleic acid molecule is an expression vector that is suitable for expression in a selected host cell. In some embodiments, the protein can be produced by gene synthesis methods.

In some embodiments, a nucleic acid encoding C19orf16 may be contained in an expression vector. Vectors comprising nucleic acids that encode C19orf16 polypeptides described herein are provided. Such vectors include, but are not limited to, DNA vectors, phage vectors, viral vectors, retroviral vectors, etc. In some embodiments, a vector is selected that is optimized for expression of polypeptides in a desired cell type, such as CHO or CHO-derived cells, or in NSO cells. Exemplary such vectors are described, for example, in Running Deer et al., Biotechnol. Prog. 20:880-889 (2004).

In particular, a DNA vector that encodes C19orf66 can be can be used to facilitate the the expression and recombinant production of C19orf66. The DNA sequence can be inserted into an appropriate expression vector, i.e., a vector which contains the necessary elements for the transcription and translation of the inserted protein-coding sequence. A variety of host-vector systems may be utilized to express the protein-coding sequence. These include mammalian cell systems infected with virus (e.g., vaccinia virus, adenovirus, etc.); insect cell systems infected with virus (e.g., baculovirus); microorganisms such as yeast containing yeast vectors, or bacteria transformed with bacteriophage DNA, plasmid DNA or cosmid DNA. Depending on the host-vector system utilized, any one of a number of suitable transcription and translation elements may be used. The methods producing a C 19orf66 polypeptide may include culturing a cell under conditions that lead to expression of the polypeptide, wherein the cell comprises a nucleic acid molecule encoding C19orf66 described herein, and/or vectors that include these nucleic acid sequences. In some embodiments, a C19orf66 may be expressed in prokaryotic cells, such as bacterial cells; or in eukaryotic cells, such as fungal cells (such as yeast), plant cells, insect cells, and mammalian cells.

In provided embodiment, the C19orf66 is administered as an agent for delivery to a cell in a subject, such as a subject that is known or is likely to be infected, or that may be susceptible to infection with, a virus (e.g. a coronavirus). In some embodiments, a C19orf66 is contained in a vehicle, such as viral-particles, viral-like particles, or non-viral particles, for delivering the protein to the subject. Exemplary vehicles for delivery are described in Section III. In some embodiments, a C19orf66 may be administered as a fusion protein for targeted delivery to a cell in the subject. For example, a C19orf66 may be fused to a cell penetrating peptide to enhance delivery to a cell.

Provided herein is a fusion protein comprising (1) a Chromosome 19 Open Reading Frame 66 (C19orf66) protein; and (2) a cell penetrating peptide. Also provided herein is a fusion protein, wherein the C19orf66 protein is linked indirectly to the cell penetrating peptide via a peptide linker.

Cell-penetrating peptides are a class of short peptide sequences that can cross the cytoplasmic membrane efficiently. In some aspects, when coupled to a cargo payload (e.g., C19orf66 peptide or regulatory factors thereof), they facilitate cellular uptake. Cell penetrating peptides have a broad range of possible applications in drug delivery and molecular biology (Fonseca at al., Adv Drug Deliv Rev. (11), 2009). There are three main theories regarding the mechanism of cell penetrating peptides: direct penetration in the membrane, endocytosis-mediated entry, and translocation through the formation of a transitory structure.

The term “capable of being internalized into a cell”, as used herein, refers to the ability of the peptides to pass cellular membranes (including inter alia the outer “limiting” cell membrane (also commonly referred to as “plasma membrane”), endosomal membranes, and membranes of the endoplasmatic reticulum) and/or to direct the passage of a given agent or cargo through these cellular membranes. Such passage through cellular membranes is herein also referred to as “cell penetration”. Accordingly, peptides having said ability to pass through cellular membranes are herein referred to as “cell penetrating peptides”. In the context of the present invention, any possible mechanism of internalization is envisaged including both energy-dependent (i.e. active) transport mechanisms (e.g., endocytosis) and energy-independent (i.e. passive) transport mechanism (e.g., diffusion). As used herein, the term “internalization” is to be understood as involving the localization of at least a part of the peptides that passed through the plasma cellular membrane into the cytoplasm (in contrast to localization in different cellular compartments such as vesicles, endosomes or in the nucleus).

Cell-penetrating peptides include peptides such as melittin and the classic pore forming peptides magainin and alamethicin (Ludtke, S. J., et al., Biochemistry (1996) 35:13723-13728; He, K., et al., Biophys. J. (1996) 70:2659-2666). Pore forming peptides can also be derived from membrane active proteins, e.g., granulysin, prion proteins (Ramamoorthy, A., et al., Biochim Biophys Acta (2006) 1758:154-163; Andersson, A., et al., Eur. Biophys. J. (2007) DOI 10.1007/s00249-007-0131-9). Other cell perpetrating or pore forming peptides include naturally occurring membrane active peptides such as the defensins (Hughes, A. L., Cell Mol Life Sci (1999) 56:94-103), and synthetic membrane lytic peptides (Gokel, G. W., et al., Bioorganic & Medicinal Chemistry (2004) 12:1291 1304). Included as generally synthetic peptides are the D-amino acid analogs of the conventional L forms, especially peptides that have all of the L-amino acids replaced by the D-enantiomers. Peptidomimetics that display cell penetrating properties may be used as well. Thus “cell penetrating peptides” include both natural and synthetic peptides and peptidomimetics.

Small branched synthetic peptide conjugates were developed as vehicles for the delivery of diagnostic probes and cytotoxic agents into the cytoplasm and the nucleus (see for example, WO 1995/33766), which are particularly suitable as transfection agents. For example, WO 2003/103718 discloses multimeric peptide transporters with a poly-lysine core and cell-penetrating peptides as “transporter units”, which further contain effectors, such as antibodies. A table of exemplary cell penetrating peptides is set forth in Table 2.

Table 2

Exemplary Cell Penetrating Peptides

| | |
|---|---|
| Cell Penetrating Peptide | Sequence |
| TAT (SEQ ID NO: 13) | GRKKRRQRRRPPQ |
| Penetratin (SEQ ID NO: 14) | RQIKIWFQNRRMKWKK |
| Transporant (SEQ ID NO: 15) | GWTLNSAGYLLGKINLKALAALAKKIL |
| Pept 1 (SEQ ID NO: 16) | PLILLRLLRGQF |
| Pept 2 (SEQ ID NO: 17) | PLIYLRLLRGQF |
| Transportan (SEQ ID NO: 18) | GWTLNSAGYLLGKINLKALAALAKKIL |
| IgV (SEQ ID NO: 19 ) | MGLGLHLLVLAAALQGAKKKRKV |

The linkage between the cell penetrating peptide and the one or more polypeptides provided herein may be directly or indirectly, i.e. the cell penetrating peptide and the nucleic acid or amino acid molecules directly adjoin or they may be indirectly linked by an additional component of the complex, e.g. a spacer or a linker. In some embodiments, the cell penetrating peptide is linked to a polypeptide encoding C19orf66.

A direct linkage may be realized by an amide bridge, such as if the components to be linked have reactive amino or carboxy groups. More specifically, if the components to be linked are peptides, polypeptides or proteins, linkage may be via a peptide bond. Such a peptide bond may be formed using a chemical synthesis involving both components (an N-terminal end of one component and the C-terminal end of the other component) to be linked, or may be formed directly via a protein synthesis of the entire peptide sequence of both components, wherein both (protein or peptide) components are preferably synthesized in one step. Such protein synthesis methods include e.g., without being limited thereto, liquid phase peptide synthesis methods or solid peptide synthesis methods, e.g. solid peptide synthesis methods according to Merrifield, t-Boc solid-phase peptide synthesis, Fmoc solid-phase peptide synthesis, BOP (Benzotriazole-1-yl-oxy-tris-(dimethylamino)-phosphonium hexafluorophosphate) based solid-phase peptide synthesis, etc. Alternatively, ester or ether linkages are possible.

Moreover, in particular if the components to be linked are peptides, polypeptides or proteins, a linkage may occur via the side chains, e.g. by a disulfide bridge. The linkage via a side chain is based on a side chain amino, thiol or hydroxyl group, e.g. via an amide or ester or ether linkage. A linkage of a peptidic main chain with a peptidic side chain of another component may also be via an isopeptide bond. An isopeptide bond is an amide bond that is not present on the main chain of a protein. The bond forms between the carboxyl terminus of one peptide or protein and the amino group of a lysine residue on another (target) peptide or protein.

The fusion proteins provided herein may optionally comprise a spacer or linker, which are non-immunologic moieties, which can be cleavable, and which link the cell penetrating peptide and the polypeptides provided herein, and/or which can be placed at the N-and/or C-terminal part of the components of the complex and/or at the N- and/or C-terminal part of the fusion protein itself. A linker or spacer may preferably provide further functionalities in addition to linking of the components, and preferably being cleavable, more preferably naturally cleavable inside the target cell, e.g. by enzymatic cleavage. However, such further functionalities do in particular not include any immunological functionalities. Examples of further functionalities, in particular regarding linkers in fusion proteins, can be found in Chen X. et al., 2013: Fusion Protein Linkers: Property, Design and Functionality. Adv Drug Deliv Rev. 65(10): 1357-1369, wherein for example also in vivo cleavable linkers are disclosed. Moreover, Chen X. et al., 2013: Fusion Protein Linkers: Property, Design and Functionality. Adv Drug Deliv Rev. 65(10): 1357-1369 also discloses various linkers, e.g. flexible linkers and rigid linkers, and linker designing tools and databases, which can be useful in the complex according to the present invention or to design a linker to be used in the complex according to the present invention.

In some embodiments, the protein is linked indirectly to the cell penetrating peptide via a peptide linker. In some embodiments, the peptide linker is up to 65 amino acids in length. In some embodiments, the peptide linker comprises from or from about 2 to 65 amino acids, 2 to 60 amino acids, 2 to 56 amino acids, 2 to 52 amino acids, 2 to 48 amino acids, 2 to 44 amino acids, 2 to 40 amino acids, 2 to 36 amino acids, 2 to 32 amino acids, 2 to 28 amino acids, 2 to 24 amino acids, 2 to 20 amino acids, 2 to 18 amino acids, 2 to 14 amino acids, 2 to 12 amino acids, 2 to 10 amino acids, 2 to 8 amino acids, 2 to 6 amino acids, 6 to 65 amino acids, 6 to 60 amino acids, 6 to 56 amino acids, 6 to 52 amino acids, 6 to 48 amino acids, 6 to 44 amino acids, 6 to 40 amino acids, 6 to 36 amino acids, 6 to 32 amino acids, 6 to 28 amino acids, 6 to 24 amino acids, 6 to 20 amino acids, 6 to 18 amino acids, 6 to 14 amino acids, 6 to 12 amino acids, 6 to 10 amino acids, 6 to 8 amino acids, 8 to 65 amino acids, 8 to 60 amino acids, 8 to 56 amino acids, 8 to 52 amino acids, 8 to 48 amino acids, 8 to 44 amino acids, 8 to 40 amino acids, 8 to 36 amino acids, 8 to 32 amino acids, 8 to 28 amino acids, 8 to 24 amino acids, 8 to 20 amino acids, 8 to 18 amino acids, 8 to 14 amino acids, 8 to 12 amino acids, 8 to 10 amino acids, 10 to 65 amino acids, 10 to 60 amino acids, 10 to 56 amino acids, 10 to 52 amino acids, 10 to 48 amino acids, 10 to 44 amino acids, 10 to 40 amino acids, 10 to 36 amino acids, 10 to 32 amino acids, 10 to 28 amino acids, 10 to 24 amino acids, 10 to 20 amino acids, 10 to 18 amino acids, 10 to 14 amino acids, 10 to 12 amino acids, 12 to 65 amino acids, 12 to 60 amino acids, 12 to 56 amino acids, 12 to 52 amino acids, 12 to 48 amino acids, 12 to 44 amino acids, 12 to 40 amino acids, 12 to 36 amino acids, 12 to 32 amino acids, 12 to 28 amino acids, 12 to 24 amino acids, 12 to 20 amino acids, 12 to 18 amino acids, 12 to 14 amino acids, 14 to 65 amino acids, 14 to 60 amino acids, 14 to 56 amino acids, 14 to 52 amino acids, 14 to 48 amino acids, 14 to 44 amino acids, 14 to 40 amino acids, 14 to 36 amino acids, 14 to 32 amino acids, 14 to 28 amino acids, 14 to 24 amino acids, 14 to 20 amino acids, 14 to 18 amino acids, 18 to 65 amino acids, 18 to 60 amino acids, 18 to 56 amino acids, 18 to 52 amino acids, 18 to 48 amino acids, 18 to 44 amino acids, 18 to 40 amino acids, 18 to 36 amino acids, 18 to 32 amino acids, 18 to 28 amino acids, 18 to 24 amino acids, 18 to 20 amino acids, 20 to 65 amino acids, 20 to 60 amino acids, 20 to 56 amino acids, 20 to 52 amino acids, 20 to 48 amino acids, 20 to 44 amino acids, 20 to 40 amino acids, 20 to 36 amino acids, 20 to 32 amino acids, 20 to 28 amino acids, 20 to 26 amino acids, 20 to 24 amino acids, 24 to 65 amino acids, 24 to 60 amino acids, 24 to 56 amino acids, 24 to 52 amino acids, 24 to 48 amino acids, 24 to 44 amino acids, 24 to 40 amino acids, 24 to 36 amino acids, 24 to 32 amino acids, 24 to 30 amino acids, 24 to 28 amino acids, 28 to 65 amino acids, 28 to 60 amino acids, 28 to 56 amino acids, 28 to 52 amino acids, 28 to 48 amino acids, 28 to 44 amino acids, 28 to 40 amino acids, 28 to 36 amino acids, 28 to 34 amino acids, 28 to 32 amino acids, 32 to 65 amino acids, 32 to 60 amino acids, 32 to 56 amino acids, 32 to 52 amino acids, 32 to 48 amino acids, 32 to 44 amino acids, 32 to 40 amino acids, 32 to 38 amino acids, 32 to 36 amino acids, 36 to 65 amino acids, 36 to 60 amino acids, 36 to 56 amino acids, 36 to 52 amino acids, 36 to 48 amino acids, 36 to 44 amino acids, 36 to 40 amino acids, 40 to 65 amino acids, 40 to 60 amino acids, 40 to 56 amino acids, 40 to 52 amino acids, 40 to 48 amino acids, 40 to 44 amino acids, 44 to 65 amino acids, 44 to 60 amino acids, 44 to 56 amino acids, 44 to 52 amino acids, 44 to 48 amino acids, 48 to 65 amino acids, 48 to 60 amino acids, 48 to 56 amino acids, 48 to 52 amino acids, 50 to 65 amino acids, 50 to 60 amino acids, 50 to 56 amino acids, 50 to 52 amino acids, 54 to 65 amino acids, 54 to 60 amino acids, 54 to 56 amino acids, 58 to 65 amino acids, 58 to 60 amino acids, or 60 to 65 amino acids. In some embodiments, the peptide linker is a polypeptide that is 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, or 65 amino acids in length.

In particular embodiments, the linker is a flexible peptide linker. In some such embodiments, the linker is 1-20 amino acids, such as 1-20 amino acids predominantly composed of glycine. In some embodiments, the linker is 1-20 amino acids, such as 1-20 amino acids predominantly composed of glycine and serine. In some embodiments, the linker is a flexible peptide linker containing amino acids Glycine and Serine, referred to as GS-linkers. In some embodiments, the peptide linker includes the sequences GS, GGS, GGGGS (SEQ ID NO:20), GGGGGS (SEQ ID NO:21) or combinations thereof. In some embodiments, the polypeptide linker has the sequence (GGS)n, wherein n is 1 to 10. In some embodiments, the polypeptide linker has the sequence (GGGGS)n, (SEQ ID NO:22) wherein n is 1 to 10. In some embodiments, the polypeptide linker has the seqence (GGGGGS)n (SEQ ID NO:23), wherein n is 1 to 6.

### II. Regulatory Factors for Expression of C19orf66

Provided herein are polynucleotides (nucleic acid molecules) encoding regulatory factors of C19orf66. Also provided here are polypeptides (proteins) encoding regulatory factors of C19orf66. The polynucleotides or polypeptides can be administered to subjects for treating a viral infection, or reducing a viral infection, according to the provided embodiments and methods.

Provided in some embodiments are polynucleotides encoding regulatory factors, such as those containing a site specific binding domain and transcriptional activator. Provided in some embodiments are polypeptides encoding regulatory factors, such as those containing a site specific binding domain and transcriptional activator. In some embodiments, the regulatory factor comprises a modified nuclease. In some aspects, the regulatory factor increases C19orf66 gene expression.

Provided herein are methods for delivering a regulatory factor for expression of C19orf66 for treating a viral infection (e.g. a coronavirus infection), or for reducing the likelihood of a viral infection (e.g. coronavirus). In some embodiments, the regulatory factor for expression of C 19orf66 is delivered as a polypeptide. In some embodiments, the regulatory factors for expression of C19orf66 is delivered as a polynucleotide. Also provided are vehicles, such as viral-particles, viral-like particles, or non-viral particles, for delivering the polypeptide or the polynucleotides to a subject that is known or suspected to be infected, or at risk of being infected, with a virus (e.g. coronavirus). In some embodiments, the subject is at risk of being exposed or known to have been exposed to a virus (e.g. coronavirus).

### A. Fusion Protein or Complex for Transcriptional Activation

In provided embodiments, the regulatory factor is a fusion protein or a protein complex containing (1) a site-specific binding domain specific for the C19orf66 gene and (2) a transcriptional activator.

### 1. Site-specific Binding Domain

In some embodiments, the regulatory factor is comprised of a site specific DNA-binding nucleic acid molecule, such as a guide RNA (gRNA). In some embodiments, the method is achieved by site specific DNA-binding targeted proteins, such as zinc finger proteins (ZFP) or fusion proteins containing ZFP.

In some aspects, the regulatory factor comprises a site-specific binding domain, such as using a DNA binding protein or DNA-binding nucleic acid, which specifically binds to or hybridizes to the gene at a targeted region, such as at C19orf66. In some aspects, the provided polynucleotides or polypeptides are coupled to or complexed with a site-specific nuclease, such as a modified nuclease. For example, in some embodiments, the administration is effected using a fusion comprising a DNA-targeting protein of a modified nuclease, such as a meganuclease, or an RNA-guided nuclease such as a clustered regularly interspersed short palindromic nucleic acid (CRISPR)-Cas system, such as CRISPR-Cas9 system, specific for C19orf66. In some embodiments, the nuclease is modified to lack nuclease activity. In some embodiments, the modified nuclease is a catalytically dead dCas9.

In some embodiments, the site specific binding domain may be derived from a nuclease. For example, the recognition sequences of homing endonucleases and meganucleases such as I-SceI, I-CeuI, PI-PspI, PI-Sce, I-SceIV, I-CsmI, I-PanI, I-SceII, I-PpoI, I-SceIII, I-CreI, I-TevI, I-TevII and I-TevIII. See also U.S. Pat, No. 5,420,032; U.S. Pat. No. 6,833,252; Belfort et al. , (1997) Nucleic Acids Res. 25:3379-3388; Dujon et al., (1989) Gene 82:115-118; Perler et al, (1994) Nucleic Acids Res. 22, 1125-1127; Jasin (1996) Trends Genet. 12:224-228; Gimble et al., (1996) J. Mol. Biol. 263:163-180; Argast et al, (1998) J. Mol. Biol. 280:345-353 and the New England Biolabs catalogue. In addition, the DNA-binding specificity of homing endonucleases and meganucleases can be engineered to bind non-natural target sites. See, for example, Chevalier et al, (2002) Molec. Cell 10:895-905; Epinat et al, (2003) Nucleic Acids Res. 31 :2952-2962; Ashworth et al, (2006 ) Nature 441 :656-659; Paques et al, (2007) Current Gene Therapy 7:49-66; U.S. Pat. Publication No. 2007/0117128.

Zinc finger, TALE, and CRISPR system binding domains can be “engineered” to bind to a predetermined nucleotide sequence, for example via engineering (altering one or more amino acids) of the recognition helix region of a naturally occurring zinc finger or TALE protein. Engineered DNA binding proteins (zinc fingers or TALEs) are proteins that are non-naturally occurring. Rational criteria for design include application of substitution rules and computerized algorithms for processing information in a database storing information of existing ZFP and/or TALE designs and binding data. See, for example, U.S. Pat. Nos. 6,140,081; 6,453,242; and 6,534,261; see also WO 98/53058; WO 98/53059; WO 98/53060; WO 02/016536 and WO 03/016496 and U.S. Publication No. 20110301073.

In some embodiments, the site-specific binding domain comprises one or more zinc-finger proteins (ZFPs) or domains thereof that bind to DNA in a sequence-specific manner. A ZFP or domain thereof is a protein or domain within a larger protein that binds DNA in a sequence-specific manner through one or more zinc fingers, regions of amino acid sequence within the binding domain whose structure is stabilized through coordination of a zinc ion.

Among the ZFPs are artificial ZFP domains targeting specific DNA sequences, typically 9-18 nucleotides long, generated by assembly of individual fingers. ZFPs include those in which a single finger domain is approximately 30 amino acids in length and contains an alpha helix containing two invariant histidine residues coordinated through zinc with two cysteines of a single beta turn, and having two, three, four, five, or six fingers. Generally, sequence-specificity of a ZFP may be altered by making amino acid substitutions at the four helix positions (-1, 2, 3 and 6) on a zinc finger recognition helix. Thus, in some embodiments, the ZFP or ZFP-containing molecule is non-naturally occurring, e.g., is engineered to bind to a target site of choice. See, for example, Beerli et al. (2002) Nature Biotechnol. 20:135-141; Pabo et al. (2001) Ann. Rev. Biochem. 70:313-340; Isalan et al. (2001) Nature Biotechnol. 19:656-660; Segal et al. (2001) Curr. Opin. Biotechnol. 12:632-637; Choo et al. (2000) Curr. Opin. Struct. Biol. 10:411-416; U.S. Pat. Nos. 6,453,242; 6,534,261; 6,599,692; 6,503,717; 6,689,558; 7,030,215; 6,794,136; 7,067,317; 7,262,054; 7,070,934; 7,361,635; 7,253,273; and U.S. Pat. Publication Nos. 2005/0064474; 2007/0218528; 2005/0267061, all incorporated herein by reference in their entireties.

Many gene-specific engineered zinc fingers are available commercially. For example, Sangamo Biosciences (Richmond, CA, USA) has developed a platform (CompoZr) for zinc-finger construction in partnership with Sigma-Aldrich (St. Louis, MO, USA), allowing investigators to bypass zinc-finger construction and validation altogether, and provides specifically targeted zinc fingers for thousands of proteins (Gaj et al., Trends in Biotechnology, 2013, 31(7), 397-405). In some embodiments, commercially available zinc fingers are used or are custom designed.

In some embodiments, the site-specific binding domain comprises a naturally occurring or engineered (non-naturally occurring) transcription activator-like protein (TAL) DNA binding domain, such as in a transcription activator-like protein effector (TALE) protein, See, e.g., U.S. Pat. Publication No. 20110301073, incorporated by reference in its entirety herein.

A TALE DNA binding domain or TALE is a polypeptide comprising one or more TALE repeat domains/units. The repeat domains are involved in binding of the TALE to its cognate target DNA sequence. A single “repeat unit” (also referred to as a “repeat”) is typically 33-35 amino acids in length and exhibits at least some sequence homology with other TALE repeat sequences within a naturally occurring TALE protein. Each TALE repeat unit includes 1 or 2 DNA-binding residues making up the Repeat Variable Diresidue (RVD), typically at positions 12 and/or 13 of the repeat. The natural (canonical) code for DNA recognition of these TALEs has been determined such that an HD sequence at positions 12 and 13 leads to a binding to cytosine (C), NG binds to T, NI to A, NN binds to G or A, and NG binds to T and non-canonical (atypical) RVDs are also known. See, U.S. Pat. Publication No. 20110301073. In some embodiments, TALEs may be targeted to any gene by design of TAL arrays with specificity to the target DNA sequence. The target sequence generally begins with a thymidine.

In some embodiments, TALE repeats are assembled to specifically target a gene. (Gaj et al., Trends in Biotechnology, 2013, 31(7), 397-405). A library of TALENs targeting 18,740 human protein-coding genes has been constructed (Kim et al., Nature Biotechnology. 31, 251-258 (2013)). Custom-designed TALE arrays are commercially available through Cellectis Bioresearch (Paris, France), Transposagen Biopharmaceuticals (Lexington, KY, USA), and Life Technologies (Grand Island, NY, USA).

In some embodiments, the site-specific binding domain is derived from the CRISPR/Cas system. In general, “CRISPR system” refers collectively to transcripts and other elements involved in the expression of or directing the activity of CRISPR-associated (“Cas”) genes, including sequences encoding a Cas gene, a tracr (trans-activating CRISPR) sequence (e.g. tracrRNA or an active partial tracrRNA), a tracr-mate sequence (encompassing a “direct repeat” and a tracrRNA-processed partial direct repeat in the context of an endogenous CRISPR system), a guide sequence (also referred to as a “spacer” in the context of an endogenous CRISPR system, or a “targeting sequence”), and/or other sequences and transcripts from a CRISPR locus.

In general, a CRISPR system is characterized by elements that promote the formation of a CRISPR complex at the site of a target sequence. In some embodiments, the target sequence or target site is the C19orf66 gene. Typically, in the context of formation of a CRISPR complex, “target sequence” generally refers to a sequence, e.g., a gene or a genomic sequence, to which a guide sequence is designed to have complementarity, where hybridization between the target sequence and a guide sequence promotes the formation of a CRISPR complex. Full complementarity is not necessarily required, provided there is sufficient complementarity to cause hybridization and promote formation of a CRISPR complex. In some embodiments, a guide sequence is selected to reduce the degree of secondary structure within the guide sequence. Secondary structure may be determined by any suitable polynucleotide folding algorithm.

In general, a guide sequence includes a targeting domain comprising a polynucleotide sequence having sufficient complementarity with a target polynucleotide sequence to hybridize with the target sequence and direct sequence-specific binding of the CRISPR complex to the target sequence. In some embodiments, the degree of complementarity between a guide sequence and its corresponding target sequence, when optimally aligned using a suitable alignment algorithm, is about or more than about 50%, 60%, 75%, 80%, 85%, 90%, 95%, 97.5%, 99%, or more. In some examples, the targeting domain of the gRNA is complementary, e.g., at least 80, 85, 90, 95, 98 or 99% complementary, e.g., fully complementary, to the target sequence on the target nucleic acid, such as the target sequence in the C19orf66 gene.

Optimal alignment may be determined with the use of any suitable algorithm for aligning sequences, non-limiting example of which include the Smith-Waterman algorithm, the Needleman-Wunsch algorithm, algorithms based on the Burrows-Wheeler Transform (e.g. the Burrows Wheeler Aligner), ClustalW, Clustal X, BLAT, Novoalign (Novocraft Technologies, ELAND (Illumina, San Diego, Calif.), SOAP (available at soap.genomics.org.cn), and Maq (available at maq.sourceforge.net). In some embodiments, a guide sequence is about or more than about 5, 10, 11, 12, 13, 14, 15, 16, 17, 18, 19, 20, 21, 22, 23, 24, 25, 26, 27, 28, 29, 30, 35, 40, 45, 50, 75, or more nucleotides in length. In some embodiments, a guide sequence is less than about 75, 50, 45, 40, 35, 30, 25, 20, 15, 12, or fewer nucleotides in length. The ability of a guide sequence to direct sequence-specific binding of the CRISPR/Cas complex to a target sequence may be assessed by any suitable assay.

In some embodiments, the target site is upstream of a transcription initiation site of the gene. In some aspects, the target site is adjacent to a transcription initiation site of the gene (e.g., C19orf66). In some aspects, the target site is adjacent to an RNA polymerase pause site downstream of a transcription initiation site of the gene.

In some embodiments, the targeting domain is configured to target the promoter region of the C19orf66 gene to promote transcription initiation, binding of one or more transcription enhancers or activators, and/or RNA polymerase. One or more gRNA can be used to target the promoter region of the C19orf66 gene. In some embodiments, one or more regions of C19orf66 can be targeted. In certain aspects, the target sites that are within 600 base pairs on either side of the transcription start site (TSS) of C19orf66.

It is within the level of a skilled artisan to design or identify a gRNA sequence that is or comprises a sequence targeting C19orf66, including the exon sequence and sequences of regulatory regions, including promoters and activators. A genome-wide gRNA database for CRISPR genome editing is publicly available, which contains exemplary single guide RNA (sgRNA) target sequences in constitutive exons of genes in the human genome or mouse genome (see e.g., genescript.com/gRNA-database.html; see also, Sanjana et al. (2014) Nat. Methods, 11:783-4; http://www.e-crisp.org/E-CRISP/; http://crispr.mit.edu/). In some embodiments, the gRNA sequence is or comprises a sequence with minimal off-target binding to a non-target gene.

### 2. Transcriptional Activator

In some embodiments, the regulatory factor further comprises a functional domain, e.g., a transcriptional activator.

In some embodiments, the transcriptional activator is or contains one or more regulatory elements, such as one or more transcriptional control elements of a target gene, whereby a site-specific domain as provided above is recognized to drive expression of such gene. In some embodiments, the transcriptional activator drives expression of C19orf66. In some cases, the transcriptional activator, can be or contain all or a portion of an heterologous transactivation domain. For example, in some embodiments, the transcriptional activator is selected from Herpes simplex-derived transactivation domain, Dnmt3a methyltransferase domain, p65, VP16, and VP64.

In some embodiments, the regulatory factor is a zinc finger transcription factor (ZF-TF). In some embodiments, the regulatory factor is VP64-p65-Rta (VPR).

In certain embodiments, the regulatory factor of C19orf66 expression further comprises a transcriptional regulatory domain. Common domains include, e.g., transcription factor domains (activators, repressors, co-activators, co-repressors), silencers, oncogenes (e.g., myc, jun, fos, myb, max, mad, rel, ets, bcl, myb, mos family members etc.); DNA repair enzymes and their associated factors and modifiers; DNA rearrangement enzymes and their associated factors and modifiers; chromatin associated proteins and their modifiers (e.g. kinases, acetylases and deacetylases); and DNA modifying enzymes (e.g., methyltransferases such as members of the DNMT family (e.g., DNMT1, DNMT3A, DNMT3B, DNMT3L, etc., topoisomerases, helicases, ligases, kinases, phosphatases, polymerases, endonucleases) and their associated factors and modifiers. See, e.g,, U.S. Publication No. 2013/0253040, incorporated by reference in its entirety herein. Suitable domains for achieving activation include the HSV VP 16 activation domain (see, e.g., Hagmann et al, J. Virol. 71, 5952-5962 (1 97)) nuclear hormone receptors (see, e.g., Torchia et al., Curr. Opin. Cell. Biol. 10:373-383 (1998)); the p65 subunit of nuclear factor kappa B (Bitko & Bank, J. Virol. 72:5610-5618 (1998) and Doyle & Hunt, Neuroreport 8:2937-2942 (1997)); Liu et al., Cancer Gene Ther. 5:3-28 (1998)), or artificial chimeric functional domains such as VP64 (Beerli et al., (1998) Proc. Natl. Acad. Sci. USA 95:14623-33), and degron (Molinari et al., (1999) EMBO J. 18, 6439-6447). Additional exemplary activation domains include, Oct 1, Oct-2A, Spl, AP-2, and CTF1 (Seipel etal, EMBOJ. 11, 4961-4968 (1992) as well as p300, CBP, PCAF, SRC1 PvALF, AtHD2A and ERF-2. See, for example, Robyr et al, (2000) Mol. Endocrinol. 14:329-347; Collingwood et al, (1999) J. Mol. Endocrinol 23:255-275; Leo et al, (2000) Gene 245:1-11 ; Manteuffel-Cymborowska (1999) Acta Biochim. Pol. 46:77-89; McKenna et al, (1999) J. Steroid Biochem. Mol. Biol. 69:3-12; Malik et al, (2000) Trends Biochem. Sci. 25:277-283; and Lemon et al, (1999) Curr. Opin. Genet. Dev. 9:499-504. Additional exemplary activation domains include, but are not limited to, OsGAI, HALF-1, Cl, AP1, ARF-5, -6,-1, and -8, CPRF1, CPRF4, MYC-RP/GP, and TRAB1 , See, for example, Ogawa et al, (2000) Gene 245:21-29; Okanami et al, (1996) Genes Cells 1 :87-99; Goff et al, (1991) Genes Dev. 5:298-309; Cho et al, (1999) Plant Mol Biol 40:419-429; Ulmason et al, (1999) Proc. Natl. Acad. Sci. USA 96:5844-5849; Sprenger-Haussels et al, (2000) Plant J. 22:1-8; Gong et al, (1999) Plant Mol. Biol. 41:33-44; and Hobo etal. , (1999) Proc. Natl. Acad. Sci. USA 96:15,348-15,353.

Exemplary repression domains that can be used to make genetic repressors include, but are not limited to, KRAB A/B, KOX, TGF-beta-inducible early gene (TIEG), v-erbA, SID, MBD2, MBD3, members of the DNMT family (e.g., DNMT1, DNMT3A, DNMT3B, DNMT3L, etc.), Rb, and MeCP2. See, for example, Bird et al, (1999) Cell 99:451-454; Tyler et al, (1999) Cell 99:443-446; Knoepfler et al, (1999) Cell 99:447-450; and Robertson et al, (2000) Nature Genet. 25:338-342. Additional exemplary repression domains include, but are not limited to, ROM2 and AtHD2A. See, for example, Chem et al, (1996) Plant Cell 8:305-321; and Wu et al, (2000) Plant J. 22:19-27.

In some instances, the domain is involved in epigenetic regulation of a chromosome. In some embodiments, the domain is a histone acetyltransferase (HAT), e.g. type- A, nuclear localized such as MYST family members MOZ, Ybf2/Sas3, MOF, and Tip60, GNAT family members Gcn5 or pCAF, the p300 family members CBP, p300 or Rttl09 (Bemdsen and Denu (2008) Curr Opin Struct Biol 18(6):682-689). In other instances the domain is a histone deacetylase (HD AC) such as the class I (HDAC-1, 2, 3, and 8), class II (HDAC IIA (HDAC-4, 5, 7 and 9), HD AC IIB (HDAC 6 and 10)), class IV (HDAC-1 1), class III (also known as sirtuins (SIRTs); SIRT1-7) (see Mottamal et al., (2015) Molecules 20(3):3898-3941). Another domain that is used in some embodiments is a histone phosphorylase or kinase, where examples include MSK1, MSK2, ATR, ATM, DNA-PK, Bubl, VprBP, IKK-a, PKCpi, Dik/Zip, JAK2, PKC5, WSTF and CK2. In some embodiments, a methylation domain is used and may be chosen from groups such as Ezh2, PRMT1/6, PRMT5/7, PRMT 2/6, CARM1, set7/9, MLL, ALL-1, Suv 39h, G9a, SETDB1, Ezh2, Set2, Dotl, PRMT1/6, PRMT 5/7, PR-Set7 and Suv4-20h, Domains involved in sumoylation and biotinylation (Lys9, 13, 4, 18 and 12) may also be used in some embodiments (review see Kousarides (2007) Cell 128:693-705).

Fusion molecules are constructed by methods of cloning and biochemical conjugation that are well known to those of skill in the art. Fusion molecules comprise a DNA-binding domain and a functional domain (e.g., a transcriptional activation or repression domain). Fusion molecules also optionally comprise nuclear localization signals (such as, for example, that from the SV40 medium T-antigen) and epitope tags (such as, for example, FLAG and hemagglutinin). Fusion proteins (and nucleic acids encoding them) are designed such that the translational reading frame is preserved among the components of the fusion.

Fusions between a polypeptide component of a functional domain (or a functional fragment thereof) on the one hand, and a non-protein DNA-binding domain (e.g., antibiotic, intercalator, minor groove binder, nucleic acid) on the other, are constructed by methods of biochemical conjugation known to those of skill in the art. See, for example, the Pierce Chemical Company (Rockford, IL) Catalogue. Methods and compositions for making fusions between a minor groove binder and a polypeptide have been described. Mapp et al, (2000) Proc. Natl. Acad. Sci. USA 97:3930-3935. Likewise, CRISPR/Cas TFs and nucleases comprising a sgRNA nucleic acid component in association with a polypeptide component function domain are also known to those of skill in the art and detailed herein.

### B. Delivery of Polynucleotides

In provided embodiments, the methods for treating a viral infection (e.g. a coronavirus infection), or for reducing the likelihood of a viral infection (e.g. coronavirus), include delivering an agent containing a polynucleotide encoding a regulatory factor for C19orf66. The polynucleotides can be administered as a naked nucleic acid (e.g. plasmid DNA or mRNA) or can be delivered in a carrier or vehicle for delivery to the subject. In provided embodiments, the polynucleotide encodes any regulatory factor for C19orf66 as described above.

In some embodiments, a polynucleotide encoding a regulatory factor for C19orf66 is contained in a vehicle, such as viral-particles, viral-like particles, or non-viral particles, for delivering the polynucleotides to the subject. Exemplary vehicles for delivery are described in Section III.

In some embodiments, the polynucleotide is delivered as a naked nucleic acid. In some embodiments, the polynucleotide is administered as an mRNA. In some embodiments, the polynucleotide is administered as plasmid DNA.

Polynucleotides encoding a regulatory factor for C19orf66 of the present invention may be delivered to a cell naked. As used herein in, “naked” refers to delivering a regulatory factor for C19orf66 free from agents which promote internalization. For example, the polynucleotide delivered to the cell may contain no modifications. The naked polynucleotides may be delivered to the cell using routes of administration known in the art and described herein.

In some aspects, mRNAs may be delivered as packaged particles (e.g., encapsulated in a delivery vehicle) or unpackaged (i.e., naked). In some aspects, mRNA may be transcribed within host cells. Exogenous mRNA delivery was first investigated in 1990, wherein Wolff and colleagues observed protein expression in mice following injection of mRNA encoding a reporter gene (Wolff et al., Science (247) 1465, 1990). Once exogenous mRNA has been transmitted to the cytosol, in some aspects, host cellular machinery can produce a mature polypeptide. In some embodiments, the polypeptide can be subject to post-translational modifications. In some aspects, proteins produced from exogenous mRNA delivery are degraded by normal physiological processes. In some embodiments, mRNA delivery reduces risk of metabolite toxicity (Pardi et al., Nat Rev Drug Discov (17) 4, 2018).

According to the present invention, polynucleotides administered as mRNA may have a capping region. The capping region may comprise a single cap or a series of nucleotides forming the cap. In this embodiment the capping region may be from 1 to 10, e.g. 2-9, 3-8, 4-7, 1-5, 5-10, or at least 2, or 10 or fewer nucleotides in length. In some embodiments, the cap is absent.

Wild type untranslated regions (UTRs) of a gene are transcribed but not translated. In mRNA, the 5′UTR starts at the transcription start site and continues to the start codon but does not include the start codon; whereas, the 3′UTR starts immediately following the stop codon and continues until the transcriptional termination signal. There is growing body of evidence about the regulatory roles played by the UTRs in terms of stability of the nucleic acid molecule and translation. The regulatory features of a UTR can be incorporated into the polynucleotides of the present invention to, among other things, enhance the stability of the molecule. In some aspects, the in vivo half-life of mRNA can be regulated via modifications to the 3′ poly-adenosine tail. The specific features can also be incorporated to ensure controlled down-regulation of the transcript in case they are misdirected to undesired organs sites.

In some embodiments, the polynucleotide encoding a regulatory factor for C19orf66 is operably linked to a promoter to control expression.

In some embodiments, promoter elements regulate the frequency of transcriptional initiation. A promoter may be one naturally associated with a gene or polynucleotide sequence, as may be obtained by isolating the 5′ non-coding sequences located upstream of the coding segment and/or exon. Such a promoter can be referred to as “endogenous.” Alternatively, certain advantages will be gained by positioning the coding polynucleotide segment under the control of a recombinant or heterologous promoter, which refers to a promoter that is not normally associated with a polynucleotide sequence in its natural environment. Such promoters may include promoters of other genes, and promoters or enhancers isolated from any other prokaryotic, viral, or eukaryotic cell, and promoters or enhancers not “naturally occurring,” i.e., containing different elements of different transcriptional regulatory regions, and/or mutations that alter expression. In addition to producing nucleic acid sequences of promoters and enhancers synthetically, sequences may be produced using recombinant cloning and/or nucleic acid amplification technology, including PCR, in connection with the compositions disclosed herein (U.S. Pat. Nos. 4,683,202 and 5,928,906).

In some embodiments, a suitable promoter is the immediate early cytomegalovirus (CMV) promoter sequence. In some embodiments, the promoter sequence is a strong constitutive promoter sequence capable of driving high levels of expression of any polynucleotide sequence operatively linked thereto. In some embodiments, a suitable promoter is Elongation Growth Factor- 1a (EF-1 a). In some embodiments, other constitutive promoter sequences may also be used, including, but not limited to the simian virus 40 (SV40) early promoter, mouse mammary tumor virus (MMTV), human immunodeficiency virus (HIV) long terminal repeat (LTR) promoter, MoMuLV promoter, an avian leukemia virus promoter, an Epstein-Barr virus immediate early promoter, a Rous sarcoma virus promoter, as well as human gene promoters such as, but not limited to, the actin promoter, the myosin promoter, the hemoglobin promoter, and the creatine kinase promoter.

In some embodiments, the nucleotide sequence is operably linked to a promoter to control expression in the lung. Some promoters confer specificity to the genes they regulate. For example, proteins expressed exclusively in one organ are often under the control of organ specific promoters. In a particular embodiment, it may be desirable to use a tissue-specific promoter to achieve cell type specific, lineage specific, or tissue-specific expression of the polynucleotides provided herein (e.g., polynucleotides encoding C19orf66).

According to certain embodiments, the cell type specific promoter is specific for cell types found in the lung (e.g., pulmonary epithelial cells), brain (e.g., neurons, glial cells), liver (e.g., hepatocytes), pancreas, skeletal muscle (e.g., myocytes), immune system (e.g., T cells, B cells, macrophages), heart (e.g., cardiac myocytes), retina, skin (e.g., keratinocytes), bone (e.g., osteoblasts or osteoclasts), etc.

In some embodiments, the polynucleic acid sequence provided herein is operably linked to a promoter to control expression in the lung. In some embodiments, the promoter is a human surfactant A promoter , a human surfactant B promoter, a human surfactant C promoter, a human surfactant D promoter, human ROBO4 promoter. In some embodiments, the promoter is a human CDH1 gene. In some embodiments, the promoter is the human surfactant B promoter set forth in SEQ ID NO: 10.

In some embodiments, the promoter is a constitutive promoter. In some aspects, a constitutive promoters may be a ubiquitous promoter that allows expression in a wide variety of cell and tissue types. In some embodiments, the promoter is a human Ubiquitin C (UbC) promoter, a human elongation factor 1α (EF1α) promoter, an SV40 promoter, a Cytomegalovirus (CMV) promoter, or a PGK-1 promoter, or other promoter disclosed supra.

In some embodiments, the the promoter is an inducible promoter. In some embodiments, the inducible promoter is a chemically inducible promoter that responsive to a chemical agent. In some embodiments, the inducible promoter is responsive to tetracycline or an analog thereof (e.g. doxycycline). In some embodiments, the inducible promoter is a tetracycline minimal promoter. In some embodiments, the inducible promoter is part of an inducible expression system, such as any described herein above.

In some embodiments, any of the provided polynucleotides encoding a regulatory factor for C19orf66 can be modified to remove CpG motifs and/or to optimize codons for translation in a particular species, such as human, canine, feline, equine, ovine, bovine, etc. species. In some embodiments, the polynucleotides are optimized for human codon usage (i.e., human codon-optimized). In some embodiments, the polynucleotides are modified to remove CpG motifs. In other embodiments, the provided polynucleotides are modified to remove CpG motifs and are codon-optimized, such as human codon-optimized. Methods of codon optimization and CpG motif detection and modification are well-known. Typically, polynucleotide optimization enhances transgene expression, increases transgene stability and preserves the amino acid sequence of the encoded polypeptide.

In some embodiments, a nucleic acid encoding one protein may be co-expressed with a nucleic acid encoding another protein, such as the nucleic acid encoding a site-specific binding domain and the nucleic acid encoding a transcriptional activator. In some cases, a polynucleotide can contain a single promoter that drives the expression of one or more nucleic acid molecules. In some embodiments, such promoters can be multicistronic (bicistronic or tricistronic, see e.g., U.S. Pat. No. 6,060,273). For example, in some embodiments, transcription units can be engineered as a bicistronic unit containing an IRES (internal ribosome entry site), which allows coexpression of gene products (e.g. encoding a first and second recombinant receptor) by a message from a single promoter. Alternatively, in some cases, a single promoter may direct expression of an RNA that contains, in a single open reading frame (ORF), two or three genes (e.g. encoding the molecule involved in modulating a metabolic pathway and encoding the recombinant receptor) separated from one another by sequences encoding a self-cleavage peptide (e.g., 2A sequences) or a protease recognition site (e.g., furin). The ORF thus encodes a single polypeptide, which, either during (in the case of 2A) or after translation, is processed into the individual proteins. In some cases, the peptide, such as T2A, can cause the ribosome to skip (ribosome skipping) synthesis of a peptide bond at the C-terminus of a 2A element, leading to separation between the end of the 2A sequence and the next peptide downstream (see, for example, de Felipe. Genetic Vaccines and Ther. 2:13 (2004) and deFelipe et al. Traffic 5:616-626 (2004)). Many 2A elements are known in the art. Examples of 2A sequences that can be used in the methods and nucleic acids disclosed herein, without limitation, 2A sequences from the foot-and-mouth disease virus , equine rhinitis A virus , Thosea asigna virus, and porcine teschovirus-1 as described in U.S. Patent Publication No. 20070116690. Thus, in some embodiments, the first and second nucleotide sequences are separated by a bicistronic element. In some embodiments, the IRES or is a ribosomal skipping sequence, optionally wherein the ribosomal skipping sequence is selected from the group consisting of T2A, P2A, F2A and E2A.

### C. Delivery of Polypeptides

In provided embodiments, the methods for treating a viral infection (e.g. a coronavirus infection), or for reducing the likelihood of a viral infection (e.g. coronavirus), include delivering a regulatory factor for C19orf66 to a subject as a protein. In some embodiments, the protein can be administered as a recombinant or purified protein. In some embodiments, the protein can be delivered in a carrier or vehicle for delivery to the subject. In provided embodiments, the regulatory factor for C19orf66 expression can have the amino acid sequence of any of C19orf66 regulatory factors as described above, such as those described in Section II.A and II.B.

Provided herein is a fusion protein comprising (1) a regulatory factor which increases expression of Chromosome 19 Open Reading Frame 66 (C19orf66) gene; and (2) a cell penetrating peptide. Also provided herein is a fusion protein, wherein the C19orf66 regulatory factor is linked indirectly to the cell penetrating peptide via a peptide linker.

Cell-penetrating peptides are a relatively new class of short peptide sequences that cross the cytoplasmic membrane efficiently. In some aspects, when coupled to a cargo payload (e.g. regulatory factor of C19orf66 expression) they facilitate cellular uptake of the cargo. They have a broad range of possible applications in drug delivery and molecular biology (Fonseca at al., Adv Drug Deliv Rev. (11), 2009). There are three theories of the mechanism of cell penetration by cell penetrating peptides: direct penetration in the membrane, endocytosis-mediated entry, and translocation through the formation of a transitory structure.

The term “capable of being internalized into a cell”, as used herein, refers to the ability of the peptides to pass cellular membranes (including inter alia the outer “limiting” cell membrane (also commonly referred to as “plasma membrane”), endosomal membranes, and membranes of the endoplasmatic reticulum) and/or to direct the passage of a given agent or cargo through these cellular membranes. Such passage through cellular membranes is herein also referred to as “cell penetration”. Accordingly, peptides having said ability to pass through cellular membranes are herein referred to as “cell penetrating peptides”. In the context of the present invention, any possible mechanism of internalization is envisaged including both energy-dependent (i.e. active) transport mechanisms (e.g., endocytosis) and energy-independent (i.e. passive) transport mechanism (e.g., diffusion). As used herein, the term “internalization” is to be understood as involving the localization of at least a part of the peptides that passed through the plasma cellular membrane into the cytoplasm (in contrast to localization in different cellular compartments such as vesicles, endosomes or in the nucleus).

Cell-penetrating peptides include peptides such as melittin and the classic pore forming peptides magainin and alamethicin (Ludtke, S. J., et al., Biochemistry (1996) 35:13723-13728; He, K., et al., Biophys. J. (1996) 70:2659-2666). Pore forming peptides can also be derived from membrane active proteins, e.g., granulysin, prion proteins (Ramamoorthy, A., et al., Biochim Biophys Acta (2006) 1758:154-163; Andersson, A., et al., Eur. Biophys. J. (2007) DOI 10.1007/s00249-007-0131-9). Other cell perpetrating or pore forming peptides include naturally occurring membrane active peptides such as the defensins (Hughes, A. L., Cell Mol Life Sci (1999) 56:94-103), and synthetic membrane lytic peptides (Gokel, G. W., et al., Bioorganic & Medicinal Chemistry (2004) 12:1291 1304). Included as generally synthetic peptides are the D-amino acid analogs of the conventional L forms, especially peptides that have all of the L-amino acids replaced by the D-enantiomers. Peptidomimetics that display cell penetrating properties may be used as well. Thus “cell penetrating peptides” include both natural and synthetic peptides and peptidomimetics.

Small branched synthetic peptide conjugates were developed as vehicles for the delivery of diagnostic probes and cytotoxic agents into the cytoplasm and the nucleus (WO 95/33766 ), which are particularly suitable as transfection agents. For example, WO 03/103718 discloses multimeric peptide transporters with a poly-lysine core and cell-penetrating peptides (CPPs) as “transporter units”, which further contain effectors, such as antibodies. A table of exemplary cell penetrating peptides is set forth in Table 2, in Section I.B above.

The linkage between the cell penetrating peptide and the one or more polypeptides provided herein may be directly or indirectly, i.e. the cell penetrating peptide and the nucleic acid or amino acid molecules directly adjoin or they may be linked by an additional component of the complex, e.g. a spacer or a linker. In some embodiments, the cell penetrating peptide is linked to a polypeptide encoding a regulatory factor of C19orf66 gene expression.

A direct linkage may be realized by an amide bridge, such as if the components to be linked have reactive amino or carboxy groups. More specifically, if the components to be linked are peptides, polypeptides or proteins, linkage may be via a peptide bond. Such a peptide bond may be formed using a chemical synthesis involving both components (an N-terminal end of one component and the C-terminal end of the other component) to be linked, or may be formed directly via a protein synthesis of the entire peptide sequence of both components, wherein both (protein or peptide) components are preferably synthesized in one step. Such protein synthesis methods include e.g., without being limited thereto, liquid phase peptide synthesis methods or solid peptide synthesis methods, e.g. solid peptide synthesis methods according to Merrifield, t-Boc solid-phase peptide synthesis, Fmoc solid-phase peptide synthesis, BOP (Benzotriazole-1-yl-oxy-tris-(dimethylamino)-phosphonium hexafluorophosphate) based solid-phase peptide synthesis, etc. Alternatively, ester or ether linkages are possible.

Moreover, in particular if the components to be linked are peptides, polypeptides or proteins, a linkage may occur via the side chains, e.g. by a disulfide bridge. The linkage via a side chain is based on a side chain amino, thiol or hydroxyl group, e.g. via an amide or ester or ether linkage. A linkage of a peptidic main chain with a peptidic side chain of another component may also be via an isopeptide bond. An isopeptide bond is an amide bond that is not present on the main chain of a protein. The bond forms between the carboxyl terminus of one peptide or protein and the amino group of a lysine residue on another (target) peptide or protein.

The fusion proteins provided herein may optionally comprise a spacer or linker, which are non-immunologic moieties, which are preferably cleavable, and which link the cell penetrating peptide and the polypeptides provided herein, and/or which can be placed at the N-and/or C-terminal part of the components of the complex and/or at the N- and/or C-terminal part of the fusion protein itself. A linker or spacer may preferably provide further functionalities in addition to linking of the components, and preferably being cleavable, more preferably naturally cleavable inside the target cell, e.g. by enzymatic cleavage. However, such further functionalities do in particular not include any immunological functionalities. Examples of further functionalities, in particular regarding linkers in fusion proteins, can be found in Chen X. et al., 2013: Fusion Protein Linkers: Property, Design and Functionality. Adv Drug Deliv Rev. 65(10): 1357-1369, wherein for example also in vivo cleavable linkers are disclosed. Moreover, Chen X. et al., 2013: Fusion Protein Linkers: Property, Design and Functionality. Adv Drug Deliv Rev. 65(10): 1357-1369 also discloses various linkers, e.g. flexible linkers and rigid linkers, and linker designing tools and databases, which can be useful in the complex according to the present invention or to design a linker to be used in the complex according to the present invention.

In some embodiments, the protein is linked indirectly to the cell penetrating peptide via a peptide linker. In some embodiments, the peptide linker is up to 65 amino acids in length. In some embodiments, the peptide linker comprises from or from about 2 to 65 amino acids, 2 to 60 amino acids, 2 to 56 amino acids, 2 to 52 amino acids, 2 to 48 amino acids, 2 to 44 amino acids, 2 to 40 amino acids, 2 to 36 amino acids, 2 to 32 amino acids, 2 to 28 amino acids, 2 to 24 amino acids, 2 to 20 amino acids, 2 to 18 amino acids, 2 to 14 amino acids, 2 to 12 amino acids, 2 to 10 amino acids, 2 to 8 amino acids, 2 to 6 amino acids, 6 to 65 amino acids, 6 to 60 amino acids, 6 to 56 amino acids, 6 to 52 amino acids, 6 to 48 amino acids, 6 to 44 amino acids, 6 to 40 amino acids, 6 to 36 amino acids, 6 to 32 amino acids, 6 to 28 amino acids, 6 to 24 amino acids, 6 to 20 amino acids, 6 to 18 amino acids, 6 to 14 amino acids, 6 to 12 amino acids, 6 to 10 amino acids, 6 to 8 amino acids, 8 to 65 amino acids, 8 to 60 amino acids, 8 to 56 amino acids, 8 to 52 amino acids, 8 to 48 amino acids, 8 to 44 amino acids, 8 to 40 amino acids, 8 to 36 amino acids, 8 to 32 amino acids, 8 to 28 amino acids, 8 to 24 amino acids, 8 to 20 amino acids, 8 to 18 amino acids, 8 to 14 amino acids, 8 to 12 amino acids, 8 to 10 amino acids, 10 to 65 amino acids, 10 to 60 amino acids, 10 to 56 amino acids, 10 to 52 amino acids, 10 to 48 amino acids, 10 to 44 amino acids, 10 to 40 amino acids, 10 to 36 amino acids, 10 to 32 amino acids, 10 to 28 amino acids, 10 to 24 amino acids, 10 to 20 amino acids, 10 to 18 amino acids, 10 to 14 amino acids, 10 to 12 amino acids, 12 to 65 amino acids, 12 to 60 amino acids, 12 to 56 amino acids, 12 to 52 amino acids, 12 to 48 amino acids, 12 to 44 amino acids, 12 to 40 amino acids, 12 to 36 amino acids, 12 to 32 amino acids, 12 to 28 amino acids, 12 to 24 amino acids, 12 to 20 amino acids, 12 to 18 amino acids, 12 to 14 amino acids, 14 to 65 amino acids, 14 to 60 amino acids, 14 to 56 amino acids, 14 to 52 amino acids, 14 to 48 amino acids, 14 to 44 amino acids, 14 to 40 amino acids, 14 to 36 amino acids, 14 to 32 amino acids, 14 to 28 amino acids, 14 to 24 amino acids, 14 to 20 amino acids, 14 to 18 amino acids, 18 to 65 amino acids, 18 to 60 amino acids, 18 to 56 amino acids, 18 to 52 amino acids, 18 to 48 amino acids, 18 to 44 amino acids, 18 to 40 amino acids, 18 to 36 amino acids, 18 to 32 amino acids, 18 to 28 amino acids, 18 to 24 amino acids, 18 to 20 amino acids, 20 to 65 amino acids, 20 to 60 amino acids, 20 to 56 amino acids, 20 to 52 amino acids, 20 to 48 amino acids, 20 to 44 amino acids, 20 to 40 amino acids, 20 to 36 amino acids, 20 to 32 amino acids, 20 to 28 amino acids, 20 to 26 amino acids, 20 to 24 amino acids, 24 to 65 amino acids, 24 to 60 amino acids, 24 to 56 amino acids, 24 to 52 amino acids, 24 to 48 amino acids, 24 to 44 amino acids, 24 to 40 amino acids, 24 to 36 amino acids, 24 to 32 amino acids, 24 to 30 amino acids, 24 to 28 amino acids, 28 to 65 amino acids, 28 to 60 amino acids, 28 to 56 amino acids, 28 to 52 amino acids, 28 to 48 amino acids, 28 to 44 amino acids, 28 to 40 amino acids, 28 to 36 amino acids, 28 to 34 amino acids, 28 to 32 amino acids, 32 to 65 amino acids, 32 to 60 amino acids, 32 to 56 amino acids, 32 to 52 amino acids, 32 to 48 amino acids, 32 to 44 amino acids, 32 to 40 amino acids, 32 to 38 amino acids, 32 to 36 amino acids, 36 to 65 amino acids, 36 to 60 amino acids, 36 to 56 amino acids, 36 to 52 amino acids, 36 to 48 amino acids, 36 to 44 amino acids, 36 to 40 amino acids, 40 to 65 amino acids, 40 to 60 amino acids, 40 to 56 amino acids, 40 to 52 amino acids, 40 to 48 amino acids, 40 to 44 amino acids, 44 to 65 amino acids, 44 to 60 amino acids, 44 to 56 amino acids, 44 to 52 amino acids, 44 to 48 amino acids, 48 to 65 amino acids, 48 to 60 amino acids, 48 to 56 amino acids, 48 to 52 amino acids, 50 to 65 amino acids, 50 to 60 amino acids, 50 to 56 amino acids, 50 to 52 amino acids, 54 to 65 amino acids, 54 to 60 amino acids, 54 to 56 amino acids, 58 to 65 amino acids, 58 to 60 amino acids, or 60 to 65 amino acids. In some embodiments, the peptide linker is a polypeptide that is 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, or 65 amino acids in length.

In particular embodiments, the linker is a flexible peptide linker. In some such embodiments, the linker is 1-20 amino acids, such as 1-20 amino acids predominantly composed of glycine. In some embodiments, the linker is 1-20 amino acids, such as 1-20 amino acids predominantly composed of glycine and serine. In some embodiments, the linker is a flexible peptide linker containing amino acids Glycine and Serine, referred to as GS-linkers. In some embodiments, the peptide linker includes the sequences GS, GGS, GGGGS (SEQ ID NO:20), GGGGGS (SEQ ID NO:21) or combinations thereof. In some embodiments, the polypeptide linker has the sequence (GGS)n, wherein n is 1 to 10. In some embodiments, the polypeptide linker has the sequence (GGGGS)n, (SEQ ID NO:22) wherein n is 1 to 10. In some embodiments, the polypeptide linker has the seqence (GGGGGS)n (SEQ ID NO:23), wherein n is 1 to 6.

In some embodiments, a regulatory factor for C19orf66 expression is contained in a vehicle, such as viral-particles, viral-like particles, or non-viral particles, for delivering the polynucleotides to the subject. Exemplary vehicles for delivery are described in Section III.

### III. Vehicles for Delivery

Provided herein are polynucleotides and polypeptides encoding C19orf66 or a regulatory factor of C19orf66 gene expression. In some embodiments, C19orf66 or a regulatory factor thereof is introduced to a cell in the subject. In some embodiments, the nucleotides or polypeptides provided herein are comprised within a vehicle.

In some embodiments, the vehicle is a viral vector or is derived from a viral vector. In other embodiments, the vehicle is a non-viral vector, such as a cellular particle, liposome, nanoparticle, or other synthetic particle. In some embodiments, the vehicle is a non-lipid particle, such as a synthetic particle. In some embodiments, the non-lipid particle is a nanoparticle not comprised of lipids, a polymeric nanoparticle, a nanocapsule, a nanorod or nanosohere, a nanogel, a dendrimer, or other synthetic or inorganic particle.

Non-viral vectors and methods employing the use of polymers, surfactants, and/or excipients have been employed to introduce polynucleotides and polypeptides into cells including conjugation with a targeting moiety, conjugation with a cell penetrating peptide, derivatization with a lipid and incorporation into liposomes, lipid nanoparticles, and cationic liposomes. The majority of non-viral vectors consist of plasmid DNA complexed with lipids or polycations. Many different lipids with ability to deliver plasmid DNA to cells in vitro and in vivo have been reported (Gao, et al., Gene Therapy 2:710-722 (1995)).

In particular embodiments, the polynucleotide or polypeptide is encapsulated within the lumen of a lipid particle in which the lipid particle contains a lipid bilayer, a lumen surrounded by the lipid bilayer. In some embodiments, the lipid particle can be a viral particle, a virus-like particle, a nanoparticle, a vesicle, an exosome, a dendrisome, a lentivirus, a viral vector, an enucleated cell, a microvesicle, a membrane vesicle, an extracellular membrane vesicle, a plasma membrane vesicle, a giant plasma membrane vesicle, an apoptotic body, a mitoparticle, a micelle, a liposome, a pyrenocyte, a lysosome, another membrane enclosed vesicle, or a lentiviral vector, a viral based particle, a virus like particle (VLP) or a cell derived particle. In some embodiments, the vehicle may further contain a fusogen, in which the fusogen is embedded within the lipid bilayer. In some cases, the fusogen may be a retargeted fusogen in which the fusogen is modified to direct targeting of the lipid particle to a target cell, e.g. a lung cell or a cell expressing a coreceptor recognized by the virus (e.g. ACE2). In other cases, the lipid particle may contain a targeted envelope glycoprotein (e.g. G protein) that directs targeting of the lipid particle to a target cell, e.g. a lung cell or a cell expressing a coreceptor recognized by the virus (e.g. ACE2).

In some embodiments, the lipid bilayer includes membrane components of the host cell from which the lipid bilayer is derived, e.g., phospholipids, membrane proteins, etc. In some embodiments, the lipid bilayer includes a cytosol that includes components found in the cell from which the vehicle is derived, e.g., solutes, proteins, nucleic acids, etc., but not all of the components of a cell, e.g., lacking a nucleus. In some embodiments, the lipid bilayer is considered to be exosome-like. The lipid bilayer may vary in size, and in some instances have a diameter ranging from 30 and 300 nm, such as from 30 and 150 nm, and including from 40 to 100 nm.

In some embodiments, the lipid bilayer is a viral envelope. In some embodiments, the viral envelope is obtained from a host cell. In some embodiments, the viral envelope is obtained by the viral capsid from the source cell plasma membrane. In some embodiments, the lipid bilayer is obtained from a membrane other than the plasma membrane of a host cell. In some embodiments, the viral envelope lipid bilayer is embedded with viral proteins, including viral glycoproteins.

In other aspects, the lipid bilayer includes synthetic lipid complex. In some embodiments, the synthetic lipid complex is a liposome. In some embodiments, the lipid bilayer is a vesicular structure characterized by a phospholipid bilayer membrane and an inner aqueous medium. In some embodiments, the lipid bilayer has multiple lipid layers separated by aqueous medium. In some embodiments, the lipid bilayer forms spontaneously when phospholipids are suspended in an excess of aqueous solution. In some examples, the lipid components undergo self-rearrangement before the formation of closed structures and entrap water and dissolved solutes between the lipid bilayers.

In some embodiments, a targeted envelope protein, such as any described in Section III.D, including any that are exogenous or overexpressed relative to the source cell, is disposed in the lipid bilayer.

In some embodiments, the lipid particle comprises several different types of lipids. In some embodiments, the lipids are amphipathic lipids. In some embodiments, the amphipathic lipids are phospholipids. In some embodiments, the phospholipids comprise phosphatidylcholine, phosphatidylethanolamine, phosphatidylinositol, and phosphatidylserine. In some embodiments, the lipids comprise phospholipids such as phosphocholines and phosphoinositols. In some embodiments, the lipids comprise DMPC, DOPC, and DSPC.

### A. Viral Vectors

Provided herein are vehicles containing an agent, such as any of the polynucleotides or polypeptides described here (See Sections I and II) that are derived from virus, such as viral particles. In some embodiment the viral particles include those derived from retroviruses or lentiviruses. In some embodiments, the viral particle’s bilayer of amphipathic lipids is or comprises the viral envelope. In some embodiments, the viral particle’s bilayer of amphipathic lipids is or comprises lipids derived from an infected host cell. In some embodiments, the viral vector envelope may comprise a fusogen, e.g., a fusogen that is endogenous to the virus or a pseudotyped fusogen.

Biological methods for introducing an exogenous agent to a host cell include the use of DNA and RNA vectors. DNA and RNA vectors can also be used to house and deliver polynucleotides and polypeptides. Viral vectors, and especially retroviral vectors, have become the most widely used method for inserting genes into mammalian, e.g., human cells. Other viral vectors can be derived from lentivirus, poxviruses, herpes simplex virus I, adenoviruses and adeno-associated viruses, and the like. See, for example, U.S. Pat. Nos. 5,350,674 and 5,585,362. Methods for producing cells comprising vectors and/or exogenous acids are well-known in the art. See, for example, Sambrook et al., 2001, Molecular Cloning: A Laboratory Manual, Cold Spring Harbor Laboratory, New York.

In some embodiments, the polynucleotides and polypeptides are comprised within a viral vector. In some embodiments, the polynucleotides or polypeptides provided herein (e.g., C19orf66 and/or regulatory factors thereof) are administered host cells using recombinant virus particles, such as, e.g., vectors derived from adenoviruses and adeno-associated virus (AAV). In some embodiments, the AAV vector is of serotype 1, 2, 5, or 6.

The AAV is a single-stranded DNA parvovirus which is capable of host genome integration during the latent phase of infectivity. For example, AAV of serotype 2 is largely endemic to the human and primate populations and frequently integrates site-specifically into human chromosome 19 q13.3.

In some aspects, AAV is considered a dependent virus because it requires helper functions from either adenovirus or herpes-virus in order to replicate. In the absence of either of these helper viruses, AAV has been observed to integrate its genome into the host cell chromosome. However, these virions are not capable of propagating infection to new cells.

In some embodiment, suitable host cells for producing AAV derived vehicles include microorganisms, yeast cells, insect cells, and mammalian cells. In some embodiments, the term host cell includes the progeny of the original cell which has been transfected. Thus, as indicated above, a “host cell,” or “producer cell,” as used herein, generally refers to a cell which has been transfected with a vector vehicle as described herein. For example, cells from the stable human cell line, 293 (ATCC Accession No. CRL1573) are familiar to those in the art as a producer cell for AAV vectors. The 293 cell line is a human embryonic kidney cell line that has been transformed with adenovirus type-5 DNA fragments (Graham et al., J. Gen. Virol., 36:59 (1977)), and expresses the adenoviral E1a and E1b genes (Aiello et al., Virol., 94:460 (1979)). The 293 cell line is readily transfected, and thus provides a particularly useful system in which to produce AAV virions.

Producer cells as described above containing the AAV vehicles provided herein must be rendered capable of providing AAV helper functions. In some embodiments, producer cells allow AAV vectors to replicate and encapsulate polynucleotide sequences, such as those encoding C 19orf66 or a regulatory factor thereof as provided in Sections I and II. In some embodiments, producer cells yield AAV virions. AAV helper functions are generally AAV-derived coding sequences that may be expressed to provide AAV gene products that, in turn, function for productive AAV replication. In some embodiments, AAV helper functions are used to complement necessary AAV functions that are missing from the AAV vectors. In some embodiments, AAV helper functions include at least one of the major AAV ORFs. In some embodiments, the helper functions include at least the rep coding region, or a functional homolog thereof. In some embodiments, the helper function includes at least the cap coding region, or a functional homolog thereof.

In some embodiments, the AAV helper functions are introduced into the host cell by transfecting the host cell with a mixture of AAV helper constructs either prior to, or concurrently with, the transfection of the AAV vector. In some embodiments, the AAV helper constructs are used to provide transient expression of AAV rep and/or cap genes. In some embodiments, the AAV helper constructs lack AAV packaging sequences and can neither replicate nor package themselves.

In some embodiments, an AAV genome can be cross-packaged with a heterologous virus. Cross-genera packing of the rAAV2 genome into the human bocavirus type 1 (HBoV1) capsid (rAAV2/HBoV1 hybrid vector), for example, results in a hybrid vector that is highly tropic for airway epithelium (Yan et al., 2013, Mol. Ther., 21:2181-94).

In some embodiments, the virus particles are lentivirus. In some embodiments, the lentiviral vector particle is Human Immunodeficiency Virus-1 (HIV-1).

In some embodiments, the retroviral vector has a long terminal repeat sequence (LTR), e.g., a retroviral vector derived from the Moloney murine leukemia virus (MoMLV), myeloproliferative sarcoma virus (MPSV), murine embryonic stem cell virus (MESV), murine stem cell virus (MSCV), spleen focus forming virus (SFFV), or adeno-associated virus (AAV). Most retroviral vectors are derived from murine retroviruses. In some embodiments, the retroviruses include those derived from any avian or mammalian cell source. The retroviruses typically are amphotropic, meaning that they are capable of infecting host cells of several species, including humans. In one embodiment, the gene to be expressed replaces the retroviral gag, pol and/or env sequences. A number of illustrative retroviral systems have been described (e.g., U.S. Pat. Nos. 5,219,740; 6,207,453; 5,219,740)

Methods of lentiviral transduction are known. Exemplary methods are described in, e.g., Wang et al., J. Immunother. 35(9): 689-701, 2012; Cooper et al., Blood. 101:1637-1644, 2003; Verhoeyen et al., Methods Mol Biol. 506: 97-114, 2009; and Cavalieri et al., Blood. 102(2): 497-505, 2003.

A number of preclinical studies have demonstrated therapeutic and prophylactic efficacy of viral vector based gene delivery in animal models and in clinical trials. In some aspects, viral and virally derived vectors capable of replication provide consistent gene expression over time. In some aspects, replication competent viruses can result in undesired immunogenicity, toxicity, and cell death. In some embodiments, vectors capable of insertion are efficient for transduction of a variety of cells. However, in some aspects, they can pose a risk of insertional mutagenesis. Integration-deficient vectors can persist episomaly but can also retain the transduction efficiency of standard integrating vectors. Thus, in some embodiments, the vector particle is replication deficient. In some embodiments, the vector particle is integration deficient. Various methods of rendering a vector insertional or replication deficient are known in the art. Various replication-defective vaccine vectors have been produced with many other viruses, including adeno-associated virus (AAV), poliovirus, and Sendai virus.

### B. Virus-Like Particles

Also provided herein are virus-like particles (VLP) containing an agent, such as any of the polynucleotides or polypeptides described here (See Sections I and II). The VLPS include those derived from retroviruses or lentiviruses. While VLPs mimic native virion structure, they lack the viral genomic information necessary for independent replication within a host cell. Therefore, in some aspects, VLPs are non-infectious. In some embodiments, the VLP’s bilayer of amphipathic lipids is or comprises the viral envelope. In some embodiments, the targeted lipid particle’s bilayer of amphipathic lipids is or comprises lipids derived from a cell. A VLP typically comprises at least one type of structural protein from a virus. In most cases this protein will form a proteinaceous capsid (e.g. VLPs comprising a lentivrus, adenovirus or paramyxovirus structural protein). In some cases the capsid will also be enveloped in a lipid bilayer originating from the cell from which the assembled VLP has been released (e.g. VLPs comprising a human immunodeficiency virus structural protein such as GAG). In some embodiments, the VLP further comprises a targeting moiety as an envelope protein within the lipid bilayer.

In some embodiments, the VLP comprises supramolecular complexes formed by viral proteins that self-assemble into capsids. In some embodiments, the VLP is derived from viral capsids. In some embodiments, the VLP is derived from viral nucleocapsids. In some embodiments, the VLP is nucleocapsid-derived and retains the property of packaging nucleic acids. In some embodiments, the VLP includes only viral structural glycoproteins. In some embodiments, the VLP does not contain a viral genome.

Provided herein are VLPs that are derived from virus, such as those derived from retroviruses or lentiviruses. In some embodiments, the viral particles are derived from paramyxoviruses. Thus, in some examples, the viral-like particle is derived from Nipah, Hendra, or Rubeola viruses.

In some embodiments, the VLP is produced utilizing proteins (e.g., envelope proteins) from a virus within the Paramyxoviridae family. In some embodiments, the Paramyxoviridae family comprises members within the Henipavirus genus. In some embodiments, the Henipavirus is or comprises a Hendra (HeV) or a Nipah (NiV) virus. In particular embodiments, the VLPs futher comprise a fusogen.

### C. Non-viral Vectors

In some embodiments, the polynucleotides or polypeptides provided herein are not comprised within a viral or virally derived vector. Provided herein are non-viral vectors containing an agent, such as any of the polynucleotides or polypeptides described here (See Sections I and II). In some embodiments, the C19orf66 peptide or encoding polynucleotide is comprised within a non-viral vector or delivery vehicle. In some embodiments, the C19orf66 regulatory factor or encoding polynucleotide is comprised within a non-viral vector or delivery vehicle.

Among provided non-viral vectors are lipid particles. In some embodiments, the lipid particle comprises a naturally derived bilayer of amphipathic lipids with a surface targeting moiety (e.g., fusogen). In some embodiments, the lipid particle comprises (a) a lipid bilayer, (b) a lumen (e.g., comprising cytosol) surrounded by the lipid bilayer; and (c) a surface targeting moiety that is exogenous or overexpressed relative to the source cell. In some embodiments, the surface targeting moiety is a fusogen. In some embodiments, the fusogen is disposed in the lipid bilayer. In some embodiments, the fusosome comprises several different types of lipids, e.g., amphipathic lipids, such as phospholipids. In some embodiments, the fusosome comprises a lipid bilayer as the outermost surface. In some embodiments, the bilayer may be comprised of one or more lipids of the same or different type. In some embodiments, the lipids comprise phospholipids such as phosphocholines and phosphoinositols. In some embodiments, the lipids comprise DMPC, DOPC, and DSPC. In some embodiments, the polynucleotides or polypeptides provided herein are comprised in a non-viral vector, such as a lipid particle.

Nanoparticles are solid, spherical structures ranging to about 100 nm in size and can be prepared from natural or synthetic polymers. In some aspects, nanoparticles display the ability to target specific tissues or cells, protect target genes against nuclease degradation, improve DNA stability, and increase transformation efficiency or safety. In some embodiments, the non-viral vector is a nanoparticle. In some embodiments, the nanoparticle is one or more of any of biodegradable polymer, tetrapod quantum dot, tetrapod article, multi-legged luminescent nanoparticle, tetrapod nanocrystal, biodegradable nanoparticle, liposome, nanocarrier, or dendrimer.

Cationic lipids are amphiphilic molecules that have a cationic head group and a hydrophobic tail group connected by either stable or degradable linkages. Felgner and colleagues were the first to demonstrate the use of cationic lipids for DNA delivery in 1987 (Felgner et al. PNAS (84) 21:7413-7417, 1987). Many cationic lipids since then have been synthesized and evaluated for nucleic acid delivery, including for example GL67A. Thus, in some embodiments, the non-viral vector is a lipid complex. In some embodiments, the non-viral vector is a plasmid. In some embodiments, the non-viral vector is naked nucleic acid.

In some embodiments, the vector is mRNA. Non-viral delivery of mRNA can be obtained using injection of naked nucleic acid, polyplex, lipoplex or liposome-encapsulated mRNA, biolistic delivery by gene gun, microparticle carrier mediated delivery, and electroporation.

In some aspects, the basic components of an mRNA molecule include at least a coding region, a 5′UTR, a 3′UTR, a 5′ cap and a poly-A tail. In some embodiments, the first and second flanking regions range independently from 15-1,000 nucleotides in length (e.g., greater than 30, 40, 45, 50, 55, 60, 70, 80, 90, 100, 120, 140, 160, 180, 200, 250, 300, 350, 400, 450, 500, 600, 700, 800, and 900 nucleotides or at least 30, 40, 45, 50, 55, 60, 70, 80, 90, 100, 120, 140, 160, 180, 200, 250, 300, 350, 400, 450, 500, 600, 700, 800, 900, and 1,000 nucleotides). In some embodiments, the tailing sequence ranges from absent to 500 nucleotides in length (e.g., at least 60, 70, 80, 90, 120, 140, 160, 180, 200, 250, 300, 350, 400, 450, or 500 nucleotides). Where the tailing region is a polyA tail, the length may be determined in units of or as a function of polyA Binding Protein binding. In some embodiments, the polyA tail is long enough to bind at least 4 monomers of PolyA Binding Protein. PolyA Binding Protein monomers bind to stretches of approximately 38 nucleotides. As such, it has been observed that polyA tails of about 80 nucleotides and 160 nucleotides are functional.

The 5′ cap structure of an mRNA is involved in nuclear export, increasing mRNA stability. In some aspects, the 5′ cap binds the mRNA Cap Binding Protein (CBP), which in turn associates with poly(A) binding protein to form the mature cyclic mRNA species. In some aspects, the cap further assists the removal of 5′ proximal introns removal during mRNA splicing. Endogenous mRNA molecules may be 5′-end capped generating a 5′-ppp-5′-triphosphate linkage between a terminal guanosine cap residue and the 5‘-terminal transcribed sense nucleotide of the mRNA molecule. This 5′-guanylate cap may then be methylated to generate an N7-methyl-guanylate residue. The ribose sugars of the terminal and/or anteterminal transcribed nucleotides of the 5′ end of the mRNA may optionally also be 2′-0-methylated. 5′-decapping through hydrolysis and cleavage of the guanylate cap structure may target a nucleic acid molecule, such as an mRNA molecule, for degradation.

In some embodiments, modifications to the polynucleotides provided herein, such as an mRNA vector encoding C19orf66 or a regulatory factor thereof, may generate a non-hydrolyzable cap structure preventing decapping and thus increasing mRNA half-life. Because cap structure hydrolysis requires cleavage of 5′-ppp-5′ phosphorodiester linkages, in some aspects modified nucleotides may be used during the capping reaction. For example, a Vaccinia Capping Enzyme from New England Biolabs (Ipswich, MA) may be used with a-thio-guanosine nucleotides according to the manufacturer’s instructions to create a phosphorothioate linkage in the 5′-ppp-5′ cap.

In some embodiments, additional modified guanosine nucleotides may be used such as a-methyl-phosphonate and seleno-phosphate nucleotides. In some aspects, additional modifications include 2′-O-methylation of the ribose sugars of 5 ‘-terminal and/or 5′-anteterminal nucleotides of the mRNA (as mentioned above) on the 2′-hydroxyl group of the sugar ring. Multiple distinct 5 ‘-cap structures can be used to generate the 5 ‘-cap of a nucleic acid molecule, such as an mRNA molecule.

Cap analogs, also referred to as synthetic cap analogs, chemical caps, chemical cap analogs, or structural or functional cap analogs, differ from natural (i.e. endogenous, wild-type or physiological) 5′-caps in their chemical structure, while retaining cap function. In some aspects, cap analogs may be chemically (i.e. non-enzymatically) or enzymatically synthesized and/or linked to a nucleic acid molecule.

In some embodiments, the capping region may comprise a single cap or a series of nucleotides forming the cap. In this embodiment the capping region may be from 1 to 10, e.g. 2-9, 3-8, 4-7, 1-5, 5-10, or at least 2, or 10 or fewer nucleotides in length. In some embodiments, the cap is absent.

In some aspects, the polynucleotides provided herein, such as a non-viral mRNA vector, comprises a region of polynucleic acid sequence that is partially or substantially not translatable, e.g., having a noncoding region. Such molecules are generally not translated, but can exert an effect on protein production by one or more of binding to and sequestering one or more translational machinery components such as a ribosomal protein or a transfer R A (tR A), thereby effectively reducing protein expression in the cell or modulating one or more pathways or cascades in a cell which in turn alters protein levels. In some aspects, the polynucleotides provided herein in Sections I and II contain a noncoding region. In some embodiments, the noncoding region encodes one or more long noncoding RNA (IncRNA, or lincRNA) or portion thereof, a small nucleolar RNA (sno-RNA), micro RNA (miRNA), small interfering RNA (siRNA) or Piwi-interacting RNA (piRNA).

In some embodiments, modification of 3′ untranslated region AU rich elements (AREs) is used to modulate the stability of polynucleotides. When engineering specific polynucleotides, such as those encoding C19orf66 or regulatory factors thereof, one or more copies of an ARE can be introduced to make polynucleotides provided herein less stable. In some aspects, reducing stability of the polynucleotide reduces cognate protein expression. In some aspects, AREs can be identified and removed or mutated to increase the intracellular stability and thus increase translation and production of the resultant protein. Transfection experiments can be conducted in relevant cell lines, using polynucleotides provided herein and protein production can be assayed at various time points post-transfection. For example, cells can be transfected with different ARE-engineering molecules and by using an ELISA kit to the relevant protein and assaying protein produced at 6 hour, 12 hour, 24 hour, 48 hour, and 7 days post-transfection.

In some embodiments, the polynucleotides provided herein may further comprise, in addition to a Start and/or Stop codon, one or more signal and/or restriction sequences.

In some aspects, non-viral nucleic acid transfer or delivery vehicles can be less toxic, less antigenic, easier, and less expensive to prepare than viral vectors for delivery of nucleic acids. Certain delivery vehicles, such as cationic lipid or polymer delivery vehicles, can also help protect from endogenous RNAse during nucleic acid transfer.

### D. Methods of Generating Lipid Particles

Provided herein is a lipid particle comprising an agent, such as any of the polynucleotides or polypeptides described here (See Sections I and II). In some embodiments, the lipid particle can be a viral particle, a virus-like particle, a nanoparticle, a vesicle, an exosome, a dendrimer, a lentivirus, a viral vector, an enucleated cell, a microvesicle, a membrane vesicle, an extracellular membrane vesicle, a plasma membrane vesicle, a giant plasma membrane vesicle, an apoptotic body, a mitoparticle, a pyrenocyte, a lysosome, another membrane enclosed vesicle, or a lentiviral vector, a viral-based particle, a virus-like particle (VLP) or a cell derived particle.

In some embodiments, lipid particles may be produced in multiple cell culture systems including bacteria, mammalian cell lines, insect cell lines, yeast and plant cells.

In some embodiments, the assembly of a lipid particle is initiated by binding of the core protein to a unique encapsidation sequence within the viral genome (e.g. UTR with stem-loop structure). In some embodiments, the interaction of the core with the encapsidation sequence facilitates oligomerization.

In some embodiments, the vehicle is a targeted lipid particle which comprises a sequence that is devoid of or lacking viral RNA, which in some aspects may be the result of removing or eliminating the viral RNA from the sequence. In some embodiments, this may be achieved by using an endogenous packaging signal binding site on gag. In some embodiments, the endogenous packaging signal binding site is on pol. In some embodiments, the polynucleotides provided herein, such as described in sections I.A and II.C, will contain a cognate packaging signal. In some embodiments, a heterologous binding domain (which is heterologous to gag) located on the polynucleotides provided herein to be delivered, and a cognate binding site located on gag or pol, can be used to ensure packaging of the polynucleotides provided herein to be delivered. In some embodiments, the vector particles could be used to deliver the polynucleotides or polypeptides provided herein, in which case functional integrase and/or reverse transcriptase is not required. In some embodiments, the vector particles could also be used to deliver a therapeutic gene, such as C19orf66, of interest, in which case pol is typically included.

### 1. Transfer Vectors

In some embodiments, a vector particle comprises a nucleic acid molecule (e.g., a transfer plasmid) that includes virus-derived nucleic acid elements that typically facilitate transfer of the nucleic acid molecule or integration into the genome of a cell or to a viral particle that mediates nucleic acid transfer. In some aspects, vector particles will typically include various viral components and sometimes also host cell components in addition to nucleic acid(s). In some embodiments, a vector comprises e.g., a virus or viral particle capable of transferring a nucleic acid into a cell, or to the transferred nucleic acid (e.g., as naked mRNA). In some embodiments, viral vectors and transfer plasmids comprise structural and/or functional genetic elements that are primarily derived from a virus. A retroviral vector can comprise a viral vector or plasmid containing structural and functional genetic elements, or portions thereof, that are primarily derived from a retrovirus. A lentiviral vector can comprise a viral vector or plasmid containing structural and functional genetic elements, or portions thereof, including LTRs that are primarily derived from a lentivirus.

In embodiments, a lentiviral vector (e.g., lentiviral expression vector) may comprise a lentiviral transfer plasmid (e.g., as naked DNA) or an infectious lentiviral particle. With respect to elements such as cloning sites, promoters, regulatory elements, heterologous nucleic acids, etc., it is to be understood that the sequences of these elements can be present in RNA form in lentiviral particles and can be present in DNA form in DNA plasmids.

In some embodiments, in the vectors described herein at least part of one or more protein coding regions that contribute to or are essential for replication may be absent compared to the corresponding wild-type virus. In some embodiments, the viral vector replication-defective. In some embodiments, the vector is capable of transducing a target non-dividing host cell and/or integrating its genome into a host genome.

In some embodiments, the structure of a wild-type retrovirus genome often comprises a 5′ long terminal repeat (LTR) and a 3′ LTR, between or within which are located a packaging signal to enable the genome to be packaged, a primer binding site, integration sites to enable integration into a host cell genome and gag, pol and env genes encoding the packaging components which promote the assembly of viral particles. More complex retroviruses have additional features, such as rev and RRE sequences in HIV, which enable the efficient export of RNA transcripts of the integrated provirus from the nucleus to the cytoplasm of an infected target cell. In the provirus, the viral genes are flanked at both ends by regions called long terminal repeats (LTRs). In some embodiments, the LTRs are involved in proviral integration and transcription. In some embodiments, LTRs serve as enhancer-promoter sequences and can control the expression of the viral genes. In some embodiments, encapsidation of the retroviral RNAs occurs by virtue of a psi sequence located at the 5′ end of the viral genome.

In some embodiments, LTRs are similar sequences that can be divided into three elements, which are called U3, R and U5. U3 is derived from the sequence unique to the 3′ end of the RNA. R is derived from a sequence repeated at both ends of the RNA and U5 is derived from the sequence unique to the 5′ end of the RNA. The sizes of the three elements can vary considerably among different retroviruses.

In some embodiments, for the viral genome, the site of transcription initiation is typically at the boundary between U3 and R in one LTR and the site of poly (A) addition (termination) is at the boundary between R and U5 in the other LTR. U3 contains most of the transcriptional control elements of the provirus, which include the promoter and multiple enhancer sequences responsive to cellular and in some cases, viral transcriptional activator proteins. In some embodiments, retroviruses comprise any one or more of the following genes that code for proteins that are involved in the regulation of gene expression: tat, rev, tax and rex.

In some embodiments, the structural genes gag, pol and env, gag encodes the internal structural protein of the virus. In some embodiments, Gag protein is proteolytically processed into the mature proteins MA (matrix), CA (capsid) and NC (nucleocapsid). In some embodiments, the pol gene encodes the reverse transcriptase (RT), which contains DNA polymerase, associated RNase H and integrase (IN), which mediate replication of the genome. In some embodiments, the env gene encodes the surface (SU) glycoprotein and the transmembrane (TM) protein of the virion, which form a complex that interacts specifically with cellular receptor proteins. In some embodiments, the interaction promotes infection by fusion of the viral membrane with the cell membrane.

In some embodiments, a replication-defective retroviral vector genome gag, pol and env may be absent or not functional. In some embodiments, the R regions at both ends of the RNA are typically repeated sequences. In some embodiments, U5 and U3 represent unique sequences at the 5′ and 3′ ends of the RNA genome respectively.

In some embodiments, retroviruses may also contain additional genes which code for proteins other than gag, pol and env. Examples of additional genes include (in HIV), one or more of vif, vpr, vpx, vpu, tat, rev and nef. EIAV has (amongst others) the additional gene S2. In some embodiments, proteins encoded by additional genes serve various functions, some of which may be duplicative of a function provided by a cellular protein. In EIAV, for example, tat acts as a transcriptional activator of the viral LTR (Derse and Newbold 1993 Virology 194:530-6; Maury et al. 1994 Virology 200:632-42). It binds to a stable, stem-loop RNA secondary structure referred to as TAR. Rev regulates and co-ordinates the expression of viral genes through rev-response elements (RRE) (Martarano et al. 1994 J. Virol. 68:3102-11).

In some embodiments, in addition to protease, reverse transcriptase and integrase, non-primate lentiviruses contain a fourth pol gene product which codes for a dUTPase. In some embodiments, this a role in the ability of these lentiviruses to infect certain non-dividing or slowly dividing cell types.

In embodiments, a recombinant lentiviral vector (RLV) is a vector with sufficient retroviral genetic information to allow packaging of an RNA genome, in the presence of packaging components, into a viral particle capable of infecting a target cell. In some embodiments, infection of the target cell can comprise reverse transcription and integration into the target cell genome. In some embodiments, the RLV typically carries non-viral coding sequences which are to be delivered by the vector to the target cell. In some embodiments, an RLV is incapable of independent replication to produce infectious retroviral particles within the target cell. In some embodiments, the RLV lacks a functional gag-pol and/or env gene and/or other genes involved in replication. In some embodiments, the vector may be configured as a split-intron vector, e.g., as described in PCT Patent Application WO 99/15683, which is herein incorporated by reference in its entirety.

In some embodiments, the lentiviral vector comprises a minimal viral genome, e.g., the viral vector has been manipulated so as to remove the non-essential elements and to retain the essential elements in order to provide the required functionality to infect, transduce and deliver a nucleotide sequence of interest to a target host cell, e.g., as described in WO 98/17815, which is herein incorporated by reference in its entirety.

In some embodiments, a minimal lentiviral genome may comprise, e.g., (5′)R-U5-one or more first nucleotide sequences-U3-R(3′). In some embodiments, the plasmid vector used to produce the lentiviral genome within a source cell can also include transcriptional regulatory control sequences operably linked to the lentiviral genome to direct transcription of the genome in a source cell. In some embodiments, the regulatory sequences may comprise the natural sequences associated with the transcribed retroviral sequence, e.g., the 5′ U3 region, or they may comprise a heterologous promoter such as another viral promoter, for example the CMV promoter. In some embodiments, lentiviral genomes comprise additional sequences to promote efficient virus production. In some embodiments, in the case of HIV, rev and RRE sequences may be included. In some embodiments, alternatively or combination, codon optimization may be used, e.g., the gene encoding the exogenous agent may be codon optimized, e.g., as described in WO 01/79518, which is herein incorporated by reference in its entirety. In some embodiments, alternative sequences which perform a similar or the same function as the rev/RRE system may also be used. In some embodiments, a functional analogue of the rev/RRE system is found in the Mason Pfizer monkey virus. In some embodiments, this is known as CTE and comprises an RRE-type sequence in the genome which is believed to interact with a factor in the infected cell. The cellular factor can be thought of as a rev analogue. In some embodiments, CTE may be used as an alternative to the rev/RRE system. In some embodiments, the Rex protein of HTLV-I can functionally replace the Rev protein of HIV-I . Rev and Rex have similar effects to IRE-BP.

In some embodiments, a retroviral nucleic acid (e.g., a lentiviral nucleic acid, e.g., a primate or non-primate lentiviral nucleic acid) (1) comprises a deleted gag gene wherein the deletion in gag removes one or more nucleotides downstream of about nucleotide 350 or 354 of the gag coding sequence; (2) has one or more accessory genes absent from the retroviral nucleic acid; (3) lacks the tat gene but includes the leader sequence between the end of the 5′ LTR and the ATG of gag; and (4) combinations of (1), (2) and (3). In an embodiment the lentiviral vector comprises all of features (1) and (2) and (3). This strategy is described in more detail in WO 99/32646, which is herein incorporated by reference in its entirety.

In some embodiments, a primate lentivirus minimal system requires none of the HIV/SIV additional genes vif, vpr, vpx, vpu, tat, rev and nef for either vector production or for transduction of dividing and non-dividing cells. In some embodiments, an EIAV minimal vector system does not require S2 for either vector production or for transduction of dividing and non-dividing cells.

In some embodiments, the deletion of additional genes may permit vectors to be produced without the genes associated with disease in lentiviral (e.g. HIV) infections. In some embodiments, tat is associated with disease. In some embodiments, the deletion of additional genes permits the vector to package more heterologous DNA. In some embodiments, genes whose function is unknown, such as S2, may be omitted, thus reducing the risk of causing undesired effects. Examples of minimal lentiviral vectors are disclosed in WO 99/32646 and in WO 98/17815.

In some embodiments, the retroviral nucleic acid is devoid of at least tat and S2 (if it is an EIAV vector system), and possibly also vif, vpr, vpx, vpu and nef. In some embodiments, the retroviral nucleic acid is also devoid of rev, RRE, or both.

In some embodiments the retroviral nucleic acid comprises vpx. The Vpx polypeptide binds to and induces the degradation of the SAMHD1 restriction factor, which degrades free dNTPs in the cytoplasm. In some embodiments, the concentration of free dNTPs in the cytoplasm increases as Vpx degrades SAMHD1 and reverse transcription activity is increased, thus facilitating reverse transcription of the retroviral genome and integration into the target cell genome.

In some embodiments, different cells differ in their usage of particular codons. In some embodiments, this codon bias corresponds to a bias in the relative abundance of particular tRNAs in the cell type. In some embodiments, by altering the codons in the sequence so that they are tailored to match with the relative abundance of corresponding tRNAs, it is possible to increase expression. In some embodiments, it is possible to decrease expression by deliberately choosing codons for which the corresponding tRNAs are known to be rare in the particular cell type. In some embodiments, an additional degree of translational control is available. An additional description of codon optimization is found, e.g., in WO 99/41397, which is herein incorporated by reference in its entirety.

In some embodiments viruses, including HIV and other lentiviruses, use a large number of rare codons and by changing these to correspond to commonly used mammalian codons, increased expression of the packaging components in mammalian producer cells can be achieved.

In some embodiments, codon optimization has a number of other advantages. In some embodiments, by virtue of alterations in their sequences, the nucleotide sequences encoding the packaging components may have RNA instability sequences (INS) reduced or eliminated from them. At the same time, the amino acid sequence coding sequence for the packaging components is retained so that the viral components encoded by the sequences remain the same, or at least sufficiently similar that the function of the packaging components is not compromised. In some embodiments, codon optimization also overcomes the Rev/RRE requirement for export, rendering optimized sequences Rev independent. In some embodiments, codon optimization also reduces homologous recombination between different constructs within the vector system (for example between the regions of overlap in the gag-pol and env open reading frames). In some embodiments, codon optimization leads to an increase in viral titer and/or improved safety.

In some embodiments, only codons relating to INS are codon optimized. In other embodiments, the sequences are codon optimized in their entirety, with the exception of the sequence encompassing the frameshift site of gag-pol.

The gag-pol gene comprises two overlapping reading frames encoding the gag-pol proteins. The expression of both proteins depends on a frameshift during translation. This frameshift occurs as a result of ribosome “slippage” during translation. This slippage is thought to be caused at least in part by ribosome-stalling RNA secondary structures. Such secondary structures exist downstream of the frameshift site in the gag-pol gene. For HIV, the region of overlap extends from nucleotide 1222 downstream of the beginning of gag (wherein nucleotide 1 is the A of the gag ATG) to the end of gag (nt 1503). Consequently, a 281 bp fragment spanning the frameshift site and the overlapping region of the two reading frames is preferably not codon optimized. In some embodiments, retaining this fragment will enable more efficient expression of the gag-pol proteins. For EIAV, the beginning of the overlap is at nt 1262 (where nucleotide 1 is the A of the gag ATG). The end of the overlap is at nt 1461. In order to ensure that the frameshift site and the gag-pol overlap are preserved, the wild type sequence may be retained from nt 1156 to 1465.

In some embodiments, derivations from optimal codon usage may be made, for example, in order to accommodate convenient restriction sites, and conservative amino acid changes may be introduced into the gag-pol proteins.

In some embodiments, codon optimization is based on codons with poor codon usage in mammalian systems. The third and sometimes the second and third base may be changed.

In some embodiments, due to the degenerate nature of the genetic code, it will be appreciated that numerous gag-pol sequences can be achieved by a skilled worker. Also, there are many retroviral variants described which can be used as a starting point for generating a codon optimized gag-pol sequence. Lentiviral genomes can be quite variable. For example there are many quasi-species of HIV-I which are still functional. This is also the case for EIAV. These variants may be used to enhance particular parts of the transduction process. Examples of HIV-I variants may be found in the HIV databases maintained by Los Alamos National Laboratory. Details of EIAV clones may be found at the NCBI database maintained by the National Institutes of Health.

In some embodiments, the strategy for codon optimized gag-pol sequences can be used in relation to any retrovirus, e.g., EIAV, FIV, BIV, CAEV, VMR, SIV, HIV-I and HIV -2. In addition this method could be used to increase expression of genes from HTLV-I, HTLV-2, HFV, HSRV and human endogenous retroviruses (HERV), MLV and other retroviruses.

In embodiments, the retroviral vector comprises a packaging signal that comprises from 255 to 360 nucleotides of gag in vectors that still retain env sequences, or about 40 nucleotides of gag in a particular combination of splice donor mutation, gag and env deletions. In some embodiments, the retroviral vector includes a gag sequence which comprises one or more deletions, e.g., the gag sequence comprises about 360 nucleotides derivable from the N-terminus.

In some embodiments, the retroviral vector, helper cell, helper virus, or helper plasmid may comprise retroviral structural and accessory proteins, for example gag, pol, env, tat, rev, vif, vpr, vpu, vpx, or nef proteins or other retroviral proteins. In some embodiments the retroviral proteins are derived from the same retrovirus. In some embodiments the retroviral proteins are derived from more than one retrovirus, e.g. 2, 3, 4, or more retroviruses.

In some embodiments, the gag and pol coding sequences are generally organized as the Gag-Pol Precursor in native lentivirus. The gag sequence codes for a 55-kD Gag precursor protein, also called p55. The p55 is cleaved by the virally encoded protease (a product of the pol gene) during the process of maturation into four smaller proteins designated MA (matrix [p17]), CA (capsid [p24]), NC (nucleocapsid [p9]), and p6. The pol precursor protein is cleaved away from Gag by a virally encoded protease, and further digested to separate the protease (p10), RT (p50), RNase H (p15), and integrase (p31) activities.

In some embodiments, the lentiviral vector is integration-deficient. In some embodiments, the pol is integrase deficient, such as by encoding due to mutations in the integrase gene. For example, the pol coding sequence can contain an inactivating mutation in the integrase, such as by mutation of one or more of amino acids involved in catalytic activity, i.e. mutation of one or more of aspartic 64, aspartic acid 116 and/or glutamic acid 152. In some embodiments, the integrase mutation is a D64V mutation. In some embodiments, the mutation in the integrase allows for packaging of viral RNA into a lentivirus. In some embodiments, the mutation in the integrase allows for packaging of viral proteins into a letivirus. In some embodiments, the mutation in the integrase reduces the possibility of insertional mutagenesis. In some embodiments, the mutation in the integrase decreases the possibility of generating replication-competent recombinants (RCRs) (Wanisch et al. 2009. Mol Ther. 1798):1316-1332).In some embodiments, native Gag-Pol sequences can be utilized in a helper vector (e.g., helper plasmid or helper virus), or modifications can be made. These modifications include, chimeric Gag-Pol, where the Gag and Pol sequences are obtained from different viruses (e.g., different species, subspecies, strains, clades, etc.), and/or where the sequences have been modified to improve transcription and/or translation, and/or reduce recombination.

In some embodiments, the retroviral nucleic acid includes a polynucleotide encoding a 150-250 (e.g., 168) nucleotide portion of a gag protein that (i) includes a mutated INS 1 inhibitory sequence that reduces restriction of nuclear export of RNA relative to wild-type INS 1, (ii) contains two nucleotide insertion that results in frame shift and premature termination, and/or (iii) does not include INS2, INS3, and INS4 inhibitory sequences of gag.

In some embodiments, a vector described herein is a hybrid vector that comprises both retroviral (e.g., lentiviral) sequences and non-lentiviral viral sequences. In some embodiments, a hybrid vector comprises retroviral e.g., lentiviral, sequences for reverse transcription, replication, integration and/or packaging.

In some embodiments, most or all of the viral vector backbone sequences are derived from a lentivirus, e.g., HIV-1. However, it is to be understood that many different sources of retroviral and/or lentiviral sequences can be used or combined and numerous substitutions and alterations in certain of the lentiviral sequences may be accommodated without impairing the ability of a transfer vector to perform the functions described herein. A variety of lentiviral vectors are described in Naldini et al., (1996a, 1996b, and 1998); Zufferey et al., (1997); Dull et al., 1998, U.S. Pat. Nos. 6,013,516; and 5,994,136, many of which may be adapted to produce a retroviral nucleic acid.

In some embodiments, at each end of the provirus, long terminal repeats (LTRs) are typically found. An LTR typically comprises a domain located at the ends of retroviral nucleic acid which, in their natural sequence context, are direct repeats and contain U3, R and U5 regions. LTRs generally promote the expression of retroviral genes (e.g., promotion, initiation and polyadenylation of gene transcripts) and viral replication. The LTR can comprise numerous regulatory signals including transcriptional control elements, polyadenylation signals and sequences for replication and integration of the viral genome. The viral LTR is typically divided into three regions called U3, R and U5. The U3 region typically contains the enhancer and promoter elements. The U5 region is typically the sequence between the primer binding site and the R region and can contain the polyadenylation sequence. The R (repeat) region can be flanked by the U3 and U5 regions. The LTR is typically composed of U3, R and U5 regions and can appear at both the 5′ and 3′ ends of the viral genome. In some embodiments, adjacent to the 5′ LTR are sequences for reverse transcription of the genome (the tRNA primer binding site) and for efficient packaging of viral RNA into particles (the Psi site).

In some embodiments, a packaging signal can comprise a sequence located within the retroviral genome which mediate insertion of the viral RNA into the viral capsid or particle, see e.g., Clever et al., 1995. J. of Virology, Vol. 69, No. 4; pp. 2101-2109. Several retroviral vectors use a minimal packaging signal (a psi [Ψ] sequence) for encapsidation of the viral genome.

In various embodiments, retroviral nucleic acids comprise modified 5′ LTR and/or 3′ LTRs. Either or both of the LTR may comprise one or more modifications including, but not limited to, one or more deletions, insertions, or substitutions. Modifications of the 3′ LTR are often made to improve the safety of lentiviral or retroviral systems by rendering viruses replication-defective, e.g., virus that is not capable of complete, effective replication such that infective virions are not produced (e.g., replication-defective lentiviral progeny).

In some embodiments, a vector is a self-inactivating (SIN) vector, e.g., replication-defective vector, e.g., retroviral or lentiviral vector, in which the right (3′) LTR enhancer-promoter region, known as the U3 region, has been modified (e.g., by deletion or substitution) to prevent viral transcription beyond the first round of viral replication. This is because the right (3′) LTR U3 region can be used as a template for the left (5′) LTR U3 region during viral replication and, thus, absence of the U3 enhancer-promoter inhibits viral replication. In embodiments, the 3′ LTR is modified such that the U5 region is removed, altered, or replaced, for example, with an exogenous poly(A) sequence The 3′ LTR, the 5′ LTR, or both 3′ and 5′ LTRs, may be modified LTRs.

In some embodiments, the U3 region of the 5′ LTR is replaced with a heterologous promoter to drive transcription of the viral genome during production of viral particles. Examples of heterologous promoters which can be used include, for example, viral simian virus 40 (SV40) (e.g., early or late), cytomegalovirus (CMV) (e.g., immediate early), Moloney murine leukemia virus (MoMLV), Rous sarcoma virus (RSV), and herpes simplex virus (HSV) (thymidine kinase) promoters. In some embodiments, promoters are able to drive high levels of transcription in a Tat-independent manner. In certain embodiments, the heterologous promoter has additional advantages in controlling the manner in which the viral genome is transcribed. For example, the heterologous promoter can be inducible, such that transcription of all or part of the viral genome will occur only when the induction factors are present. Induction factors include, but are not limited to, one or more chemical compounds or the physiological conditions such as temperature or pH, in which the host cells are cultured.

In some embodiments, viral vectors comprise a TAR (trans-activation response) element, e.g., located in the R region of lentiviral (e.g., HIV) LTRs. This element interacts with the lentiviral trans-activator (tat) genetic element to enhance viral replication. However, this element is not required, e.g., in embodiments wherein the U3 region of the 5′ LTR is replaced by a heterologous promoter.

In some embodiments, the R region, e.g., the region within retroviral LTRs beginning at the start of the capping group (i.e., the start of transcription) and ending immediately prior to the start of the poly A tract can be flanked by the U3 and U5 regions. The R region plays a role during reverse transcription in the transfer of nascent DNA from one end of the genome to the other.

In some embodiments, the retroviral nucleic acid can also comprise a FLAP element, e.g., a nucleic acid whose sequence includes the central polypurine tract and central termination sequences (cPPT and CTS) of a retrovirus, e.g., HIV-1 or HIV-2. Suitable FLAP elements are described in U.S. Pat. No. 6,682,907 and in Zennou, et al., 2000, Cell, 101:173, which are herein incorporated by reference in their entireties. During HIV-1 reverse transcription, central initiation of the plus-strand DNA at the central polypurine tract (cPPT) and central termination at the central termination sequence (CTS) can lead to the formation of a three-stranded DNA structure: the HIV-1 central DNA flap. In some embodiments, the retroviral or lentiviral vector backbones comprise one or more FLAP elements upstream or downstream of the gene encoding the exogenous agent. For example, in some embodiments a transfer plasmid includes a FLAP element, e.g., a FLAP element derived or isolated from HIV-1.

In embodiments, a retroviral or lentiviral nucleic acid comprises one or more export elements, e.g., a cis-acting post-transcriptional regulatory element which regulates the transport of an RNA transcript from the nucleus to the cytoplasm of a cell. Examples of RNA export elements include, but are not limited to, the human immunodeficiency virus (HIV) rev response element (RRE) (see e.g., Cullen et al., 1991. J. Virol. 65: 1053; and Cullen et al., 1991. Cell 58: 423), and the hepatitis B virus post-transcriptional regulatory element (HPRE), which are herein incorporated by reference in their entireties. Generally, the RNA export element is placed within the 3′ UTR of a gene, and can be inserted as one or multiple copies.

In some embodiments, expression of heterologous sequences in viral vectors is increased by incorporating one or more of, e.g., all of, posttranscriptional regulatory elements, polyadenylation sites, and transcription termination signals into the vectors. A variety of posttranscriptional regulatory elements can increase expression of a heterologous nucleic acid at the protein, e.g., woodchuck hepatitis virus posttranscriptional regulatory element (WPRE; Zufferey et al., 1999, J. Virol., 73:2886); the posttranscriptional regulatory element present in hepatitis B virus (HPRE) (Huang et al., Mol. Cell. Biol., 5:3864); and the like (Liu et al., 1995, Genes Dev., 9:1766), each of which is herein incorporated by reference in its entirety. In some embodiments, a retroviral nucleic acid described herein comprises a posttranscriptional regulatory element such as a WPRE or HPRE.

In some embodiments, a retroviral nucleic acid described herein lacks or does not comprise a posttranscriptional regulatory element such as a WPRE or HPRE.

In some embodiments, elements directing the termination and polyadenylation of the heterologous nucleic acid transcripts may be included, e.g., to increases expression of the exogenous agent. Transcription termination signals may be found downstream of the polyadenylation signal. In some embodiments, vectors comprise a polyadenylation sequence 3′ of a polynucleotide encoding the exogenous agent. A polyA site may comprise a DNA sequence which directs both the termination and polyadenylation of the nascent RNA transcript by RNA polymerase II. Polyadenylation sequences can promote mRNA stability by addition of a polyA tail to the 3′ end of the coding sequence and thus, contribute to increased translational efficiency. Illustrative examples of polyA signals that can be used in a retroviral nucleic acid, include AATAAA, ATTAAA, AGTAAA, a bovine growth hormone polyA sequence (BGHpA), a rabbit β-globin polyA sequence (rβgpA), or another suitable heterologous or endogenous polyA sequence.

In some embodiments, a retroviral or lentiviral vector further comprises one or more insulator elements, e.g., an insulator element described herein.

In various embodiments, the vectors comprise a promoter operably linked to a polynucleotide encoding an exogenous agent. The vectors may have one or more LTRs, wherein either LTR comprises one or more modifications, such as one or more nucleotide substitutions, additions, or deletions. The vectors may further comprise one of more accessory elements to increase transduction efficiency (e.g., a cPPT/FLAP), viral packaging (e.g., a Psi (Ψ) packaging signal, RRE), and/or other elements that increase exogenous gene expression (e.g., poly (A) sequences), and may optionally comprise a WPRE or HPRE.

In some embodiments, a lentiviral nucleic acid comprises one or more of, e.g., all of, e.g., from 5′ to 3′, a promoter (e.g., CMV), an R sequence (e.g., comprising TAR), a U5 sequence (e.g., for integration), a PBS sequence (e.g., for reverse transcription), a DIS sequence (e.g., for genome dimerization), a psi packaging signal, a partial gag sequence, an RRE sequence (e.g., for nuclear export), a cPPT sequence (e.g., for nuclear import), a promoter to drive expression of the exogenous agent, a gene encoding the exogenous agent, a WPRE sequence (e.g., for efficient transgene expression), a PPT sequence (e.g., for reverse transcription), an R sequence (e.g., for polyadenylation and termination), and a U5 signal (e.g., for integration).

### 2. Packing Vectors

Large scale vector particle production is often useful to achieve a desired concentration of vector particles. Particles can be produced by transfecting a transfer vector as described above into a packaging cell line that comprises viral structural and/or accessory genes, e.g., gag, pol, env, tat, rev, vif, vpr, vpu, vpx, or nef genes or other retroviral genes.

In some embodiments, the packaging vector is an expression vector or viral vector that lacks a packaging signal and comprises a polynucleotide encoding one, two, three, four or more viral structural and/or accessory genes. Typically, the packaging vectors are included in a producer cell, and are introduced into the cell via transfection, transduction or infection. A retroviral, e.g., lentiviral, transfer vector can be introduced into a producer cell line, via transfection, transduction or infection, to generate a source cell or cell line. The packaging vectors can be introduced into human cells or cell lines by standard methods including, e.g., calcium phosphate transfection, lipofection or electroporation. In some embodiments, the packaging vectors are introduced into the cells together with a dominant selectable marker, such as neomycin, hygromycin, puromycin, blastocidin, zeocin, thymidine kinase, DHFR, Gln synthetase or ADA, followed by selection in the presence of the appropriate drug and isolation of clones. A selectable marker gene can be linked physically to genes encoding by the packaging vector, e.g., by IRES or self-cleaving viral peptides.

In some embodiments, producer cell lines include cell lines that do not contain a packaging signal, but do stably or transiently express viral structural proteins and replication enzymes (e.g., gag, pol and env) which can package viral particles. Any suitable cell line can be employed, e.g., mammalian cells, e.g., human cells. Suitable cell lines which can be used include, for example, CHO cells, BHK cells, MDCK cells, C3H 10T1/2 cells, FLY cells, Psi-2 cells, BOSC 23 cells, PA317 cells, WEHI cells, COS cells, BSC 1 cells, BSC 40 cells, BMT 10 cells, VERO cells, W138 cells, MRC5 cells, A549 cells, HT1080 cells, 293 cells, 293T cells, B-50 cells, 3T3 cells, NIH3T3 cells, HepG2 cells, Saos-2 cells, Huh7 cells, HeLa cells, W163 cells, 211 cells, and 211A cells. In embodiments, the packaging cells are 293 cells, 293T cells, or A549 cells.

In some embodiments, a source cell line includes a cell line which is capable of producing recombinant retroviral particles, comprising a producer cell line and a transfer vector construct comprising a packaging signal. Methods of preparing viral stock solutions are illustrated by, e.g., Y. Soneoka et al. (1995) Nucl. Acids Res. 23:628-633, and N. R. Landau et al. (1992) J. Virol. 66:5110-5113, which are incorporated herein by reference. Infectious virus particles may be collected from the producer cells, e.g., by cell lysis, or collection of the supernatant of the cell culture. Optionally, the collected virus particles may be enriched or purified.

In some embodiments, the source cell comprises one or more plasmids coding for viral structural proteins and replication enzymes (e.g., gag, pol and env) which can package viral particles. In some embodiments, the sequences coding for at least two of the gag, pol, and env precursors are on the same plasmid. In some embodiments, the sequences coding for the gag, pol, and env precursors are on different plasmids. In some embodiments, the sequences coding for the gag, pol, and env precursors have the same expression signal, e.g., promoter. In some embodiments, the sequences coding for the gag, pol, and env precursors have a different expression signal, e.g., different promoters. In some embodiments, expression of the gag, pol, and env precursors is inducible. In some embodiments, the plasmids coding for viral structural proteins and replication enzymes are transfected at the same time or at different times. In some embodiments, the plasmids coding for viral structural proteins and replication enzymes are transfected at the same time or at a different time from the packaging vector.

In some embodiments, the source cell line comprises one or more stably integrated viral structural genes. In some embodiments expression of the stably integrated viral structural genes is inducible.

In some embodiments, expression of the viral structural genes is regulated at the transcriptional level. In some embodiments, expression of the viral structural genes is regulated at the translational level. In some embodiments, expression of the viral structural genes is regulated at the post-translational level.

In some embodiments, expression of the viral structural genes is regulated by a tetracycline (Tet)-dependentsystem, in which a Tet-regulated transcriptional repressor (Tet-R) binds to DNA sequences included in a promoter and represses transcription by steric hindrance (Yao et al, 1998; Jones et al, 2005). Upon addition of doxycycline (dox), Tet-R is released, allowing transcription.

In some embodiments, expression of the viral structural genes is regulated by a Tet-on system, which utilizes a transactivator rtTA (reverse tetracycline-controlled transactivator). In some embodiments, the rtTA includes the Tet-R repressor fused with VP16. In some embodiments, a tetracycline responsive promoter for expression was constructed by fusing a minimal cytomegalovirus (CMV) promoter to a sequence corresponding with the tetracycline operator sequence (TetO).

In some aspects, a derivative of tetracycline is the preffered effector for expression of the viral structural genes. Without wishing to be bound by theory, doxycycline has been observed to have high affinity for Tet-R, rtTA, and other regulatory factors associated with tetracycline binding. In some aspects, doxycycline is associated with lower toxicity, a known half-life of 24 hours, and a more favorable tissue distribution in comparison to tetracycline.

Multiple other suitable transcriptional regulatory promoters, transcription factors, and small molecule inducers are suitable to regulate transcription of viral structural genes.

In some embodiments, the third-generation lentivirus components, human immunodeficiency virus type 1 (HIV) Rev, Gag/Pol, and an envelope under the control of Tet-regulated promoters and coupled with antibiotic resistance cassettes are separately integrated into the source cell genome. In some embodiments the source cell only has one copy of each of Rev, Gag/Pol, and an envelope protein integrated into the genome.

In some embodiments a nucleic acid encoding the exogenous agent (e.g., a retroviral nucleic acid encoding the exogenous agent) is also integrated into the source cell genome.

In some embodiments, a retroviral nucleic acid described herein is unable to undergo reverse transcription. Such a nucleic acid, in embodiments, is able to transiently express an exogenous agent. The retrovirus or VLP, may comprise a disabled reverse transcriptase protein, or may not comprise a reverse transcriptase protein. In embodiments, the retroviral nucleic acid comprises a disabled primer binding site (PBS) and/or att site. In embodiments, one or more viral accessory genes, including rev, tat, vif, nef, vpr, vpu, vpx and S2 or functional equivalents thereof, are disabled or absent from the retroviral nucleic acid. In embodiments, one or more accessory genes selected from S2, rev and tat are disabled or absent from the retroviral nucleic acid

### E. Vehicle Targeting and Retargeting

In some embodiments, the vehicle further comprises a vector-surface targeting moiety which specifically binds to a target ligand. It will be recognized by those skilled in the art that, the vehicles provided herein harbor the attachment and/or fusion glycoproteins and are capable of binding to target cells and delivering the vehicle contents to the cytoplasm of the target cells. It will also be recognized by those skilled in the art that this is due to the natural viral entry mechanism that involves fusion of the viral membrane directly with the target cell plasma membrane.

It will further be recognized by those skilled in the art that many viruses, such as paramyxoviruses, bind to sialic acid receptors and hence the corresponding derivative vehicles can deliver their contents generically to nearly any kind of cell that expresses sialic acid bearing receptors. Other viruses such as Nipah virus and HIV bind to protein receptors, and hence the corresponding vehicles have a specificity that matches the natural tropisms for each virus and its surface proteins, respectively.

Furthermore, it will be recognized that technology exists to “re-target” attachment proteins, making it so that the vehicles only interact with particular cells or cell types that express a marker protein of interest (Msaouel et al., Meths Mol Biol 797: 141-162, 2012). Thus, vehicle surface glycoproteins proteins can be supplemented with or replaced by other targeting proteins, including but not necessarily limited to antibodies and antigen binding fragments thereof, receptor ligands, and other approaches that will be apparent to those skilled in the art given the benefit of the present disclosure. In some embodiments, the vector-surface targeting moiety is a polypeptide. In some embodiments, the polypeptide is a fusogen.

### 1. Fusogens

In some embodiments, the provided vehicles, e.g. lipid particles, such as viral vectors or viral-like particles, contain one or more fusogens. In some embodiments, the lipid particle, e.g viral vector or viral-like particle, contains an exogenous or overexpressed fusogen. In some embodiments, the fusogen is disposed in the lipid bilayer. In some embodiments, the fusogen facilitates the fusion of the lipid particle to a membrane. In some embodiments, the membrane is a plasma cell membrane. In some embodiments, the lipid particle, such as a viral or non-viral vector, comprising the fusogen integrates into the membrane into a lipid bilayer of a target cell. In some embodiments, the fusogen results in mixing between lipids of the lipid particle and lipids of the target cell. In some embodiments, the fusogen results in formation of one or more pores between the interior of the non-cell particle and the cytosol of the target cell.

In some embodiments, fusogens are protein based, lipid based, and chemical based fusogens. In some embodiments, the lipid particle, e.g. viral vector or viral-like particle, contain a first fusogen that is a protein fusogen and a second fusogen that is a lipid fusogen or chemical fusogen. In some embodiments, the fusogen binds a fusogen binding partner on a target cell surface. In some embodiments, the lipid particle is a viral vector or viral-like particle that is pseudotyped with the fusogen. In some examples, a virus of viral-like particle has a modification to one or more of its envelope proteins, e.g., an envelope protein is substituted with an envelope protein from another virus. In some embodiments, retroviral envelope proteins, e.g. lentiviral envelope proteins, are pseudotyped with a fusogen.

In some embodiments, the fusogen is a protein fusogen, e.g., a mammalian protein or a homologue of a mammalian protein (e.g., having 50%, 60%, 70%, 80%, 85%, 90%, 95%, 96%, 97%, 98%, 99%, or greater identity), a non-mammalian protein such as a viral protein or a homologue of a viral protein (e.g., having 50%, 60%, 70%, 80%, 85%, 90%, 95%, 96%, 97%, 98%, 99%, or greater identity), a native protein or a derivative of a native protein, a synthetic protein, a fragment thereof, a variant thereof, a protein fusion comprising one or more of the fusogens or fragments, and any combination thereof.

In some embodiments, the fusogen may include a mammalian protein. Examples of mammalian fusogens may include, but are not limited to, a SNARE family protein such as vSNAREs and tSNAREs, a syncytin protein such as Syncytin-1 (DOI: 10.1128/JVI.76.13.6442-6452.2002), and Syncytin-2, myomaker (biorxiv.org/content/early/2017/04/02/123158, doi.org/10.1101/123158, doi: 10.1096/fj.201600945R, doi:10.1038/nature12343), myomixer (www.nature.com/nature/journal/v499/n7458/full/nature12343.html, doi:10.1038/nature12343), myomerger (science.sciencemag.org/content/early/2017/04/05/science.aam9361, DOI: 10.1126/science.aam9361), FGFRL1 (fibroblast growth factor receptor-like 1), Minion (doi.org/10.1101/122697), an isoform of glyceraldehyde-3-phosphate dehydrogenase (GAPDH) (e.g., as disclosed in US 6,099,857A), a gap junction protein such as connexin 43, connexin 40, connexin 45, connexin 32 or connexin 37 (e.g., as disclosed in US 2007/0224176, Hap2, any protein capable of inducing syncytium formation between heterologous cells, any protein with fusogen properties, a homologue thereof, a fragment thereof, a variant thereof, and a protein fusion comprising one or more proteins or fragments thereof. In some embodiments, the fusogen is encoded by a human endogenous retroviral element (hERV) found in the human genome. Additional exemplary fusogens are disclosed in US 6,099,857A and US 2007/0224176, the entire contents of which are hereby incorporated by reference.

In some embodiments, the fusogen may include a non-mammalian protein, e.g., a viral protein. In some embodiments, a viral fusogen is a Class I viral membrane fusion protein, a Class II viral membrane protein, a Class III viral membrane fusion protein, a viral membrane glycoprotein, or other viral fusion proteins, or a homologue thereof, a fragment thereof, a variant thereof, or a protein fusion comprising one or more proteins or fragments thereof.

In some embodiments, Class I viral membrane fusion proteins include, but are not limited to, Baculovirus F protein, e.g., F proteins of the nucleopolyhedrovirus (NPV) genera, e.g., Spodoptera exigua MNPV (SeMNPV) F protein and Lymantria dispar MNPV (LdMNPV), and paramyxovirus F proteins.

In some embodiments, Class II viral membrane proteins include, but are not limited to, tick bone encephalitis E (TBEV E), Semliki Forest Virus E1/E2.

In some embodiments, Class III viral membrane fusion proteins include, but are not limited to, rhabdovirus G (e.g., fusogenic protein G of the Vesicular Stomatatis Virus (VSV-G)), herpesvirus glycoprotein B (e.g., Herpes Simplex virus 1 (HSV-1) gB)), Epstein Barr Virus glycoprotein B (EBV gB), thogotovirus G, baculovirus gp64 (e.g., Autographa California multiple NPV (AcMNPV) gp64), and Borna disease virus (BDV) glycoprotein (BDV G).

Examples of other viral fusogens, e.g., viral membrane glycoproteins and viral fusion proteins, include, but are not limited to: viral syncytia-forming proteins such as influenza hemagglutinin (HA) or mutants, or fusion proteins thereof; human immunodeficiency virus type 1 envelope protein (HIV-1 ENV), gp120 from HIV binding LFA-1 to form lymphocyte syncytium, HIV gp41, HIV gp160, or HIV Trans-Activator of Transcription (TAT); viral glycoprotein VSV-G, viral glycoprotein from vesicular stomatitis virus of the Rhabdoviridae family; glycoproteins gB and gH-gL of the varicella-zoster virus (VZV); murine leukaemia virus (MLV)-10A1; Gibbon Ape Leukemia Virus glycoprotein (GaLV); type G glycoproteins in Rabies, Mokola, vesicular stomatitis virus and Togaviruses; murine hepatitis virus JHM surface projection protein; porcine respiratory coronavirus spike- and membrane glycoproteins; avian infectious bronchitis spike glycoprotein and its precursor; bovine enteric coronavirus spike protein; the F and H, HN or G genes of Measles virus; canine distemper virus, Newcastle disease virus, human parainfluenza virus 3, simian virus 41, Sendai virus and human respiratory syncytial virus; gH of human herpesvirus 1 and simian varicella virus, with the chaperone protein gL; human, bovine and cercopithicine herpesvirus gB; envelope glycoproteins of Friend murine leukaemia virus and Mason Pfizer monkey virus; mumps virus hemagglutinin neuraminidase, and glyoproteins F1 and F2; membrane glycoproteins from Venezuelan equine encephalomyelitis; paramyxovirus F protein; SIV gp160 protein; Ebola virus G protein; or Sendai virus fusion protein, or a homologue thereof, a fragment thereof, a variant thereof, and a protein fusion comprising one or more proteins or fragments thereof.

Non-mammalian fusogens include viral fusogens, homologues thereof, fragments thereof, and fusion proteins comprising one or more proteins or fragments thereof. Viral fusogens include class I fusogens, class II fusogens, class III fusogens, and class IV fusogens. In embodiments, class I fusogens such as human immunodeficiency virus (HIV) gp41, have a characteristic postfusion conformation with a signature trimer of α-helical hairpins with a central coiled-coil structure. Class I viral fusion proteins include proteins having a central postfusion six-helix bundle. Class I viral fusion proteins include influenza HA, parainfluenza F, HIV Env, Ebola GP, hemagglutinins from orthomyxoviruses, F proteins from paramyxoviruses (e.g. Measles, (Katoh et al. BMC Biotechnology 2010, 10:37)), ENV proteins from retroviruses, and fusogens of filoviruses and coronaviruses. In embodiments, class II viral fusogens such as dengue E glycoprotein, have a structural signature of β- sheets forming an elongated ectodomain that refolds to result in a trimer of hairpins. In embodiments, the class II viral fusogen lacks the central coiled coil. Class II viral fusogen can be found in alphaviruses (e.g., E1 protein) and flaviviruses (e.g., E glycoproteins). Class II viral fusogens include fusogens from Semliki Forest virus, Sinbis, rubella virus, and dengue virus. In embodiments, class III viral fusogens such as the vesicular stomatitis virus G glycoprotein, combine structural signatures found in classes I and II. In embodiments, a class III viral fusogen comprises α helices (e.g., forming a six-helix bundle to fold back the protein as with class I viral fusogens), and β sheets with an amphiphilic fusion peptide at its end, reminiscent of class II viral fusogens. Class III viral fusogens can be found in rhabdoviruses and herpesviruses. In embodiments, class IV viral fusogens are fusion-associated small transmembrane (FAST) proteins (doi:10.1038/sj.emboj.7600767, Nesbitt, Rae L., “Targeted Intracellular Therapeutic Delivery Using Liposomes Formulated with Multifunctional FAST proteins” (2012). Electronic Thesis and Dissertation Repository. Paper 388), which are encoded by nonenveloped reoviruses. In embodiments, the class IV viral fusogens are sufficiently small that they do not form hairpins (doi: 10.1146/annurev-cellbio-101512-122422, doi:10.1016/j.devcel.2007.12.008).

Additional exemplary fusogens are disclosed in US 9,695,446, US 2004/0028687, US 6,416,997, US 7,329,807, US 2017/0112773, US 2009/0202622, WO 2006/027202, and US 2004/0009604, the entire contents of all of which are hereby incorporated by reference.

In some embodiments, the fusogen is a poxviridae fusogen.

In some embodiments the fusogen is a paramyxovirus fusogen. In some embodiments, the fusogen may be or an envelope glycoprotein G, H and/or an F protein of the Paramyxoviridae family. In some embodiments the fusogen contains a Nipah virus protein F, a measles virus F protein, a tupaia paramyxovirus F protein, a paramyxovirus F protein, a Hendra virus F protein, a Henipavirus F protein, a Morbilivirus F protein, a respirovirus F protein, a Sendai virus F protein, a rubulavirus F protein, or an avulavirus F protein. In some embodiments, the lipid particle includes contains a henipavirus envelope attachment glycoprotein G (G protein) or a biologically active portion thereof and/or a henipavirus envelope fusion glycoprotein F (F protein) or a biologically active portion thereof.

In particular embodiments, the fusogen is glycoprotein GP64 of baculovirus, or glycoprotein GP64 variant E45K/T259A.

In some embodiments, the fusogen is a hemagglutinin-neuraminidase (HN) and/or fusion (F) proteins (F/HN) from a respiratory paramyxovirus. In some embodiments, the respiratory paramyxovirus is a Sendai virus. The HN and F glycoproteins of Sendai viruses function to attach to sialic acids via the HN protein, and to mediate cell fusion for entry to cells via the F protein. In some embodiments, the sequence of the F protein is as set forth in SEQ ID. NO 88. In some embodiments, the F protein is truncated and lacks up to 42 contiguous amino acids, such as up to 42, 41, 40, 30, 38, 37, 36, 35, 34, 33, 32, 31, 30, 29, 28, 27, 26, 25, 24, 23, 22, 21, 20, 19, 18, 17, 16, 15, 14, 13, 12, 11, 10, 9, 8, 7, 6, 5, 4, 3, 2 or 1 contiguous amino acids at the C-terminus of SEQ ID. NO 88.

In some embodiments, the sequence of the HN protein is as set forth in SEQ ID. NO 89. In some embodiments, the HN protein is modified, such as by modification to the C-terminal domain. In some embodiments, the sequence of the HN protein is as set forth in SEQ ID. NO 90.

In some embodiments, the fusogen is a F and/or HN protein from the murine parainfluenza virus type 1 (See eg., US Pat. No. 10704061).

### A. G Proteins

In some embodiments the G protein is a Henipavirus G protein or a biologically active portion thereof. In some embodiments, the Henipavirus G protein is a Hendra (HeV) virus G protein, a Nipah (NiV) virus G-protein (NiV-G), a Cedar virus G-protein(CedPV), a Mojiang virus G-protein, a bat Paramyxovirus G-protein, or a biologically active portion thereof. A non-limited list of exemplary G proteins is shown in Table 3.

The attachment G proteins are type II transmembrane glycoproteins containing an N-terminal cytoplasmic tail (e.g. corresponding to amino acids 1-49 of SEQ ID NO:44), a transmembrane domain (e.g. corresponding to amino acids 50-70 of SEQ ID NO:44, and an extracellular domain containing an extracellular stalk (e.g. corresponding to amino acids 71-187 of SEQ ID NO:44), and a globular head (corresponding to amino acids 188-602 of SEQ ID NO:44). The N-terminal cytoplasmic domain is within the inner lumen of the lipid bilayer and the C-terminal portion is the extracellular domain that is exposed on the outside of the lipid bilayer. Regions of the stalk in the C-terminal region (e.g. corresponding to amino acids 159-167 of NiV-G) have been shown to be involved in interactions with F protein and triggering of F protein fusion (Liu et al. 2015 J of Virology 89:1838). In wild-type G protein, the globular head mediates receptor binding to henipavirus entry receptors eprhin B2 and ephrin B3, but is dispensable for membrane fusion (Brandel-Tretheway et al. Journal of Virology. 2019. 93(13)e00577-19).

In particular embodiments herein, tropism of the G protein is modified. Binding of the G protein to a binding partner can trigger fusion mediated by a compatible F protein or biologically active portion thereof. G protein sequences disclosed herein are predominantly disclosed as expressed sequences including an N-terminal methionine required for start of translation. As such N-terminal methionines are commonly cleaved co- or post-translationally, the mature protein sequences for all G protein sequences disclosed herein are also contemplated as lacking the N-terminal methionine.

G glycoproteins are highly conserved between henipavirus species. For example, the G protein of NiV and HeV viruses share 79% amino acids identity. Studies have shown a high degree of compatibility among G proteins with F proteins of different species as demonstrated by heterotypic fusion activation (Brandel-Tretheway et al. Journal of Virology. 2019). As described below, a re-targeted lipid particle can contain heterologous proteins from different species.

Table 3

Exemplary Henipavirus G Proteins

| Viral G Protein | Sequence | SEQ ID NO | SEQ ID NO (without N-terminal methionine) |
|---|---|---|---|
| Hendra Virus G Protein | MMADSKLVSLNNNLSGKIKDQGKVIKNYYGTMDIKKINDGLLDSKILGAFNTVIALLGSIIIIVMNIMIIQNYTRTTDNQALIKESLQSVQQQIKALTDKIGTEIGPKVSLIDTSSTITIPANIGLLGSKISQSTSSINENVNDKCKFTLPPLKIHECNISCPNPLPFREYRPISQGVSDLVGLPNQICLQKTTSTILKPRLISYTLPINTREGVCITDPLLAVDNGFFAYSHLEKIGSCTRGIAKQRIIGVGEVLDRGDKVPSMFMTNVWTPPNPSTIHHCSSTYHEDFYYTLCAVSHVGDPILNSTSWTESLSLIRLAVRPKSDSGDYNQKYIAITKVERGKYDKVMPYGPSGIKQGDTLYFPAVGFLPRTEFQYNDSNCPIIHCKYSKAENCRLSMGVNSKSHYILRSGLLKYNLSLGGDIILQFIEIADNRLTIGSPSKIYNSLGQPVFYQASYSWDTMIKLGDVDTVDPLRVQWRNNSVISRPGQSQCPRFNVCPEVCWEGTYNDAFLIDRLNWVSAGVYLNSNQTAENPVFAVFKDNEILYQVPLAEDDTNAQKTITDCFLLENVIWCISLVEIYDTGDSVIRPKLFAVKIPAQCSES | 24 | 25 |
| Nipah Virus G Protein | MPAENKKVRFENTTSDKGKIPSKVIKSYYGTMDIKKIN EGLLDSKILSAFNTVIALLGSIVIIVMNIMIIQNYTRSTDNQAVIKDALQGIQQQIKGLADKIGTEIGPKVSLIDTSSTITIPANIGLLGSKISQSTASINENVNEKCKFTLPPLKIHECNISCPNPLPFREYRPQTEGVSNLVGLPNNICLQKTSNQILKPKLISYTLPVVGQSGTCITDPLLAMDEGYFAYSHLERIGSCSRGVSKQRIIGVGEVLDRGDEVPSLFMTNVWTPPNPNTVYHCSAVYNNEFYYVLCAVSTVGDPILNSTYWSGSLMMTRLAVKPKSNGGGYNQHQLALRSIEKGRYDKVMPYGPSGIKQGDTLYFPAVGFLVRTEFKYNDSNCPITKCQYSKPENCRLSMGIRPNSHYILRSGLLKYNLSDGENPKVVFIEISDQRLSIGSPSKIYDSLGQPVFYQASFSWDTMIKFGDVLTVNPLVVNWRNNTVISRPGQSQCPRFNTCPEICWEGVYNDAFLIDRINWISAGVFLDSNQTAENPVFTVFKDNEILYRAQLASEDTNAQKTITNCFLLKNKIWCISLVEIYDTGDNVIRPKLFAVKIPEQCT | 26 | 27 |
| Cedar Virus G Protein | MLSQLQKNYLDNSNQQGDKMNNPDKKLSVNFNPLEL DKGQKDLNKSYYVKNKNYNVSNLLNESLHDIKFCIYC IFSLLIIITIINIITISIVITRLKVHEENNGMESPNLQSIQDSLSSLTNMINTEITPRIGILVTATSVTLSSSINYVGTKTNQLVNELKDYITKSCGFKVPELKLHECNISCADPKISKSAMYSTNAYAELAGPPKIFCKSVSKDPDFRLKQIDYVIPVQQDRSICMNNPLLDISDGFFTYIHYEGINSCKKSDSFKVLLSHGEIVDRGDYRPSLYLLSSHYHPYSMQVINCVPVTCNQSSFVFCHISNNTKTLDNSDYSSDEYYITYFNGIDRPKTKKIPINNMTADNRYIHFTFSGGGGVCLGEEFIIPVTTVINTDVFTHDYCESFNCSVQTGKSLKEICSESLRSPTNSSRYNLNGIMIISQNNMTDFKIQLNGITYNKLSFGSPGRLSKTLGQVLYYQSSMSWDTYLKAGFVEKWKPFTPNWMNNTVISRPNQGNCPRYHKCPEICYGGTYNDIAPLDLGKDMYVSVILDSDQLAENPEITVFNSTTILYKERVSKDELNTRSTTTSCFLFLDEPWCISVLETNRFNGKSIRPEIYSYKIPKYC | 28 | 29 |
| Bat Paramyxovirus G Protein, Eid_hel/GH-M74a/GHA/2009 | MPQKTVEFINMNSPLERGVSTLSDKKTLNQSKITKQG YFGLGSHSERNWKKQKNQNDHYMTVSTMILEILVVL GIMFNLIVLTMVYYQNDNINQRMAELTSNITVLNLNL NQLTNKIQREIIPRITLIDTATTITIPSAITYILATLTTRISELLPSINQKCEFKTPTLVLNDCRINCTPPLNPSDGVKMSSLATNLVAHGPSPCRNFSSVPTIYYYRIPGLYNRTALDERCILNPRLTISSTKFAYVHSEYDKNCTRGFKYYELMTFGEILEGPEKEPRMFSRSFYSPTNAVNYHSCTPIVTVNEGYFLCLECTSSDPLYKANLSNSTFHLVILRHNKDEKIVSMPSFNLSTDQEYVQIIPAEGGGTAESGNLYFPCIGRLLHKRVTHPLCKKSNCSRTDDESCLKSYYNQGSPQHQVVNCLIRIRNAQRDNPTWDVITVDLTNTYPGSRSRIFGSFSKPMLYQSSVSWHTLLQVAEITDLDKYQLDWLDTPYISRPGGSECPFGNYCPTVCWEGTYNDVYSLTPNNDLFVTVYLKSEQVAENPYFAIFSRDQILKEFPLDAWISSARTTTISCFMFNNEIWCIAALEITRLNDDIIRPIYYSFWLPTDCRTPYPHTGKMTRVPLRSTYNY | 30 | 31 |
| Mojiang virus, Tongguan 1 G Protein | MATNRDNTITSAEVSQEDKVKKYYGVETAEKVADSIS GNKVFILMNTLLILTGAIITITLNITNLTAAKSQQNMLKIIQDDVNAKLEMFVNLDQLVKGEIKPKVSLINTAVSVSIPGQISNLQTKFLQKYVYLEESITKQCTCNPLSGIFPTSGPTYPPTDKPDDDTTDDDKVDTTIKPIEYPKPDGCNRTGDHFTMEPGANFYTVPNLGPASSNSDECYTNPSFSIGSSIYMFSQEIRKTDCTAGEILSIQIVLGRIVDKGQQGPQASPLLVWAVPNPKIINSCAVAAGDEMGWVLCSVTLTAASGEPIPHMFDGFWLYKLEPDTEVVSYRITGYAYLLDKQ YDSVFIGKGGGIQKGNDLYFQMYGLSRNRQSFKALCE HGSCLGTGGGGYQVLCDRAVMSFGSEESLITNAYLKV NDLASGKPVIIGQTFPPSDSYKGSNGRMYTIGDKYGLY LAPSSWNRYLRFGITPDISVRSTTWLKSQDPIMKILSTCTNTDRDMCPEICNTRGYQDIFPLSEDSEYYTYIGITPNNGGTKNFVAVRDSDGHIASIDILQNYYSITSATISCFMYKDEIWCIAITEGKKQKDNPQRIYAHSYKIRQMCYNMKSATVTVGNAKNITIRRY | 32 | 33 |

In some embodiments, the G protein has a sequence set forth in any of SEQ ID NOS: 44, 24, 26, 30, 32, 27, 25, 29, 31, or 33 or is a functionally active variant or biologically active portion thereof that has a sequence that is at least at or about 80%, at least at or about 81%, at least at or about 82%, at least at or about 83%, at least at or about 84%, at least at or about 85%, at least at or about 86%, at least at or about 87%, at least at or about 88%, at least at or about 89%, at least at or about 90%, at least at or about 91%, at least at or about 92%, at least at or about 93%, at least at or about 94%, at least at or about 95%, at least at or about 96%, at least at or about 97%, at least at or about 98%, or at least at or about 99% identical to any one of SEQ ID NOS: 44, 24, 26, 30, 32, 27, 25, 29, 31, or 33

In particular embodiments, the G protein or functionally active variant or biologically active portion is a protein that retains fusogenic activity in conjunction with a Henipavirus F protein, e.g. NiV-F or HeV-F. Fusogenic activity includes the activity of the G protein in conjunction with a Henipavirus F protein to promote or facilitate fusion of two membrane lumens, such as the lumen of the targeted lipid particle having embedded in its lipid bilayer a henipavirus F and G protein, and a cytoplasm of a target cell, e.g. a cell that contains a surface receptor or molecule that is recognized or bound by the targeted envelope protein. In some embodiments, the F protein and G protein are from the same Henipavirus species (e.g. NiV-G and NiV-F, or HeV-G and HeV-F). In some embodiments, the F protein and G protein are from different Henipavirus species (e.g. NiV-G and HeV-F, or HeV-G and NiV-F).

In particular embodiments, the G protein has the sequence of amino acids set forth in SEQ ID NO: 44, SEQ ID NO: 24, SEQ ID NO: 26, SEQ ID NO: 30, SEQ ID NO: 32, SEQ ID NO: 27, SEQ ID NO: 25, SEQ ID NO: 29, SEQ ID NO: 30 or SEQ ID NO: 31 or is a functionally active variant thereof or a biologically active portion thereof that retains fusogenic activity. In some embodiments, the functionally active variant comprises an amino acid sequence having at least at or about 80%, at least at or about 85%, at least at or about 90%, at least at or about 91%, at least at or about 92%, at least at or about 93%, at least at or about 94%, at least at or about 95%, at or about 96%, at least at or about 97%, at least at or about 98%, or at least at or about 99% sequence identity to SEQ ID NO: 44, SEQ ID NO: 24, SEQ ID NO: 26, SEQ ID NO: 30, SEQ ID NO: 32, SEQ ID NO: 27, SEQ ID NO: 25, SEQ ID NO: 29, SEQ ID NO: 30 or SEQ ID NO: 31 and retains fusogenic activity in conjunction with a Henipavirus F protein (e.g., NiV-F or HeV-F). In some embodiments, the biologically active portion has an amino acid sequence having at least at or about 80%, at least at or about 85%, at least at or about 90%, at least at or about 91%, at least at or about 92%, at least at or about 93%, at least at or about 94%, at least at or about 95%, at or about 96%, at least at or about 97%, at least at or about 98%, or at least at or about 99% sequence identity to SEQ ID NO: 44, SEQ ID NO: 24, SEQ ID NO: 26, SEQ ID NO: 30, SEQ ID NO: 32, SEQ ID NO: 27, SEQ ID NO: 25, SEQ ID NO: 29, SEQ ID NO: 30 or SEQ ID NO: 31 and retains fusogenic activity in conjunction with a Henipavirus F protein (e.g., NiV-F or HeV-F).

Reference to retaining fusogenic activity includes activity (in conjunction with a Henipavirus F protein) that is between at or about 10% and at or about 150% or more of the level or degree of binding of the corresponding wild-type G protein, such as set forth in SEQ ID NO: 44, SEQ ID NO: 24, SEQ ID NO: 26, SEQ ID NO: 30, SEQ ID NO: 32, SEQ ID NO: 27, SEQ ID NO: 25, SEQ ID NO: 29, SEQ ID NO: 30 or SEQ ID NO: 31 such as at least or at least about 10% of the level or degree of fusogenic activity of the corresponding wild-type G protein, such as at least or at least about 15% of the level or degree of fusogenic activity of the corresponding wild-type G protein, such as at least or at least about 20% of the level or degree of fusogenic activity of the corresponding wild-type G protein, such as at least or at least about 25% of the level or degree of fusogenic activity of the corresponding wild-type G protein, such as at least or at least about 30% of the level or degree of fusogenic activity of the corresponding wild-type G protein, such as at least or at least about 35% of the level or degree of fusogenic activity of the corresponding wild-type G protein, such as at least or at least about 40% of the level or degree of fusogenic activity of the corresponding wild-type G protein, such as at least or at least about 45% of the level or degree of fusogenic activity of the corresponding wild-type G protein, such as at least or at least about 50% of the level or degree of fusogenic activity of the corresponding wild-type G protein, such as at least or at least about 55% of the level or degree of fusogenic activity of the corresponding wild-type G protein, such as at least or at least about 60% of the level or degree of fusogenic activity of the corresponding wild-type G protein, such as at least or at least about 65% of the level or degree of fusogenic activity of the corresponding wild-type G protein, such as at least or at least about 70% of the level or degree of fusogenic activity of the corresponding wild-type G protein, such as at least or at least about 75% of the level or degree of fusogenic activity of the corresponding wild-type G protein, such as at least or at least about 80% of the level or degree of fusogenic activity of the corresponding wild-type G protein, such as at least or at least about 85% of the level or degree of fusogenic activity of the corresponding wild-type G protein, such as at least or at least about 90% of the level or degree of fusogenic activity of the corresponding wild-type G protein, such as at least or at least about 95% of the level or degree of fusogenic activity of the corresponding wild-type G protein, such as at least or at least about 100% of the level or degree of fusogenic activity of the corresponding wild-type G protein, such as at least or at least about 120% of the level or degree of fusogenic activity of the corresponding wild-type G protein, such as at least or at least about 130% of the level or degree of fusogenic activity of the corresponding wild-type G protein, such as at least or at least about 140% of the level or degree of fusogenic activity of the corresponding wild-type G protein, or such as at least or at least about 150% of the level or degree of fusogenic activity of the corresponding wild-type G protein.

In some embodiments the G protein is a mutant G protein that is a functionally active variant or biologically active portion containing one or more amino acid mutations, such as one or more amino acid insertions, deletions, substitutions or truncations. In some embodiments, the mutations described herein relate to amino acid insertions, deletions, substitutions or truncations of amino acids compared to a reference G protein sequence. In some embodiments, the reference G protein sequence is the wild-type sequence of a G protein or a biologically active portion thereof. In some embodiments, the functionally active variant or the biologically active portion thereof is a mutant of a wild-type Hendra (HeV) virus G protein, a wild-type Nipah (NiV) virus G-protein (NiV-G), a wild-type Cedar (CedPV) virus G-protein, a wild-type Mojiang virus G-protein, a wild-type bat Paramyxovirus G-protein or biologically active portion thereof. In some embodiments, the wild-type G protein has the sequence set forth in any one of SEQ ID NO: 44, SEQ ID NO: 24, SEQ ID NO: 26, SEQ ID NO: 30, SEQ ID NO: 32, SEQ ID NO: 27, SEQ ID NO: 25, SEQ ID NO: 29, SEQ ID NO: 30 or SEQ ID NO: 31.

In some embodiments, the G protein is a mutant G protein that is a biologically active portion that is an N-terminally and/or C-terminally truncated fragment of a wild-type Hendra (HeV) virus G protein, a wild-type Nipah (NiV) virus G-protein (NiV-G), a wild-type Cedar (CedPV) virus G-protein, a wild-type Mojiang virus G-protein, a wild-type bat Paramyxovirus G-protein. In particular embodiments, the truncation is an N-terminal truncation of all or a portion of the cytoplasmic domain. In some embodiments, the mutant G protein is a biologically active portion that is truncated and lacks up to 49 contiguous amino acid residues at or near the N-terminus of the wild-type G protein, such as a wild-type G protein set forth in any one of SEQ ID NO: 44, SEQ ID NO: 24, SEQ ID NO: 26, SEQ ID NO: 30, SEQ ID NO: 32, SEQ ID NO: 27, SEQ ID NO: 25, SEQ ID NO: 29, SEQ ID NO: 30 or SEQ ID NO: 31. In some embodiments, the mutant F protein is truncated and lacks up to 49 contiguous amino acids, such as up to 49, 48, 47, 46, 45, 44, 43, 42, 41, 40, 30, 38, 37, 36, 35, 34, 33, 32, 31, 30, 29, 28, 27, 26, 25, 24, 23, 22, 21, 20, 19, 18, 17, 16, 15, 14, 13, 12, 11, 10, 9, 8, 7, 6, 5, 4, 3, 2 or 1 contiguous amino acids at the N-terminus of the wild-type G protein.

In some embodiments, the G protein is a wild-type Nipah virus G (NiV-G) protein or a Hendra virus G protein, or is a functionally active variant or biologically active portion thereof.In some embodiments, the G protein is a NiV-G protein that has the sequence set forth inSEQ ID NO:44, SEQ ID NO:26 or SEQ ID NO:27, or is a functional variant or a biologically active portion thereof that has an amino acid sequence having at least at or about 80%, at least at or about 81%, at least at or about 82%, at least at or about 83%, at least at or about 84%, at least at or about 85%, at least at or about 86%, at least at or about 87%, at least at or about 88%, at least at or about 89%, at least at or about 90%, at least at or about 91%, at least at or about 92%, at least at or about 93%, at least at or about 94%, at least at or about 95%, at or about 96%, at least at or about 97%, at least at or about 98%, at least at or about 99% sequence identity to SEQ ID NO:44, SEQ ID NO:26 or SEQ ID NO:27.

In some embodiments, the G protein is a mutant NiV-G protein that is a biologically active portion of a wild-type NiV-G. In some embodiments, the biologically active portion is an N-terminally truncated fragment. In some embodiments, the mutant NiV-G protein is truncated and lacks up to 5 contiguous amino acid residues at or near the N-terminus of the wild-type NiV-G protein (SEQ ID NO:44, SEQ ID NO:26 or SEQ ID NO:27), up to 6 contiguous amino acid residues at or near the N-terminus of the wild-type NiV-G protein (SEQ ID NO:44, SEQ ID NO:26 or SEQ ID NO:27), up to 7 contiguous amino acid residues at or near the N-terminus of the wild-type NiV-G protein (SEQ ID NO:9, SEQ ID NO:26 or SEQ ID NO:27), up to 8 contiguous amino acid residues at or near the N-terminus of the wild-type NiV-G protein (SEQ ID NO:9, SEQ ID NO:26 or SEQ ID NO:27), up to 9 contiguous amino acid residues at or near the N-terminus of the wild-type NiV-G protein (SEQ ID NO:9, SEQ ID NO:26 or SEQ ID NO:27) up to 10 contiguous amino acid residues at or near the N-terminus of the wild-type NiV-G protein (SEQ ID NO:9, SEQ ID NO:26 or SEQ ID NO:27), up to 11 contiguous amino acid residues at or near the N-terminus of the wild-type NiV-G protein (SEQ ID NO:9, SEQ ID NO:26 or SEQ ID NO:27), up to 12 contiguous amino acid residues at or near the N-terminus of the wild-type NiV-G protein SEQ ID NO:9, SEQ ID NO:26 or SEQ ID NO:27), up to 13 contiguous amino acid residues at or near the N-terminus of the wild-type NiV-G protein (SEQ ID NO:9, SEQ ID NO:26 or SEQ ID NO:27), up to 14 contiguous amino acid residues at or near the N-terminus of the wild-type NiV-G protein (SEQ ID NO:9, SEQ ID NO:26 or SEQ ID NO:27), up to 15 contiguous amino acid residues at or near the N-terminus of the wild-type NiV-G protein (SEQ ID NO:9, SEQ ID NO:26 or SEQ ID NO:27) up to 16 contiguous amino acid residues at or near the N-terminus of the wild-type NiV-G protein (SEQ ID NO:9, SEQ ID NO:26 or SEQ ID NO:27), up to 17 contiguous amino acid residues at or near the N-terminus of the wild-type NiV-G protein (SEQ ID NO:9, SEQ ID NO:26 or SEQ ID NO:27), up to 18 contiguous amino acid residues at or near the N-terminus of the wild-type NiV-G protein (SEQ ID NO:9, SEQ ID NO:26 or SEQ ID NO:27), up to 19 contiguous amino acid residues at or near the N-terminus of the wild-type NiV-G protein (SEQ ID NO:9, SEQ ID NO:26 or SEQ ID NO:27), up to 20 contiguous amino acid residues at or near the N-terminus of the wild-type NiV-G protein (SEQ ID NO:9, SEQ ID NO:26 or SEQ ID NO:27), up to 21 contiguous amino acid residues at or near the N-terminus of the wild-type NiV-G protein (SEQ ID NO:9, SEQ ID NO:26 or SEQ ID NO:27), up to 22 contiguous amino acid residues at or near the N-terminus of the wild-type NiV-G protein (SEQ ID NO:9, SEQ ID NO:26 or SEQ ID NO:27), up to 23 contiguous amino acid residues at or near the N-terminus of the wild-type NiV-G protein (SEQ ID NO:9, SEQ ID NO:26 or SEQ ID NO:27), up to 24 contiguous amino acid residues at or near the N-terminus of the wild-type NiV-G protein (SEQ ID NO:9, SEQ ID NO:26 or SEQ ID NO:27), up to 25 contiguous amino acid residues at or near the N-terminus of the wild-type NiV-G protein (SEQ ID NO:9, SEQ ID NO:26 or SEQ ID NO:27), up to 26 contiguous amino acid residues at or near the N-terminus of the wild-type NiV-G protein (SEQ ID NO:9, SEQ ID NO:26 or SEQ ID NO:27), up to 27 contiguous amino acid residues at or near the N-terminus of the wild-type NiV-G protein (SEQ ID NO:9, SEQ ID NO:26 or SEQ ID NO:27), up to 28 contiguous amino acid residues at or near the N-terminus of the wild-type NiV-G protein (SEQ ID NO:9, SEQ ID NO:26 or SEQ ID NO:27), up to 29 contiguous amino acid residues at or near the N-terminus of the wild-type NiV-G protein (SEQ ID NO:9, SEQ ID NO:26 or SEQ ID NO:27), up to 30 contiguous amino acid residues at or near the N-terminus of the wild-type NiV-G protein (SEQ ID NO:9, SEQ ID NO:26 or SEQ ID NO:27), up to 31 contiguous amino acid residues at or near the N-terminus of the wild-type NiV-G protein (SEQ ID NO:9, SEQ ID NO:26 or SEQ ID NO:27), up to 32 contiguous amino acid residues at or near the N-terminus of the wild-type NiV-G protein (SEQ ID NO:9, SEQ ID NO:26 or SEQ ID NO:27), up to 33 contiguous amino acid residues at or near the N-terminus of the wild-type NiV-G protein (SEQ ID NO:9, SEQ ID NO:26 or SEQ ID NO:27), up to 34 contiguous amino acid residues at or near the N-terminus of the wild-type NiV-G protein (SEQ ID NO:9, SEQ ID NO:26 or SEQ ID NO:27), up to 35 contiguous amino acid residues at or near the N-terminus of the wild-type NiV-G protein (SEQ ID NO:9, SEQ ID NO:26 or SEQ ID NO:27), up to 36 contiguous amino acid residues at or near the N-terminus of the wild-type NiV-G protein (SEQ ID NO:9, SEQ ID NO:26 or SEQ ID NO:27), up to 37 contiguous amino acid residues at or near the N-terminus of the wild-type NiV-G protein (SEQ ID NO:9, SEQ ID NO:26 or SEQ ID NO:27up to 38 contiguous amino acid residues at or near the N-terminus of the wild-type NiV-G protein (SEQ ID NO:9, SEQ ID NO:26 or SEQ ID NO:27), up to 39 contiguous amino acid residues at or near the N-terminus of the wild-type NiV-G protein (SEQ ID NO:9, SEQ ID NO:26 or SEQ ID NO:27), up to 40 contiguous amino acid residues at or near the N-terminus of the wild-type NiV-G protein (SEQ ID NO:9, SEQ ID NO:26 or SEQ ID NO:27), up to 41 contiguous amino acid residues at or near the N-terminus of the wild-type NiV-G protein (SEQ ID NO:9, SEQ ID NO:26 or SEQ ID NO:27), up to 42 contiguous amino acid residues at or near the N-terminus of the wild-type NiV-G protein (SEQ ID NO:9, SEQ ID NO:26 or SEQ ID NO:27), up to 43 contiguous amino acid residues at or near the N-terminus of the wild-type NiV-G protein (SEQ ID NO:9, SEQ ID NO:26 or SEQ ID NO:27), up to 44 contiguous amino acid residues at or near the N-terminus of the wild-type NiV-G protein (SEQ ID NO:9, SEQ ID NO:26 or SEQ ID NO:27), or up to 45 contiguous amino acid residues at or near the N-terminus of the wild-type NiV-G protein (SEQ ID NO:9, SEQ ID NO:26 or SEQ ID NO:27).

In some embodiments, the NiV-G protein is a biologically active portion that does not contain a cytoplasmic domain. In some embodiments, the NiV-G protein without the cytoplasmic domain is encoded by SEQ ID NO: 45.

In some embodiments, the mutant NiV-G protein comprises a sequence set forth in any of SEQ ID NOS: 45-65, or is a functional variant thereof that has an amino acid sequence having at least at or 80%, at least at or about 81%, at least at or about 82%, at least at or about 83%, at or about 84%, at least at or about 85%, at least at or about 86%, or at least at or about 87%, at least at or about 88%, or at least at or about 89%, about 90%, at least at or about 91%, at least at or about 92%, at least at or about 93%, at least at or about 94%, at least at or about 95%, at or about 96%, at least at or about 97%, at least at or about 98%, or at least at or about 99% sequence identity to SEQ ID NOS: 45-65.

In some embodiments, the mutant NiV-G protein has a 5 amino acid truncation at or near the N-terminus of the wild-type NiV-G protein (SEQ ID NO:27, SEQ ID NO:26 or SEQ ID NO:44), such as set forth in SEQ ID NO: 46 or a functional variant thereof having at least at or about 80%, at least at or about 81%, at least at or about 82%, at least at or about 83%, at or about 84%, at least at or about 85%, at least at or about 86%, or at least at or about 87%, at least at or about 88%, or at least at or about 89%, at least at or about 90%, at least at or about 91%, at least at or about 92%, at least at or about 93%, at least at or about 94%, at least at or about 95%, at or about 96%, at least at or about 97%, at least at or about 98%, or at least at or about 99% sequence identity to SEQ ID NO:46 or such as set forth in SEQ ID NO: 52 or a functional variant thereof having at least at or about 80%, at least at or about 81%, at least at or about 82%, at least at or about 83%, at or about 84%, at least at or about 85%, at least at or about 86%, or at least at or about 87%, at least at or about 88%, or at least at or about 89%, at least at or about 90%, at least at or about 91%, at least at or about 92%, at least at or about 93%, at least at or about 94%, at least at or about 95%, at or about 96%, at least at or about 97%, at least at or about 98%, or at least at or about 99% sequence identity to SEQ ID NO:52 or such as set forth in SEQ ID NO: 58 or a functional variant thereof having at least at or about 80%, at least at or about 81%, at least at or about 82%, at least at or about 83%, at or about 84%, at least at or about 85%, at least at or about 86%, or at least at or about 87%, at least at or about 88%, or at least at or about 89%, at least at or about 90%, at least at or about 91%, at least at or about 92%, at least at or about 93%, at least at or about 94%, at least at or about 95%, at or about 96%, at least at or about 97%, at least at or about 98%, or at least at or about 99% sequence identity to SEQ ID NO:58. In some embodiments, the mutant NiV-G protein has a 10 amino acid truncation at or near the N-terminus of the wild-type NiV-G protein (SEQ ID NO:26, SEQ ID NO:27 or SEQ ID NO:44), such as set forth in SEQ ID NO: 47 or a functional variant thereof having at least at or about 80%, at least at or about 81%, at least at or about 82%, at least at or about 83%, at or about 84%, at least at or about 85%, at least at or about 86%, or at least at or about 87%, at least at or about 88%, or at least at or about 89%, at least at or about 90%, at least at or about 91%, at least at or about 92%, at least at or about 93%, at least at or about 94%, at least at or about 95%, at or about 96%, at least at or about 97%, at least at or about 98%, or at least at or about 99% sequence identity to SEQ ID NO:47, or such as set forth in SEQ ID NO: 53 or a functional variant thereof having at least at or about 80%, at least at or about 81%, at least at or about 82%, at least at or about 83%, at or about 84%, at least at or about 85%, at least at or about 86%, or at least at or about 87%, at least at or about 88%, or at least at or about 89%, at least at or about 90%, at least at or about 91%, at least at or about 92%, at least at or about 93%, at least at or about 94%, at least at or about 95%, at or about 96%, at least at or about 97%, at least at or about 98%, or at least at or about 99% sequence identity to SEQ ID NO:53 or such as set forth in SEQ ID NO: 59 or a functional variant thereof having at least at or about 80%, at least at or about 81%, at least at or about 82%, at least at or about 83%, at or about 84%, at least at or about 85%, at least at or about 86%, or at least at or about 87%, at least at or about 88%, or at least at or about 89%, at least at or about 90%, at least at or about 91%, at least at or about 92%, at least at or about 93%, at least at or about 94%, at least at or about 95%, at or about 96%, at least at or about 97%, at least at or about 98%, or at least at or about 99% sequence identity to SEQ ID NO:59.

In some embodiments, the mutant NiV-G protein has a 15 amino acid truncation at or near the N-terminus of the wild-type NiV-G protein (SEQ ID NO:26, SEQ ID NO:27 or SEQ ID NO:44), such as set forth in SEQ ID NO: 48 or a functional variant thereof that has an amino acid sequence having at least at or about 80%, at least at or about 81%, at least at or about 82%, at least at or about 83%, at or about 84%, at least at or about 85%, at least at or about 86%, or at least at or about 87%, at least at or about 88%, or at least at or about 89%, at least at or about 90%, at least at or about 91%, at least at or about 92%, at least at or about 93%, at least at or about 94%, at least at or about 95%, at or about 96%, at least at or about 97%, at least at or about 98%, or at least at or about 99% sequence identity to SEQ ID NO:48 or such as set forth in SEQ ID NO: 54 or a functional variant thereof having at least at or about 80%, at least at or about 81%, at least at or about 82%, at least at or about 83%, at or about 84%, at least at or about 85%, at least at or about 86%, or at least at or about 87%, at least at or about 88%, or at least at or about 89%, at least at or about 90%, at least at or about 91%, at least at or about 92%, at least at or about 93%, at least at or about 94%, at least at or about 95%, at or about 96%, at least at or about 97%, at least at or about 98%, or at least at or about 99% sequence identity to SEQ ID NO:54 or such as set forth in SEQ ID NO: 60 or a functional variant thereof having at least at or about 80%, at least at or about 81%, at least at or about 82%, at least at or about 83%, at or about 84%, at least at or about 85%, at least at or about 86%, or at least at or about 87%, at least at or about 88%, or at least at or about 89%, at least at or about 90%, at least at or about 91%, at least at or about 92%, at least at or about 93%, at least at or about 94%, at least at or about 95%, at or about 96%, at least at or about 97%, at least at or about 98%, or at least at or about 99% sequence identity to SEQ ID NO:60. In some embodiments, the mutant NiV-G protein has a 20 amino acid truncation at or near the N-terminus of the wild-type NiV-G protein (SEQ ID NO:26, SEQ ID NO:27 or SEQ ID NO:44) such as set forth in SEQ ID NO: 49, or a functional variant thereof having at least at or about 80%, at least at or about 81%, at least at or about 82%, at least at or about 83%, at or about 84%, at least at or about 85%, at least at or about 86%, or at least at or about 87%, at least at or about 88%, or at least at or about 89%, at least at or about 90%, at least at or about 91%, at least at or about 92%, at least at or about 93%, at least at or about 94%, at least at or about 95%, at or about 96%, at least at or about 97%, at least at or about 98%, or at least at or about 99% sequence identity to SEQ ID NO:49 or such as set forth in SEQ ID NO: 55 or a functional variant thereof having at least at or about 80%, at least at or about 81%, at least at or about 82%, at least at or about 83%, at or about 84%, at least at or about 85%, at least at or about 86%, or at least at or about 87%, at least at or about 88%, or at least at or about 89%, at least at or about 90%, at least at or about 91%, at least at or about 92%, at least at or about 93%, at least at or about 94%, at least at or about 95%, at or about 96%, at least at or about 97%, at least at or about 98%, or at least at or about 99% sequence identity to SEQ ID NO: or such as set 55forth in SEQ ID NO: 61 or a functional variant thereof having at least at or about 80%, at least at or about 81%, at least at or about 82%, at least at or about 83%, at or about 84%, at least at or about 85%, at least at or about 86%, or at least at or about 87%, at least at or about 88%, or at least at or about 89%, at least at or about 90%, at least at or about 91%, at least at or about 92%, at least at or about 93%, at least at or about 94%, at least at or about 95%, at or about 96%, at least at or about 97%, at least at or about 98%, or at least at or about 99% sequence identity to SEQ ID NO:61. In some embodiments, the mutant NiV-G protein has a 25 amino acid truncation at or near the N-terminus of the wild-type NiV-G protein (SEQ ID NO:26, SEQ ID NO:27 or SEQ ID NO:44), such as set forth in SEQ ID NO: 50 or a functional variant thereof having at least at or about 80%, at least at or about 81%, at least at or about 82%, at least at or about 83%, at or about 84%, at least at or about 85%, at least at or about 86%, or at least at or about 87%, at least at or about 88%, or at least at or about 89%, at least at or about 90%, at least at or about 91%, at least at or about 92%, at least at or about 93%, at least at or about 94%, at least at or about 95%, at or about 96%, at least at or about 97%, at least at or about 98%, or at least at or about 99% sequence identity to SEQ ID NO:50 or such as set forth in SEQ ID NO: 56 or a functional variant thereof having at least at or about 80%, at least at or about 81%, at least at or about 82%, at least at or about 83%, at or about 84%, at least at or about 85%, at least at or about 86%, or at least at or about 87%, at least at or about 88%, or at least at or about 89%, at least at or about 90%, at least at or about 91%, at least at or about 92%, at least at or about 93%, at least at or about 94%, at least at or about 95%, at or about 96%, at least at or about 97%, at least at or about 98%, or at least at or about 99% sequence identity to SEQ ID NO:56 or such as set forth in SEQ ID NO: 62 or a functional variant thereof having at least at or about 80%, at least at or about 81%, at least at or about 82%, at least at or about 83%, at or about 84%, at least at or about 85%, at least at or about 86%, or at least at or about 87%, at least at or about 88%, or at least at or about 89%, at least at or about 90%, at least at or about 91%, at least at or about 92%, at least at or about 93%, at least at or about 94%, at least at or about 95%, at or about 96%, at least at or about 97%, at least at or about 98%, or at least at or about 99% sequence identity to SEQ ID NO:62. In some embodiments, the mutant NiV-G protein has a 30 amino acid truncation at or near the N-terminus of the wild-type NiV-G protein (SEQ ID NO:26, SEQ ID NO:27 or SEQ ID NO:44), such as set forth in SEQ ID NO: 51 or a functional variant thereof having at least at or about 80%, at least at or about 81%, at least at or about 82%, at least at or about 83%, at or about 84%, at least at or about 85%, at least at or about 86%, or at least at or about 87%, at least at or about 88%, or at least at or about 89%, at least at or about 90%, at least at or about 91%, at least at or about 92%, at least at or about 93%, at least at or about 94%, at least at or about 95%, at or about 96%, at least at or about 97%, at least at or about 98%, or at least at or about 99% sequence identity to SEQ ID NO:51 or such as set forth in SEQ ID NO: 57 or a functional variant thereof having at least at or about 80%, at least at or about 81%, at least at or about 82%, at least at or about 83%, at or about 84%, at least at or about 85%, at least at or about 86%, or at least at or about 87%, at least at or about 88%, or at least at or about 89%, at least at or about 90%, at least at or about 91%, at least at or about 92%, at least at or about 93%, at least at or about 94%, at least at or about 95%, at or about 96%, at least at or about 97%, at least at or about 98%, or at least at or about 99% sequence identity to SEQ ID NO:57, or such as set forth in SEQ ID NO: 63 or a functional variant thereof having at least at or about 80%, at least at or about 81%, at least at or about 82%, at least at or about 83%, at or about 84%, at least at or about 85%, at least at or about 86%, or at least at or about 87%, at least at or about 88%, or at least at or about 89%, at least at or about 90%, at least at or about 91%, at least at or about 92%, at least at or about 93%, at least at or about 94%, at least at or about 95%, at or about 96%, at least at or about 97%, at least at or about 98%, or at least at or about 99% sequence identity to SEQ ID NO:63. In some embodiments, the mutant NiV-G protein has a 34 amino acid truncation at or near the N-terminus of the wild-type NiV-G protein (SEQ ID NO:26, SEQ ID NO:27 or SEQ ID NO:44), such as set forth in SEQ ID NO: 64 or a functional variant thereof having at least at or about 80%, at least at or about 81%, at least at or about 82%, at least at or about 83%, at or about 84%, at least at or about 85%, at least at or about 86%, or at least at or about 87%, at least at or about 88%, or at least at or about 89%, at least at or about 90%, at least at or about 91%, at least at or about 92%, at least at or about 93%, at least at or about 94%, at least at or about 95%, at or about 96%, at least at or about 97%, at least at or about 98%, or at least at or about 99% sequence identity to SEQ ID NO:64 or such as set forth in SEQ ID NO: 65 or a functional variant thereof having at least at or about 80%, at least at or about 81%, at least at or about 82%, at least at or about 83%, at or about 84%, at least at or about 85%, at least at or about 86%, or at least at or about 87%, at least at or about 88%, or at least at or about 89%, at least at or about 90%, at least at or about 91%, at least at or about 92%, at least at or about 93%, at least at or about 94%, at least at or about 95%, at or about 96%, at least at or about 97%, at least at or about 98%, or at least at or about 99% sequence identity to SEQ ID NO:65. In some embodiments, the mutant NiV-G protein lacks the N-terminal cytoplasmic domain of the wild-type NiV-G protein (SEQ ID NO:26, SEQ ID NO:27 or SEQ ID NO:44), such as set forth in SEQ ID NO:45 or a functional variant thereof having at least at or about 80%, at least at or about 81%, at least at or about 82%, at least at or about 83%, at or about 84%, at least at or about 85%, at least at or about 86%, or at least at or about 87%, at least at or about 88%, or at least at or about 89%, at least at or about 90%, at least at or about 91%, at least at or about 92%, at least at or about 93%, at least at or about 94%, at least at or about 95%, at or about 96%, at least at or about 97%, at least at or about 98%, or at least at or about 99% sequence identity to SEQ ID NO:45.

In some embodiments, the G protein is a mutant HeV-G protein that is a biologically active portion of a wild-type HeV-G. In some embodiments, the biologically active portion is an N-terminally truncated fragment.

In some embodiments, the mutant G protein is a mutant HeV-G protein that has the sequence set forth in SEQ ID NO:66 or 67, or is a functional variant or biologically active portion thereof that has an amino acid sequence having at least at or about 80%, at least at or about 81%, at least at or about 82%, at least at or about 83%, at least at or about 84%, at or about 85%, at least at or about 86%, at least at or about 87%, at or about 88%, at least at or about 89%, at least at or about 90%, at least at or about 91%, at least at or about 92%, at least at or about 93%, at least at or about 94%, at least at or about 95%, at or about 96%, at least at or about 97%, at least at or about 98%, or at least at or about 99% sequence identity to SEQ ID NO:66 or 67.

In some embodiments, the G protein is a mutant HeV-G protein that is a biologically active portion of a wild-type HeV-G. In some embodiments, the biologically active portion is an N-terminally truncated fragment. In some embodiments, the mutant HeV-G protein is truncated and lacks up to 5 contiguous amino acid residues at or near the N-terminus of the wild-type HeV-G protein (SEQ ID NO:66 or 67), up to 6 contiguous amino acid residues at or near the N-terminus of the wild-type HeV-G protein (SEQ ID NO:66 or 67), up to 7 contiguous amino acid residues at or near the N-terminus of the wild-type HeV-G protein (SEQ ID NO:66 or 67up to 8 contiguous amino acid residues at or near the N-terminus of the wild-type HeV-G protein (SEQ ID NO:66 or 67), up to 9 contiguous amino acid residues at or near the N-terminus of the wild-type HeV-G protein (SEQ ID NO:66 or 67), up to 10 contiguous amino acid residues at or near the N-terminus of the wild-type HeV-G protein (SEQ ID NO:66 or 67), up to 11 contiguous amino acid residues at or near the N-terminus of the wild-type HeV-G protein (SEQ ID NO:66 or 67), up to 12 contiguous amino acid residues at or near the N-terminus of the wild-type HeV-G protein (SEQ ID NO:66 or 67), up to 13 contiguous amino acid residues at or near the N-terminus of the wild-type HeV-G protein (SEQ ID NO:66 or 67), up to 14 contiguous amino acid residues at or near the N-terminus of the wild-type HeV-G protein (SEQ ID NO:66 or 67), up to 15 contiguous amino acid residues at or near the N-terminus of the wild-type HeV-G protein (SEQ ID NO:66 or 67), up to 16 contiguous amino acid residues at or near the N-terminus of the wild-type HeV-G protein (SEQ ID NO:66 or 67), up to 17 contiguous amino acid residues at or near the N-terminus of the wild-type HeV-G protein (SEQ ID NO:66 or 67), up to 18 contiguous amino acid residues at or near the N-terminus of the wild-type HeV-G protein (SEQ ID NO:66 or 67), up to 19 contiguous amino acid residues at or near the N-terminus of the wild-type HeV-G protein (SEQ ID NO:66 or 67), up to 20 contiguous amino acid residues at or near the N-terminus of the wild-type HeV-G protein (SEQ ID NO:66 or 67), up to 21 contiguous amino acid residues at or near the N-terminus of the wild-type HeV-G protein (SEQ ID NO:66 or 67), up to 22 contiguous amino acid residues at or near the N-terminus of the wild-type HeV-G protein (SEQ ID NO:66 or 67), up to 23 contiguous amino acid residues at or near the N-terminus of the wild-type HeV-G protein (SEQ ID NO:66 or 67), up to 24 contiguous amino acid residues at or near the N-terminus of the wild-type HeV-G protein (SEQ ID NO:66 or 67), up to 25 contiguous amino acid residues at or near the N-terminus of the wild-type HeV-G protein (SEQ ID NO:66 or 67), up to 26 contiguous amino acid residues at or near the N-terminus of the wild-type HeV-G protein (SEQ ID NO:66 or 67), up to 27 contiguous amino acid residues at or near the N-terminus of the wild-type HeV-G protein (SEQ ID NO:66 or 67), up to 28 contiguous amino acid residues at or near the N-terminus of the wild-type HeV-G protein (SEQ ID NO:66 or 67), up to 29 contiguous amino acid residues at or near the N-terminus of the wild-type HeV-G protein (SEQ ID NO:66 or 67), up to 30 contiguous amino acid residues at or near the N-terminus of the wild-type HeV-G protein (SEQ ID NO:66 or 67), up to 31 contiguous amino acid residues at or near the N-terminus of the wild-type HeV-G protein (SEQ ID NO:66 or 67), up to 32 contiguous amino acid residues at or near the N-terminus of the wild-type HeV-G protein (SEQ ID NO:66 or 67), up to 33 contiguous amino acid residues at or near the N-terminus of the wild-type HeV-G protein (SEQ ID NO:66 or 67), up to 34 contiguous amino acid residues at or near the N-terminus of the wild-type HeV-G protein (SEQ ID NO:66 or 67), up to 35 contiguous amino acid residues at or near the N-terminus of the wild-type HeV-G protein (SEQ ID NO:66 or 67), up to 36 contiguous amino acid residues at or near the N-terminus of the wild-type HeV-G protein (SEQ ID NO:66 or 67), up to 37 contiguous amino acid residues at or near the N-terminus of the wild-type HeV-G protein (SEQ ID NO:66 or 67), up to 38 contiguous amino acid residues at or near the N-terminus of the wild-type HeV-G protein (SEQ ID NO:66 or 67), up to 39 contiguous amino acid residues at or near the N-terminus of the wild-type HeV-G protein (SEQ ID NO:66 or 67), up to 40 contiguous amino acid residues at or near the N-terminus of the wild-type HeV-G protein (SEQ ID NO:66 or 67), up to 41 contiguous amino acid residues at or near the N-terminus of the wild-type HeV-G protein (SEQ ID NO:66 or 67), up to 42 contiguous amino acid residues at or near the N-terminus of the wild-type HeV-G protein (SEQ ID NO:66 or 67), up to 43 contiguous amino acid residues at or near the N-terminus of the wild-type HeV-G protein (SEQ ID NO:66 or 67), up to 44 contiguous amino acid residues at or near the N-terminus of the wild-type HeV-G protein (SEQ ID NO:66 or 67), or up to 45 contiguous amino acid residues at or near the N-terminus of the wild-type HeV-G protein (SEQ ID NO:66 or 67). In some embodiments, the HeV-G protein is a biologically active portion that does not contain a cytoplasmic domain. In some embodiments, the mutant HeV-G protein lacks the N-terminal cytoplasmic domain of the wild-type HeV-G protein (SEQ ID NO:66 or 67), such as set forth in SEQ ID NO:68 or a functional variant thereof having at least at or about 80%, at least at or about 81%, at least at or about 82%, at least at or about 83%, at or about 84%, at least at or about 85%, at least at or about 86%, or at least at or about 87%, at least at or about 88%, or at least at or about 89%, at least at or about 90%, at least at or about 91%, at least at or about 92%, at least at or about 93%, at least at or about 94%, at least at or about 95%, at or about 96%, at least at or about 97%, at least at or about 98%, or at least at or about 99% sequence identity to SEQ ID NO:68.

In some embodiments, the G protein or the functionally active variant or biologically active portion thereof binds to Ephrin B2 or Ephrin B3. In some aspects, the G protein has the sequence of amino acids set forth in any one of SEQ ID NO:44, SEQ ID NO:66 or SEQ ID NO:26, SEQ ID NO:28, SEQ ID NO: 27, SEQ ID NO:30 or SEQ ID NO:32, or is a functionally active variant thereof or a biologically active portion thereof that is able to bind to Ephrin B2 or Ephrin B3. In some embodiments, the functionally active variant or biologically active portion has an amino acid sequence having at least at or about 80%, at least at or about 81%, at least at or about 82%, at least at or about 83%, at least at or about 84%, at least at or about 85%, at or about 86%, at least at or about 87%, at least at or about 88%, or at least at or about 89% , at least at or about 90%, at least at or about 91%, at least at or about 92%, at least at or about 93%, at least at or about 94%, at least at or about 95%, at or about 96%, at least at or about 97%, at least at or about 98%, or at least at or about 99% sequence identity to any of SEQ ID NO:44, SEQ ID NO:66 or SEQ ID NO:26, SEQ ID NO:28, SEQ ID NO: 27, SEQ ID NO:30 or SEQ ID NO:32, or a functionally active variant or biologically active portion thereof, and retains binding to Ephrhin B2 or B3.

In some embodiments, the functionally active variant or biologically active portion has an amino acid sequence having at least about 80%, at least about 85%, at least at or about 90%, at least at or about 91%, at least at or about 92%, at least at or about 93%, at least at or about 94%, at least at or about 95%, at or about 96%, at least at or about 97%, at least at or about 98%, or at least at or about 99% sequence identity to SEQ ID NO:44, SEQ ID NO:66 or SEQ ID NO:26, SEQ ID NO:28, SEQ ID NO: 27, SEQ ID NO:30 or SEQ ID NO:32, or a functionally active variant or biologically active portion thereof, and retains binding to Ephrhin B2 or B3. Reference to retaining binding to Ephrin B2 or B3 includes binding that is at least or at least about 5% of the level or degree of binding of the corresponding wild-type G protein, such as set forth in SEQ ID NO:44, SEQ ID NO:66 or SEQ ID NO:26, SEQ ID NO:28, SEQ ID NO: 27, SEQ ID NO:30 or SEQ ID NO:32, or a functionally active variant or biologically active portion thereof, 10% of the level or degree of binding of the corresponding wild-type G protein, such as set forth in SEQ ID NO:44, SEQ ID NO:66 or SEQ ID NO:26, SEQ ID NO:28, SEQ ID NO: 27, SEQ ID NO:30 or SEQ ID NO:32, or a functionally active variant or biologically active portion thereof, 15% of the level or degree of binding of the corresponding wild-type G protein, such as set forth in SEQ ID NO:44, SEQ ID NO:66 or SEQ ID NO:26, SEQ ID NO:28, SEQ ID NO: 27, SEQ ID NO:30 or SEQ ID NO:32, or a functionally active variant or biologically active portion thereof, 20% of the level or degree of binding of the corresponding wild-type G protein, such as set forth in SEQ ID NO:44, SEQ ID NO:66 or SEQ ID NO:26, SEQ ID NO:28, SEQ ID NO: 27, SEQ ID NO:30 or SEQ ID NO:32, or a functionally active variant or biologically active portion thereof, 25% of the level or degree of binding of the corresponding wild-type G protein, such as set forth in SEQ ID NO:44, SEQ ID NO:66 or SEQ ID NO:26, SEQ ID NO:28, SEQ ID NO: 27, SEQ ID NO:30 or SEQ ID NO:32, or a functionally active variant or biologically active portion, 30% of the level or degree of binding of the corresponding wild-type G protein, such as set forth in SEQ ID NO:44, SEQ ID NO:66 or SEQ ID NO:26, SEQ ID NO:28, SEQ ID NO: 27, SEQ ID NO:30 or SEQ ID NO:32, or a functionally active variant or biologically active portion thereof, 35% of the level or degree of binding of the corresponding wild-type G protein, such as set forth in SEQ ID NO:44, SEQ ID NO:66 or SEQ ID NO:26, SEQ ID NO:28, SEQ ID NO: 27, SEQ ID NO:30 or SEQ ID NO:32, or a functionally active variant or biologically active portion thereof, 40% of the level or degree of binding of the corresponding wild-type G protein, such as set forth in SEQ ID NO:44, SEQ ID NO:66 or SEQ ID NO:26, SEQ ID NO:28, SEQ ID NO: 27, SEQ ID NO:30 or SEQ ID NO:32, or a functionally active variant or biologically active portion thereof, 45% of the level or degree of binding of the corresponding wild-type G protein, such as set forth in SEQ ID NO:44, SEQ ID NO:66 or SEQ ID NO:26, SEQ ID NO:28, SEQ ID NO: 27, SEQ ID NO:30 or SEQ ID NO:32, or a functionally active variant or biologically active portion thereof, 50% of the level or degree of binding of the corresponding wild-type G protein, such as set forth in SEQ ID NO:44, SEQ ID NO:66 or SEQ ID NO:26, SEQ ID NO:28, SEQ ID NO: 27, SEQ ID NO:30 or SEQ ID NO:32, or a functionally active variant or biologically active portion thereof, 55% of the level or degree of binding of the corresponding wild-type G protein, such as set forth in SEQ ID NO:44, SEQ ID NO:66 or SEQ ID NO:26, SEQ ID NO:28, SEQ ID NO: 27, SEQ ID NO:30 or SEQ ID NO:32, or a functionally active variant or biologically active portion thereof, 60% of the level or degree of binding of the corresponding wild-type G protein, such as set forth in SEQ ID NO:44, SEQ ID NO:66 or SEQ ID NO:26, SEQ ID NO:28, SEQ ID NO: 27, SEQ ID NO:30 or SEQ ID NO:32or a functionally active variant or biologically active portion thereof, 65% of the level or degree of binding of the corresponding wild-type G protein, such as set forth in SEQ ID NO:44, SEQ ID NO:66 or SEQ ID NO:26, SEQ ID NO:28, SEQ ID NO: 27, SEQ ID NO:30 or SEQ ID NO:32, or a functionally active variant or biologically active portion thereof, 70% of the level or degree of binding of the corresponding wild-type G protein, such as set forth in SEQ ID NO:44, SEQ ID NO:66 or SEQ ID NO:26, SEQ ID NO:28, SEQ ID NO: 27, SEQ ID NO:30 or SEQ ID NO:32, or a functionally active variant or biologically active portion thereof, such as at least or at least about 75% of the level or degree of binding of the corresponding wild-type G protein, such as set forth in SEQ ID NO:44, SEQ ID NO:66 or SEQ ID NO:26, SEQ ID NO:28, SEQ ID NO: 27, SEQ ID NO:30 or SEQ ID NO:32, or a functionally active variant or biologically activ portion thereof, such as at least or at least about 80% of the level or degree of binding of the corresponding wild-type G protein, such as set forth in SEQ ID NO:44, SEQ ID NO:66 or SEQ ID NO:26, SEQ ID NO:28, SEQ ID NO: 27, SEQ ID NO:30 or SEQ ID NO:32, or a functionally active variant or biologically active portion thereof, such as at least or at least about 85% of the level or degree of binding of the corresponding wild-type G protein, such as set forth in SEQ ID NO:44, SEQ ID NO:66 or SEQ ID NO:26, SEQ ID NO:28, SEQ ID NO: 27, SEQ ID NO:30 or SEQ ID NO:32, or a functionally active variant or biologically active portion thereof, such as at least or at least about 90% of the level or degree of binding of the corresponding wild-type G protein, such as set forth in SEQ ID NO:44, SEQ ID NO:66 or SEQ ID NO:26, SEQ ID NO:28, SEQ ID NO: 27, SEQ ID NO:30 or SEQ ID NO:32, or a functionally active variant or biologically active portion thereof, or such as at least or at least about 95% of the level or degree of binding of the corresponding wild-type protein, such as set forth in SEQ ID NO:44, SEQ ID NO:66 or SEQ ID NO:26, SEQ ID NO:28, SEQ ID NO: 27, SEQ ID NO:30 or SEQ ID NO:32, or a functionally active variant or biologically active portion thereof.In some embodiments, the G protein is NiV-G or a functionally active variant or biologically active portion thereof and binds to Ephrin B2 or Ephrin B3. In some aspects, the NiV-G has the sequence of amino acids set forth in SEQ ID NO:26, SEQ ID NO:27 or SEQ ID NO:44, or is a functionally active variant thereof or a biologically active portion thereof that is able to bind to Ephrin B2 or Ephrin B3. In some embodiments, the functionally active variant or biologically active portion has an amino acid sequence having at least about 80%, at least about 85%, at least at or about 90%, at least at or about 91%, at least at or about 92%, at least at or about 93%, at least at or about 94%, at least at or about 95%, at or about 96%, at least at or about 97%, at least at or about 98%, or at least at or about 99% sequence identity to SEQ ID NO:26, SEQ ID NO:27 or SEQ ID NO:44and retains binding to Eprhin B2 or B3. Exemplary biologically active portions include N-terminally truncated variants lacking all or a portion of the cytoplasmic domain, e.g. 1 or more, such as 1 to 49 contiguous N-terminal amino acid residues, e.g. set forth in any one of SEQ ID NOS: 45-63. Reference to retaining binding to Ephrin B2 or B3 includes binding that is at least or at least about 5% of the level or degree of binding of the corresponding wild-type NiV-G, such as set forth in SEQ ID NO:26, SEQ ID NO:27 or SEQ ID NO:44, 10% of the level or degree of binding of the corresponding wild-type NiV-G, such as set forth in SEQ ID NO:26, SEQ ID NO:27 or SEQ ID NO:44, 15% of the level or degree of binding of the corresponding wild-type NiV-G, such as set forth in SEQ ID NO:26, SEQ ID NO:27 or SEQ ID NO:44, 20% of the level or degree of binding of the corresponding wild-type NiV-G, such as set forth in SEQ ID NO:26, SEQ ID NO:27 or SEQ ID NO:44, 25% of the level or degree of binding of the corresponding wild-type NiV-G, such as set forth in SEQ ID NO:26, SEQ ID NO:27 or SEQ ID NO:44, 30% of the level or degree of binding of the corresponding wild-type NiV-G, such as set forth in SEQ ID NO:9, SEQ ID NO:28 or SEQ ID NO:44 SEQ ID NO:26, SEQ ID NO:27 or SEQ ID NO:44, 35% of the level or degree of binding of the corresponding wild-type NiV-G, such as set forth in SEQ ID NO:26, SEQ ID NO:27 or SEQ ID NO:44, 40% of the level or degree of binding of the corresponding wild-type NiV-G, such as set forth in SEQ ID NO:26, SEQ ID NO:27 or SEQ ID NO:44, 45% of the level or degree of binding of the corresponding wild-type NiV-G, such as set forth in SEQ ID NO:26, SEQ ID NO:27 or SEQ ID NO:44 50% of the level or degree of binding of the corresponding wild-type NiV-G, such as set forth in SEQ ID NO:26, SEQ ID NO:27 or SEQ ID NO:44, 55% of the level or degree of binding of the corresponding wild-type NiV-G, such as set forth in SEQ ID NO:26, SEQ ID NO:27 or SEQ ID NO:44, 60% of the level or degree of binding of the corresponding wild-type NiV-G, such as set forth in SEQ ID NO:26, SEQ ID NO:27 or SEQ ID NO:44, 65% of the level or degree of binding of the corresponding wild-type NiV-G, such as set forth in SEQ ID NO:26, SEQ ID NO:27 or SEQ ID NO:44, 70% of the level or degree of binding of the corresponding wild-type NiV-G, such as set forth in SEQ ID NO:26, SEQ ID NO:27 or SEQ ID NO:44, such as at least or at least about 75% of the level or degree of binding of the corresponding wild-type NiV-G, such as set forth in SEQ ID NO:26, SEQ ID NO:27 or SEQ ID NO:44, such as at least or at least about 80% of the level or degree of binding of the corresponding wild-type NIV-G, such as set forth in SEQ ID NO:26, SEQ ID NO:27 or SEQ ID NO:44, such as at least or at least about 85% of the level or degree of binding of the corresponding wild-type NiV-G, such as set forth in SEQ ID NO:26, SEQ ID NO:27 or SEQ ID NO:44, such as at least or at least about 90% of the level or degree of binding of the corresponding wild-type NiV-G, such as set forth in SEQ ID NO:26, SEQ ID NO:27 or SEQ ID NO:44, or such as at least or at least about 95% of the level or degree of binding of the corresponding wild-type NiV-G, such as set forth in SEQ ID NO:26, SEQ ID NO:27 or SEQ ID NO:44.

In some embodiments, the G protein or the biologically thereof is a mutant G protein that exhibits reduced binding for the native binding partner of a wild-type G protein. In some embodiments, the mutant G protein or the biologically active portion thereof is a mutant of wild-type Niv-G and exhibits reduced binding to one or both of the native binding partners Ephrin B2 or Ephrin B3. In some embodiments, the mutant G-protein or the biologically active portion, such as amutant NiV-G protein, exhibits reduced binding to the native binding partner. In some embodiments, the reduced binding to Ephrin B2 or Ephrin B3 is reduced by greater than at or about 5%, at or about 10%, at or about 15%, at or about 20%, at or about 25%, at or about 30%, at or about 40%, at or about 50%, at or about 60%, at or about 70%, at or about 80%, at or about 90%, or at or about 100%.

In some embodiments, the mutations described herein can improve transduction efficiency. In some embodiments, the mutations described herein allow for specific targeting of other desired cell types that are not Ephrin B2 or Ephrin B3. In some embodiments, the mutations described herein result in at least the partial inability to bind at least one natural receptor, such has reduce the binding to at least one of Ephrin B2 or Ephrin B3. In some embodiments, the mutations described herein interfere with natural receptor recognition.

In some embodiments, the G protein is HeV-G or a functionally active variant or biologically active portion thereof and binds to Ephrin B2 or Ephrin B3. In some aspects, the HeV-G has the sequence of amino acids set forth in SEQ ID NO:66 or 67, or is a functionally active variant thereof or a biologically active portion thereof that is able to bind to Ephrin B2 or Ephrin B3. In some embodiments, the functionally active variant or biologically active portion has an amino acid sequence having at least about 80%, at least about 85%, at least at or about 90%, at least at or about 91%, at least at or about 92%, at least at or about 93%, at least at or about 94%, at least at or about 95%, at or about 96%, at least at or about 97%, at least at or about 98%, or at least at or about 99% sequence identity to SEQ ID NO:66 or 67and retains binding to Eprhin B2 or B3. Exemplary biologically active portions include N-terminally truncated variants lacking all or a portion of the cytoplasmic domain, e.g. 1 or more, such as 1 to 49 contiguous N-terminal amino acid residues. Reference to retaining binding to Ephrin B2 or B3 includes binding that is at least or at least about 5% of the level or degree of binding of the corresponding wild-type HeV-G, such as set forth in SEQ ID NO:66 or 67, 10% of the level or degree of binding of the corresponding wild-type HeV-G, such as set forth in SEQ ID NO:66 or 67, 15% of the level or degree of binding of the corresponding wild-type HeV-G, such as set forth in SEQ ID NO:66 or 67, 20% of the level or degree of binding of the corresponding wild-type HeV-G, such as set forth in SEQ ID NO:66 or 67, 25% of the level or degree of binding of the corresponding wild-type HeV-G, such as set forth in SEQ ID NO:66 or 67, 30% of the level or degree of binding of the corresponding wild-type HeV-G, such as set forth in SEQ ID NO:66 or 67, 35% of the level or degree of binding of the corresponding wild-type HeV-G, such as set forth in SEQ ID NO:66 or 67, 40% of the level or degree of binding of the corresponding wild-type HeV-G, such as set forth in SEQ ID NO:66 or 67, 45% of the level or degree of binding of the corresponding wild-type HeV-G, such as set forth in SEQ ID NO:66 or 67, 50% of the level or degree of binding of the corresponding wild-type HeV-G, such as set forth in SEQ ID NO:66 or 67, 55% of the level or degree of binding of the corresponding wild-type HeV-G, such as set forth in SEQ ID NO:66 or 67, 60% of the level or degree of binding of the corresponding wild-type HeV-G, such as set forth in SEQ ID NO:66 or 67, 65% of the level or degree of binding of the corresponding wild-type HeV-G, such as set forth in SEQ ID NO:66 or 67, 70% of the level or degree of binding of the corresponding wild-type HeV-G, such as set forth in SEQ ID NO:18 or 52, such as at least or at least about 75% of the level or degree of binding of the corresponding wild-type HeV-G, such as set forth in SEQ ID NO:66 or 67, such as at least or at least about 80% of the level or degree of binding of the corresponding wild-type NIV-G, such as set forth in SEQ ID NO:66 or 67, such as at least or at least about 85% of the level or degree of binding of the corresponding wild-type HeV-G, such as set forth in SEQ ID NO:66 or 67, such as at least or at least about 90% of the level or degree of binding of the corresponding wild-type HeV-G, such as set forth in SEQ ID NO:66 or 67, or such as at least or at least about 95% of the level or degree of binding of the corresponding wild-type HeV-G, such as set forth in SEQ ID NO:66 or 67.

In some embodiments, the G protein or the biologically thereof is a mutant G protein that exhibits reduced binding for the native binding partner of a wild-type G protein. In some embodiments, the mutant G protein or the biologically active portion thereof is a mutant of wild-type Niv-G and exhibits reduced binding to one or both of the native binding partners Ephrin B2 or Ephrin B3. In some embodiments, the mutant G-protein or the biologically active portion, such as amutant NiV-G protein, exhibits reduced binding to the native binding partner. In some embodiments, the reduced binding to Ephrin B2 or Ephrin B3 is reduced by greater than at or about 5%, at or about 10%, at or about 15%, at or about 20%, at or about 25%, at or about 30%, at or about 40%, at or about 50%, at or about 60%, at or about 70%, at or about 80%, at or about 90%, or at or about 100%.

In some embodiments, the G protein contains one or more amino acid substitutions in a residue that is involved in the interaction with one or both of Ephrin B2 and Ephrin B3. In some embodiments, the amino acid substitutions correspond to mutations E501A, W504A, Q530A and E533A with reference to numbering set forth in SEQ ID NO:38.

In some embodiments, the G protein is a mutant G protein containing one or more amino acid substitutions selected from the group consisting of E501A, W504A, Q530A and E533A with reference to numbering set forth in SEQ ID NO:26. In some embodiments, the G protein is a mutant G protein that contains one or more amino acid substitutions elected from the group consisting of E501A, W504A, Q530A and E533A with reference to SEQ ID NO:26 and is a biologically active portion thereof containing an N-terminal truncation. In some embodiments, the mutant NiV-G protein or the biologically active portion thereof is truncated and lacks up to 5 contiguous amino acid residues at or near the N-terminus of the wild-type NiV-G protein (SEQ ID NO:26), 6 contiguous amino acid residues at or near the N-terminus of the wild-type NiV-G protein (SEQ ID NO:26), 7 contiguous amino acid residues at or near the N-terminus of the wild-type NiV-G protein (SEQ ID NO:26), 8 contiguous amino acid residues at or near the N-terminus of the wild-type NiV-G protein (SEQ ID NO:26), 9 contiguous amino acid residues at or near the N-terminus of the wild-type NiV-G protein (SEQ ID NO:26), up to 10 contiguous amino acid residues at or near the N-terminus of the wild-type NiV-G protein (SEQ ID NO:26), 11 contiguous amino acid residues at or near the N-terminus of the wild-type NiV-G protein (SEQ ID NO:26), 12 contiguous amino acid residues at or near the N-terminus of the wild-type NiV-G protein (SEQ ID NO:26), 13 contiguous amino acid residues at or near the N-terminus of the wild-type NiV-G protein (SEQ ID NO:26), 14 contiguous amino acid residues at or near the N-terminus of the wild-type NiV-G protein (SEQ ID NO:26), up to 15 contiguous amino acid residues at or near the N-terminus of the wild-type NiV-G protein (SEQ ID NO:26), 16 contiguous amino acid residues at or near the N-terminus of the wild-type NiV-G protein (SEQ ID NO:26), 17 contiguous amino acid residues at or near the N-terminus of the wild-type NiV-G protein (SEQ ID NO:26), 18 contiguous amino acid residues at or near the N-terminus of the wild-type NiV-G protein (SEQ ID NO:26), 19 contiguous amino acid residues at or near the N-terminus of the wild-type NiV-G protein (SEQ ID NO:26), up to 20 contiguous amino acid residues at or near the N-terminus of the wild-type NiV-G protein (SEQ ID NO:26), 21 contiguous amino acid residues at or near the N-terminus of the wild-type NiV-G protein (SEQ ID NO:26), 22 contiguous amino acid residues at or near the N-terminus of the wild-type NiV-G protein (SEQ ID NO:26), 23 contiguous amino acid residues at or near the N-terminus of the wild-type NiV-G protein (SEQ ID NO:26), 24 contiguous amino acid residues at or near the N-terminus of the wild-type NiV-G protein (SEQ ID NO:26), up to 25 contiguous amino acid residues at or near the N-terminus of the wild-type NiV-G protein (SEQ ID NO:26), 26 contiguous amino acid residues at or near the N-terminus of the wild-type NiV-G protein (SEQ ID NO:26), 27 contiguous amino acid residues at or near the N-terminus of the wild-type NiV-G protein (SEQ ID NO:26), 28 contiguous amino acid residues at or near the N-terminus of the wild-type NiV-G protein (SEQ ID NO:26), 29 contiguous amino acid residues at or near the N-terminus of the wild-type NiV-G protein (SEQ ID NO:26), up to 30 contiguous amino acid residues at or near the N-terminus of the wild-type NiV-G protein (EQ ID NO:26), up to 31 contiguous amino acid residues at or near the N-terminus of the wild-type NiV-G protein (SEQ ID NO:26), 32 contiguous amino acid residues at or near the N-terminus of the wild-type NiV-G protein (SEQ ID NO:26), 33 contiguous amino acid residues at or near the N-terminus of the wild-type NiV-G protein (SEQ ID NO:26), 34 contiguous amino acid residues at or near the N-terminus of the wild-type NiV-G protein (SEQ ID NO:26), 35 contiguous amino acid residues at or near the N-terminus of the wild-type NiV-G protein (SEQ ID NO:26), up to 36 contiguous amino acid residues at or near the N-terminus of the wild-type NiV-G protein (SEQ ID NO:26), up to 37 contiguous amino acid residues at or near the N-terminus of the wild-type NiV-G protein S(EQ ID NO:26), up to 38 contiguous amino acid residues at or near the N-terminus of the wild-type NiV-G protein (SEQ ID NO:26), up to 39 contiguous amino acid residues at or near the N-terminus of the wild-type NiV-G protein (SEQ ID NO:26), or up to 40 contiguous amino acid residues at or near the N-terminus of the wild-type NiV-G protein (SEQ ID NO:26).

In some embodiments, the mutant NiV-G protein has the amino acid sequence set forth in SEQ ID NO: 69 or 70 or an amino acid sequence having at least at or about 90%, at least at or about 91%, at least at or about 92%, at least at or about 93%, at least at or about 94%, at least at or about 95%, at or about 96%, at least at or about 97%, at least at or about 98%, or at least at or about 99% sequence identity to SEQ ID NO: 69 or 70. In particular embodiments, the G protein has the sequence of amino acids set forth in SEQ ID NO: 69 or 70.

In some embodiments, the G protein is a mutant G protein containing one or more amino acid substitutions selected from the group consisting of E501A, W504A, Q530A and E533A with reference to numbering set forth in SEQ ID NO:38. In some embodiments, the G protein is a mutant G protein that contains one or more amino acid substitutions elected from the group consisting of E501A, W504A, Q530A and E533A with reference to SEQ ID NO:38 and is a biologically active portion thereof containing an N-terminal truncation.

### B. F Proteins

In some embodiments, the vector-surface targeting moiety comprises a protein with a hydrophobic fusion peptide domain. In some embodiments, the vector-surface targeting moiety comprises a henipavirus F protein molecule or biologically active portion thereof. In some embodiments, the Henipavirus F protein is a Hendra (Hev) virus F protein, a Nipah (NiV) virus F-protein, a Cedar (CedPV) virus F protein, a Mojiang virus F protein or a bat Paramyxovirus F protein or a biologically active portion thereof.

Table 4 provides non-limiting examples of F proteins. In some embodiments, the N-terminal hydrophobic fusion peptide domain of the F protein molecule or biologically active portion thereof is exposed on the outside of lipid bilayer.

F proteins of henipaviruses are encoded as F₀ precursors containing a signal peptide (e.g. corresponding to amino acid residues 1-26 of SEQ ID NO:34). Following cleavage of the signal peptide, the mature F₀ (e.g. SEQ ID NO:35) is transported to the cell surface, then endocytosed and cleaved by cathepsin L into the mature fusogenic subunits F1 and F2. The F1 and F2 subunits are associated by a disulfide bond and recycled back to the cell surface. The Fl subunit contains the fusion peptide domain located at the N terminus of the Fl subunit, where it is able to insert into a cell membrane to drive fusion. In some aspects, fusion is blocked by association of the F protein with G protein, until the G protein engages with a target molecule resulting in its disassociation from F and exposure of the fusion peptide to mediate membrane fusion.

Among different henipavirus species, the sequence and activity of the F protein is highly conserved. For examples, the F protein of NiV and HeV viruses share 89% amino acid sequence identity. Further, in some cases, the henipavirus F proteins exhibit compatibility with G proteins from other species to trigger fusion (Brandel-Tretheway et al. Journal of Virology. 2019. 93(13):e00577-19). In some aspects or the provided re-targeted lipid particles, the F protein is heterologous to the G protein, i.e. the F and G protein or biologically active portions are from different henipavirus species. For example, the F protein is from Hendra virus and the G protein is from Nipah virus. In other aspects, the F protein can be a chimeric F protein containing regions of F proteins from different species of Henipavirus. In some embodiments, switching a region of amino acid residues of the F protein from one species of Henipavirus to another can result in fusion to the G protein of the species comprising the amino acid insertion. (Brandel-Tretheway et al. Journal of Virology. 2019. 93(13):e00577-19). In some cases, the chimeric F protein contains an extracellular domain from one henipavirus species and a transmembrane and/or cytoplasmic domain from a different henipavirus species. For example, the F protein contains an extracellular domain of Hendra virus and a transmembrane/cytoplasmic domain of Nipah virus. F protein sequences disclosed herein are predominantly disclosed as expressed sequences including an N-terminal signal sequence. As such N-terminal signal sequences are commonly cleaved co- or post-translationally, the mature protein sequences for all F protein sequences disclosed herein are also contemplated as lacking the N-terminal signal sequence.

Table 4

F proteins

| Full Gene Name | Sequence | SEQ ID | SEQ ID (without signal sequence) |
|---|---|---|---|
| Hendra virus F Protein | MATQEVRLKCLLCGIIVLVLSLEGLGILHYEKLSKIGLVKGITRKYKIKSNPLTKDIVIKMIPNVSNVSKCTGTVMENYKSRLTGILSPIKGAIELYNNNTHDLVGDVKLAGVVMAGIAIGIATAAQITAGVALYEAMKNADNINKLKSSIESTNEAVVKLQETAEKTVYVLTALQDYINTNLVPTIDQISCKQTELALDLALSKYLSDLLFVFGPNLQDPVSNSMTIQAISQAFGGNYETLLRTLGYATEDFDDLLESDSIAGQIVYVDLSSYYIIVRVYFPILTEIQQAYVQELLPVSFNNDNSEWISIVPNFVLIRNTLISNIEVKYCLITKKSVICNQDYATPMTASVRECLTGSTDKCPRELVVSSHVPRFALSGGVLFANCISVTCQCQTTGRAISQSGEQTLLMIDNTTCTTVVLGNIIISLGKYLGSINYNSESIAVGPPVYTDKVDISSQISSMNQSLQQSKDYIKEAQHILDTVNPSLISMLSMIILYVLSIAALCIGLITFISFVIVEKKRGN YSRLDDRQVRPVSNGDLYYIGT | 34 | 35 |
| Nipah virus F Protein | MVVILDKRCYCNLLILILMISECSVGILHYEKLSKIGLVKGVTRKYKIKSNPLTKDIVIKMIPNVSNMSQCTGSVMENYKTRLNGILTPIKGALEIYKNNTHDLVGDVRLAGVIMAGVAIGIATAAQITAGVALYEAMKNADNINKLKSSIESTNEAVVKLQETAEKTVYVLTALQDYINTNLVPTIDKISCKQTELSLDLALSKYLSDLLFVFGPNLQDPVSNSMTIQAISQAFGGNYETLLRTLGYATEDFDDLLESDSITGQIIYVDLSSYYIIVRVYFPILTEIQQAYIQELLPVSFNNDNSEWISIVPNFILVRNTLISNIEIGFCLITKRSVICNQDYATPMTNNMRECLTGSTEKCPRELVVSSHVPRFALSNGVLFANCISVTCQCQTTGRAISQSGEQTLLMIDNTTCPTAVLGNVIISLGKYLGSVNYNSEGIAIGPPVFTDKVDISSQISSMNQSLQQSKDYIKEAQRLLDTVNPSLISMLSMIILYVLSIASLCIGLITFISFIIVEKKRNTYSRLEDRRVRPTSSGDLYYIGT | 36 | 37 |
| Cedar Virus F Protein | MSNKRTTVLIIISYTLFYLNNAAIVGFDFDKLNKIGVVQGRVLNYKIKGDPMTKDLVLKFIPNIVNITECVREPLSRYNETVRRLLLPIHNMLGLYLNNTNAKMTGLMIAGVIMGGIAIGIATAAQITAGFALYEAKKNTENIQKLTDSIMKTQDSIDKLTDSVGTSILILNKLQTYINNQLVPNLELLSCRQNKIEFDLMLTKYLVDLMTVIGPNINNPVNKDMTIQSLSLLFDGNYDIMMSELGYTPQDFLDLIESKSITGQIIYVDMENLYVVIRTYLPTLIEVPDAQIYEFNKITMSSNGGEYLSTIPNFILIRGNYMSNIDVATCYMTKASVICNQDYSLPMSQNLRSCYQGETEYCPVEAVIASHSPRFALTNGVIFANCINTICRCQDNGKTITQNINQFVSMIDNSTCNDVMVDKFTIKVGKYMGRKDINNINIQIGPQIIIDKVDLSNEINKMNQSLKDSIFYLREAKRILDSVNISLISPSVQLFLIIISVLSFIILLIIIVYLYCKSKHSYKYNKFIDDPDYYNDYKRERINGKASKSNNIYYVGD | 38 | 39 |
| Mojiang virus, Tongguan 1 F Protein | MALNKNMFSSLFLGYLLVYATTVQSSIHYDSLSKVGVIKGLTYNYKIKGSPSTKLMVVKLIPNIDSVKNCTQKQYDEYKNLVRKALEPVKMAIDTMLNNVKSGNNKYRFAGAIMAGVALGVATAATVTAGIALHRSNENAQAIANMKSAIQNTNEAVKQLQLANKQTLAVIDTIRGEINNNIIPVINQLSCDTIGLSVGIRLTQYYSEIITAFGPALQNPVNTRITIQAISSVFNGNFDELLKIMGYTSGDLYEILHSELIRGNIIDVDVDAGYIALEIEFPNLTLVPNAVVQELMPISYNIDGDEWVTLVPRFVLTRTTLLSNIDTSRCTITDSSVICDNDYALPMSHELIGCLQGDTSKCAREKVVSSYVPKFALSDGLVYANCLNTICRCMDTDTPISQSLGATVSLLDNKRCSVYQVGDVLISVGSYLGDGEYNADNVELGPPIVIDKIDIGNQLAGINQTLQEAEDYIEKSEEFLKGVNPSIITLGSMVVLYIFMILIAIVSVIALVLSIKLTVKGNVVRQQFTYTQHVPSMENINYVSH | 41 | 41 |
| Bat Paramyxoviru s Eid_hel/GH-M74a/GHA/2 009 F protein | MKKKTDNPTISKRGHNHSRGIKSRALLRETDNYSNGLIVENLVRNCHHPSKNNLNYTKTQKRDSTIPYRVEERKGHYPKIKHLIDKSYKHIKRGKRRNGHNGNIITIILLLILILKTQMSEGAIHYETLSKIGLIKGITREYKVKGTPSSKDIVIKLIPNVTGLNKCTNISMENYKEQLDKILIPINNIIELYANSTKSAPGNARFAGVIIAGVALGVAAAAQITAGIALHEARQNAERINLLKDSISATNNAVAELQEATGGIVNVITGMQDYINTNLVPQIDKLQCSQIKTALDISLSQYYSEILTVFGPNLQNPVTTSMSIQAISQSFGGNIDLLLNLLGYTANDLLDLLESKSITGQITYINLEHYFMVIRVYYPIMTTISNAYVQELIKISFNVDGSEWVSLVPSYILIRNSYLSNIDISECLITKNSVICRHDFAMPMSYTLKECLTGDTEKCPREAVVTSYVPRFAISGGVIYANCLSTTCQCYQTGKVIAQDGSQTLMMIDNQTCSIVRIEEILISTGKYLGSQEYNTMHVSVGNPVFTDKLDITSQISNINQSIEQSKFYLDKSKAILDKINLNLIGSVPISILFIIAILSLILSIITFVIVMIIVRRYNKYTPLINSDPSSRRSTIQDVYIIPNPGEHSIRSAARSIDRDRD | 42 | 43 |

In some embodiments, the F protein is encoded by a nucleotide sequence that encodes the sequence set forth by any one of SEQ ID NOS: 36, 37, 34, 38, 40, 42, 39, 43, 35, or 41 or is a functionally active variant or a biologically active portion thereof that has a sequence that is at least at or about 80%, at least at or about 85%, at least at or about 90%, at least at or about 91%, at least at or about 92%, at least at or about 93%, at least at or about 94%, at least at or about 95%, at least at or about 96%, at least at or about 97%, at least at or about 98%, or at least at or about 99% identical to any one of SEQ ID NOS: 36, 37, 34, 38, 40, 42, 39, 43, 35, or 41

In particular embodiments, the F protein or the functionally active variant or biologically active portion thereof retains fusogenic activity in conjunction with a Henipavirus G protein, such as a G protein set forth in Section III.C.1 (e.g. NiV-G or HeV-G). Fusogenic activity includes the activity of the F protein in conjunction with a G protein to promote or facilitate fusion of two membrane lumens, such as the lumen of the targeted lipid particle having embedded in its lipid bilayer a henipavirus F and G protein, and a cytoplasm of a target cell, e.g. a cell that contains a surface receptor or molecule that is recognized or bound by the targeted envelope protein. In some embodiments, the F protein and G protein are from the same Henipavirus species (e.g. NiV-G and NiV-F). In some embodiments, the F protein and G protein are from different Henipavirus species (e.g. NiV-G and HeV-F). In particular embodiments, the F protein of the functionally active variant or biologically active portion retains the cleavage site cleaved by cathepsin L(e.g. corresponding to the cleavage site between amino acids 109-110 of SEQ ID NO:36).

In particular embodiments, the F protein has the sequence of amino acids set forth in SEQ ID NO: 36, SEQ ID NO:37, SEQ ID NO:34, SEQ ID NO: 38, SEQ ID NO:40, SEQ ID NO: 43, SEQ ID NO: 39, SEQ ID NO: 43, SEQ ID NO: 35, or SEQ ID NO: 41, or is a functionally active variant thereof or a biologically active portion thereof that retains fusogenic activity. In some embodiments, the functionally active variant comprises an amino acid sequence having at least at or about 80%, at least at or about 85%, at least at or about 90%, at least at or about 91%, at least at or about 92%, at least at or about 93%, at least at or about 94%, at least at or about 95%, at or about 96%, at least at or about 97%, at least at or about 98%, or at least at or about 99% sequence identity to SEQ ID NO: 36, SEQ ID NO:37, SEQ ID NO:34, SEQ ID NO: 38, SEQ ID NO:40, SEQ ID NO: 43, SEQ ID NO: 39, SEQ ID NO: 43, SEQ ID NO: 35, or SEQ ID NO: 41, and retains fusogenic activity in conjunction with a Henipavirus G protein (e.g., NiV-G or HeV-G). In some embodiments, the biologically active portion has an amino acid sequence having at least at or about 80%, at least at or about 85%, at least at or about 90%, at least at or about 91%, at least at or about 92%, at least at or about 93%, at least at or about 94%, at least at or about 95%, at or about 96%, at least at or about 97%, at least at or about 98%, or at least at or about 99% sequence identity to SEQ ID NO: 36, SEQ ID NO:37, SEQ ID NO:34, SEQ ID NO: 38, SEQ ID NO:40, SEQ ID NO: 43, SEQ ID NO: 39, SEQ ID NO: 43, SEQ ID NO: 35, or SEQ ID NO: 41, and retains fusogenic activity in conjunction with a Henipavirus G protein (e.g., NiV-G or HeV-G).

Reference to retaining fusogenic activity includes activity (in conjunction with a Henipavirus G protein) that between at or about 10% and at or about 150% or more of the level or degree of binding of the corresponding wild-type F protein, such as set forth in SEQ ID NO: 36, SEQ ID NO:37, SEQ ID NO:34, SEQ ID NO: 38, SEQ ID NO:40, SEQ ID NO: 43, SEQ ID NO: 39, SEQ ID NO: 43, SEQ ID NO: 35, or SEQ ID NO: 41, such as at least or at least about 10% of the level or degree of fusogenic activity of the corresponding wild-type F protein, such as at least or at least about 15% of the level or degree of fusogenic activity of the corresponding wild-type F protein, such as at least or at least about 20% of the level or degree of fusogenic activity of the corresponding wild-type F protein, such as at least or at least about 25% of the level or degree of fusogenic activity of the corresponding wild-type F protein, such as at least or at least about 30% of the level or degree of fusogenic activity of the corresponding wild-type F protein, such as at least or at least about 35% of the level or degree of fusogenic activity of the corresponding wild-type F protein, such as at least or at least about 40% of the level or degree of fusogenic activity of the corresponding wild-type F protein, such as at least or at least about 45% of the level or degree of fusogenic activity of the corresponding wild-type F protein, such as at least or at least about 50% of the level or degree of fusogenic activity of the corresponding wild-type F protein, such as at least or at least about 55% of the level or degree of fusogenic activity of the corresponding wild-type f protein, such as at least or at least about 60% of the level or degree of fusogenic activity of the corresponding wild-type F protein, such as at least or at least about 65% of the level or degree of fusogenic activity of the corresponding wild-type F protein, such as at least or at least about 70% of the level or degree of fusogenic activity of the corresponding wild-type F protein, such as at least or at least about 75% of the level or degree of fusogenic activity of the corresponding wild-type F protein, such as at least or at least about 80% of the level or degree of fusogenic activity of the corresponding wild-type F protein, such as at least or at least about 85% of the level or degree of fusogenic activity of the corresponding wild-type F protein, such as at least or at least about 90% of the level or degree of fusogenic activity of the corresponding wild-type F protein, such as at least or at least about 95% of the level or degree of fusogenic activity of the corresponding wild-type F protein, such as at least or at least about 100% of the level or degree of fusogenic activity of the corresponding wild-type F protein, or such as at least or at least about 120% of the level or degree of fusogenic activity of the corresponding wild-type F protein.

In some embodiments, the F protein is a mutant F protein that is a functionally active fragment or a biologically active portion containing one or more amino acid mutations, such as one or more amino acid insertions, deletions, substitutions or truncations. In some embodiments, the mutations described herein relate to amino acid insertions, deletions, substitutions or truncations of amino acids compared to a reference F protein sequence. In some embodiments, the reference F protein sequence is the wild-type sequence of an F protein or a biologically active portion thereof. In some embodiments, the mutant F protein or the biologically active portion thereof is a mutant of a wild-type Hendra (Hev) virus F protein, a Nipah (NiV) virus F-protein, a Cedar (CedPV) virus F protein, a Mojiang virus F protein or a bat Paramyxovirus F protein. In some embodiments, the wild-type F protein is encoded by a sequence of nucleotides that encodes any one of SEQ ID NO: 36, SEQ ID NO:37, SEQ ID NO:34, SEQ ID NO: 38, SEQ ID NO:40, SEQ ID NO: 43, SEQ ID NO: 39, SEQ ID NO: 43, SEQ ID NO: 35, or SEQ ID NO: 41,

In some embodiments, the mutant F protein is a biologically active portion of a wild-type F protein that is an N-terminally and/or C-terminally truncated fragment. In some embodiments, the mutant F protein or the biologically active portion of a wild-type F protein thereof comprises one or more amino acid substitutions. In some embodiments, the mutations described herein can improve transduction efficiency. In some embodiments, the mutations described herein can increase fusogenic capacity. Exemplary mutations include any as described, see e.g. Khetawat and Broder 2010 Virology Journal 7:312; Witting et al. 2013 Gene Therapy 20:997-1005; published international; patent application No. WO/2013/148327.

In some embodiments, the mutant F protein is a biologically active portion that is truncated and lacks up to 20 contiguous amino acid residues at or near the C-terminus of the wild-type F protein, such as a wild-type F protein encoded by a sequence of nucleotides encoding the F protein set forth in any one of SEQ ID NOS: 34-43. In some embodiments, the mutant F protein is truncated and lacks up to 19 contiguous amino acids, such as up to 18 , 17, 16, 15, 14, 13, 12, 11, 10, 9, 8, 7, 6, 5, 4, 3, 2, or 1 contiguous amino acids at the C-terminus of the wild-type F protein.

In some embodiments, the F protein or the functionally active variant or biologically active portion thereof comprises an Fl subunit or a fusogenic portion thereof. In some embodiments, the Fl subunit is a proteolytically cleaved portion of the F₀ precursor. In some embodiments, the F₀ precursor is inactive. In some embodiments, the cleavage of the F₀ precursor forms a disulfide-linked F1+F2 heterodimer. In some embodiments, the cleavage exposes the fusion peptide and produces a mature F protein. In some embodiments, the cleavage occurs at or around a single basic residue. In some embodiments, the cleavage occurs at Arginine 109 of NiV-F protein. In some embodiments, cleavage occurs at Lysine 109 of the Hendra virus F protein.

In some embodiments, the F protein is a wild-type Nipah virus F (NiV-F) protein or is a functionally active variant or biologically active porteion thereof. In some embodiments, the F₀ precursor is encoded by a sequence of nucleotides encoding the sequence set forth in SEQ ID NO: 36. The encoding nucleic acid can encode a signal peptide sequence that has the sequence MVVILDKRCY CNLLILILMI SECSVG (SEQ ID NO: 71). In some embodiments, the F protein has the sequence set forth in SEQ ID NO:37. In some examples, the F protein is cleaved into an Fl subunit comprising the sequence set forth in SEQ ID NO:73 and an F2 subunit comprising the sequence set forth in SEQ ID NO: 72.

In some embodiments, the F protein is a NiV-F protein that is encoded by a sequence of nucleotides encoding the sequence set forth in SEQ ID NO:71, or is a functionally active variant or biologically active portion thereof that has an amino acid sequence having at least at or about 80%, at least at or about 81%, at least at or about 82%, at least at or about 83%, at least at or about 84%, at least at or about 85%, at or about 86%, at least at or about 87%, at least at or about 88%, or at least at or about 89%, at least at or about 90%, at least at or about 91%, at least at or about 92%, at least at or about 93%, at least at or about 94%, at least at or about 95%, at or about 96%, at least at or about 97%, at least at or about 98%, or at least at or about 99% sequence identity to SEQ ID NO: 71. In some embodiments, the NiV-F-protein has the sequence of set forth in SEQ ID NO: 52, or is a functionally active variant or a biologically active portion thereof that has an amino acid sequence having at least at or about 80%, at least at or about 81%, at least at or about 82%, at least at or about 83%, at least at or about 84%, at least at or about 85%, at or about 86%, at least at or about 87%, at least at or about 88%, or at least at or about 89%, at least at or about 90%, at least at or about 91%, at least at or about 92%, at least at or about 93%, at least at or about 94%, at least at or about 95%, at or about 96%, at least at or about 97%, at least at or about 98%, or at least at or about 99% sequence identity to SEQ ID NO: 52. In particular embodiments, the F protein or the functionally active variant or biologically active portion thereof retains the cleavage site cleaved by cathepsin L.

In some embodiments, the F protein or the functionally active variant or the biologically active portion thereof includes an Fl subunit that has the sequence set forth in SEQ ID NO: 37, or an amino acid sequence having, at least at or about 90%, at least at or about 91%, at least at or about 92%, at least at or about 93%, at least at or about 94%, at least at or about 95%, at or about 96%, at least at or about 97%, at least at or about 98%, or at least at or about 99% sequence identity to SEQ ID NO:4.

In some embodiments, the F protein or the functionally active variant or biologically active portion thereof includes an F2 subunit that has the sequence set forth in SEQ ID NO: 36, or an amino acid sequence having, at least at or about 90%, at least at or about 91%, at least at or about 92%, at least at or about 93%, at least at or about 94%, at least at or about 95%, at or about 96%, at least at or about 97%, at least at or about 98%, or at least at or about 99% sequence identity to SEQ ID NO:36.

In some embodiments, the F protein or the functionally active variant or the biologically active portion thereof includes an Fl subunit that has the sequence set forth in SEQ ID NO: 72, or an amino acid sequence having, at least at or about 80%, at least at or about 81%, at least at or about 82%, at least at or about 83%, at least at or about 84%, at least at or about 85%, at or about 86%, at least at or about 87%, at least at or about 88%, or at least at or about 89% at least at or about 90%, at least at or about 91%, at least at or about 92%, at least at or about 93%, at least at or about 94%, at least at or about 95%, at or about 96%, at least at or about 97%, at least at or about 98%, or at least at or about 99% sequence identity to SEQ ID NO:72.

In some embodiments, the F protein or the functionally active variant or biologically active portion thereof includes an F2 subunit that has the sequence set forth in SEQ ID NO: 73, or an amino acid sequence having, at least at or about 80%, at least at or about 81%, at least at or about 82%, at least at or about 83%, at least at or about 84%, at least at or about 85%, at or about 86%, at least at or about 87%, at least at or about 88%, or at least at or about 89% at least at or about 90%, at least at or about 91%, at least at or about 92%, at least at or about 93%, at least at or about 94%, at least at or about 95%, at or about 96%, at least at or about 97%, at least at or about 98%, or at least at or about 99% sequence identity to SEQ ID NO:73.

In some embodiments, the F protein is a mutant NiV-F protein that is a biologically active portion thereof that is truncated and lacks up to 20 contiguous amino acid residues at or near the C-terminus of the wild-type NiV-F protein (e.g. set forth SEQ ID NO:37). In some embodiments, the mutant NiV-F protein comprises an amino acid sequence set forth in SEQ ID NO:55. In some embodiments, the mutant NiV-F protein has a sequence that has at least at or about 90%, at least at or about 91%, at least at or about 92%, at least at or about 93%, at least at or about 94%, at least at or about 95%, at or about 96%, at least at or about 97%, at least at or about 98%, or at least at or about 99% sequence identity to SEQ ID NO: 55. In some embodiments, the mutant F protein contains an Fl protein that has the sequence set forth in SEQ ID NO:56. In some embodiments, the mutant F protein has a sequence that has at least at or about 90%, at least at or about 91%, at least at or about 92%, at least at or about 93%, at least at or about 94%, at least at or about 95%, at or about 96%, at least at or about 97%, at least at or about 98%, or at least at or about 99% sequence identity to SEQ ID NO: 56.

In some embodiments, the F protein is a mutant NiV-F protein that is a biologically active portion thereof that comprises a 20 amino acid truncation at or near the C-terminus of the wild-type NiV-F protein (SEQ ID NO:37); and a point mutation on an N-linked glycosylation site. In some embodiments, the mutant NiV-F protein comprises an amino acid sequence set forth in SEQ ID NO: 74. In some embodiments, the mutant NiV-F protein has a sequence that has at least at or about 90%, at least at or about 91%, at least at or about 92%, at least at or about 93%, at least at or about 94%, at least at or about 95%, at or about 96%, at least at or about 97%, at least at or about 98%, or at least at or about 99% sequence identity to SEQ ID NO: 74.

In some embodiments, the F protein is a mutant NiV-F protein that is a biologically active portion thereof that comprises a 22 amino acid truncation at or near the C-terminus of the wild-type NiV-F protein (SEQ ID NO:37). In some embodiments, the NiV-F protein is encoded by a nucleotide sequence that encodes the sequence set forth in SEQ ID NO: 75. In some embodiments, the NiV-F proteins is encoded by a nucleotide sequence that encodes sequence having at least at or about 90%, at least at or about 91%, at least at or about 92%, at least at or about 93%, at least at or about 94%, at least at or about 95%, at or about 96%, at least at or about 97%, at least at or about 98%, or at least at or about 99% sequence identity to SEQ ID NO: 75.

In some embodiments, the F protein is a mutant NiV-F protein that is a biologically active portion thereof that comprises a 22 amino acid truncation at or near the C-terminus of the wild-type NiV-F protein (SEQ ID NO:37). In some embodiments, the NiV-F protein is encoded by a nucleotide sequence that encodes the sequence set forth in SEQ ID NO: 79. In some embodiments, the NiV-F proteins is encoded by a nucleotide sequence that encodes sequence having at least at or about 90%, at least at or about 91%, at least at or about 92%, at least at or about 93%, at least at or about 94%, at least at or about 95%, at or about 96%, at least at or about 97%, at least at or about 98%, or at least at or about 99% sequence identity to SEQ ID NO: 79.

In some embodiments, the F protein is a mutant NiV-F protein that is a biologically active portion thereof that comprises a 22 amino acid truncation at or near the C-terminus of the wild-type NiV-F protein (SEQ ID NO:37). In some embodiments, the NiV-F protein is encoded by a nucleotide sequence that encodes the sequence set forth in SEQ ID NO: 80. In some embodiments, the NiV-F proteins is encoded by a nucleotide sequence that encodes sequence having at least at or about 90%, at least at or about 91%, at least at or about 92%, at least at or about 93%, at least at or about 94%, at least at or about 95%, at or about 96%, at least at or about 97%, at least at or about 98%, or at least at or about 99% sequence identity to SEQ ID NO: 80.

### 2. Retargeting Moieties

In some embodiments, the fusogen is a targeted envelope protein that contains a vector-surface targeting moiety. In some embodiments, the vector-surface targeting moiety binds a target ligand. In some embodiments, the target ligand can be expressed in an organ or cell type of interest, e.g., the lung. In particular embodiments, the fusogen (e.g. G protein) is mutated to reduce binding for the native binding partner of the fusogen. In some embodiments, the fusogen is or contains a mutant G protein or a biologically active portion thereof that is a mutant of wild-type Niv-G and exhibits reduced binding to one or both of the native binding partners Ephrin B2 or Ephrin B3, including any as described above. Thus, in some aspects, a fusogen can be retargeted to display altered tropism. In some embodiments, the binding confers re-targeted binding compared to the binding of a wild-type surface glycoprotein protein in which a new or different binding activity is conferred. In particular embodiments, the binding confers re-targeted binding compared to the binding of a wild-type G protein in which a new or different binding activity is conferred.

In some embodiments, protein fusogens may be re-targeted by covalently conjugating a targeting-moiety to the fusion protein. In some embodiments, the fusogen and targeting moiety are covalently conjugated by expression of a chimeric protein comprising the fusogen linked to the targeting moiety. In some embodiments, a target includes any peptide (e.g. a receptor) that is displayed on a target cell. In some embodiments, the target is expressed at higher levels on a target cell than non-target cells. In some embodiments, a single-chain variable fragment (scFv) can be conjugated to fusogens to redirect fusion activity towards cells that display the scFv binding target (doi:10.1038/nbtl060, DOI 10.1182/blood-2012-11-468579, doi:10.1038/nmeth.1514, doi:10.1006/mthe.2002.0550, HUMAN GENE THERAPY 11:817- 826, doi:10.1038/nbt942, doi:10.1371/journal.pone.0026381, DOI 10.1186/s 12896-015-0142-z). In some embodiments, designed ankyrin repeat proteins (DARPin) can be conjugated to fusogens to redirect fusion activity towards cells that display the DARPin binding target (doi:10.1038/mt.2013.16, doi:10.1038/mt.2010.298, doi: 10.4049/jimmunol. 1500956), as well as combinations of different DARPins (doi:10.1038/mto.2016.3). In some embodiments, receptor ligands and antigens can be conjugated to fusogens to redirect fusion activity towards cells that display the target receptor (DOI: 10.1089/hgtb.2012.054, DOI: 10.1128/JVI.76.7.3558-3563.2002). In some embodiments, a targeting protein can also include an antibody or an antigen-binding fragment thereof (e.g., Fab, Fab′, F(ab′)2, Fv fragments, scFv antibody fragments, disulfide-linked Fvs (sdFv), a Fd fragment consisting of the VH and CH1 domains, linear antibodies, single domain antibodies such as sdAb (either VL or VH), nanobodies, or camelid VHH domains), an antigen-binding fibronectin type III (Fn3) scaffold such as a fibronectin polypeptide minibody, a ligand, a cytokine, a chemokine, or a T cell receptor (TCRs). In some embodiments, protein fusogens may be re-targeted by non-covalently conjugating a targeting moiety to the fusion protein or targeting protein (e.g. the hemagglutinin protein). In some embodiments, the fusion protein can be engineered to bind the Fc region of an antibody that targets an antigen on a target cell, redirecting the fusion activity towards cells that display the antibody’s target (DOI: 10.1128/JVI.75.17.8016-8020.2001, doi:10.1038/nmll92). In some embodiments, altered and non-altered fusogens may be displayed on the same retroviral vector or VLP (doi: 10.1016/j.biomaterials.2014.01.051).

In some embodiments, a targeting moiety comprises a humanized antibody molecule, intact IgA, IgG, IgE or IgM antibody; bi- or multi- specific antibody (e.g., Zybodies®, etc); antibody fragments such as Fab fragments, Fab′ fragments, F(ab′)2 fragments, Fd′ fragments, Fd fragments, and isolated CDRs or sets thereof; single chain Fvs; polypeptide-Fc fusions; single domain antibodies (e.g., shark single domain antibodies such as IgNAR or fragments thereof); cameloid antibodies; masked antibodies (e.g., Probodies®); Small Modular ImmunoPharmaceuticals (“SMIPsTM”); single chain or Tandem diabodies (TandAb®); VHHs; Anticalins®; Nanobodies®; minibodies; BiTE®s; ankyrin repeat proteins or DARPINs®; Avimers®; DARTs; TCR-like antibodies;, Adnectins®; Affilins®; Trans-bodies®; Affibodies®; TrimerX®; MicroProteins; Fynomers®, Centyrins®; and KALBITOR®s. In embodiments, the re-targeted fusogen binds a cell surface marker on the target cell, e.g., a protein, glycoprotein, receptor, cell surface ligand, agonist, lipid, sugar, class I transmembrane protein, class II transmembrane protein, or class III transmembrane protein.

In some embodiments, vector-surface targeting moiety is a peptide. In some embodiments, vector-surface targeting moiety is an antibody, such as a single domain antibody. In some embodiments, the antibody can be human or humanized. In some embodiments, antibody or portion thereof is naturally occurring. In some embodiments, the antibody or portion thereof is synthetic.

In some embodiments, the antibody can be generated from phage display libraries to have specificity for a desired target ligand. In some embodiments, the target ligand is a receptor for viral entry, such as ACE2. In some embodiments the target ligand is expressed in the lung, such as ACE2. In some embodiments, the phage display libraries are generated from a VHH repertoire of camelids immunized with various antigens, as described in Arbabi et al., FEBS Letters, 414, 521-526 (1997); Lauwereys et al., EMBO J., 17, 3512-3520 (1998); Decanniere et al., Structure, 7, 361-370 (1999). In some embodiments, the phage display library is generated comprising antibody fragments of a non-immunized camelid. In some embodiments, single domain antibodies a library of human single domain antibodies is synthetically generated by introducing diversity into one or more scaffolds.

In some embodiments, the C-terminus of the vector-surface targeting moiety is attached to the C-terminus of the G protein (e.g., fusogen) or biologically active portion thereof. In some embodiments, the N-terminus of the vector-surface targeting moiety is exposed on the exterior surface of the lipid bilayer. In some embodiments, the N-terminus of the vector-surface targeting moiety binds to a cell surface molecule of a target cell. In some embodiments, the vector-surface targeting moiety specifically binds to a cell surface molecule present on a target cell. In some embodiments, the vector-surface targeting moiety is a protein, glycan, lipid or low molecular weight molecule.

In some embodiments, the vector-surface targeting moiety is derived from a coronavirus. Coronaviruses typically bind to target cells through Spike-receptor (S) interactions and enter cells by receptor mediated endocytosis or fusion with the plasma membrane. The S-receptor interaction is a strong determinant of species specificity as demonstrated for both group 1 and group 2 coronaviruses. The receptor for group 1 coronaviruses, including human coronavirus 229E (HCoV-229E), feline coronavirus (FCoV) and porcine coronavirus (PCoV) has been identified as aminopeptidase N (APN/CD13) (Delmas, et al., 1992, Nature 357:417-420; Tresnan, et al., 1996, J. Virol. 70:8669-8674; Yeager, et al., 1992, Nature 357:420-422). APN/CD13 is a 150- to 160-kDa type II protein that is a membrane peptidase (Look, et al., 1989, J. Clin. Invest 83:1299-1307). In some embodiments, the S protein binds ACE2. In some embodiments, the S protein binds DPP4.

In some embodiments, the vector-surface targeting moiety is derived from a coronavirus S protein. The coronavirus S glycoprotein is exemplified, but not limited to, those encoded by the genomic sequences in gi;31416292;gb;AY278487.3; SARS coronavirus BJ02,gi;30248028;gb;AY274119.3; SARS coronavirus TOR2, gi;30698326;gb;AY291451.1; SARS coronavirus TW1, gi;33115118;gb;AY323977.2; SARS coronavirus HSR 1, gi;35396382;gb;AY394850.1; SARS coronavirus WHU, gi;33411459;dbj;AP006561.1; SARS coronavirus TWY, gi;33411444;dbj;AP006560.1; SARS coronavirus TWS, gi;33411429;dbj;AP006559.1; SARS coronavirus TWK, gi;33411414;dbj;AP006558.1; SARS coronavirus TWJ, gi:33411399:dbj:AP006557.1: SARS coronavirus TWH, gi;30023963;gb;AY278491.2; SARS coronavirus HKU-39849, gi;33578015;gb;AY310120.1; SARS coronavirus FRA, gi|33518725|gb|AY362699.1 ; SARS coronavirus TWC3, gi;33518724;gb;AY362698; SARS coronavirus TWC2, gi;30027617;gb;AY278741.1; SARS coronavirus Urbani, gi;31873092;gb;AY321118.1; SARS coronavirus TWC, gi;33304219;gb;AY351680.1; SARS coronavirus ZMY 1, gi;31416305;gb;AY278490.3° SARS coronavirus BJ03, gi;30910859;gb;AY297028.1; SARS coronavirus ZJ01, gi;30421451;gb;AY282752.1; SARS coronavirus CUHK-SulO, SARS coronavirus SZ16, gi;34482137;gb;AY304486.1; SARS coronavirus SZ3 gi;30027610;gb;AY278554.2; SARS coronavirus CUHK-W1, gi;31416306;gb;AY279354.2; SARS coronavirus BJ04, gi|37576845|gb|AY427439.1|SARS coronavirus AS, gi;37361915;gb;AY283798.2; SARS coronavirus Sin2774, gi;31416290;gb;AY278489.2; SARS coronavirus GD01, gi;30468042;gb;AY283794.1; SARS coronavirus Sin2500, gi;30468043;gb;AY283795.1; SARS coronavirus Sin2677, gi;30468044;gb;AY283796.1; SARS coronavirus Sin2679, gi;30468045;gb;AY283797.1; SARS coronavirus Sin2748, gi;31982987;gb;AY286320.2; SARS coronavirus isolate ZJ-HZ01, and gi;30275666;gb;AY278488.2; SARS coronavirus BJ01.

In some embodiments, the vector-surface targeting moiety is Severe Acute Respiratory Syndrome (SARS) coronavirus 1 (SARS CoV-1) spike glycoprotein or variants thereof. In some embodiments, the vector-surface targeting moiety is Severe Acute Respiratory Syndrome (SARS) coronavirus 2 (SARS CoV-2) spike glycoprotein or vairants thereof. In some embodiments, the vector-surface targeting moiety is syncytin.

In some embodiments, the cell surface ligand of a target cell is an antigen or portion thereof. In some embodiments, the vector-surface targeting moiety or portion thereof is an antibody having a single monomeric domain antigen binding/recognition domain that is able to bind selectively to a specific antigen. In some embodiments, the single domain antibody binds an antigen present on a target cell.

Exemplary cells include lung stem cells, bronchiolar epithelial cells, alveolar epithelial cells, stromal cells, type 1 and II pneumocytes also known as alveolar type I and II epithelial cells, basal cells, secretory cells, club cells, clara cells, ciliated cells, capillary cells, alveolar macrophages, and lung epithelial cells. In some embodiments, the target cell is an epithelial cell. In some embodiments, the ligand is expressed on a host cell, such as an epithelial cell. In some embodiments, the ligand is ACE2.

In some embodiments, the target cell is a cell of a target tissue. The target tissue can include liver, lungs, heart, spleen, pancreas, gastrointestinal tract, kidney, testes, ovaries, brain, reproductive organs, central nervous system, peripheral nervous system, skeletal muscle, endothelium, inner ear, or eye. In some embodiments, the target tissue is the lung.

### IV. Pharmaceutical Composition and Methods of Manufacture

Also provided are compositions containing the polynucleotides or vectors comprising the polynucleotides provided herein, including pharmaceutical compositions and formulations. Also provided are methods of using and uses of the compositions, such as in the treatment of coronavirus infection or reducing the likelihood of coronavirus infection.

The present disclosure also provides, in some aspects, a pharmaceutical composition comprising the composition described herein and pharmaceutically acceptable carrier. The pharmaceutical compositions can include any of the described polynucleotides or vehicles for delivery.

The term “pharmaceutical formulation” refers to a preparation which is in such form as to permit the biological activity of an active ingredient contained therein to be effective, and which contains no additional components which are unacceptably toxic to a subject to which the formulation would be administered.

A “pharmaceutically acceptable carrier” refers to an ingredient in a pharmaceutical formulation, other than an active ingredient, which is nontoxic to a subject. A pharmaceutically acceptable carrier includes, but is not limited to, a buffer, excipient, stabilizer, or preservative.

In some aspects, the choice of carrier is determined in part by the particular cell and/or by the method of administration. Accordingly, there are a variety of suitable formulations. For example, the pharmaceutical composition can contain preservatives. Suitable preservatives may include, for example, methylparaben, propylparaben, sodium benzoate, and benzalkonium chloride. In some aspects, a mixture of two or more preservatives is used. The preservative or mixtures thereof are typically present in an amount of about 0.0001% to about 2% by weight of the total composition. Carriers are described, e.g., by Remington’s Pharmaceutical Sciences 16th edition, Osol, A. Ed. (1980). Pharmaceutically acceptable carriers are generally nontoxic to recipients at the dosages and concentrations employed, and include, but are not limited to: buffers such as phosphate, citrate, and other organic acids; antioxidants including ascorbic acid and methionine; preservatives (such as octadecyldimethylbenzyl ammonium chloride; hexamethonium chloride; benzalkonium chloride; benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (e.g. Zn-protein complexes); and/or non-ionic surfactants such as polyethylene glycol (PEG).

A pharmaceutical composition may be prepared, packaged, and/or sold in a formulation suitable for pulmonary administration via the buccal cavity, such as via inhalation. Delivery systems currently available include pressurized metered dose inhalator, nebulisers, and dry powder or aerosolized inhalers. In some aspects, it has been found that medicaments for administration by inhalation should be of a controlled particle size in order to achieve maximum penetration into the lungs, preferably in the range of 1 to 10 micrometers in diameter.

Pharmaceutical compositions formulated for pulmonary delivery may provide an active ingredient in the form of droplets of a solution and/or suspension. Such compositions may be prepared, packaged, and/or sold as aqueous and/or dilute alcoholic solutions and/or suspensions, optionally sterile, comprising active ingredient, and may conveniently be administered using any nebulization and/or atomization device. Such compositions may further comprise one or more additional ingredients including, but not limited to, a flavoring agent such as saccharin sodium, a volatile oil, a buffering agent, a surface active agent, and/or a preservative such as methylhydroxybenzoate. Droplets provided by this route of administration may have an average diameter in the range from about 0.1 nm to about 200 nm.

In some aspects, the polynucleotides, polypeptides, and vehicles provided herein can be formulated in compositions suitable for inhalation, including, for example, inhalable powders, lyophilized compositions, propellant-containing aerosols and propellant-free inhalation solutions. In certain embodiments, the inhalable powder is administered to the subject via a dry powder inhaler (DPI). In certain embodiments, a propellant-containing aerosol is administered to a subject via a metered dose inhaler (MDI). In certain embodiments, the propellant-free inhalation solution is administered to the subject via a nebulizer.

In some embodiments, the compsotion containing any of the provided agent or vehicle, e.g. polynucleotides, fusion proteins or vehicles for delivery, are lyophilized or freeze dried, such as in the form of a dry powder. Such a formulation may comprise dry particles which comprise the active ingredient and which have a diameter in the range from about 0.5 nm to about 7 nm or from about 1 nm to about 6 nm. Such compositions are suitably in the form of dry powders for administration using a device comprising a dry powder reservoir to which a stream of propellant may be directed to disperse the powder and/or using a self propelling solvent/powder dispensing container such as a device comprising the active ingredient dissolved and/or suspended in a low-boiling propellant in a sealed container. Such powders comprise particles wherein at least 98% of the particles by weight have a diameter greater than 0.5 nm and at least 95% of the particles by number have a diameter less than 7 nm. Alternatively, at least 95% of the particles by weight have a diameter greater than 1 nm and at least 90% of the particles by number have a diameter less than 6 nm. Dry powder compositions may include a solid fine powder diluent such as sugar and are conveniently provided in a unit dose form.

Low boiling propellants generally include liquid propellants having a boiling point of below 65° F. at atmospheric pressure. Generally the propellant may constitute 50%) to 99.9%) (w/w) of the composition, and active ingredient may constitute 0.1 %> to 20% (w/w) of the composition. A propellant may further comprise additional ingredients such as a liquid non-ionic and/or solid anionic surfactant and/or a solid diluent (which may have a particle size of the same order as particles comprising the active ingredient).

In some aspects, a pharmaceutical composition comprising the polynucleotides, poplypeptides, and/or vehicles provided herein can be prepared for inhalation with an amount of at least one surfactant, such as is sufficicent to facilitate the absorption of inhalted particles. To obtain these absorbable compositions, any surfactant that facilitates inhalation of any of of the compositions disclosed herein. Surfactants suitable for use in promoting absorption of inhaled compositions include, but are not limited to, polyoxyethylene sorbitol esters such as polysorbate 80 (Tween 80) and polysorbate 20 (Tween 20); Propylene-polyoxyethylene esters such as poloxamer 188; polyoxyethylene alcohols such as Brij35; mixtures of polysorbate surfactants with phospholipids such as phosphatidylcholine and derivatives (dipalmitoyl, dioleoyl, dimyristyl, or 1-palmitoyl, 2 Mixed derivatives such as olcoyl), dimyristol glycerol and other members of a series of phospholipid glycerols; lysophosphatidylcholine and derivatives thereof; lysolecithin A mixture of polysorbate with cholesterol; a mixture of polysorbate surfactant with sorbitan surfactant (such as sorbitan monooleate, dioleate, trioleate or others from this class); poloxamer surfactants; bile salts and their Derivatives such as sodium cholate, sodium deoxycholate, sodium glycodeoxycholate, sodium taurocholate, etc .; mixed micelles of TNFa inhibitors with bile salts and phospholipids; Brij surfactant (such as Brij35-PEG923) lauryl alcohol, etc. ) Is included. The amount of surfactant to be added is about 0.005% to about 1.0% (w / v), preferably about 0.005% to about 0.5%, more preferably about 0.01%. To about 0.4%, even more preferably from about 0.03% to about 0.3%, and most preferably from about 0.05% to about 0.2%.

Sterile inhlation solutions can be prepared by incorporating the required amount of the active compound (ie, polynucleotides, polypeptides, and/or vehicles comprising C19orf66 or a regaultory factor thereof) in an appropriate solvent. Solutions can then be sterilized by filtration. Generally, dispersions are prepared by incorporating the active compound into a sterile vehicle that contains a basic dispersion medium and the required other ingredients from those enumerated above. The correct fluidity of the solution can be maintained, for example, by the use of a coating such as lecithin, by the maintenance of the required particle size in the case of dispersion and by the use of surfactants. Inclusion of agents that delay absorption, such as monostearate salts and gelatin, in the composition may provide sustained absorption of the inhalable composition.

Compositions described herein as being useful for pulmonary delivery are useful for intranasal delivery of a pharmaceutical composition. Another compositions suitable for intranasal administration is a coarse powder comprising the active ingredient and having an average particle from about 0.2 µm to 500 µm. Such a compositions is administered in the manner in which snuff is taken, i.e. by rapid inhalation through the nasal passage from a container of the powder held close to the nose. Compositions suitable for nasal administration may, for example, comprise from about as little as 0.1% (w/w) and as much as 100%) (w/w) of active ingredient, and may comprise one or more of the additional ingredients described herein.

A pharmaceutical composition may be prepared, packaged, and/or sold in a formulation suitable for buccal administration. Such compositions may, for example, be in the form of tablets and/or lozenges made using conventional methods, and may, for example, 0.1% to 20% (w/w) active ingredient, the balance comprising an orally dissolvable and/or degradable composition and, optionally, one or more of the additional ingredients described herein. Alternately, compositions suitable for buccal administration may comprise a powder and/or an aerosolized and/or atomized solution and/or suspension comprising active ingredient. Such powdered, aerosolized, and/or aerosolized formulations, when dispersed, may have an average particle and/or droplet size in the range from about 0.1 nm to about 200 nm, and may further comprise one or more of any additional ingredients described herein.

Buffering agents in some aspects are included in the compositions. Suitable buffering agents include, for example, citric acid, sodium citrate, phosphoric acid, potassium phosphate, and various other acids and salts. In some aspects, a mixture of two or more buffering agents is used. The buffering agent or mixtures thereof are typically present in an amount of about 0.001% to about 4% by weight of the total composition. Methods for preparing administrable pharmaceutical compositions are known. Exemplary methods are described in more detail in, for example, Remington: The Science and Practice of Pharmacy, Lippincott Williams & Wilkins; 21st ed. (May 1, 2005).

Active ingredients may be entrapped in microcapsules, in colloidal drug delivery systems (for example, liposomes, albumin microspheres, microemulsions, nanoparticles and nanocapsules) or in macroemulsions. In certain embodiments, the pharmaceutical composition is formulated as an inclusion complex, such as cyclodextrin inclusion complex, or as a liposome. Liposomes can serve to target the polynucleotides (e.g., delivery vehicles) to a particular tissue. Many methods are available for preparing liposomes, such as those described in, for example, Szoka et al., Ann. Rev. Biophys. Bioeng., 9: 467 (1980), and U.S. Pat. 4,235,871, 4,501,728, 4,837,028, and 5,019,369.

The pharmaceutical composition in some embodiments contains polynucleotides or vehicles for their delivery in amounts effective to treat or reduce the likelihood of a coronavirus infection, such as a therapeutically effective or prophylactically effective amount. Therapeutic or prophylactic efficacy in some embodiments is monitored by periodic assessment of treated subjects. For repeated administrations over several days or longer, depending on the condition, the treatment is repeated until a desired suppression of disease symptoms occurs. However, other dosage regimens may be useful and can be determined. The desired dosage can be delivered by a single bolus administration of the composition, by multiple bolus administrations of the composition, or by continuous infusion administration of the composition.

The polynucleotides and/or delivery vehicle may be administered using standard administration techniques, formulations, and/or devices. Formulations include those for oral, intravenous, intraperitoneal, subcutaneous, pulmonary, transdermal, intramuscular, intranasal, buccal, sublingual, or suppository administration. In some embodiments, the cell populations are administered parenterally. The term “parenteral,” as used herein, includes intravenous, intramuscular, subcutaneous, rectal, vaginal, and intraperitoneal administration. In some embodiments, the polynucleotides and/or delivery vehicle are administered by nebulization. In some embodiments, the polynucleotides and/or delivery vehicle are administered by inhalation.

In some embodiments, the vehicle for delivery of a polynucleotide or polypeptide, such as encoding C19orf66 or a regulatory factor thereof, is a viral vector or virus-like particle (E.g., Sections IIIA and IIIB). In some embodiments, the compositions provided herein can be formulated in dosage units of genome copies (GC). Suitable method for determining GC have been described and include, e.g., qPCR or digital droplet PCR (ddPCR) as described in, e.g., M. Lock et al, Hu Gene Therapy Methods, Hum Gene Ther Methods 25(2):115-25. 2014, which is incorporated herein by reference. In some embodiments, the dosage of administration of a viral vector or virus-like particle is from about 10⁴ to about 10¹⁰ GC units, inclusive. In some embodiments, the dosage of administration of a viral vector or virus-like particle is from about 10⁹ to about 10¹⁵ GC units, inclusive. In some embodiments, the dosage of administration of a viral vector or virus-like particle is from about 10⁵ to about 10⁹ GC units, inclusive. In some embodiments, the dosage of administration of a viral vector or virus-like particle is from about 10⁶ to about 10⁹ GC units, inclusive. In some embodiments, the dosage of administration of a viral vector or virus-like particle is from about 10¹² to about 10¹⁴ GC units, inclusive. In some embodiments, the dosage of administration is 1.0×10⁹ GC units, 5.0×10⁹ GC units, 1.0×10¹⁰ GC units, 5.0×10¹⁰ GC units, 1.0×10¹¹ GC units, 5.0×10¹¹ GC units, 1.0×10¹² GC units, 5.0×10¹² GC units, or 1.0x 10¹³ GC units, 5.0x10¹³ GC units, 1.0×10¹⁴ GC units, 5.0x 10¹⁴ GC units, or 1.0x10¹⁵ GC units.

In some embodiments, the dosage of administration of a viral vector or virus-like particle is from about 10⁴ to about 10¹⁰ infectious units, inclusive. In some embodiments, the dosage of administration of a viral vector or virus-like particle is from about 10⁹ to about 10¹⁵ infectious units, inclusive In some embodiments, the dosage of administration of a viral vector or virus-like particle is from about 10⁵ to about 10⁹ infectious units. In some embodiments, the dosage of administration of a viral vector or virus-like particle is from about 10⁶ to about 10⁹ infectious units. In some embodiments, the dosage of administration of a viral vector or virus-like particle is from about 10¹² to about 10¹⁴ infectious units, inclusive. In some embodiments, the dosage of administration is 1.0×10⁹ infectious units, 5.0×10⁹ infectious units, l.OxlO¹⁰ infectious units, 5.0×10¹⁰ infectious units, 1.0×10¹¹ infectious units, 5.0×10¹¹ infectious units, 1.0×10¹² infectious units, 5.0×10¹² infectious units, or 1.0×10¹³ infectious units, 5.0×10¹³ infectious units, 1.0×10¹⁴ infectious units, 5.0×10¹⁴ infectious units, or 1.0x10¹⁵ infectious units. The techniques available for quantifing infectious units are routine in the art and include viral particle number determination, fluorescence microscopy, and titer by plaque assay. For example, the number of adenovirus particles can be determined by measuring the absorbance at A260. Similarly, infectious units can also be determined by quantitative immunofluorescence of vector specific proteins using monoclonal antibodes or by plaque assay.

In some embodiments, methods that calculate the infectious units include the plaque assay, in which titrations of the virus are grown on cell monolayers and the number of plaques is counted after several days to several weeks. For example, the infectious titer is determined, such as by plaque assay, for example an assay to assess cytopathic effects (CPE). In some embodiments, a CPE assay is performed by serially diluting virus on monolayers of cells, such as HFF cells, that are overlaid with agarose. After incubation for a time period to achieve a cytopathic effect, such as for about 3 to 28 days, generally 7 to 10 days, the cells can be fixed and foci of absent cells visualized as plaques are determined. In some embodiments, infectious units can be determined using an endpoint dilution (TCID₅₀) method, which determines the dilution of virus at which 50% of the cell cultures are infected and hence, generally, can determine the titer within a certain range, such as one log.

In some embodiments, the dosage of administration of a viral vector or virus-like particle is from about 10⁴ to about 10¹⁰ plaque forming units (pfu), inclusive. In some embodiments, the dosage of administration of a viral vector or virus-like particle is from about 10⁹ to about 10¹⁵ pfu, inclusive In some embodiments, the dosage of administration of a viral vector or virus-like particle is from about 10″ to about 10⁹ pfu. In some embodiments, the dosage of administration of a viral vector or virus-like particle is from about 10⁶ to about 10⁹ pfu. In some embodiments, the dosage of administration of a viral vector or virus-like particle is from about 10¹² to about 10¹⁴ pfu, inclusive. In some embodiments, the dosage of administration is 1.0×10⁹ pfu, 5.0×10⁹ pfu, 1.0×10¹⁰ pfu, 5.0×10¹⁰ pfu, 1.0×10¹¹ pfu, 5.0×10¹¹ pfu, 1.0×10¹² pfu, 5.0×10¹² pfu, or 1.0×10¹³ pfu, 5.0×10¹³ pfu, 1.0×10¹⁴ pfu, 5.0×10¹⁴ pfu, or 1.0×10¹⁵ pfu.

In some embodiments, the vehicle for delivery of a polynucleotide or polypeptide, such as encoding C19orf66 or a regulatory factor thereof, is an adenovirus vector. In some aspects, the dosage for administration of adenovirus to humans can range from about 10⁷ to 10⁹, inclusive, plaque forming units (pfu) per injection.

In some aspects, the dosage of administration of a vehicle within the pharmaceutical compositions provided herein varies depending on a subject’s body weight. For example, a composition may be formulated as GC/kg, infectious units/kg, pfu/kg, etc. In some aspects, the dosage at which a therapeutic effect is obtained is from at or about 10⁸ GC/kg to at or about 10¹⁴ GC/kg of the subject’s body weight, inclusive. In some aspects, the dosage at which a therapeutic effect is obtained is at or about 10⁸ GC/kg of the subject’s body weight (GC/kg).

In some embodiments, the subject will receive a single injection. In some embodiments, administration can be repeated at daily/weekly/monthly intervals for an indefinite period and/or until the efficacy of the treatment has been established. As set forth herein, the efficacy of treatment can be determined by evaluating the symptoms and clinical parameters described herein in Section V and/or by detecting a desired response.

The exact amount of the polynucleotide, polypeptide, or vector vehicle required will vary from subject to subject, depending on the species, age, weight and general condition of the subject, the particular poylnucleic acid, polypeptide, or vector used, its mode of administration etc. Thus, it is not possible to specify an exact amount for every polynucleic acid, polynucleotide, or vector vehicle provided herein. However, an appropriate amount can be determined by one of ordinary skill in the art using only routine experimentation given the teachings herein.

Compositions in some embodiments are provided as sterile liquid preparations, e.g., isotonic aqueous solutions, suspensions, emulsions, dispersions, or viscous compositions, which may in some aspects be buffered to a selected pH. Liquid preparations are normally easier to prepare than gels, other viscous compositions, and solid compositions. Additionally, liquid compositions are somewhat more convenient to administer, especially by injection. Viscous compositions, on the other hand, can be formulated within the appropriate viscosity range to provide longer contact periods with specific tissues. Liquid or viscous compositions can comprise carriers, which can be a solvent or dispersing medium containing, for example, water, saline, phosphate buffered saline, polyol (for example, glycerol, propylene glycol, liquid polyethylene glycol) and suitable mixtures thereof.

Sterile injectable solutions can be prepared by incorporating the cells in a solvent, such as in admixture with a suitable carrier, diluent, or excipient such as sterile water, physiological saline, glucose, dextrose, or the like. The compositions can also be lyophilized. The compositions can contain auxiliary substances such as wetting, dispersing, or emulsifying agents (e.g., methylcellulose), pH buffering agents, gelling or viscosity enhancing additives, preservatives, flavoring agents, colors, and the like, depending upon the route of administration and the preparation desired. Standard texts may in some aspects be consulted to prepare suitable preparations.

Injectables can be prepared in conventional forms, either as liquid solutions or suspensions, solid forms suitable for solution of suspension in liquid prior to injection, or as emulsions. As used herein, “parenteral administration” includes intradermal, intranasal, subcutaneous, intramuscular, intraperitoneal, intravenous and intratracheal routes, as well as a slow release or sustained release system such that a constant dosage is maintained.

Various additives which enhance the stability and sterility of the compositions, including antimicrobial preservatives, antioxidants, chelating agents, and buffers, can be added. Prevention of the action of microorganisms can be ensured by various antibacterial and antifungal agents, for example, parabens, chlorobutanol, phenol, sorbic acid, and the like. Prolonged absorption of the injectable pharmaceutical form can be brought about by the use of agents delaying absorption, for example, aluminum monostearate and gelatin.

Sustained-release preparations may be prepared. Suitable examples of sustained-release preparations include semipermeable matrices of solid hydrophobic polymers containing the antibody, which matrices are in the form of shaped articles, e.g. films, or microcapsules.

In some embodiments, vehicle formulations may comprise cyroprotectants. As used herein, there term “cryoprotectant” refers to one or more agent that when combined with a given substance, helps to reduce or eliminate damage to that substance that occurs upon freezing. In some embodiments, cryoprotectants are combined with vector vehicles in order to stabilize them during freezing. In some aspects, Frozen storage of RNA between -20° C. and -80° C. may be advantageous for long term (e.g. 36 months) stability of polynucleotide. In some embodiments, the RNA species is mRNA. In some embodiments, cryoprotectants are included in vehicle formulations to stabilize polynucleotide through freeze/thaw cycles and under frozen storage conditions. Cryoprotectants of the present invention may include, but are not limited to sucrose, trehalose, lactose, glycerol, dextrose, raffinose and/or mannitol. Trehalose is listed by the Food and Drug Administration as being generally regarded as safe (GRAS) and is commonly used in commercial pharmaceutical formulations.

The formulations to be used for in vivo administration are generally sterile. Sterility may be readily accomplished, e.g., by filtration through sterile filtration membranes.

### V. Methods of Use and Therapeutic Applications

Provided herein are methods and uses of treating or reducing the likelihood of a virus infections, such as a coronavirus infection. Among provided methods and uses are those involving administering compositions containing the polynucleotides, proteins (e.g. fusion proteins or complexes), vehicles for delivery, and compositions, and uses of such polynucleotides, proteins (e.g. fusion proteins or complexes) and vehicles for delivery, to treat or reduce the likelihood of a virus infection, such as a coronavirus infection. In some embodiments, the polynucleotides, vehicles for delivery, and compositions are administered to a subject or patient having the particular disease or condition to be treated. In some embodiments, provided cells and compositions are administered to a subject, such as a subject having or at risk for the disease or condition. In some embodimetns, the disease or condition is a virus infection, and the subject is known, suspected, or predicted to have been exposed to a virus causing the infection. In provided embodiments, the virus infection can be caused by or associated with any virus as described herein. In some embodiments, the virus is a coronavirus. For example, in some aspects, the subject is known, suspected, or predicted to have been exposed to a SARS coronavirus. Thus, in some embodiments, the subject is known, suspected, or predicted to have been exposed to a SARS CoV-2 virus. In some embodiments, the subject is known or suspected of having Severe Acute Respiratory Syndrome (SARS).

In some embodiments, the polynucleotides, fusion proteins, vehicles for delivery, or compositions is administered in an effective amount to effect a therapeutic effect, such as to reduce or prevent a virus infection or severity of symptoms associated with a virus infection. Uses include uses of the polynucleotides, proteins (e.g. fusion proteins or complexes), vehicles for delivery, or compositions in the preparation of a medicament in order to carry out such therapeutic methods. In some embodiments, the methods are carried out by administering a provided polynucleotide, protein (e.g. fusion protein or complex), or vehicles for delivery, or compositions comprising the same, to the subject having or suspected of having a virus infection or being at risk of exposure to a virus causing a virus infection, e.g. a coronavirus infection, such as an infection with a SARS-CoV-2 virus. In some embodiments, the methods thereby treat the virus infection in the subject.

It is therefore an object of the present invention to provide methods of treatment, such as methods comprising compositions for delivery of polynucleotides or polypeptides, for the treatment of an infection, such as a coronavirus infection.

In some embodiments, the provided methods or uses involve administration of a pharmaceutical composition comprising oral, inhaled, transdermal or parenteral (including intravenous, intratumoral, intraperitoneal, intramuscular, intracavity, and subcutaneous) administration. In some embodiments, the vehicle particle may be administered alone or formulated as a pharmaceutical composition. In some embodiments, the vehicle particle or compositions described herein can be administered to a subject, e.g., a mammal, e.g., a human. In some of any embodiments, the subject may be at risk of, may have a symptom of, or may be diagnosed with or identified as having, a particular disease or condition (e.g., a coronavirus infection). In some embodiments, the disease is a disease or disorder. In some embodiments, the disease is Severe Acute Respiratory Syndrome (SARS).

In some embodiments, the provided compostions are administered orally. Oral administration may include administration by inhalation, such as to produce an aerosol for delivery to the lungs. In some embodimetns, the compositions may be administered using a dry-powder inhaler (DPI), pressurized metered-dose inhaler (pMDI) or a nebulizer.Any of the provided compositions formulated for oral administration, such as by inhalation, described herein can be administered in accord with the provided methos.

Aerosol delivery is an attractive approach because it is non-invasive and has the potential for delivering high concentrations of the therapeutic polynucleotide or polypeptide, such as those encoding C 19orf66 or regulatory factors thereof. Aerosol delivery of nucleic acids to the lungs using viral vectors, polymers, surfactants, or excipients has been described. McDonald, et al., describes aerosol delivery of an adenoviral vector encoding the cystic fibrosis transmembrane conductance regulator protein (CFTR) to non-human primates (McDonald, et al., Human Gene Therapy 8:411-422 (1997)). Canonico, et al., describes the in vivo gene transfer of a plasmid containing recombinant human alpha 1-antitrpsin gene and a cytomegalovirus promoter complexed to cationic liposomes to the lungs by aerosol to rabbits (Canonico, et al., Am. J. Respir. Cell Mol. Biol, 10:24-29 (1994)). Stribling, et al., describes that the aerosol delivery of a chloramphenicol acetyltransferase reporter gene complexed to a cationic liposome carrier can produce CAT gene expression in mouse lungs (Stribling, et al., Proc. Natl Acad. Sci. USA 89:11277-11281 (1992)).

Massaro, et al., describes delivery of small inhibitory RNA molecules complexed to the lipoprotein pulmonary surfactant, known as surface active material or SAM, to the pulmonary alveoli in mice via liquid deposition into the nasal orifice (Massaro, et al., Am. J. Physiol. Lung Cell Mol. Physiol. 287:L1066-L1070 (2004)). U.S. Pat. Application No. 2005/0008617 by Chen, et al., describes delivery of RNAi-inducing agents including short-interfering RNA (siRNA), short hairpin RNA (shRNA), and RNAi-inducing vectors complexed with cationic polymers, modified cationic polymers, lipids, and/or surfactants suitable for introduction into the lung. U.S. Pat. Application No. 2003/0157030 by Davis, et al., describes administration of RNAi constructs such as siRNAs or nucleic acids that produce siRNAs complexed with polymers for nasal delivery. Any of such methods can be used in the provided embodiments.

In some embodiments, the provided polynucleotides, proteins (e.g. fusion proteins or complexes), vehicles for delivery, or compositions containing any of the same, may be administered in the form of a unit-dose composition, such as a unit dose oral, parenteral, transdermal or inhaled composition. In some embodiments, the compositions are prepared by admixture and are adapted for oral, inhaled, transdermal or parenteral administration, and as such may be in the form of tablets, capsules, oral liquid preparations, powders, granules, lozenges, reconstitutable powders, injectable and infusable solutions or suspensions or suppositories or aerosols.

In some aspects, a “therapeutically effective time” refers to the period of time during which a pharmaceutically effective amount of a compound is administered, and that is sufficient to reduce one or more symptoms associated with a disease or disorder, such as SARS-coronavirus infection.

In some embodiments, the provided polynucleotides, proteins (e.g. fusion proteins or complexes), vehicles for delivery, or compositions containing any of the same,,may be administered before, concomitantly with, and/or after detection of symptoms of infection, such as with SARS-coronavirus. The term “concomitant” when in reference to the relationship between administration of a compound and symptoms means that administration occurs at the same time as, or during, manifestation of symptom associated with SARS-coronavirus infection. In some embodiments, the provided polynucleotides, proteins (e.g. fusion proteins or complexes), vehicles for delivery, or compositions containing any of the same, provided herein may be administered before, concomitantly with, and/or after administration of another type of drug or therapeutic procedure.

In some embodiments, the composition may be administered to a subject as frequently as several times daily, or it may be administered less frequently, such as once a day, once a week, once every two weeks, once a month, or even less frequently, such as once every several months or even once a year or less. In some embodiments, the amount of compound dosed per day may be administered, in non-limiting examples, every day, every other day, every 2 days, every 3 days, every 4 days, or every 5 days. In some embodiments, with every other day administration, a 5 mg per day dose may be initiated on Monday with a first subsequent 5 mg per day dose administered on Wednesday, a second subsequent 5 mg per day dose administered on Friday, and so on. The frequency of the dose will be readily apparent to the skilled artisan and will depend upon any number of factors, such as, but not limited to, the type and severity of the disease being treated, the type and age of the subject, etc.

As used herein, a “subject” is a mammal, such as a human or other animal, and typically is human. In some embodiments, the subject, e.g., patient, to whom the polynucleotides, vehicles for delivery, and compositions are administered, is a mammal, typically a primate, such as a human. In some embodiments, the primate is a monkey or an ape. The subject can be male or female and can be any suitable age, including infant, juvenile, adolescent, adult, and geriatric subjects.

As used herein, “treatment” (and grammatical variations thereof such as “treat” or “treating”) refers to complete or partial amelioration or reduction of a disease or condition or disorder, or a symptom, adverse effect or outcome, or phenotype associated therewith, such as is associated with coronavirus infection. Desirable effects of treatment include, but are not limited to, preventing occurrence or recurrence of infection, alleviation of symptoms, diminishment of any direct or indirect pathological consequences of the infection, preventing Severe Acute Respiratory Syndrome (SARS), decreasing the rate of disease progression, amelioration or palliation of the disease state, and remission or improved prognosis. The terms do not imply complete curing of a disease or complete elimination of any symptom or effect(s) on all symptoms or outcomes.

In some embodiments, the subject is known, suspected, or predicted to have been exposed to a SARS CoV-2 virus. In some embodiments, the subject has pneumonia, such as bilateral pneumonia. In some embodiments, the subject has ground-glass opacities, such as can be imaged with chest computed tomography (CT) scan.

In some embodiments, the subject is known, suspected, or predicted to have been exposed to a SARS CoV-2 virus. In some embodiments, the subject is known or suspected of having Severe Acute Respiratory Syndrome (SARS). SARS CoV-2 has a proposed staging system comprising three distinct disease phases. Mild or early infection is also known as Stage 1 coronavirus disease (COVID). Stage I COVID can be largely asymptomatic or presents with generally non-specific symptoms like malaise, cough, fever etc. Stage II COVID occurs with the establishment of pulmonary disease and/or pulmonary inflammation. In Stage IIa, patients do not display hypoxia associated with viral pneumonia (defined at PaO₂/FiO₂ <300 mmHg). Stage IIb COVID is characterized by hypoxia and often necessitates mechanical ventilation. Stage III COVID is observed in the minority of patients and is strongly associated with mortality. In some aspects, Stage III COVID is characterized by systemic inflammation, such as is observed during a “cytokine storm”. There are no FDA approved treatments for COVID at any stage.

As used herein, “delaying development of a disease” means to defer, hinder, slow, retard, stabilize, suppress and/or postpone development of the disease (such as cancer). This delay can be of varying lengths of time, depending on the history of the disease and/or individual being treated. As is evident to one skilled in the art, a sufficient or significant delay can, in effect, encompass prevention, in that the individual does not develop the disease.

“Preventing,” as used herein, includes providing prophylaxis with respect to the occurrence or recurrence of a disease in a subject that may be predisposed or exposed to the disease or disease causing agent but has not yet been diagnosed with the disease. In some embodiments, the provided the polynucleotides, vehicles for delivery, and compositions are used to delay development of a disease or to slow the progression of a disease.

An “effective amount”, e.g., a pharmaceutical formulation, or composition, including any containing provided polynucleotides, proteins (e.g. fusion proteins or complexes), or vehicles for delivery, in the context of administration, refers to an amount effective, at dosages/amounts and for periods of time necessary, to achieve a desired result, such as a therapeutic or prophylactic result.

A “therapeutically effective amount”, e.g., a pharmaceutical formulation or compostion, including any containing provided polynucleotides, proteins (e.g. fusion proteins or complexes), or vehicles for delivery, refers to an amount effective, at dosages and for periods of time necessary, to achieve a desired therapeutic result, such as for treatment of a disease, condition, or disorder, and/or pharmacokinetic or pharmacodynamic effect of the treatment. The therapeutically effective amount may vary according to factors such as the disease state, age, sex, and weight of the subject, and the polynucleotides administered. In some embodiments, the provided methods involve administering the provided polynucleotides, proteins (e.g. fusion proteins or complexes), vehicles for delivery, or compositions containing any of the same, at effective amounts, e.g., therapeutically effective amounts.

A “prophylactically effective amount” refers to an amount effective, at dosages and for periods of time necessary, to achieve the desired prophylactic result. Typically but not necessarily, since a prophylactic dose is used in subjects prior to or at an earlier stage of disease, the prophylactically effective amount will be less than the therapeutically effective amount.

In some embodiments, the provided polynucleotides, proteins (e.g. fusion proteins or complexes), vehicles for delivery, or compositions containing any of the same, may ameliorate, or reduce, the severity of symptoms relative to baseline (Day 1 of administration of any of the provided polynucleotides, proteins (e.g. fusion proteins or complexes), vehicles for delivery, or compositions containing any of the same, provided herein in any of Sections I, II, III and IV). In some embodiments, subjects are assessed for symptoms of coronavirus infection, including anyone of the following: fever, sore throat, cough, shortness of breath, myalgia. In some embodiments, the subjects self-assess. In some embodiments, subjects will be assessed on day 5 for when symptoms abate compared to baseline. In some embodiments, subjects will be assessed on day 7 for when symptoms abate compared to baseline. In some embodiments, subjects will be assessed on day 14 for when symptoms abate compared to baseline. In some embodiments, subjects will be assessed on day 21 for when symptoms completely abate to baseline. In some embodiments, subjects will be assessed on day 30 for when symptoms abate compared to baseline.

In some embodiments, the provided polynucleotides, proteins (e.g. fusion proteins or complexes), vehicles for delivery, or compositions containing any of the same,, may ameliorate, or reduce, the time to resolution of symptoms relative to baseline (Day 1 of administration of any of the polynucleotides, polypeptide, or vehicles provided herein in any of Sections I, II, III, and IV). In some embodiments, subjects will be assessed on day 5 for when symptoms completely resolve compared to baseline. In some embodiments, subjects will be assessed on day 7 for when symptoms completely resolve compared to baseline. In some embodiments, subjects will be assessed on day 14 for when symptoms completely resolve compared to baseline. In some embodiments, subjects will be assessed on day 21 for when symptoms completely resolve compared to baseline. In some embodiments, subjects will be assessed on day 30 for when symptoms completely resolve compared to baseline.

In some embodiments, the provided polynucleotides, proteins (e.g. fusion proteins or complexes), vehicles for delivery, or compositions containing any of the same, may ameliorate, or increase, the odds ratio for improvement on a 7-point ordinal scale on day 14. In some aspects, the odds ratio represents the odds of improvement in the ordinal scale between treatment groups. In some aspects, the ordinal scale is an assessment of the clinical status at a given day. The scale is set forth as follows: 1. Death 2. Hospitalized, on invasive mechanical ventilation or Extracorporeal Membrane Oxygenation (ECMO) 3. Hospitalized, on non-invasive ventilation or high flow oxygen devices 4. Hospitalized, requiring low flow supplemental oxygen 5. Hospitalized, not requiring supplemental oxygen - requiring ongoing medical care (coronavirus related or otherwise) 6. Hospitalized, not requiring supplemental oxygen - no longer required ongoing medical care 7. Not hospitalized.

In some embodiments, the provided polynucleotides, proteins (e.g. fusion proteins or complexes), vehicles for delivery, or compositions containing any of the same, may ameliorate, or prevent, the need for an ER visit.

In some embodiments, the provided polynucleotides, proteins (e.g. fusion proteins or complexes), vehicles for delivery, or compositions containing any of the same, may ameliorate, or reduce, the number of repeat ER visits following a first ER visit.

In some embodiments, the provided polynucleotides, proteins (e.g. fusion proteins or complexes), vehicles for delivery, or compositions containing any of the same, may ameliorate, or reduce, the number of days in the intensive care unit (ICU). In some embodiments, the provided polynucleotides, proteins (e.g. fusion proteins or complexes), vehicles for delivery, or compositions containing any of the same, may ameliorate, or reduce, the number of days a subject is placed on a ventilator, such as a mechanical ventilator or ECMO.

In some embodiments, the provided polynucleotides, proteins (e.g. fusion proteins or complexes), vehicles for delivery, or compositions containing any of the same, may ameliorate, or reduce, the number of days in the intensive care unit (ICU). In some embodiments, the provided polynucleotides, proteins (e.g. fusion proteins or complexes), vehicles for delivery, or compositions containing any of the same, may ameliorate, or reduce, the number of days a subject is administered vasopressors.

In some embodiments, the provided polynucleotides, proteins (e.g. fusion proteins or complexes), vehicles for delivery, or compositions containing any of the same, may ameliorate, or reduce, the number of days in the intensive care unit (ICU). In some embodiments, the provided polynucleotides, proteins (e.g. fusion proteins or complexes), vehicles for delivery, or compositions containing any of the same, may ameliorate, or reduce, the number of days a subject is administered renal replacement therapy.

In some embodiments, the provided polynucleotides, proteins (e.g. fusion proteins or complexes), vehicles for delivery, or compositions containing any of the same, may ameliorate, or reduce, the number of days needed for recovery from a coronavirus infection. In some aspects, subjects will be assed for recovery based on the following clinical criteria: normalization of pyrexia, respiratory rate and SPO₂, and relief of cough (where there are relevant abnormal symptoms at enrolment) that is maintained for at least 72 hours. In some embodiments, the provided polynucleotides, proteins (e.g. fusion proteins or complexes), vehicles for delivery, or compositions containing any of the same,may ameliorate, or reduce, the number of days to resolution of pyrexia.

In some embodiments, the provided polynucleotides, proteins (e.g. fusion proteins or complexes), vehicles for delivery, or compositions containing any of the same, may ameliorate, or reduce, number of adverse events as measured by Cancer Institute’s Common Terminology Criteria for Adverse Events (NCI-CTCAE) v5.0, for at least one month, two months, three months, four months, five months, or six months following treatment.

In some embodiments, the provided polynucleotides, proteins (e.g. fusion proteins or complexes), vehicles for delivery, or compositions containing any of the same, may ameliorate, or reduce, the number of days to resolution of cough. In some aspects, a subjects cough made be graded according to NCI-CTCAE v5.0, as set forth in Table 5 below.

Table 5

NCI-CTCAE v5.0 Cough Grading Scale

| | |
|---|---|
| Mild | Requires non-prescription treatment |
| Moderate | Requires treatment with medication, limits instrumental activities of daily living |
| Severe | Limits self-care activities of daily living |

In some embodiments, the provided polynucleotides, proteins (e.g. fusion proteins or complexes), vehicles for delivery, or compositions containing any of the same,may ameliorate, or reduce, the number of incidences of deterioration and/or aggravation of pneumonia. In some aspects, subjects will be assed for deteriorating/aggravated pneumonia based on the presence of at least one of the following clinical criteria: SPO₂≤93%, PaO₂/FiO₂ <_300 mmHg, or distressed RR≥30/min without oxygen inhalation and requiring oxygen therapy or more advanced breath support.

In some aspects, subjects will be evaluated for QTc prolongation. A QT interval is a measurement of the electrical properties of the heart, assessed by an electrocardiogram. In some aspects, QT prolongation can result in tachycardia, such as Torsades de Pointes. In some aspects, a corrected QT (QT_{c}) of >500 ms confers a high risk of a cardiac event. In some aspects, an increase in a basleine QT_{c} of >60 ms confers a high risk of a cardiac event. In some aspects, a normal QT_{c} for an adult made is <430 ms. In some aspects, a normal QT_{c} for an adult female is <450 ms. In some aspects, a normal QT_{c} for a child under 15 is <440 ms.

In some embodiments, the provided polynucleotides, proteins (e.g. fusion proteins or complexes), vehicles for delivery, or compositions containing any of the same,may ameliorate, or reduce, the severity of a symptoms relative to baseline (Day 1 of administration of any of the polynucleotides, polypeptide, or vehicles provided herein in any of Sections I, II, and III). In some embodiments, subjects are assessed for QT_{c}prologation. In some embodiments, the subjects self-assess. In some embodiments, subjects will be assessed on day 5 for when symptoms abate compared to baseline as described above. In some embodiments, subjects will be assessed on day 7 for when symptoms abate compared to baseline. In some embodiments, subjects will be assessed on day 14 for when symptoms abate compared to baseline. In some embodiments, subjects will be assessed on day 21 for when symptoms abate to baseline. In some embodiments, subjects will be assessed on day 30 for when symptoms abate compared to baseline.

In some embodiments, the provided polynucleotides, proteins (e.g. fusion proteins or complexes), vehicles for delivery, or compositions containing any of the same, may ameliorate, or reduce, viral load. In some embodiments, the provided polynucleotides, proteins (e.g. fusion proteins or complexes), vehicles for delivery, or compositions containing any of the same, may prevent virologic failure, as defined as an increase in viral load of >0.5 log on two consecutive days, or > 1 log increase in one day. In some aspects, wherein the viral load increase is not consistant with any baseline trend during the pre-treatment viral testing

In some embodiments, the provided polynucleotides, proteins (e.g. fusion proteins or complexes), vehicles for delivery, or compositions containing any of the same, may be used for delivery to a cell tissue or subject. In some embodiments, delivery of a cargo by administration of provided polynucleotides, proteins (e.g. fusion proteins or complexes), vehicles for delivery, or compositions containing any of the same, described herein may modify cellular protein expression levels, such via the delivery of regulatory factors of C19orf66 expression. In certain embodiments, the administered composition directs upregulation of (via expression in the cell, delivery in the cell, or induction within the cell) of one or more cargo (e.g., a C19orf66 polypeptide) that provide a functional activity.

In some of any embodiments, the provided polynucleotides, proteins (e.g. fusion proteins or complexes), vehicles for delivery, or compositions containing any of the same, (e.g., any as described in Section IV, a composition comprising the polynucleotides and polypeptides described in Sections I and II, and/or a composition comprising the vehicles described in Section III) mediates an effect on a target cell, and the effect lasts for at least 1, 2, 3, 4, 5, 6, or 7 days, 2, 3, or 4 weeks, or 1, 2, 3, 6, or 12 months. In some embodiments (e.g., wherein the vehicle particle composition comprises an exogenous polypeptide, such as encoding C19orf66 or a regulatory factor thereof), the effect lasts for less than 1, 2, 3, 4, 5, 6, or 7 days, 2, 3, or 4 weeks, or 1, 2, 3, 6, or 12 months.

In some of any embodiments, the provided polynucleotides, proteins (e.g. fusion proteins or complexes), vehicles for delivery, or compositions containing any of the same, described herein is delivered ex vivo to a cell or tissue, e.g., a human cell or tissue. In some embodiments, the composition is delivered to an ex vivo tissue that is in an injured state (e.g., from trauma, disease, hypoxia, ischemia or other damage). In some embodiments, the composition is delivered to an ex vivo transplant (e.g., a tissue explant or tissue for transplantation, e.g., a human vein, a musculoskeletal graft such as bone or tendon, cornea, skin, heart valves, nerves; or an isolated or cultured organ, e.g., an organ to be transplanted into a human, e.g., a human heart, liver, lung, kidney, pancreas, intestine, thymus, eye). In some embodiments, the composition is delivered to the tissue or organ before, during and/or after transplantation.

In some embodiments, the provided polynucleotides, proteins (e.g. fusion proteins or complexes), vehicles for delivery, or compositions containing any of the same, described herein can be administered to a subject, e.g., a mammal, e.g., a human. In such embodiments, the subject may be at risk of, may have a symptom of, or may be diagnosed with or identified as having, a particular disease or condition (e.g., known or suspects of having a coronavirus infection). In some embodiments, the disease or condition is a respiratory syndrome, such as Severe Acute Respiratory Syndrome (SARS).

### VI. Definitions

Unless defined otherwise, all terms of art, notations and other technical and scientific terms or terminology used herein are intended to have the same meaning as is commonly understood by one of ordinary skill in the art to which the claimed subject matter pertains. In some cases, terms with commonly understood meanings are defined herein for clarity and/or for ready reference, and the inclusion of such definitions herein should not necessarily be construed to represent a substantial difference over what is generally understood in the art.Unless indicated otherwise, abbreviations and symbols for chemical and biochemical names is per IUPAC-IUB nomenclature. Unless indicated otherwise, all numerical ranges are inclusive of the values defining the range as well as all integer values in-between.

As used herein, the articles “a” and “an” refer to one or to more than one (i.e. to at least one) of the grammatical object of the article. By way of example, “an element” means one element or more than one element.

As used herein, the term “about” will be understood by persons of ordinary skill in the art and will vary to some extent on the context in which it is used. As used herein, “about” when referring to a measurable value such as an amount, a temporal duration, and the like, is meant to encompass variations of ±20% or ±10%, more preferably ±5%, even more preferably ±1%, and still more preferably ±0.1 % from the specified value, as such variations are appropriate to perform the disclosed methods.

As used herein, “lipid particle” refers to any biological or synthetic particle that contains a bilayer of amphipathic lipids enclosing a lumen or cavity. Typically a lipid particle does not contain a nucleus. Examples of lipid particles include solid particles such as nanoparticles, viral-derived particles or cell-derived particles. Such lipid particles include, but are not limited to, viral particles (e.g. lentiviral particles), virus-like particles, viral vectors (e.g., lentiviral vectors) exosomes, enucleated cells, various vesicles, such as a microvesicle, a membrane vesicle, an extracellular membrane vesicle, a plasma membrane vesicle, a giant plasma membrane vesicle, an apoptotic body, a mitoparticle, a pyrenocyte, or a lysosome. In some embodiments, a lipid particle can be a fusosome. In some embodiments, the lipid particle is not a platelet.

As used herein a “biologically active portion,” such as with reference to a protein such as a G protein or an F protein, refers to a portion of the protein that exhibits or retains an activity or property of the full-length of the protein. For example, a biologically active portion of an F protein retains fusogenic activity in conjunction with the G protein when each are embedded in a lipid bilayer. A biologically active portion of the G protein retains fusogenic activity in conjunction with an F protein when each is embedded in a lipid bilayer. The retained activity and include 10%-150% or more of the activity of a full-length or wild-type F protein or G protein. Examples of biologically active portions of F and G proteins include truncations of the cytoplasmic domain, e.g. truncations of up to 1, 2, 3, 4, 5, 6, 7, 8 9, 10, 11, 12, 13, 14, 15, 20,25, 30, 35 or more contiguous amino acids, see e.g. Khetawat and Broder 2010 Virology Journal 7:312; Witting et al. 2013 Gene Therapy 20:997-1005; published international; patent application No. WO/2013/148327.

As used herein, “fusosome” refers to a particle containing a bilayer of amphipathic lipids enclosing a lumen or cavity and a fusogen that interacts with the amphipathic lipid bilayer. In embodiments, the fusosome comprises a nucleic acid. In some embodiments, the fusosome is a membrane enclosed preparation. In some embodiments, the fusosome is derived from a source cell.

As used herein, “fusosome composition” refers to a composition comprising one or more fusosomes.

As used herein, “fusogen” refers to an agent or molecule that creates an interaction between two membrane enclosed lumens. In embodiments, the fusogen facilitates fusion of the membranes. In other embodiments, the fusogen creates a connection, e.g., a pore, between two lumens (e.g., a lumen of a retroviral vector and a cytoplasm of a target cell). In some embodiments, the fusogen comprises a complex of two or more proteins, e.g., wherein neither protein has fusogenic activity alone. In some embodiments, the fusogen comprises a targeting domain.

As used herein, a “re-targeted fusogen” refers to a fusogen that comprises a targeting moiety having a sequence that is not part of the naturally-occurring form of the fusogen. In embodiments, the fusogen comprises a different targeting moiety relative to the targeting moiety in the naturally-occurring form of the fusogen. In embodiments, the naturally-occurring form of the fusogen lacks a targeting domain, and the re-targeted fusogen comprises a targeting moiety that is absent from the naturally-occurring form of the fusogen. In embodiments, the fusogen is modified to comprise a targeting moiety. In embodiments, the fusogen comprises one or more sequence alterations outside of the targeting moiety relative to the naturally-occurring form of the fusogen, e.g., in a transmembrane domain, fusogenically active domain, or cytoplasmic domain.

As used herein, a “targeted envelope protein” refers to a polypeptide that contains a henipavirus G protein attached to a single domain antibody (sdAb) variable domain, such as a VL or VH only sdAb, nanobodies, camelid VHH domains, shark IgNAR or fragments thereof, that targets a molecule on a desired cell type. In some such embodiments, the attachment may be directly or indirectly via a linker, such as a peptide linker.

As used herein, a “targeted lipid particle” refers to a lipid particle that contains a targeted envelope protein embedded in the lipid bilayer.

As used herein, a “retroviral nucleic acid” refers to a nucleic acid containing at least the minimal sequence requirements for packaging into a retrovirus or retroviral vector, alone or in combination with a helper cell, helper virus, or helper plasmid. In some embodiments, the retroviral nucleic acid further comprises or encodes an exogenous agent, a positive target cell-specific regulatory element, a non-target cell-specific regulatory element, or a negative TCSRE. In some embodiments, the retroviral nucleic acid comprises one or more of (e.g., all of) a 5′ LTR (e.g., to promote integration), U3 (e.g., to activate viral genomic RNA transcription), R (e.g., a Tat-binding region), U5, a 3′ LTR (e.g., to promote integration), a packaging site (e.g., psi (El)), RRE (e.g., to bind to Rev and promote nuclear export). The retroviral nucleic acid can comprise RNA (e.g., when part of a virion) or DNA (e.g., when being introduced into a source cell or after reverse transcription in a recipient cell). In some embodiments, the retroviral nucleic acid is packaged using a helper cell, helper virus, or helper plasmid which comprises one or more of (e.g., all of) gag, pol, and env.

As used herein, a “target cell” refers to a cell of a type to which it is desired that a targeted lipid particle delivers an exogenous agent. In embodiments, a target cell is a cell of a specific tissue type or class, e.g., an immune effector cell, e.g., a T cell. In some embodiments, a target cell is a diseased cell, e.g., a cancer cell. In some embodiments, the fusogen, e.g., retargeted fusogen leads to preferential delivery of the exogenous agent to a target cell compared to a non-target cell.

As used herein a “non-target cell” refers to a cell of a type to which it is not desired that a targeted lipid particle delivers an exogenous agent. In some embodiments, a non-target cell is a cell of a specific tissue type or class. In some embodiments, a non-target cell is a non-diseased cell, e.g., a non-cancerous cell. In some embodiments, the fusogen, e.g., re-targeted fusogen leads to lower delivery of the exogenous agent to a non-target cell compared to a target cell.

As used herein, the term “specifically binds” to a target molecule, such as an antigen, means that a binding molecule, such as a single domain antibody, reacts or associates more frequently, more rapidly, with greater duration and/or with greater affinity with a particular target molecule than it does with alternative molecules. A binding molecule, such as a sdAb variable domain, “specifically binds” to a target molecule if it binds with greater affinity, avidity, more readily, and/or with greater duration than it binds to other molecules. It is understood that a binding molecule, such as a sdAb, that specifically binds to a first target may or may not specifically bind to a second target. As such, “specific binding” does not necessarily require (although it can include) exclusive binding.

As used herein, “percent (%) amino acid sequence identity” and “homology” with respect to a peptide, polypeptide or antibody sequence are defined as the percentage of amino acid residues in a candidate sequence that are identical with the amino acid residues in the specific peptide or polypeptide sequence, after aligning the sequences and introducing gaps, if necessary, to achieve the maximum percent sequence identity, and not considering any conservative substitutions as part of the sequence identity. Alignment for purposes of determining percent amino acid sequence identity can be achieved in various ways that are within the skill in the art, for instance, using publicly available computer software such as BLAST, BLAST-2, ALIGN or MEGALIGNTM (DNASTAR) software. Those skilled in the art can determine appropriate parameters for measuring alignment, including any algorithms needed to achieve maximal alignment over the full length of the sequences being compared.

An amino acid substitution may include but are not limited to the replacement of one amino acid in a polypeptide with another amino acid. Amino acid substitutions may be introduced into an antibody of interest and the products screened for a desired activity, for example, retained/improved binding. Non-conservative substitutions will entail exchanging a member of one of these classes for another class.

The term, “corresponding to” with reference to positions of a protein, such as recitation that nucleotides or amino acid positions “correspond to” nucleotides or amino acid positions in a disclosed sequence, such as set forth in the Sequence listing, refers to nucleotides or amino acid positions identified upon alignment with the disclosed sequence based on structural sequence alignment or using a standard alignment algorithm, such as the GAP algorithm. For example, corresponding residues of a similar sequence (e.g. fragment or species variant) can be determined by alignment to a reference sequence by structural alignment methods. By aligning the sequences, one skilled in the art can identify corresponding residues, for example, using conserved and identical amino acid residues as guides.

The term “isolated” as used herein refers to a molecule that has been separated from at least some of the components with which it is typically found in nature or produced. For example, a polypeptide is referred to as “isolated” when it is separated from at least some of the components of the cell in which it was produced. Where a polypeptide is secreted by a cell after expression, physically separating the supernatant containing the polypeptide from the cell that produced it is considered to be “isolating” the polypeptide. Similarly, a polynucleotide is referred to as “isolated” when it is not part of the larger polynucleotide (such as, for example, genomic DNA or mitochondrial DNA, in the case of a DNA polynucleotide) in which it is typically found in nature, or is separated from at least some of the components of the cell in which it was produced, for example, in the case of an RNA polynucleotide. Thus, a DNA polynucleotide that is contained in a vector inside a host cell may be referred to as “isolated”.

The term “effective amount” as used herein means an amount of a pharmaceutical composition which is sufficient enough to significantly and positively modify the symptoms and/or conditions to be treated (e.g., provide a positive clinical response). The effective amount of an active ingredient for use in a pharmaceutical composition will vary with the particular condition being treated, the severity of the condition, the duration of treatment, the nature of concurrent therapy, the particular active ingredient(s) being employed, the particular pharmaceutically-acceptable excipient(s) and/or carrier(s) utilized, and like factors with the knowledge and expertise of the attending physician.

An “exogenous agent” as used herein with reference to a targeted lipid particle, refers to an agent that is neither comprised by nor encoded in the corresponding wild-type virus or fusogen made from a corresponding wild-type source cell. In some embodiments, the exogenous agent does not naturally exist, such as a protein or nucleic acid that has a sequence that is altered (e.g., by insertion, deletion, or substitution) relative to a naturally occurring protein. In some embodiments, the exogenous agent does not naturally exist in the source cell. In some embodiments, the exogenous agent exists naturally in the source cell but is exogenous to the virus. In some embodiments, the exogenous agent does not naturally exist in the recipient cell. In some embodiments, the exogenous agent exists naturally in the recipient cell, but is not present at a desired level or at a desired time. In some embodiments, the exogenous agent comprises RNA or protein.

As used herein, “operably linked” or “operably associated” includes reference to a functional linkage of at least two sequences. For example, operably linked includes linkage between a promoter and a second sequence, wherein the promoter sequence initiates and mediates transcription of the DNA sequence corresponding to the second sequence. Operably associated includes linkage between an inducing or repressing element and a promoter, wherein the inducing or repressing element acts as a transcriptional activator of the promoter.

As used herein, a “promoter” refers to a cis- regulatory DNA sequence that, when operably linked to a gene coding sequence, drives transcription of the gene. The promoter may comprise a transcription factor binding sites. In some embodiments, a promoter works in concert with one or more enhancers which are distal to the gene.

As used herein, a “vehicle” refers to a biological carrier for delivering genes or proteins to cells to facilitate their recognition or uptake by cells. Examples of delivery vehicles include, but are not limited to, lipid and non-lipid particles, such as virus or virus like particles, liposomes, microparticles, nanoparticles, nanogels, dendrimer or dendrisomes.

As used herein, a composition refers to any mixture of two or more products, substances, or compounds, including cells. It may be a solution, a suspension, liquid, powder, a paste, aqueous, non-aqueous or any combination thereof.

As used herein, the term “pharmaceutically acceptable” refers to a material, such as a carrier or diluent, which does not abrogate the biological activity or properties of the compound, and is relatively nontoxic, i.e., the material may be administered to an individual without causing undesirable biological effects or interacting in a deleterious manner with any of the components of the composition in which it is contained.

As used herein, the term “pharmaceutical composition” refers to a mixture of at least one compound of the invention with other chemical components, such as carriers, stabilizers, diluents, dispersing agents, suspending agents, thickening agents, and/or excipients. The pharmaceutical composition facilitates administration of the compound to an organism. Multiple techniques of administering a compound exist in the art including, but not limited to, intravenous, oral, aerosol, parenteral, ophthalmic, pulmonary and topical administration.

A “disease” or “disorder” as used herein refers to a condition where treatment is needed and/or desired.

As used herein, the terms “treat,” “treating,” or “treatment” refer to ameliorating a disease or disorder, e.g., slowing or arresting or reducing the development of the disease or disorder or reducing at least one of the clinical symptoms thereof. For purposes of this disclosure, ameliorating a disease or disorder can include obtaining a beneficial or desired clinical result that includes, but is not limited to, any one or more of: alleviation of one or more symptoms, diminishment of extent of disease, preventing or delaying spread (for example, metastasis, for example metastasis to the lung or to the lymph node) of disease, preventing or delaying recurrence of disease, delay or slowing of disease progression, amelioration of the disease state, inhibiting the disease or progression of the disease, inhibiting or slowing the disease or its progression, arresting its development, and remission (whether partial or total).

The terms “individual” and “subject” are used interchangeably herein to refer to an animal; for example a mammal. The term patient includes human and veterinary subjects. In some embodiments, methods of treating mammals, including, but not limited to, humans, rodents, simians, felines, canines, equines, bovines, porcines, ovines, caprines, mammalian laboratory animals, mammalian farm animals, mammalian sport animals, and mammalian pets, are provided. The subject can be male or female and can be any suitable age, including infant, juvenile, adolescent, adult, and geriatric subjects. In some examples, an “individual” or “subject” refers to an individual or subject in need of treatment for a disease or disorder. In some embodiments, the subject to receive the treatment can be a patient, designating the fact that the subject has been identified as having a disorder of relevance to the treatment, or being at adequate risk of contracting the disorder. In particular embodiments, the subject is a human, such as a human patient.

### VII. Exemplary Embodiments

Among the provided emobodiments are:

1. A method of treating a coronavirus infection in a subject, the method comprising administering to a subject known or suspected of having a coronavirus infection a composition comprising an agent for delivery of a Chromosome 19 Open Reading Frame 66 (C l9orf66) protein to the subject or an agent for delivery of a regulatory factor that increases expression of the gene encoding C19orf66 in a cell in the subject.

2. A method of reducing the likelihood of a coronavirus infection in a subject, the method comprising administering to a subject known or suspected of being exposed to a coronavirus a composition comrpising an agent for delivery of a Chromosome 19 Open Reading Frame 66 (C19orf66) protein to the subject or an agent for delivery of a regulatory factor that increases expression of the gene encoding C19orf66 in a cell in the subject.

3. The method of embodiment 1 or embodiment 2, wherein the agent for delivery is heterologous to the subject.

4. The method of any of embodiments 1-3, wherein the cell in the subject is infected with the coronavirus.

5. The method of any of embodiments 1-4, wherein the subject is administered an agent for delivery of a C19orf66 protein to the subject, wherein the agent is a nucleotide sequence encoding the C19orf66 protein.

6. The method of any of embodiments 1-4, wherein the subject is administered an agent for delivery of a regulatory factor that increases expression of the gene encoding C 19orf66, wherein the agent is a nucleotide sequence encoding the regulatory factor.

7. The method of embodiment 5 or embodiment 6, wherein the nucleotide sequence is operably linked to a promoter to control expression.

8. The method of embodiment 7, wherein the promoter is a constitutive promoter.

9. The method of embodiment 7 or embodiment 8, wherein the promoter is a human Ubiquitin C (UbC) promoter, a human elongation factor 1α (EFla) promoter, an SV40 promoter, a Cytomegalovirus (CMV) promoter, or a PGK-1 promoter.

10. The method of any of embodiments 5-7, wherein the nucleotide sequence is operably linked to a promoter to control expression in the lung.

11. The method of embodiment 7 or 10, wherein the promoter is a human surfactant A promoter , a human surfactant B promoter, a human surfactant C promoter, a human surfactant D promoter, human ROB04 promoter, or a human CDH1 gene.

12. The method of embodiment 7, 10 or 11, wherein the promoter is the human surfactant B promoter set forth in SEQ ID NO: 10.

13. The method of any of embodiments 1-4, wherein the subject is administered an agent for delivery of a C19orf66 protein to the subject, wherein the agent is the C19orf66 protein.

14. The method of embodiment 13, wherein the C19orf66 protein is a recombinant protein.

15. The method of embodiment 13 or embodiment 14, wherein the C19orf66 protein is linked to a cell penetrating peptide.

16. The method of embodiment 15, wherein the protein is linked indirectly to the cell penetrating peptide via a peptide linker.

17. The method of any of embodiments 1-4, wherein the subject is administered an agent for delivery of a regulatory factor that increases expression of the gene encoding C 19orf66, wherein the agent is a regulatory factor protein or a protein complex.

18. The method of embodiment 17, wherein the regulatory factor protein or protein complex is linked to a cell penetrating peptide.

19. The method of embodiment 18, wherein the protein or protein complex is linked indirectly to the cell penetrating peptide via a peptide linker.

20. The method of embodiment 15, embodiment 16, embodiment 18 or embodiment 19, wherein the cell penetrating peptide is a peptide that facilitates delivery to the interior of a cell.

21. The method of any of embodiments 15, 16 and 18-20, wherein the cell penetrating peptide is selected from the group consisting of:
TAT (SEQ ID NO: 13)
Penetratin (SEQ ID NO: 14)
Transporant (SEQ ID NO: 15)
Pept 1 (SEQ ID NO: 16)
Pept 2 (SEQ ID NO: 17)
Transportan (SEQ ID NO: 18)
IgV (SEQ ID NO: 19 )

22. The method of any of embodiments 1-21, wherein the administration of the agent inhibits or prevents viral replication of the coronavirus in the subject.

23. The method of any of embodiments 1-22, wherein the administration of the agent inhibits or prevents ribosomal frameshifting in the subject.

24. The method of any of embodiments 1-23, wherein the administration of the agent inhibits or prevents viral RNA processing in the subject.

25. The method of any of embodiments 1-24, wherein the C19orf66 protein is or comprises the sequence of amino acids set forth in SEQ ID NO: 1, or a sequence of amino acids that has at least 90%, at least 92%, at least 95%, or at least 98% sequence identity to the sequence of amino acids set forth in SEQ ID NO: 1.

26. The method of any of embodiments 1-25, wherein the C19orf66 protein is or comprises the sequence set forth in SEQ ID NO: 1.

27. The method of any of embodiments 1-26, wherein the C19orf66 protein is encoded by the nucleotide sequence set forth in SEQ ID NO: 2, or a nucleotide sequence that has at least 90%, at least 92%, at least 95%, or at least 98% sequence identity to SEQ ID NO:2.

28. The method of any of embodiments 1-27, wherein the C19orf66 protein is encoded by the nucleotide sequence set forth in SEQ ID NO:2.

29. The method of any of embodiments 1-24, wherein the C19orf66 protein is or comprises the sequence of amino acids set forth in SEQ ID NO: 3, or a sequence of amino acids that has at least 90%, at least 92%, at least 95%, or at least 98% sequence identity to the sequence of amino acids set forth in SEQ ID NO:3.

30. The method of any of embodiments 1-24 and 29, wherein the C19orf66 protein is or comprises the sequence set forth in SEQ ID NO:3.

31. The method of any of embodiments 1-24, 29 and 30, wherein the C19orf66 protein is encoded by the nucleotide sequence set forth in SEQ ID NO: 4, or a nucleotide sequence that has at least 90%, at least 92%, at least 95%, or at least 98% sequence identity to the sequence set forth in SEQ ID NO:4.

32. The method of any of embodiments 1-24, and 29-31 1, wherein the C19orf66 protein is encoded by the sequence set forth in SEQ ID NO:4.

33. The method of any of embodiments 1-24 , wherein the C19orf66 protein is or comprises the sequence of amino acids set forth in SEQ ID NO: 5, or a sequence of amino acids that has at least 90%, at least 92%, at least 95%, or at least 98% sequence identity to the sequence of amino acids set forth in SEQ ID NO:5.

34. The method of any of embodiments 1-24 and 33, wherein the C19orf66 protein is or comprises the sequence set forth in SEQ ID NO:5.

35. The method of any of embodiments 1-24, 33 and 34, wherein the C19orf66 is encoded by the nucleotide sequence set forth in SEQ ID NO: 6, or a nucleotide sequence that has at least 90%, at least 92%, at least 95%, or at least 98% sequence identity to the sequence set forth in SEQ ID NO:6.

36. The method of any of embodiments 1-24, and 33-35, wherein the C19orf66 protein is encoded by the sequence set forth in SEQ ID NO:6.

37. The method of any of embodiments 1-24, wherein the C19orf66 protein is or comprises the sequence of amino acids set forth in SEQ ID NO: 7, or a sequence of amino acids that has at least 90%, at least 92%, at least 95%, or at least 98% sequence identity to the sequence of amino acids set forth in SEQ ID NO:7.

38. The method of any of embodiments 1-24 and 37, wherein the C19orf66 protein is or comprises the sequence set forth in SEQ ID NO:7.

39. The method of any of embodiments 1-24, 37 and 38, wherein the C19orf66 protein is encoded by the nucleotide sequence set forth in SEQ ID NO: 8, or a sequence of nucleic acids that has at least 90%, at least 92%, at least 95%, or at least 98% sequence identity to the sequence set forth in SEQ ID NO:.8

40. The method of any of embodiments 1-24, and 37-39, wherein the C19orf66 protein is encoded by the nucleotide sequence set forth in SEQ ID NO:.8

41. The method of any of embodiments 1-40, wherein the C 19orf66 protein comprises a nuclear localization signal.

42. The method of embodiment 41, wherein the nuclear localization signal is selected from the group consisting of:

KKRXKR (SEQ ID NO: 81),

KRPAATKKAGQAKKKK (SEQ ID NO: 82)

PAAKRBKLD (SEQ ID NO: 83),

PKKKRKVEDP (SEQ ID NO: 84), and

RRVPQRKEVSRCRKCRK (SEQ ID: NO 86)

43. The method of embodiment 41 or embodiment 42, wherein the nuclear localization signal has the sequence of nucleic acids set forth in SEQ ID NO: 86.

44. The method of any of embodiments 1-43, wherein the C19orf66 protein comprises a nuclear export signal.

45. The method of embodiment 44, wherein the nuclear export signal is selected from LXXXLXXLXL (SEQ ID NO:85) or LEDLDNLIL (SEQ ID: NO 87).

46. The method of embodiment 44 or embodiment 45, where in the nuclear export signal has the sequence of nucleic acids set forth in SEQ ID NO: 87.

47. The method of any of embodiments 1-4, 6-12, 17-46, wherein the regulatory factor controls targeted transcriptional activation of the gene encoding C19orf66.

48. The method of any of embodiments 1-4, 6-12, 17-47, wherein the regulatory factor is a fusion protein comprising a site-specific binding domain specific for the C19orf66 gene, and a transcriptional activator.

49. The method of embodiment 48, wherein the site-specific binding domain is selected from the group consisting of: zinc fingers, transcription activation like (TAL) effectors, meganucleases, and CRISPR/Cas system, or a modified form thereof.

50. The method of embodiment 48 or embodiment 49, wherein the regulatory factor is a zinc finger transcription factor (ZF-TF).

51. The method of embodiment 49, wherein the site-specific binding domain is a CRISPR/Cas system, wherein the CRISPR/Cas system comprises a modified Cas nuclease that lacks nuclease activity and a guide RNA (gRNA).

52. The method of embodiment 51, wherein the modified nuclease is a catalytically dead Cas9 (dCas9).

53. The method of any of embodiments 47-52, wherein the transcriptional activator is selected from Herpes simplex-derived transactivation domain, Dnmt3a methyltransferase domain, p65, VP16, and VP64.

54. The method of any of embodiments 57-53, wherein the transcriptional activator is the tripartite activator VP64-p65-Rta (VPR).

55.The method of any of embodiments 1-54, wherein the agent is comprised in a vehicle that is a lipid particle or a non-lipid particle.

56. The method of embodiment 55, wherein the vehicle is a lipid particle that is a viral vector or a viral-like particle.

57. The method of embodiment 56, wherein the viral vector or viral-like particle is derived from an Adeno-associated virus (AAV).

58. The method of embodiment 57, wherein the AAV is of serotype 1, 2, 5, 6. \

59. The method of embodiment 57 or embodiment 58, wherein the AAV is of serotype 5.

60. The method of embodiment 57 or embodiment 58, wherein the AAV is of serotype 6.

61. The method of embodiment 56, wherein the viral vector or viral-like particle is derived from a lentivirus.

62. The method of embodiment 61, wherein the lentivirus is Human Immunodeficiency Virus-1 (HIV-1).

63. The method of any of embodiments 56-62, wherein the viral vector or viral-like particle is a virus-like particle.

64. The method of embodiment 63, wherein the virus-like particle is replication defective.

65. The method of any of embodiments 56-64, wherein the viral vector or viral-like particle comprises a fusogen.

66. The method of embodiment 55, wherein the vehicle is a lipid particle, wherein the lipid particle comprises (i) a lipid bilayer enclosing a lumen, and (ii) a fusogen, wherein the fusogen is embedded in the lipid bilayer.

67. The method of embodiment 66, wherein the lipid bilayer is derived from a membrane of a host cell used for producing a virus or virus-like particle.

68. The method of embodiment 66 or embodiment 67, wherein the lipid bilayer is derived from a membrane of a host cell used for producing a virus-like particle, wherein the virus-like particle is replication defective.

69. The method of embodiment 67 or embodiment 68, wherein the virus or virus-like particle is a retrovirus.

70.The method of embodiment 69, wherein the retrovirus is a lentivirus.

71. The method of embodiment 67 or embodiment 68, wherein the virus or virus-like particle is an adenovirus.

72. The method of any of embodiments 65-71, wherein the fusogen is a viral fusogen selected from a Class I viral membrane fusion protein, a Class II viral membrane protein, a Class II viral membrane fusion protein, a viral membrane glycoprotein, or a viral envelope protein.

73. The method of any of embodiments 65-72, wherein the fusogen is a vesicular stomatitis virus envelope glycoprotein (VSV-G).

74. The method of any of embodiments any of embodiments 65-72, wherein the fusogen is a syncytin.

75. The method of any of embodiments 65-74, wherein the fusogen is from a coronavirus.

76. The method of any of embodiments any of embodiments 65-71 and 75, wherein the the fusogen is a Severe Acute Respiratory Syndrome (SARS) coronavirus 1 (SARS CoV-1) spike glycoprotein.

77. The method of any of embodiments any of embodiments 65-71 and 76, wherein the fusogen is a Severe Acute Respiratory Syndrome (SARS) coronavirus 2 (SARS CoV-2) spike glycoprotein.

78. The method of any of embodiments any of embodiments 65-71 and 75, wherein the fusogen is an alpha coronavirus CD13 protein.

79. The method of any of embodiments 65-71, wherein the fusogen comprises an F protein molecule or a biologically active portion thereof from a Paramyxovirus and/or a glycoprotein G (G protein) or a biologically active portion thereof from a Paramyxovirus.

80. The method of any of embodiments 65-72, wherein the fusogen is derived from an F protein molecule or a biologically active portion thereof from a Paramyxovirus and/or a glycoprotein G (G protein) or a biologically active portion thereof from a Paramyxovirus.

81. The method of embodiment 79 or embodiment 80, wherein the Paramyxovirus is a henipavirus.

82. The method of any of embodiments 79-81, wherein the Paramyxovirus is Nipah virus.

83. The method of any of embodiments 79-81, wherein the Paramyxovirus is Hendra virus.

84. The method of any one of embodiments 65-83, wherein the fusogen is a re-targeted fusogen comprising a targeting moiety that binds to a molecule on a target cell.

85. The method of embodiment 84, wherein the targeting moiety is a Design ankyrin repeat proteins (DARPin), a single domain antibody (sdAb), a single chain variable fragment (scFv), or an antigen-binding fibronectin type III (Fn3) scaffold.

86. The method of embodiment 84 or embodiment 85, wherein the target cell is known or suspected of being infected by a coronavirus.

87. The method of any of embodiments 84-86, wherein targeting moiety binds a receptor of a coronavirus.

88. The method of any of embodiments 84-87, wherein the targeting moiety binds angiotensin-converting enzyme 2 (ACE2).

89. The method of any of embodiments 84-87, wherein the targeting moiety binds to transmembrane proteinase, serine 2 (TMPRSS2).

90. The method of any of embodiments 84-87, wherein the targeting moiety binds to dipeptidyl peptidase 4 (DPP4).

91. The method of any one of embodiments 65-90, wherein the fusogen is modified to reduce its native binding tropism.

92. The method of any of embodiments 79-91, wherein the G protein or the biologically active portion thereof is a mutant NiV-G protein that exhibits reduced binding to Ephrin B2 or Ephrin B3.

93. The method of embodiment 92, wherein the mutant NiV-G protein comprises one or more amino acid substitutions corresponding to amino acid substitutions selected from the group consisting of E501A, W504A, Q530A and E533A with reference to numbering set forth in SEQ ID NO:26.

94. The method of any of embodiments 79-93, wherein the mutant NiV-G protein has the amino acid sequence set forth in SEQ ID NO: 69 or an amino acid sequence having at or about 80%, at least at or about 81%, at least at or about 82%, at least at or about 83%, at or about 84%, at least at or about 85%, at least at or about 86%, or at least at or about 87%, at least at or about 88%, or at least at or about 89%, at least at or about 90%, at least at or about 91%, at least at or about 92%, at least at or about 93%, at least at or about 94%, at least at or about 95%, at or about 96%, at least at or about 97%, at least at or about 98%, or at least at or about 99% sequence identity to SEQ ID NO:69.

95. The method of any of embodiments 79-93, wherein the mutant NiV-G protein has the amino acid sequence set forth in SEQ ID NO: 70 or an amino acid sequence having at or about 80%, at least at or about 81%, at least at or about 82%, at least at or about 83%, at or about 84%, at least at or about 85%, at least at or about 86%, or at least at or about 87%, at least at or about 88%, or at least at or about 89%, at least at or about 90%, at least at or about 91%, at least at or about 92%, at least at or about 93%, at least at or about 94%, at least at or about 95%, at or about 96%, at least at or about 97%, at least at or about 98%, or at least at or about 99% sequence identity to SEQ ID NO:70.

96. The method of any of embodiments 79-95, wherein the NiV-F protein is a biologically active portion thereof that has a 20 amino acid truncation at or near the C-terminus of the wild-type NiV-F protein (SEQ ID NO:37).

97. The method of embodiment 96, wherein the NiV-F protein has an amino acid sequence set forth in SEQ ID NO:76 or an amino acid sequence having at or about 80%, at least at or about 81%, at least at or about 82%, at least at or about 83%, at or about 84%, at least at or about 85%, at least at or about 86%, or at least at or about 87%, at least at or about 88%, or at least at or about 89%, at least at or about 90%, at least at or about 91%, at least at or about 92%, at least at or about 93%, at least at or about 94%, at least at or about 95%, at or about 96%, at least at or about 97%, at least at or about 98%, or at least at or about 99% sequence identity to SEQ ID NO: 76.

98. The method of any of embodiments 79-95, wherein the NiV-F protein is a biologically active portion thereof that has a 22 amino acid truncation at or near the C-terminus of the wild-type NiV-F protein (SEQ ID NO:37).

99. The method of embodiment 98, wherein the NiV-F protein has an amino acid sequence set forth in SEQ ID NO:75 or a sequence having at or about 80%, at least at or about 81%, at least at or about 82%, at least at or about 83%, at or about 84%, at least at or about 85%, at least at or about 86%, or at least at or about 87%, at least at or about 88%, or at least at or about 89%, at least at or about 90%, at least at or about 91%, at least at or about 92%, at least at or about 93%, at least at or about 94%, at least at or about 95%, at or about 96%, at least at or about 97%, at least at or about 98%, or at least at or about 99% sequence identity to SEQ ID NO: 75.

100.The method of embodiment 98 or embodiment 99, wherein the NiV-F protein has an amino acid sequence set forth in SEQ ID NO:80 or a sequence having at or about 80%, at least at or about 81%, at least at or about 82%, at least at or about 83%, at or about 84%, at least at or about 85%, at least at or about 86%, or at least at or about 87%, at least at or about 88%, or at least at or about 89%, at least at or about 90%, at least at or about 91%, at least at or about 92%, at least at or about 93%, at least at or about 94%, at least at or about 95%, at or about 96%, at least at or about 97%, at least at or about 98%, or at least at or about 99% sequence identity to SEQ ID NO: 80.

101.The method of any of embodiments 78-99, wherein the NiV-F protein comprises a point mutation on an N-linked glycosylation site.

102.The method of any of embodiments 79-97 and 101, wherein the NiV-F protein is a biologically active portion thereof that comprises:
i) a 20 amino acid truncation at or near the C-terminus of the wild-type NiV-F protein (SEQ ID NO:37); and
ii) a point mutation on an N-linked glycosylation site.

103.The method of embodiment 102, wherein the NiV-F protein has an amino acid sequence set forth in SEQ ID NO:74 or an amino acid sequence having at or about 80%, at least at or about 81%, at least at or about 82%, at least at or about 83%, at or about 84%, at least at or about 85%, at least at or about 86%, or at least at or about 87%, at least at or about 88%, or at least at or about 89%, at least at or about 90%, at least at or about 91%, at least at or about 92%, at least at or about 93%, at least at or about 94%, at least at or about 95%, at or about 96%, at least at or about 97%, at least at or about 98%, or at least at or about 99% sequence identity to SEQ ID NO: 74.

104.The method of any of embodiments 1-54, wherein the agent is comprised in a non-viral vector.

105. The method of embodiment 104, wherein the non-viral particle is a liposome, a microparticle, a nanoparticle, a nanogel, a dendrimer or a dendrisome.

106. The method of embodiment 104 or embodiment 105, wherein the non-viral vector is a nanoparticle.

107.The method of embodiment 104 or embodiment 105, wherein the non-viral vector is a liposome.

108.The method of embodiment 104, wherein the non-viral vector is a plasmid.

109. The method of any of embodiments 104-108, wherein the non-viral vector further comprises a vector-surface targeting moiety that binds to a molecule on a target cell, optionally where the target cell is a lung cell.

110. The method of embodiment 109, wherein the target cell is known or suspected of being infected by a coronavirus.

111. The method of embodiment 109 or embodiment 110, wherein targeting moiety binds a receptor of a coronavirus.

112. The method of any of embodiments 109-111, wherein the targeting moiety binds angiotensin-converting enzyme 2 (ACE2).

113. The method of any of embodiments 109-111, wherein the targeting moiety binds to transmembrane proteinase, serine 2 (TMPRSS2).

114. The method of any of embodiments 109-111, wherein the targeting moiety binds to dipeptidyl peptidase 4 (DPP4).115. The method of any of embodiments 109-114, wherein the vector-surface targeting moiety is a peptide or a polypeptide.

116. The method of any of embodiments 109-115, wherein the polypeptide is an antibody or antigen-binding fragment.

117. The method of any of embodiments 104-116, wherein the non-viral vector is freeze dried.

118.The method of embodiment 117, wherein the non-viral vector is subject to freeze and thaw prior to its administration.

119.The method of any of embodiments 1-54, wherein the agent is administered as a naked nucleic acid.

120. The method of any of embodiments 1-54 and 119, wherein the agent is administered as an mRNA .

121. The method of embodiment 119 or embodiment 120, wherein the agent is freeze dried.

122. The method of embodiment 121, wherein the agent is subject to freeze and thaw prior to its administration.

123. A polynucleotide comprising a nucleotide sequence encoding Chromosome 19 Open Reading Frame 66 (C19orf66) or a nucleotide sequence encoding a regulatory factor that increases expression of the gene encoding C19orf66 in a cell, wherein the nucleotide sequence is operably linked to a promoter to control expression in the lung.

124.The polynucleotide of embodiment 123, wherein the nucleotide sequence encodes C19orf66.

125.The polynucleotide of embodiment 123, wherein the nucleotide sequence encodes a regulatory factor that that increases expression of the gene encoding C19orf66 when the polynucleotide is administered to a cell in a subject. 126. The polynucleotide of any of embodiments 123-125, wherein the encoded CWorf66 inhibits or prevents viral replication, optionally wherein the encoded C19orf66 inhibits or prevents viral replication of a Coronavirus..

127. The polynucleotide of any of embodiments 123-126, wherein the encoded C19orf66 inhibits or prevents ribosomal frameshifting.

128. The polynucleotide of any of embodiments 123-127, wherein the encoded C19orf66 inhibits or prevents viral RNA processing.

129. The polynucleotide of any of embodiments 123-128, wherein the encoded C19orf66 is or comprises the sequence of amino acids set forth in SEQ ID NO: 1, or a sequence of amino acids that has at least 90%, at least 92%, at least 95%, or at least 98% sequence identity to the sequence of amino acids set forth in SEQ ID NO: 1.

130.The polynucleotide of any of embodiments 123-129, wherein the encoded C19orf66 is or comprises the sequence set forth in SEQ ID NO:1.

131. The polynucleotide of any of embodiments 123-130 wherein the nucleotide sequence encoding C19orf66 is or comprises the sequence set forth in SEQ ID NO: 2, or a sequence that has at least 90%, at least 92%, at least 95%, or at least 98% sequence identity to the sequence set forth in SEQ ID NO:2.

132.The polynucleotide of any of embodiments 123-131, wherein the nucleotide sequence is or comprises the sequence set forth in SEQ ID NO:2.

133. The polynucleotide of any of embodiments 123-128, wherein the encoded C19orf66 is or comprises the sequence of amino acids set forth in SEQ ID NO: 3, or a sequence of amino acids that has at least 90%, at least 92%, at least 95%, or at least 98% sequence identity to the sequence of amino acids set forth in SEQ ID NO:3.

134. The polynucleotide of any of embodiments 123-128 and 133, wherein the encoded C19orf66 has the sequence set forth in SEQ ID NO:3.

135. The polynucleotide of any of embodiments 123-128, 133 and 134, wherein the nucleotide sequence is or comprises the sequence set forth in SEQ ID NO: 4, or a sequence that has at least 90%, at least 92%, at least 95%, or at least 98% sequence identity to the sequence set forth in SEQ ID NO:4.

136. The polynucleotide of any of embodiments 123-128 and 133-135, wherein the nucleotide sequence is or comprises the sequence set forth in SEQ ID NO:4.

137. The polynucleotide of any of embodiments 123-128, wherein the encoded C19orf66 is or comprises the sequence of amino acids set forth in SEQ ID NO: 5, or a sequence of amino acids that has at least 90%, at least 92%, at least 95%, or at least 98% sequence identity to the sequence of amino acids set forth in SEQ ID NO:5.

138. The polynucleotide of any of embodiments 123-128 and 137, wherein the encoded C19orf66 is or comprises the sequence set forth in SEQ ID NO:5.

139. The polynucleotide of any of embodiments 123-128, 137 and 138, wherein the nucleotide sequence is or comprises the sequence set forth in SEQ ID NO: 6, or a sequence that has at least 90%, at least 92%, at least 95%, or at least 98% sequence identity to the sequence set forth in SEQ ID NO:6.

140. The polynucleotide of any of embodiments 123-128 and 137-139, wherein the nucleotide sequence is or comprises the sequence set forth in SEQ ID NO:6.

141. The polynucleotide of any of embodiments 123-128, wherein the encoded C19orf66 is or comprises the sequence of amino acids set forth in SEQ ID NO: 7, or a sequence of amino acids that has at least 90%, at least 92%, at least 95%, or at least 98% sequence identity to the sequence of amino acids set forth in SEQ ID NO:7.

142. The polynucleotide of any of embodiments 123-128 and 141, wherein the encoded C19orf66 is or comprises the sequence set forth in SEQ ID NO:7.

143. The polynucleotide of any of embodiments 123-128, 141 and 142, wherein nucleotide sequence is or comprises the sequence set forth in SEQ ID NO: 8, or a sequence that has at least 90%, at least 92%, at least 95%, or at least 98% sequence identity to the sequence set forth in SEQ ID NO:.8

144. The polynucleotide of any of embodiments 123-128 and 141-143, wherein the nucleotide sequence is or comprises the sequence set forth in SEQ ID NO:.8

145. The polynucleotide of any of embodiments 123-144, wherein the encoded C19orf66 comprises a nuclear localization signal.

146. The polynucleotide of embodiment 145, wherein the nuclear localization signal is selected from the group consisting of:

KKRXKR (SEQ ID NO: 81),

KRPAATKKAGQAKKKK (SEQ ID NO: 82)

PAAKRBKLD (SEQ ID NO: 83),

PKKKRKVEDP (SEQ ID NO: 84), and

RRVPQRKEVSRCRKCRK (SEQ ID: NO 86)

147. The polynucleotide of embodiment 145 or embodiment 146, wherein the nuclear localization signal has the sequence of nucleic acids set forth in SEQ ID NO: 86.

148. The polynucleotide of any of embodiments 123-147, wherein the encoded C19orf66 comprises a nuclear export signal.

149. The polynucleotide of embodiment 148, wherein the nuclear export signal is selected from LXXXLXXLXL (SEQ ID NO:85) or LEDLDNLIL (SEQ ID: NO 87).

150. The polynucleotide of embodiment 148 or embodiment 149, wherein the nuclear export signal has the sequence of nucleic acids set forth in SEQ ID NO: 87.

151. The polynucleotide of any of embodiments 123-150, wherein the encoded regulatory factor controls targeted transcriptional activation of the gene encoding C19orf66.

152. The polynucleotide of embodiment 151, wherein the encoded regulatory factor is a fusion protein comprising a site-specific binding domain specific for the C 19orf66 gene and a transcriptional activator.

153. The polynucleotide of embodiment 152 , wherein the site-specific binding domain is selected from the group consisting of: zinc fingers, transcription activation like (TAL) effectors, meganucleases, and CRISPR/Cas9 system components, or a modified form thereof.

154. The method of embodiment 152 or embodiment 159, wherein the encoded regulatory factor is a zinc finger transcription factor (ZF-TF).

155. The method of embodiment 153, wherein the site-specific binding domain is a CRISPR/Cas system, wherein the CRISPR/Cas system comprises a modified Cas nuclease that lacks nuclease activity and a guide RNA (gRNA).

156. The polynucleotide of embodiments 155, wherein the modified nuclease is a catalytically dead Cas9 (dCas9).

157. The polynucleotide of any of embodiments 152-156, wherein the transcriptional activator is selected from Herpes simplex-derived transactivation domain, Dnmt3a methyltransferase domain, p65, VP16, and VP64.

158. The polynucleotide of any of embodiments 152-157, wherein the transcriptional activator is the tripartite activator VP64-p65-Rta (VPR).

159. The polynucleotide of any of embodiments 123-158, wherein the promoter is a human surfactant A promoter , a human surfactant B promoter, a human surfactant C promoter, a human surfactant D promoter, human ROBO4 promoter, or a human CDH1 gene.

160. The polynucleotide of any of embodiments 123-159, wherein the promoter is the human surfactant B promoter set forth in SEQ ID NO: 10.

161.The polynucleotide of any of embodiments 123-160, wherein the nucleotide sequence is an mRNA.

162. The polynucleotide of any of embodiments 123-161 that is freeze-dried.

163. A vehicle comprising the polynucleotide of any of embodiments 123-162.

164. A fusion protein, comprising (1) a Chromosome 19 Open Reading Frame 66 (C 19orf66) protein or a regulatory factor that increases expression of the gene encoding C19orf66 in a cell; and (2) a cell penetrating peptide.

165. The fusion protein of embodiment 164 that comprises (1) a C190rf66 protein; and (2) a cell penetrating peptide.

166. The fusion protein of embodiment 165, wherein the C19orf66 protein is linked indirectly to the cell penetrating peptide via a peptide linker.

167. The fusion protein of embodiment 164 that comprises (1) a regulatory factor that increases expression of the gene encoding C19orf66 in a cell; and (2) a cell penetrating peptide.

168. The fusion protein of embodiment 167, wherein the C19orf66 protein or the regulatory factor is linked indirectly to the cell penetrating peptide via a peptide linker.

169. The fusion protein of any of embodiments 164-168, wherein the fusion protein inhibits or prevents viral replication, optionally wherein the encoded C 19orf66 inhibits or prevents viral replication of a Coronavirus..

170. The fusion protein of any of embodiments 164-169, wherein the fusion protein inhibits or prevents ribosomal frameshifting.

171. The fusion protein of any of embodiments 164-169, wherein the fusion protein inhibits or prevents viral RNA processing.

172. The fusion protein of any of embodiments 164-171, wherein the C19orf66 protein is or comprises the sequence of amino acids set forth in SEQ ID NO: 1, or a sequence of amino acids that has at least 90%, at least 92%, at least 95%, or at least 98% sequence identity to the sequence of amino acids set forth in SEQ ID NO: 1.

173. The fusion protein of any of embodiments 164-172, wherein the C19orf66 protein is or comprises the sequence set forth in SEQ ID NO:1.

174. The fusion protein of any of embodiments 164-173, wherein the C19orf66 protein is encoded by the nucleotide sequence set forth in SEQ ID NO: 2, or a nucleotide sequence that has at least 90%, at least 92%, at least 95%, or at least 98% sequence identity to SEQ ID NO:2.

175. The fusion protein of any of embodiments 164-174, wherein the C19orf66 protein is encoded by the nucleotide sequence set forth in SEQ ID NO:2.

176. The fusion protein of any of embodiments 164-171, wherein the C19orf66 protein is or comprises the sequence of amino acids set forth in SEQ ID NO: 3, or a sequence of amino acids that has at least 90%, at least 92%, at least 95%, or at least 98% sequence identity to the sequence of amino acids set forth in SEQ ID NO:3.

177. The fusion protein of any of embodiments 164-171 and 176, wherein the C19orf66 protein is or comprises the sequence set forth in SEQ ID NO:3.

178. The fusion protein of any of embodiments 164-171, 176 and 177, wherein the C19orf66 protein is encoded by the nucleotide sequence set forth in SEQ ID NO: 4, or a nucleotide sequence that has at least 90%, at least 92%, at least 95%, or at least 98% sequence identity to the sequence set forth in SEQ ID NO:4.

179. The fusion protein of any of embodiments 164-171 and 176-178, wherein the C19orf66 protein is encoded by the sequence set forth in SEQ ID NO:4.

180. The fusion protein of any of embodiments 164-171 , wherein the C19orf66 protein is or comprises the sequence of amino acids set forth in SEQ ID NO: 5, or a sequence of amino acids that has at least 90%, at least 92%, at least 95%, or at least 98% sequence identity to the sequence of amino acids set forth in SEQ ID NO:5.

181. The fusion protein of any of embodiments 164-171 and 180, wherein the C19orf66 protein is or comprises the sequence set forth in SEQ ID NO:5.

182. The fusion protein of any of embodiments 164-171, 180, and 181, wherein the C19orf66 is encoded by the nucleotide sequence set forth in SEQ ID NO: 6, or a nucleotide sequence that has at least 90%, at least 92%, at least 95%, or at least 98% sequence identity to the sequence set forth in SEQ ID NO:6.

183. The fusion protein of any of embodiments 164-171, and 180-182, wherein the C19orf66 protein is encoded by the sequence set forth in SEQ ID NO:6.

184. The fusion protein of any of embodiments 164-171, wherein the C19orf66 protein is or comprises the sequence of amino acids set forth in SEQ ID NO: 7, or a sequence of amino acids that has at least 90%, at least 92%, at least 95%, or at least 98% sequence identity to the sequence of amino acids set forth in SEQ ID NO:7.

185. The fusion protein of any of embodiments 164-171 and 184, wherein the C19orf66 protein is or comprises the sequence set forth in SEQ ID NO:7.

186. The fusion protein of any of embodiments 164-171, 184 and 185, wherein the C19orf66 protein is encoded by the nucleotide sequence set forth in SEQ ID NO: 8, or a sequence of nucleic acids that has at least 90%, at least 92%, at least 95%, or at least 98% sequence identity to the sequence set forth in SEQ ID NO:.8

187. The fusion protein of any of embodiments 164-171 and 184-186, wherein the C19orf66 protein is encoded by the nucleotide sequence set forth in SEQ ID NO:.8

188. The fusion protein of any of embodiments 164-187, wherein the C19orf66 protein comprises a nuclear localization signal.

189. The fusion protein of embodiment 188, wherein the nuclear localization signal is selected from the group consisting of:

KKRXKR (SEQ ID NO: 81),

KRPAATKKAGQAKKKK (SEQ ID NO: 82)

PAAKRBKLD (SEQ ID NO: 83),

PKKKRKVEDP (SEQ ID NO: 84), and

RRVPQRKEVSRCRKCRK (SEQ ID: NO 86)

190. The fusion protein of embodiment 188 or embodiment 189, wherein the nuclear localization signal has the sequence of nucleic acids set forth in SEQ ID NO: 86.

191. The fusion protein of any of embodiments 164-190, wherein the C19orf66 protein comprises a nuclear export signal.

192. The fusion protein of embodiment 191, wherein the nuclear export signal is selected from LXXXLXXLXL (SEQ ID NO:85) or LEDLDNLIL (SEQ ID: NO 87).

193. The fusion protein of embodiment 191 or embodiment 192, where in the nuclear export signal has the sequence of nucleic acids set forth in SEQ ID NO: 87.

194. The fusion protein of any of embodiments 164 and 166-193, wherein the regulatory factor controls targeted transcriptional activation of the gene encoding C 19orf66.

195. The fusion protein of any of embodiments 164 and 166-194, wherein the regulatory factor is a fusion protein comprising a site-specific binding domain specific for the C 19orf66 gene, and a transcriptional activator.

196. The fusion protein of embodiment 195, wherein the site-specific binding domain is selected from the group consisting of: zinc fingers, transcription activation like (TAL) effectors, meganucleases, and CRISPR/Cas system, or a modified form thereof.

197. The fusion protein of embodiment 195 or embodiment 196, wherein the regulatory factor is a zinc finger transcription factor (ZF-TF).

198. The fusion protein of embodiment 195 or embodiment 196, wherein the site-specific binding domain is a CRISPR/Cas system, wherein the CRISPR/Cas system comprises a modified Cas nuclease that lacks nuclease activity and a guide RNA (gRNA).

199. The fusion protein of embodiment 198, wherein the modified nuclease is a catalytically dead Cas9 (dCas9).

200. The fusion protein of any of embodiments 194-199, wherein the transcriptional activator is selected from Herpes simplex-derived transactivation domain, Dnmt3a methyltransferase domain, p65, VP16, and VP64, optionally wherein the transcriptional activator is the tripartite activator VP64-p65-Rta (VPR).

201. The fusion protein of any of embodiments 164-200, wherein the cell penetrating peptide is a peptide that facilitates delivery to the interior of a cell.

202. The fusion protein of any of embodiments 164-201, wherein the cell penetrating peptide is selected from the group consisting of:
TAT (SEQ ID NO: 13)
Penetratin (SEQ ID NO: 14)
Transporant (SEQ ID NO: 15)
Pept 1 (SEQ ID NO: 16)
Pept 2 (SEQ ID NO: 17)
Transportan (SEQ ID NO: 18)
IgV (SEQ ID NO: 19 )

203. A vehicle comprising the fusion protein of any of embodiments 164-202.

204. A vehicle comprising a nucleotide sequence encoding Chromosome 19 Open Reading Frame 66 (C19orf66).

205. A vehicle comprising a Chromosome 19 Open Reading Frame 66 (C19orf66) protein.

206. The vehicle of embodiment 205, wherein the C19orf66 is a recombinant protein.

207. A vehicle comprising a nucleotide sequence encoding a regulatory factor capable of increasing expression of the gene encoding C 19orf66 in.

208. A vehicle comprising a regulatory factor protein capable of increasing expression of a gene encoding C19orf66.

209. The vehicle of embodiment 208, wherein the regulatory factor is a recombinant fusion protein or is a protein complex.

210. The vehicle of any of embodiments 204-209, wherein the C19orf66 protein is or comprises the sequence of amino acids set forth in SEQ ID NO: 1, or a sequence of amino acids that has at least 90%, at least 92%, at least 95%, or at least 98% sequence identity to the sequence of amino acids set forth in SEQ ID NO: 1.

211. The vehicle of any of embodiments 204-210, wherein the C19orf66 protein is or comprises the sequence set forth in SEQ ID NO:1.

212. The vehicle of any of embodiments 204-211, wherein the C19orf66 protein is encoded by the nucleotide sequence set forth in SEQ ID NO: 2, or a nucleotide sequence that has at least 90%, at least 92%, at least 95%, or at least 98% sequence identity to SEQ ID NO:2.

213. The vehicle of any of embodiments 208-212, wherein the C19orf66 protein is encoded by the nucleotide sequence set forth in SEQ ID NO:2.

214. The vehicle of any of embodiments 204-209, wherein the C19orf66 protein is or comprises the sequence of amino acids set forth in SEQ ID NO: 3, or a sequence of amino acids that has at least 90%, at least 92%, at least 95%, or at least 98% sequence identity to the sequence of amino acids set forth in SEQ ID NO:3.

215. The vehicle of any of embodiments 204-209 and 214, wherein the C19orf66 protein is or comprises the sequence set forth in SEQ ID NO:3.

216. The vehicle of any of embodiments 204-209, 214 and 215, wherein the C19orf66 protein is encoded by the nucleotide sequence set forth in SEQ ID NO: 4, or a nucleotide sequence that has at least 90%, at least 92%, at least 95%, or at least 98% sequence identity to the sequence set forth in SEQ ID NO:4.

217. The vehicle of any of embodiments 204-209 and 214-216, wherein the C19orf66 protein is encoded by the sequence set forth in SEQ ID NO:4.

218. The vehicle of any of embodiments 204-209 , wherein the C19orf66 protein is or comprises the sequence of amino acids set forth in SEQ ID NO: 5, or a sequence of amino acids that has at least 90%, at least 92%, at least 95%, or at least 98% sequence identity to the sequence of amino acids set forth in SEQ ID NO:5.

219. The vehicle of any of embodiments 204-209 and 218, wherein the C19orf66 protein is or comprises the sequence set forth in SEQ ID NO:5.

220. The vehicle of any of embodiments 204-209, 218 and 219, wherein the C19orf66 is encoded by the nucleotide sequence set forth in SEQ ID NO: 6, or a nucleotide sequence that has at least 90%, at least 92%, at least 95%, or at least 98% sequence identity to the sequence set forth in SEQ ID NO:6.

221. The vehicle of any of embodiments 204-209 and 218-220, wherein the C19orf66 protein is encoded by the sequence set forth in SEQ ID NO:6.

222. The vehicle of any of embodiments 204-209, wherein the C19orf66 protein is or comprises the sequence of amino acids set forth in SEQ ID NO: 7, or a sequence of amino acids that has at least 90%, at least 92%, at least 95%, or at least 98% sequence identity to the sequence of amino acids set forth in SEQ ID NO:7.

223. The vehicle of any of embodiments 204-209 and 222, wherein the C19orf66 protein is or comprises the sequence set forth in SEQ ID NO:7.

224. The vehicle of any of embodiments 204-209, 222 and 223, wherein the C19orf66 protein is encoded by the nucleotide sequence set forth in SEQ ID NO: 8, or a sequence of nucleic acids that has at least 90%, at least 92%, at least 95%, or at least 98% sequence identity to the sequence set forth in SEQ ID NO:.8

225. The vehicle of any of embodiments 204-209 and 222-224, wherein the C19orf66 protein is encoded by the nucleotide sequence set forth in SEQ ID NO:.8

226. The vehicle of any of embodiments 204-225, wherein the C19orf66 protein comprises a nuclear localization signal.

227. The vehicle of embodiment 226, wherein the nuclear localization signal is selected from the group consisting of:

KKRXKR (SEQ ID NO: 81),

KRPAATKKAGQAKKKK (SEQ ID NO: 82)

PAAKRBKLD (SEQ ID NO: 83),

PKKKRKVEDP (SEQ ID NO: 84), and

RRVPQRKEVSRCRKCRK (SEQ ID: NO 86)

228. The vehicle of embodiment 226 or embodiment 227, wherein the nuclear localization signal has the sequence of nucleic acids set forth in SEQ ID NO: 86.

229. The vehicle of any of embodiments 204-228, wherein the C19orf66 protein comprises a nuclear export signal.

230. The vehicle of embodiment 229, wherein the nuclear export signal is selected from LXXXLXXLXL (SEQ ID NO:85) or LEDLDNLIL (SEQ ID: NO 87).

231. The vehicle of embodiment 229 or embodiment 230, where in the nuclear export signal has the sequence of nucleic acids set forth in SEQ ID NO: 87.

232. The vehicle of any of embodiments 207-231, wherein the regulatory factor controls targeted transcriptional activation of the gene encoding C 19orf66.

233. The vehicle of any of embodiments 207-232, wherein the regulatory factor is a fusion protein comprising a site-specific binding domain specific for the C 19orf66 gene, and a transcriptional activator.

234. The vehicle of embodiment 233, wherein the site-specific binding domain is selected from the group consisting of: zinc fingers, transcription activation like (TAL) effectors, meganucleases, and CRISPR/Cas system, or a modified form thereof.

235. The vehicle of embodiment 233 or embodiment 234, wherein the regulatory factor is a zinc finger transcription factor (ZF-TF).

236. The vehicle of embodiment 233 or embodiment 234, wherein the site-specific binding domain is a CRISPR/Cas system, wherein the CRISPR/Cas system comprises a modified Cas nuclease that lacks nuclease activity and a guide RNA (gRNA).

237. The vehicle of embodiment 236, wherein the modified nuclease is a catalytically dead Cas9 (dCas9).

238. The vehicle of any of embodiments 232-237, wherein the transcriptional activator is selected from Herpes simplex-derived transactivation domain, Dnmt3a methyltransferase domain, p65, VP16, and VP64.

239. The vehicle of any of embodiments 232-238, wherein the transcriptional activator is the tripartite activator VP64-p65-Rta (VPR).

240. The vehicle of any of embodiments 163 and 203-239, wherein the vehicle is a lipid particle or a non-lipid particle.

241. The vehicle of embodiment 240, wherein the vehicle is a lipid particle that is a viral vector or a viral-like particle.

242. The vehicle of embodiment 242, wherein the viral vector or viral-like particle is derived from an Adeno-associated virus (AAV) vector particle.

243. The vehicle of embodiment 242, wherein the AAV is of serotype 1, 2, 5, 6.

244. The vehicle of embodiment 242 or embodiment 243, wherein the AAV is of serotype 5.

245. The vehicle of embodiment 242 or embodiment 245, wherein the AAV is of serotype 6.

246.The vehicle of embodiment 242, wherein the viral vector or viral-like particle is derived from a lentivirus.

247. The vehicle of embodiment 246, wherein the lentivirus is Human Immunodeficiency Virus-1 (HIV-1).

248. The vehicle of of any of embodiments 241-247, wherein the viral vector or viral-like particle is a virus-like particle.

249. The vehicle of any of embodiments 241-248, wherein the viral vector or viral-like particle comprises a fusogen.

250. The vehicle of embodiment 240, wherein the vehicle is a lipid particle, wherein the lipid particle comprises (i) a lipid bilayer enclosing a lumen, and (ii) a fusogen, wherein the fusogen is embedded in the lipid bilayer.

251. The vehicle of embodiment 250, wherein the lipid bilayer is derived from a membrane of a host cell used for producing a virus or virus-like particle.

252. The vehicle of embodiment 250 or embodiment 25, wherein the lipid bilayer is derived from a membrane of a host cell used for producing a virus-like particle, wherein the virus-like particle is replication defective.

253. The vehicle of embodiment 251 or embodiment 252, wherein the virus or virus-like particle is a retrovirus.

254. The vehicle of embodiment 253, wherein the retrovirus is a lentivirus.

255. The vehicle of embodiment 251 or embodiment 252, wherein the virus or virus-like particle is an adenovirus.

256. The vehicle of any of embodiments 241-255, wherein the viral vector or viral-like particle comprises a fusogen glycoprotein derived from a Paramyxovirus.

257. The vehicle of any of embodiments 249-256, wherein the fusogen is a viral fusogen selected from a Class I viral membrane fusion protein, a Class II viral membrane protein, a Class II viral membrane fusion protein, a viral membrane glycoprotein, or a viral envelope protein.

258. The vehicle of any of embodiments 249-257, wherein the fusogen is a vesicular stomatitis virus envelope glycoprotein (VSV-G).

259. The vehicle of any of embodiments 249-257, wherein the fusogen is a syncytin.

260. The vehicle of any of embodiments 249-257, wherein the fusogen is from a coronavirus.

261. The vehicle of any of embodiments 249-257 and 260, wherein the the fusogen is a Severe Acute Respiratory Syndrome (SARS) coronavirus 1 (SARS CoV-1) spike glycoprotein.

262. The vehicle of any of embodiments 249-257 and 260, wherein the fusogen is a Severe Acute Respiratory Syndrome (SARS) coronavirus 2 (SARS CoV-2) spike glycoprotein.

263. The vehicle of any of embodiments 249-257 and 260, wherein the fusogen is an alpha coronavirus CD13 protein.

264. The vehicle of any of embodiments 249-257, wherein the fusogen comprises an F protein molecule or a biologically active portion thereof from a Paramyxovirus and/or a glycoprotein G (G protein) or a biologically active portion thereof from a Paramyxovirus.

265. The vehicle of any of embodiments 249-257, wherein the fusogen is derived from an F protein molecule or a biologically active portion thereof from a Paramyxovirus and/or a glycoprotein G (G protein) or a biologically active portion thereof from a Paramyxovirus.

266. The vehicle of embodiment 264 or embodiment 265, wherein the Paramyxovirus is a henipavirus.

267. The vehicle of any of embodiments 264-266 wherein the Paramyxovirus is Nipah virus.

268. The vehicle of any of embodiments 264-267, wherein the Paramyxovirus is Hendra virus.

269. The vehicle of any one of embodiments 249-268, wherein the fusogen is a re-targeted fusogen comprising a targeting moiety that binds to a molecule on a target cell.

270. The vehicle of embodiment 269, wherein the targeting moiety is a Design ankyrin repeat proteins (DARPin), a single domain antibody (sdAb), a single chain variable fragment (scFv), or an antigen-binding fibronectin type III (Fn3) scaffold.

271. The vehicle of embodiment 269 or embodiment 270, wherein the target cell is known or suspected of being infected by a coronavirus.

272. The vehicle of any of embodiments 269-271, wherein targeting moiety binds a receptor of a coronavirus.

273. The vehicle of any of embodiments 269-272, wherein the targeting moiety binds angiotensin-converting enzyme 2 (ACE2).

274. The vehicle of any of embodiments 269-272, wherein the targeting moiety binds to transmembrane proteinase, serine 2 (TMPRSS2).

275. The vehicle of any of embodiments 269-272, wherein the targeting moiety binds to dipeptidyl peptidase 4 (DPP4).

276. The vehicle of any one of embodiments 249-275, wherein the fusogen is modified to reduce its native binding tropism.

277. The vehicle of any of embodiments 264-276, wherein the G protein or the biologically active portion thereof is a mutant NiV-G protein that exhibits reduced binding to Ephrin B2 or Ephrin B3.

278. The vehicle of embodiment 277, wherein the mutant NiV-G protein comprises one or more amino acid substitutions corresponding to amino acid substitutions selected from the group consisting of E501A, W504A, Q530A and E533A with reference to numbering set forth in SEQ ID NO:26.

279. The vehicle of any of embodiments 264-278, wherein the mutant NiV-G protein has the amino acid sequence set forth in SEQ ID NO: 69 or an amino acid sequence having at or about 80%, at least at or about 81%, at least at or about 82%, at least at or about 83%, at or about 84%, at least at or about 85%, at least at or about 86%, or at least at or about 87%, at least at or about 88%, or at least at or about 89%, at least at or about 90%, at least at or about 91%, at least at or about 92%, at least at or about 93%, at least at or about 94%, at least at or about 95%, at or about 96%, at least at or about 97%, at least at or about 98%, or at least at or about 99% sequence identity to SEQ ID NO:69.

280. The vehicle of any of embodiments 264-279, wherein the mutant NiV-G protein has the amino acid sequence set forth in SEQ ID NO: 70 or an amino acid sequence having at or about 80%, at least at or about 81%, at least at or about 82%, at least at or about 83%, at or about 84%, at least at or about 85%, at least at or about 86%, or at least at or about 87%, at least at or about 88%, or at least at or about 89%, at least at or about 90%, at least at or about 91%, at least at or about 92%, at least at or about 93%, at least at or about 94%, at least at or about 95%, at or about 96%, at least at or about 97%, at least at or about 98%, or at least at or about 99% sequence identity to SEQ ID NO:70.

281. The vehicle of any of embodiments 264-280, wherein the NiV-F protein is a biologically active portion thereof that has a 20 amino acid truncation at or near the C-terminus of the wild-type NiV-F protein (SEQ ID NO:37).

282. The vehicle of embodiment 281, wherein the NiV-F protein has an amino acid sequence set forth in SEQ ID NO:76 or a sequence having at or about 80%, at least at or about 81%, at least at or about 82%, at least at or about 83%, at or about 84%, at least at or about 85%, at least at or about 86%, or at least at or about 87%, at least at or about 88%, or at least at or about 89%, at least at or about 90%, at least at or about 91%, at least at or about 92%, at least at or about 93%, at least at or about 94%, at least at or about 95%, at or about 96%, at least at or about 97%, at least at or about 98%, or at least at or about 99% sequence identity to SEQ ID NO: 76.

283. The vehicle of any of embodiments 264-280, wherein the NiV-F protein is a biologically active portion thereof that has a 22 amino acid truncation at or near the C-terminus of the wild-type NiV-F protein (SEQ ID NO:37).

284. The vehicle of embodiment 283, wherein the NiV-F protein has an amino acid sequence set forth in SEQ ID NO:75 or a sequence having at or about 80%, at least at or about 81%, at least at or about 82%, at least at or about 83%, at or about 84%, at least at or about 85%, at least at or about 86%, or at least at or about 87%, at least at or about 88%, or at least at or about 89%, at least at or about 90%, at least at or about 91%, at least at or about 92%, at least at or about 93%, at least at or about 94%, at least at or about 95%, at or about 96%, at least at or about 97%, at least at or about 98%, or at least at or about 99% sequence identity to SEQ ID NO: 75.

285. The vehicle of embodiment 283 or claim284, wherein the NiV-F protein has an amino acid sequence set forth in SEQ ID NO:80 or a sequence having at or about 80%, at least at or about 81%, at least at or about 82%, at least at or about 83%, at or about 84%, at least at or about 85%, at least at or about 86%, or at least at or about 87%, at least at or about 88%, or at least at or about 89%, at least at or about 90%, at least at or about 91%, at least at or about 92%, at least at or about 93%, at least at or about 94%, at least at or about 95%, at or about 96%, at least at or about 97%, at least at or about 98%, or at least at or about 99% sequence identity to SEQ ID NO: 80.

286. The vehicle of any of embodiments 264-285, wherein the NiV-F protein comprises a point mutation on an N-linked glycosylation site.

287. The vehicle of any of embodiments 264-281 and 286, wherein the NiV-F protein is a biologically active portion thereof that comprises:
i) a 20 amino acid truncation at or near the C-terminus of the wild-type NiV-F protein (SEQ ID NO:37); and
ii) a point mutation on an N-linked glycosylation site.

288. The vehicle of embodiment 287, wherein the NiV-F protein has an amino acid sequence set forth in SEQ ID NO:74 or a sequence having at or about 80%, at least at or about 81%, at least at or about 82%, at least at or about 83%, at or about 84%, at least at or about 85%, at least at or about 86%, or at least at or about 87%, at least at or about 88%, or at least at or about 89%, at least at or about 90%, at least at or about 91%, at least at or about 92%, at least at or about 93%, at least at or about 94%, at least at or about 95%, at or about 96%, at least at or about 97%, at least at or about 98%, or at least at or about 99% sequence identity to SEQ ID NO: 74.

289. The vehicle of any of embodiments 163 and 203-239, wherein the vehicle is a non-viral vector.

290. The vehicle of embodiment 289, wherein the non-viral particle is a liposome, a microparticle, a nanoparticle, a nanogel, a dendrimer or a dendrisome.

291. The vehicle of embodiment 289 or embodiment 290, wherein the non-viral vector is a nanoparticle.

292. The vehicle of embodiment 289 or embodiment 290, wherein the non-viral vector is a lipsosome.

293. The vehicle of embodiment 289, wherein the non-viral vector is a plasmid.

294. The vehicle of any of embodiments 289-293, wherein the vehicle is freeze dried.

295. The vehicle of any of embodiments 289-294, wherein the non-viral vector further comprises a vector-surface targeting moiety that binds to a molecule on a target cell.

296. The vehicle of embodiment 295, wherein the vector-surface targeting moiety is a peptide or a polypeptide.

297. The vehicle of embodiment 295 or embodiment 296, wherein the polypeptide is an antibody or antigen-binding fragment.

298. The vehicle of any of embodiments 295-297, wherein the target cell is known or suspected of being infected by a coronavirus.

299. The vehicle of any of embodiments 295-298, wherein the target cell is a lung cell.

300. The vehicle of any of embodiments 295-299, wherein targeting moiety binds a receptor of a coronavirus.

301.The vehicle of any of embodiments 295-300, wherein the targeting moiety binds angiotensin-converting enzyme 2 (ACE2).

302.The vehicle of any of embodiments 295-300, wherein the targeting moiety binds to transmembrane proteinase, serine 2 (TMPRSS2).

303. The vehicle of any of embodiments 295-300, wherein the targeting moiety binds to dipeptidyl peptidase 4 (DPP4).

304. A composition comprising the polynucleotide of any of embodiments 123-162, a fusion protein of any of embodiments 164-202 or the vehicle of any of embodiments 163 and 203-303.

305. The composition of embodiment 304, further comprising a pharmaceutically acceptable carrier.

306. The composition of embodiment 304 or embodiment 305 that is a pharmaceutical composition.

307. The composition of any of embodiments 304-306, wherein the composition is sterile.

308. A method of treating a viral infection, comprising administering to a subject known or suspected of having a virus infection the composition of any of embodiments 304-307.

309. A method of reducing the likelihood a viral infection, comprising administering to a subject known or suspected of having a virus infection the composition of any of embodiments 304-307.

310. The method of embodiments 1308 or embodiment 309, wherein the virus relies on frameshifting.

311. The method of any of embodiments 308-310, wherein the viral infection is caused by a coronavirus.

312. The method of embodiment 311, wherein the viral infection is caused by SARS CoV-2.

313. The method of any of embodiments 1-122 and 308-312 wherein the composition is administered to the lung tissue of a subject.

314. The method of embodiment 313, wherein the lung tissue is the bronchial or tracheal epithelium.

315. The method of any of embodiments 1-122 and 308-314, wherein the composition is administered by nebulization.

316. The method of any of embodiments 1-122 and 308-314, wherein the composition is administered by inhalation.

317. The method of any of embodiments 1-122 and 308-314, wherein the composition is administered by topical instillation.

318. The method of any of embodiments 1-122 and 308-314, wherein the composition is administered by oral tablet.

319. The method of any of embodiments 1-122 and 308-312, wherein the composition is administered parenterally, optionally subcutaneously or intravenously.

320. The method of embodiment 319, wherein the composition is administered by injection.

321. The method of embodiment 320, wherein the composition is administered by infusion.

322. The method of any of embodiments 1-122 and 308-321, wherein the subject is known, suspected, or predicted to have been exposed to a SARS coronavirus.

323. The method of any of embodiments 1-122 and 308-322, wherein the subject is known, suspected, or predicted to have been exposed to a SARS CoV-1 virus.

324. The method of any of embodiments 1-122 and 308-322, wherein the subject is known, suspected, or predicted to have been exposed to a SARS CoV-2 virus.

325. The method of any of embodiments 1-122 and 308-324, wherein the subject is known or suspected of having Stage 1 Coronavirus disease 2019 (COVID-19).

326. The method of any of 1-122 and 308-324, wherein the subject is known or suspected of having Stage 2 Coronavirus disease 2019 (COVID-19).

327. The method of any of embodiments 1-122 and 308-324, wherein the subject is known or suspected of having Stage 2 Coronavirus disease 2019 (COVID-19).

328. The method of embodiments 1-122 and 308-321, wherein the subject is known or suspected of having Severe Acute Respiratory Syndrome (SARS).

### VIII. EXAMPLES

The following example is included for illustrative purposes only and is not intended to limit the scope of the invention.

### Example 1 Use of C19orf66 Expressing Vector in vivo

An AAV2.5T vector (Excoffon et al., 2009, Proc. Natl. Acad. Sci. U. S. A., 106:3865-70) expressing C19orf66 under control of a hybrid promoter having a human cytomegalovirus (CMV) enhancer and the elongation factor 1a promoter (hCEF) is administered by inhalation to patients having been infected with SARS-CoV-2. The AAV vector drives expression of C19orf66 in infected cells, inhibiting viral replication. The patients are evaluated for overall survival through 28 days following treatment, and evaluated for secondary outcomes (i) length of hospital stay (ii) length of ICU stay, (iii) duration of ventilator use, (iv) duration of vasopressors use, (v) duration on renal replacement therapy, (vi) viral kinetics as measured by virologic failure (defined as increase in viral load of >0.5 log on two consecutive days, or > 1 log increase in one day, not in keeping with any baseline trend of rising viral loads during the pretreatment viral testing), and (vii) number of adverse events as measured by CTCAE v. 5.0, for at least six months following treatment. Patients administered the treatment show improved outcomes.

### Example 2 Tetracyline Inducible Expression of C19orf66 and Assessment of Anti-Viral Activity

In order to assess the functionality of c19orf66, Vero-E6 cells were engineered to display inducible expression of c19orf66 as described below, and candidate clones assessed for anti-viral features in response to infection with SARS CoV-2.

### A. Generation of an Inducible C19orf66 Cell Line

An all-in-one (AIO) inducible lentiviral vector was prepared containing a construct composed of a reverse tetracycline response element (rtTA) and an inducible expression cassette containing transgene sequences for the sequence encoding c19orf66 that was separated from a sequence encoding an enhanced Green Fluorescent Protein (EGFP) by an internal ribosomal entry site (IRES). In this aspect, efficient co-expression of both EGFP and c19orf66 is enabled (turned on) from a single cassette by the addition of an analogue of tetracycline (e.g. doxycycline) that allows binding of rrTA to TRE. In the sense orientation a human PGK promoter was used to drive the expression of rtTA v10 variant (Zhou Gene Ther 13, 1382-1390 (2006)). In the antisense direction a T6 tetracycline response element (TRE, (Loew et al. BMC Biotechnol 10, 81 (2010)) was used to drive expression of the codon optimized c19orf66-IRES-GFP, followed by a BGH polyA site. A similarly designed construct is described in Heinz et al, Human Gene Therapy 2010, the contents of which are hereby incorporated by reference.

Vero-E6 cells were transduced with the lentiviral vector. Transduced cells were then screened according to two protocols.

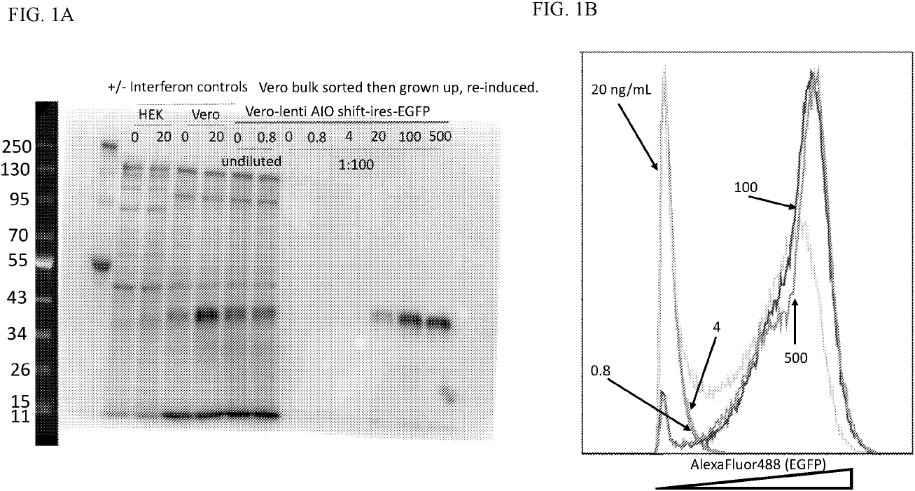

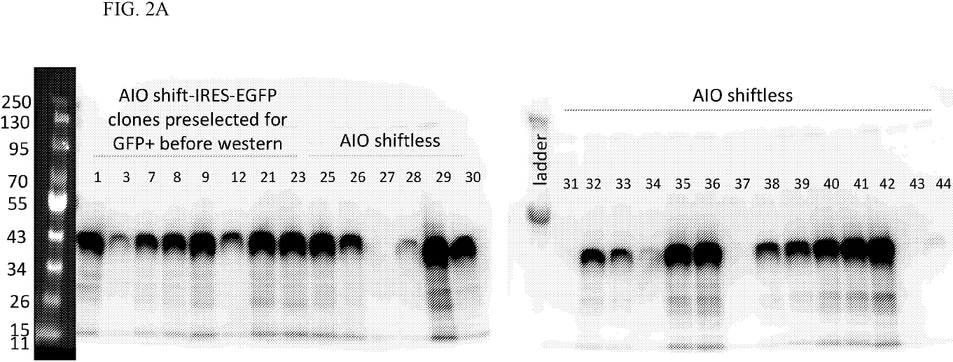

In one strategy, cells were mildly induced with doxycycline before being bulk sorted for EGFP expression via flow cytometry to obtain a population of cells which can be induced to express EGFP and C19orf66. Bulk sorted cells were then re-induced with either 0, 0.8, 4, 20, 100, or 500 ng/mL doxycycline and assayed for c19orf66 protein expression by western blot on undiluted samples or on samples that had been diluted 100-fold, as shown in FIG. 1A. As c19orf66 is an interferon stimulated gene (ISG), stimulation with interferon was chosen to serve as a control for induced c19orf66 expression. The resultant EGFP expression in the same induced cells was monitored by flow cytometry and results are shown in FIG. 1B. As shown, c19orf66 was induced in this system following addition of at least 20 ng/mL doxycycline.

In an alternative strategy, single cell clones of Vero-E6 transduced cells were first isolated and cultured before being individually induced with 1 µg/mL doxycycline and screened for c19orf66 and EGFP expression. C19orf66 expression was determined by western blot on samples that had been diluted 10-fold. As shown in FIG. 2A, inducible expression of c19orf66 as determined by western blot is shown for representative clones after preselection for EGFP expression (e.g. clones 1, 3, 7, 8, 9, 12, 21, and 23). The results of flow cytometry analysis for EGFP expression of exemplary clones 1, 3, 7, 8, 9, 12, 21, and 23 with and without doxycycline are shown in FIG. 2B. Some toxicity of transduced cells was seen following induction in clones 21, 25 and 41.

Bulk sorted cell populations and single cell clones 9 and 21 were selected for further analysis. As all of the populations are capable of producing higher levels of c 19orf66 compared to endogenous interferon-induced levels in the transduced Vero-E6 cells, experiments were carried out to titrate levels of doxycycline to levels to obtain substantial expression while minimizing toxicity. On Day 0, Vero E6 naive cells, Vero E6 transduced and bulk sorted cells, clone 9 or clone 21 were plated in 6 well cell culture plates such that 35% confluence was achieved the next day. On Day 1, cells were treated with titrated amounts of doxycycline: 0, 5, 10, 20, 50, and 100 ng/mL. Cells were observed for overall health and EGFP expression using microcopy. Results of this observation on Day 2 are shown below in a Table E1.

Table E1

Observations 48 Hours Post Doxycycline Treatment

| | | |
|---|---|---|
| Cell Line | Cell Health | EGFP |
| Naive Vero E6 | Good, almost confluent at all doxycycline concentrations | None |
| Bulk Sorted | Slight toxicity observed starting at 20 ng/mL. 20% cells death 50 ng/mL, 40% cells death 100 ng/mL. | Visible at 10 ng/mL, strong signal at 20 ng/mL |
| Clone 9 | Slight toxicity observed starting at 10 ng/mL. 50% cell death at 20 ng/mL and worse at higher concentrations. | Visible at 10 ng/mL and higher concentrations. |
| Clone 21 | 50% cell death at 10 ng/mL and worse at higher concentrations. | Visible at 5 ng/mL, strong signal at 10 ng/mL. |

In view of the observations at Day 2 above, 11 conditions were selected to continue by culture in 96-well plates in DMEM cell culture media supplemented with 2% Fetal Bovine Serum (FBS). These conditions were set forth as follows: 0 ng/mL doxycycline for each of the cell lines listed in Table E1; 5 ng/mL and 10 ng/mL doxycycline for each of the Bulk Sorted population, Clone 9, and Clone 21; and finally 20 ng/mL for the bulk sorted population. Cells were cultured until day 4 at which time cell cultures were observed for overall health and EGFP expression as described above. The results of this observation on Day 4 are shown below in Table E2.

Table E2

Observations 96 Hours Post Doxycycline Treatment

| | | | |
|---|---|---|---|
| Cell Line | Doxycycline Concentrations | Cell Health | EGFP |
| Naive Vero E6 | 0 ng/mL | Good | None |
| Bulk Sorted | 0, 5, 10, 20 ng/mL | Healthy at ≦ 10 ng/mL. Some toxicity observed at 20 ng/mL. | Visible at ≧ 10 ng/mL |
| Clone 9 | 0, 5, 10 ng/mL | Healthy at ≦ 5 ng/mL. Some toxicity observed at 10 ng/mL. | Visible at ≧ 10 ng/mL |
| Clone 21 | 0, 5, 10 ng/mL | Healthy at ≦ 5 ng/mL. Cell death observed at 10 ng/mL. | Visible at ≧ 10 ng/mL |

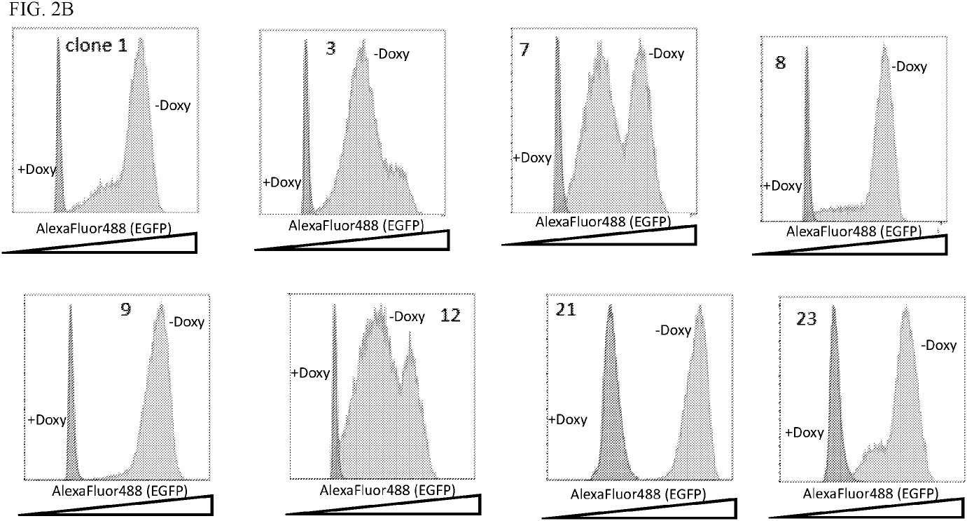

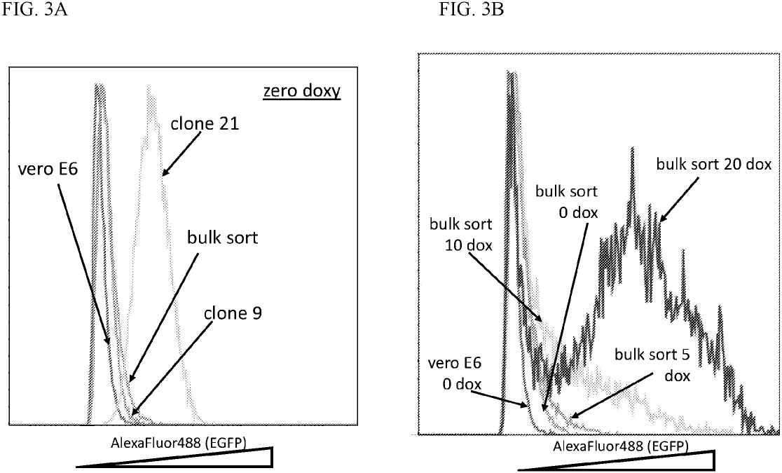

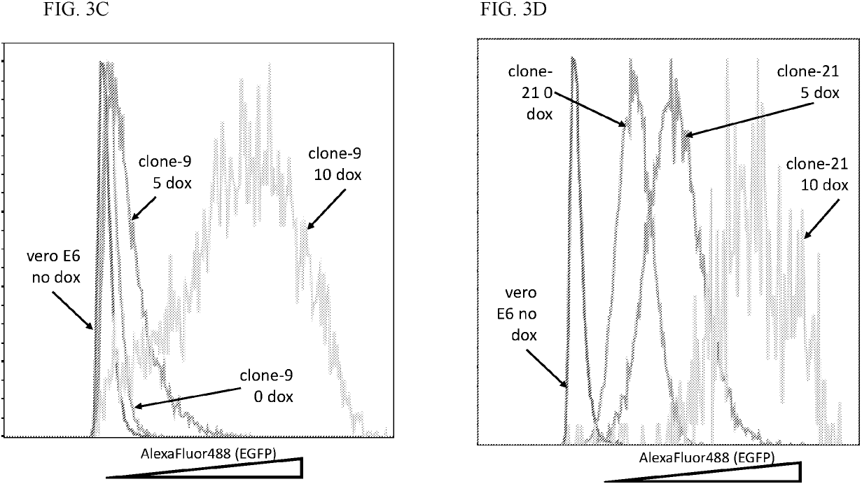

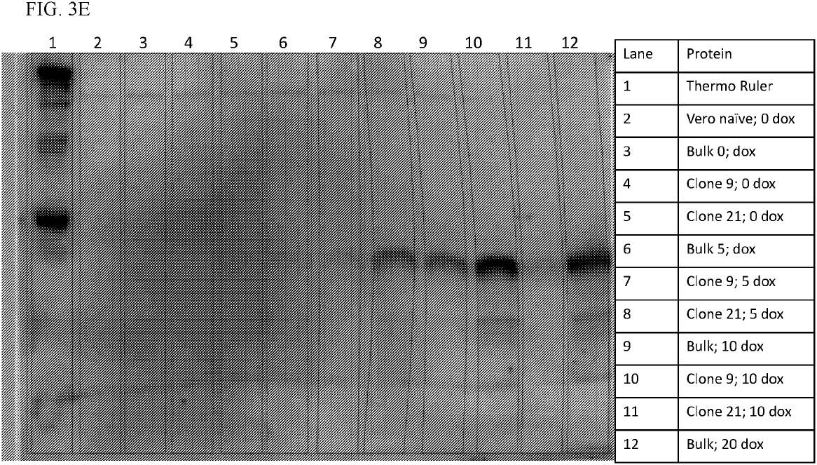

Cells Day 4 post doxycycline induction were also analyzed for EGFP expression via flow cytometry and c19orf66 expression via western blot. Flow cytometry plots for EGFP expression are shown in FIG. 3A for each cell population at 0 ng/mL doxycycline, and following induction with various concentrations of doxycycline in Bulk Sorted cell population (FIG. 3B), Clone 9 cells (FIG. 3C), and Clone 21 cells (FIG. 3D). By western blot analysis on Day 4, c19orf66 expression was not observed in cells that had not been exposed to doxycycline, but was observed in bulk cells induced with 10 ng/mL or 20 ng/mL doxycycline, in clone 21 cells induced with 5 ng/mL doxycycline and in clone 9 cells induced with 10 ng/mL doxycycline. For clone 21 cells induced with 10 ng/mL doxycycline, few cells were available for western blot due to cell death. An exemplary western blot probing for c19orf66 protein is shown in FIG. 3E. Similar results were seen in cells at Day 5.

Taken together, these results support that cell lines created as described above are a suitable model for doxycycline induced c19orf66 and EGFP expression.

### B. Protective Effect of C19orf66 Expression Against SARS CoV-2 Infection

The c19orf66 cell lines were then used to measure the effect of targeted expression of c19orf66 during SARS CoV-2 infection of Vero E6 cells.

Naive Vero cells, cells from the bulk sorted population, as well as cells of Clones 9 and 21 were first seeded into 96-well cell culture plates in DMEM media supplemented with 2% FBS. Cells were left untreated (0 ng/mL) or were exposed to 5 ng/nL, 10 ng/mL or 20 ng/mL doxycycline, as summarized in Table E3.

Table E3

Doxycycline Treatment for Viral Infection Assays

| | |
|---|---|
| Cell Line | Doxycycline Concentrations |
| Naive Vero E6 | 0 ng/mL |
| Bulk Sorted | 0, 5, 10 ng/mL |
| Clone 9 | 0, 5, 10 ng/mL |
| Clone 21 | 0, 5, 10, 20 ng/mL |

Cells were then infected with live SARS CoV-2 (USA_WA1/2020) in a BSL-4 laboratory facility. Cells were infected at a multiplicity of infection (MOI) of 10 or an MOI of 1 and harvested 1 day post infection (dpi). As a control, infected cells were treated with either of a DMSO or remdesevir control. Each plate also contained representative uninfected mock conditions, in which no virus was applied to the cellular supernatant.

Cells were harvested 1 day post infection, then fixed and stained for Indirect Fluorescent Antibody (IFA) imaging to determine percentage of cells or cell nuclei positive for SARS N protein. For staining, cells were stained with anti-SARS N primary antibody (1:2000) followed by an anti-rabbit-Cy3 secondary antibody (1:200) and DAPI ( 1:5000). The infection rate was quantified QuPath 0.2.3 with positive cell detection based on mean Cy3 fluorescence in the cytoplasm. Data was graphed using GraphPad Prism 7.05.

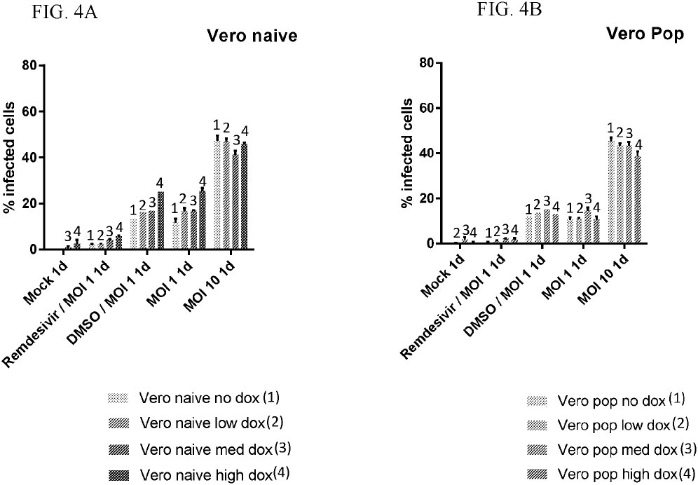

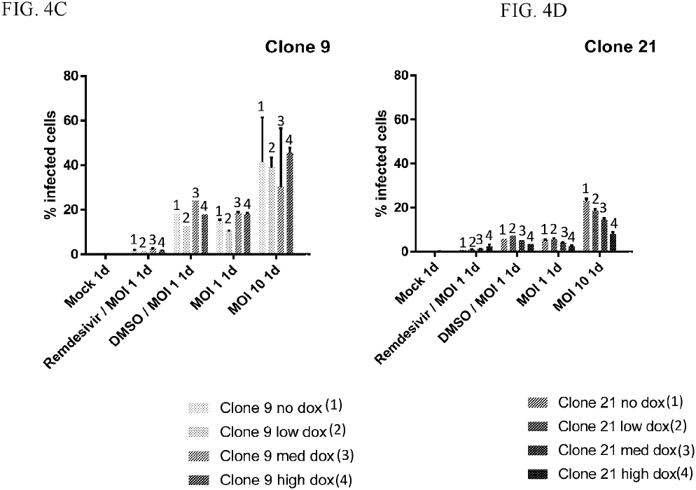

The percent of infected cells (% infected cells) for each condition is depicted in FIGS. 4A-4D. As shown, there was a reduction in the number of infected Clone 21 cells at 1 day post infection at MOI of 1 and 10 (FIG. 4D). This reduction was impressive as it was observed even with a relatively high MOI of 10. Exemplary Clone 21 was also observed to exhibit viral inhibition comparable to remdesivir, a known antiviral agent.

The present invention is not intended to be limited in scope to the particular disclosed embodiments, which are provided, for example, to illustrate various aspects of the invention. Various modifications to the compositions and methods described will become apparent from the description and teachings herein. Such variations may be practiced without departing from the true scope and spirit of the disclosure and are intended to fall within the scope of the present disclosure.

IX. SEQUENCES

| # | Sequence | Annotation |
|---|---|---|
| 1 | MSQEGVELEKSVRRLREKFHGKVSSKKAGALMRKFGSDHTGVGRSIVYGVKQKDGQELSNDLDAQDPPEDMKQDRDIQAVATSLLPLTEANLRMFQRAQDDLIPAVDRQFACSSCDHVWWRRVPQRKEVSRCRKCRKRYEPVPADKMWGLAEFHCPKCRHNFRGWAQMGSPSPCYGCGFPVYPTRILPPRWDRDPDRRSTHTHSCSAADCYNRREPHVPGTSCAHPKSRKQNHLPKVLHPSNPHISSGSTVATCLSQGGLLEDLDNLILEDLKEEEEEEEEVEDEEGGPRE | SHFL, Full length AA - Variant 1 |
| 2 | gaggcaccgc cccctgccct gcgcggctgc tggaccgacg ggcgcaccca ggtagggggg cggctgagcc gcgcagtgcg gaccctcgcg gggaactgcg ccgccgccac catgtctcag gaaggtgtgg agctggagaa gagcgtccgg cgcctccggg gaagtttca tgggaaggta tcctccaaga aggcgggggc tctgatgagg aaattcggca gcgaccacac gggagtgggg cgctccatcg tgtacggggt aaagcaaaaa gatggccaag aactaagtaa cgatctggat gcccaggatc caccagaaga tatgaagcag accgggaca ttcaggcagt ggcgacctcc ctcctgccac tgacagaagc caacctacgc atgtttcaac gtgcccagga cgaccttatc cctgctgtgg accggcagtt tgcctgctcc tcctgcgacc acgtctggtg gcgccgcgtg ccccagcgga aggaggtatc ccggtgccgg aaatgccgga agcgctacga gccagtgcca gctgacaaga tgtggggcct ggctgagttc cactgcccga agtgtcggca caacttccgg ggctgggcaca gatggggtc cccgtccccc tgctacgggt gcggcttccc cgtgtatcca acacggatcc tccccccgcg ctgggaccgg gacccggatc ccgcagcac ccacactcac tcctgctcag ctgccgactg ctacaaccgg cgagagcccc acgtgcctgg acatcctgt gctcacccca agagccggaa gcagaaccac ctgcccaaag tgctccaccc cagcaaccct cacattagca gtggctccac tgtggccacc gcttgagcc agggtggcct cctggaagac ctggacaacc tcatcctgga ggacctgaag gaggaggagg ggaagagga ggaggtggag gacgaggagg gcgggcccag ggagtgaccc ctgccaggtg cagatacaaa ccagacacgg tctgtggcta tttgtgtta ttataagata tgagctcaaa ccgagatatg aatgaccttg gggagccatc tgaggccaag atattgacgg gggggattcc tgggtcccat tttcagcgcc cagggtcaca gatccacagt gggaagttct gtgggacaca ttggcactga gccacaaaga aggtgtggcc agaacaactt gggctcctgc tgaccaatgt cctctagggc ctaggggaca gaggaacaca gagtcacagc ttcaggggcc gaatgagcat ggcggccttc ctgagagaat tgccccacc acgaaactca gcccagtaga caccatcctg gtagcggctt cggtagtggc cgccgtggtg ccacacaccg ttgaggttgg agtgggcaca ggcatggtac caccagcctc cccgctggta cagggcacag ttacctgagg ggagagagag agtccatgtc ctctcaccag aataaaagcc tctacctgca cctcacagtg caaggctttt gccaggcatc ccctggcccc tcccattctt attgaataca agccctgatc ttccatctcc tcagcaaaaa aataggagcc ctggcccccc aactttcttc agagtaatag ccttaattcc ttccctatct ccttaccaaa gtacaagtca catctttccc accttttctg caaactagga gtctaccgtt cattccttta tcaaagaaaa gtatctactt ctttctaga ataagagtac tagctctcac cctctgccct ttacttgaac aggagtcttg attctttttt tgcctcatca gagaaggaat tggactccc catcccccca ccaggataaa agtcctgacc tttgttctct tgacggaata aaagcttgct tatcctta | SHFL, Full length NA - Variant 1 |
| 3 | MSQHQQACGI CRQEDPPEDM KQDRDIQAVA TSLLPLTEAN LRMFQRAQDD LIPAVDRQFA CSSCDHVWWR RVPQRKEVSR CRKCRKRYEP VPADKMWGLA EFHCPKCRHN FRGWAQMGSP SPCYGCGFPV YPTRILPPRW DRDPDRRSTH THSCSAADCY NRREPHVPGT SCAHPKSRKQ NHLPKVLHPS NPHISSGSTV ATCLSQGGLL EDLDNLILED LKEEEEEEEE VEDEEGGPRE | SHFL, Full length AA - Variant 2 |
| 4 | gacatgtgac cgactgcaga ggccgcgccg cctcccccgt ccgaggtctg cgcgctccgc cgcagggtgc agacccgggg cgcccgcctg ggtttggggc gcaagagcag aggcggagcc agggcggagc cagcgcgccg ggtcccccct gaatcgaaag cgaaacaggg gccggggagg aagggcggag ccggccccga ggcaccgccc cctgccctgc gcggctgctg gaccgacggg cgcacccagg taggggggcg gctgagccgc gcagtgcgga ccctcgcggg gaactgcgcc gccgccacca tgtctcagga aggtgtggag ctggagaaga gcgtccggcg cctccgggag aagtttcatg ggaaggtatc ctccaagaag gcgggggctc tgatgaggaa attcggcagc gaccacacgg gagtggggcg ctccatcgtg tacggggtaa agcaaaaaga tggccaagaa ctaagtaacg atctggatgc ccagacaaag aaactgaggc cgaagtcact tgccagcaag caggggcagc tgcgatttga accctggcca cctggagtca agcctgggc tctaacatgc ctccgagcac aacgtggcct caaaggggcg atatgtcaca acatcaacaa gcatgtggca tttgcaggca ggaagatcca ccagaagata tgaagcagga ccgggacatt caggcagtgg cgacctccct cctgccactg acagaagcca acctacgcat gtttcaacgt gcccaggacg accttatccc tgctgtggac | SHFL, Full length NA - Variant 2 |
| | cggcagtttg cctgctcctc ctgcgaccac gtctggtggc gccgcgtgcc ccagcggaag gaggtatccc ggtgccggaa atgccggaag cgctacgagc cagtgccagc tgacaagatg tggggcctgg ctgagttcca ctgcccgaag tgtcggcaca acttccgggg ctgggcacag atggggtccc cgtccccctg ctacgggtgc ggcttccccg tgtatccaac acggatcctc cccccgcgct gggaccggga cccggatcgc cgcagcaccc acactcactc ctgctcagct gccgactgct acaaccggcg agagccccac gtgcctggga catcctgtgc caccccaag agccggaagc agaaccacct gcccaaagtg ctccacccca gcaaccctca cattagcagt ggctccactg tggccacctg ttgagccag ggtggcctcc tggaagacct ggacaacctc atcctggagg acctgaagga ggaggaggag gaagaggagg aggtggagga cgaggagggc gggcccaggg agtgacccct gccaggtgca gatacaaacc agacacggtc tgtggctact ttgtgttatt ataagatatg agctcaaacc gagatatgaa tgaccttggg gagccatctg aggccaagat attgacgggg gggattcctg ggtcccattt tcagcgccca gggtcacaga tccacagtgg gaagttctgt gggacacatt ggcactgagc cacaaagaag gtgtggccag aacaacttgg gctcctgctg accaatgtcc tctagggcct aggggacaga ggaacacaga gtcacagctt caggggccga atgagcatgg cggccttcct gagagaatat gccccaccac gaaactcagc ccagtagaca ccatcctggt agcggcttcg gtagtggccg ccgtggtgcc acacaccgtt gaggttggag tgggcacagg catggtacca ccagcctccc cgctggtaca gggcacagtt acctgagggg agagagagag tccatgtcct ctcaccagaa taaaagcctc tacctgcacc tcacagtgca aggcttttgc caggcatccc ctggcccctc ccattcttat tgaatacaag ccctgatctt ccatctcctc agcaaaaaaa taggagccct ggccccccaa ctttcttcag agtaatagcc ttaattcctt ccctatctcc ttaccaaagt acaagtcaca tctttcccac cttttctgca aactaggagt ctaccgttca ttcctttatc aagaaaagt atctacttcc tttctagaat aagagtacta gctctcaccc tctgcccttt acttgaacag gagtcttgat tctttttttg cctcatcaga gaaggaatct ggactcccca tccccccacc aggataaaag tcctgacctt tgttctcttg acggaataaa agcttgctta tccttatact ta | |
| 5 | SQEGVELEKSVRRLREKFHGKVSSKKAGALMRKFGSDHTGVGRSIVYGVKQKDGQELSNDLDAQDPPEDMKQDRDIQAVATSLLPLTEANLRMFQRAQDDLIPAVDRQFACSSCDHVWWRRVPQRKEVSRCRKCRKRYEPVPADKMWGLAEFHCPKCRHNFRGWAQMGSPSPCYGCGFPVYPTRILPPRWDRDPDRRSTHTHSCSAADCYNRREPHVPGTSCAHPKSRKQNHLPKVLHPSNPHISSGSTVATCLSQGGLLEDLDNLILEDLKEEEEEEEEVEDEEGGPRE | SHFL, deltaM AA -Variant 1 |
| 6 | tctcag gaaggtgtgg agctggagaa gagcgtccgg cgcctccggg agaagtttca tgggaaggta tcctccaaga aggcgggggc tctgatgagg aaattcggca gcgaccacac gggagtgggg cgctccatcg tgtacggggt aaagcaaaaa gatggccaag aactaagtaa cgatctggat gcccaggatc caccagaaga tatgaagcag gaccgggaca ttcaggcagt ggcgacctcc ctcctgccac gacagaagc caacctacgc atgtttcaac gtgcccagga cgaccttatc cctgctgtgg accggcagtt tgcctgctcc tcctgcgacc acgtctggtg gcgccgcgtg ccccagcgga aggaggtatc ccggtgccgg aaatgccgga agcgctacga gccagtgcca gctgacaaga tgtggggcct ggctgagttc cactgcccga agtgtcggca caacttccgg ggctgggcac agatggggtc cccgtccccc tgctacgggt gcggcttccc cgtgtatcca acacggatcc tccccccgcg ctgggaccgg gacccggatc gccgcagcac cacactcac tcctgctcag ctgccgactg ctacaaccgg cgagagcccc acgtgcctgg gacatcctgt gctcacccca agagccggaa cagaaccac tgcccaaag tgctccaccc cagcaaccct cacattagca tggctccac tgtggccacc tgcttgagcc agggtggcct cctggaagac ctggacaacc tcatcctgga ggacctgaag gaggaggagg aggaagagga ggaggtggag gacgaggagg gcgggcccag ggagtgaccc ctgccaggtg cagatacaaa ccagacacgg tctgtggcta ctttgtgtta ttataagata tgagctcaaa ccgagatatg aatgaccttg gggagccatc tgaggccaag atattgacgg gggggattcc tgggtcccat tttcagcgcc cagggtcaca gatccacagt gggaagttct gtgggacaca ttggcactga gccacaaaga aggtgtggcc agaacaactt gggctcctgc tgaccaatgt cctctagggc ctaggggaca gaggaacaca gagtcacagc ttcaggggcc gaatgagcat ggcggccttc ctgagagaat atgccccacc acgaaactca cccagtaga caccatcctg gtagcggctt cggtagtggc cgccgtggtg ccacacaccg ttgaggttgg agtgggcaca ggcatggtac caccagcctc cccgctggta cagggcacag ttacctgagg ggagagagag agtccatgtc ctctcaccag aataaaagcc tctacctgca cctcacagtg caaggctttt gccaggcatc ccctggcccc tcccattctt attgaataca agccctgatc ttccatctcc tcagcaaaaa aataggagcc ctggcccccc aactttcttc agagtaatag ccttaattcc ttccctatct ccttaccaaa gtacaagtca catctttccc accttttctg caaactagga gtctaccgtt cattccttta tcaaagaaaa gtatctactt cctttctaga ataagagtac tagctctcac ctctgccct ttacttgaac aggagtcttg attctttttt tgcctcatca gagaaggaat ctggactccc catcccccca ccaggataaa agtcctgacc tttgttctct tgacggaata aaagcttgct tatcctta | SHFL, deltaM NA -Variant 1 |
| 7 | PPSTTWPQRGDMSQHQQACGICRQEDPPEDMKQDRDIQAVATSLLPLTEANLRMFQRAQDDLIPAVDRQFACSSCDHVWWRRVPQRKEVSRCRKCR | SHFL, deltaM AA -Variant 2 |
| | KRYEPVPADKMWGLAEFHCPKCRHNFRGWAQMGSPSPCYGCGFPVYPTRILPPRWDRDPDRRSTHTHSCSAADCYNRREPHVPGTSCAHPKSRKQNHLPKVLHPSNPHISSGSTVATCLSQGGLLEDLDNLILEDLKEEEEEEEEVEDEEGGPRE | |
| 8 | tgac cgactgcaga ggccgcgccg cctcccccgt ccgaggtctg cgcgctccgc cgcagggtgc agacccgggg cgcccgcctg ggtttggggc gcaagagcag aggcggagcc agggcggagc cagcgcgccg ggtcccccct gaatcgaaag cgaaacaggg gccggggagg aagggcggag ccggccccga ggcaccgccc cctgccctgc gcggctgctg gaccgacggg cgcacccagg taggggggcg gctgagccgc gcagtgcgga ccctcgcggg gaactgcgcc gccgccacca tgtctcagga aggtgtggag ctggagaaga gcgtccggcg cctccgggag aagtttcatg ggaaggtatc ctccaagaag gcgggggctc tgatgaggaa attcggcagc gaccacacgg gagtggggcg ctccatcgtg tacggggtaa agcaaaaaga tggccaagaa ctaagtaacg atctggatgc ccagacaaag aaactgaggc cgaagtcact tgccagcaag caggggcagc tgcgatttga accctggcca cctggagtca gagcctgggc tctaacatgc ctccgagcac aacgtggcct caaaggggcg atatgtcaca acatcaacaa gcatgtggca tttgcaggca ggaagatcca ccagaagata tgaagcagga ccgggacatt caggcagtgg cgacctccct ctgccactg acagaagcca acctacgcat gtttcaacgt gcccaggacg accttatccc tgctgtggac cggcagtttg cctgctcctc ctgcgaccac gtctggtggc gccgcgtgcc ccagcggaag gaggtatccc ggtgccggaa atgccggaag cgctacgagc cagtgccagc tgacaagatg tggggcctgg ctgagttcca ctgcccgaag tgtcggcaca acttccgggg ctgggcacag atggggtccc cgtccccctg ctacgggtgc ggcttccccg tgtatccaac acggatcctc cccccgcgct gggaccggga cccggatcgc cgcagcaccc acactcactc ctgctcagct gccgactgct acaaccggcg agagccccac gtgcctggga catcctgtgc tcaccccaag agccggaagc agaaccacct gcccaaagtg ctccacccca gcaaccctca cattagcagt ggctccactg tggccacctg cttgagccag ggtggcctcc tggaagacct ggacaacctc atcctggagg acctgaagga ggaggaggag gaagaggagg aggtggagga cgaggagggc gggcccaggg agtgacccct gccaggtgca gatacaaacc agacacggtc tgtggctact ttgtgttatt ataagatatg agctcaaacc gagatatgaa tgaccttggg gagccatctg aggccaagat attgacgggg gggattcctg ggtcccattt tcagcgccca gggtcacaga tccacagtgg gaagttctgt gggacacatt ggcactgagc cacaaagaag gtgtggccag aacaacttgg gctcctgctg accaatgtcc tctagggcct aggggacaga ggaacacaga gtcacagctt caggggccga atgagcatgg cggccttcct gagagaatat gccccaccac gaaactcagc ccagtagaca ccatcctggt agcggcttcg gtagtggccg ccgtggtgcc acacaccgtt gaggttggag tgggcacagg catggtacca ccagcctccc cgctggtaca gggcacagtt acctgagggg agagagagag tccatgtcct ctcaccagaa taaaagcctc tacctgcacc tcacagtgca aggcttttgc caggcatccc ctggcccctc ccattcttat tgaatacaag ccctgatctt ccatctcctc agcaaaaaaa taggagccct ggccccccaa ctttcttcag agtaatagcc ttaattcctt ccctatctcc ttaccaaagt acaagtcaca tctttcccac cttttctgca aactaggagt ctaccgttca ttcctttatc aaagaaaagt atctacttcc tttctagaat aagagtacta gctctcaccc tctgcccttt acttgaacag gagtcttgat tctttttttg cctcatcaga gaaggaatct ggactcccca tccccccacc aggataaaag tcctgacctt tgttctcttg acggaataaa agcttgctta tccttatact ta | SHFL, deltaM NA -Variant 2 |
| 9 | LXXXLXXLXL | NES Consensus |
| 10 | GTATAGGGCTGTCTGGGAGCCACTCCAGGGCCACAGAAATCTTGTCTCTGACTCAGGGTATTTTGTTTTCTGTTTTGTGTAAATGCTCTTCTGACTAATGCAAACCATGTGTCCATAGAACCAGAAGATTTTTCCAGGGGAAAAGGTAAGGAGGTGGTGAGAGTGTCCTGGGTCTGCCCTTCCAGGGCTTGCCCTGGGTTAAGAGCCAGGCAGGAAGCTCTCAAGAGCATTGCTCAAGAGTAGAGGGGGCCTGGGAGGCCCAGGGAGGGGATGGGAGGGGAACACCCAGGCTGCCCCCAACCAGATGCCCTCCACCCTCCTCAACCTCCCTCCCACGGCCTGGAGAGGTGGGACCAGGTATGGAGGCTTGAGAGCCCCTGGTTGGAGGAAGCCACAAGTCCAGGAACATGGGAGTCTGGGCAGGGGGCAAAGGAGGCAGGAACAGGCCATCAGCCAGGACAGGTGGTAAGGCAGGCAGGAGTGTTCCTGCTGGGAAAAGGTGGGATCAAGCACCTGGAGGGCTCTTCAGAGCAAAGACAAACACTGAGGTCGCTGCCACTCCTACAGAGCCCCCACGCCCCGCCCAGCTATAAGGGGCCATGCACCAAGCAGGGTACCCAGGCTGCAGAGGTGCC | Promoter region of the human SB gene |
| 11 | RRVPQRKEVSRCRKCRK | SHFL AA 121-137 |
| 12 | LEDLDNLIL | SHFL AA 261-269 |
| 13 | GRKKRRQRRRPPQ | TAT |
| 14 | RQIKIWFQNRRMKWKK | Penetratin |
| 15 | GWTLNSAGYLLGKINLKALAALAKKIL | Transporant |
| 16 | PLILLRLLRGQF | Pept 1 |
| 17 | PLIYLRLLRGQF | Pept 2 |
| 18 | GWTLNSAGYLLGKINLKALAALAKKIL | Transportan |
| 19 | MGLGLHLLVLAAALQGAKKKRKV | IgV |
| 20 | GGGGS | Peptide Linker |
| 21 | GGGGGS | Peptide Linker |
| 22 | (GGGGS)n | Peptide Linker n is 1 to 6 |
| 23 | (GGGGS)n | Peptide Linker n is 1 to 10 |
| 24 | MMADSKLVSLNNNLSGKIKDQGKVIKNYYGTMDIKKINDGLLDSKILGAFNTVIALLGSIIIIVMNIMIIQNYTRTTDNQALIKESLQSVQQQIKALTDKIGTEIGPKVSLIDTSSTITIPANIGLLGSKISQSTSSINENVNDKCKFTLPPLKIHECNISCPNPLPFREYRPISQGVSDLVGLPNQICLQKTTSTILKPRLISYTLPINTREGVCITDPLLAVDNGFFAYSHLEKIGSCTRGIAKQRIIGVGEVLDRGDKVPSMFMTNVWTPPNPSTIHHCSSTYHEDFYYTLCAVSHVGDPILNSTSWTESLSLIRLAVRPKSDSGDYNQKYIAITKVERGKYDKVMPYGPSGIKQGDTLYFPAVGFLPRTEFQYNDSNCPIIHCKYSKAENCRLSMGVNSKSHYILRSGLLKYNLSLGGDIILQFIEIADNRLTIGSPSKIYNSLGQPVFYQASYSWDTMIKLGDVDTVDPLRVQWRNNSVISRPGQSQCPRFNVCPEVCWEGTYNDAFLIDRLNWVSAGVYLNSNQTAENPVFAVFKDNEILYQVPLAEDDTNAQKTITDCFLLENVIWCISLVEIYDTGDSVIRPKLFAVKIPAQCSES | Hendra Virus G Protein |
| 25 | MADSKLVSLNNNLSGKIKDQGKVIKNYYGTMDIKKINDGLLDSKILGAFNTVIALLGSIIIIVMNIMIIQNYTRTTDNQALIKESLQSVQQQIKALTDKIGTEIGPKVSLIDTSSTITIPANIGLLGSKISQSTSSINENVNDKCKFTLPPLKIHECNISCPNPLPFREYRPISQGVSDLVGLPNQICLQKTTSTILKPRLISYTLPINTREGVCITDPLLAVDNGFFAYSHLEKIGSCTRGIAKQRIIGVGEVLDRGDKVPSMFMTNVWTPPNPSTIHHCSSTYHEDFYYTLCAVSHVGDPILNSTSWTESLSLIRLAVRPKSDSGDYNQKYIAITKVERGKYDKVMPYGPSGIKQGDTLYFPAVGFLPRTEFQYNDSNCPIIHCKYSKAENCRLSMGVNSKSHYILRSGLLKYNLSLGGDIILQFIEIADNRLTIGSPSKIYNSLGQPVFYQASYSWDTMIKLGDVDTVDPLRVQWRNNSVISRPGQSQCPRFNVCPEVCWEGTYNDAFLIDRLNWVSAGVYLNSNQTAENPVFAVFKDNEILYQVPLAEDDTNAQKTITDCFLLENVIWCISLVEIYDTGDSVIRPKLFAVKIPAQCSES | Hendra Virus G Protein (No Met) |
| 26 | MPAENKKVRFENTTSDKGKIPSKVIKSYYGTMDIKKINEGLLDSKILSAFNTVIALLGSIVIIVMNIMIIQNYTRSTDNQAVIKDALQGIQQQIKGLADKIGTEIGPKVSLIDTSSTITIPANIGLLGSKISQSTASINENVNEKCKFTLPPLKIHECNISCPNPLPFREYRPQTEGVSNLVGLPNNICLQKTSNQILKPKLISYTLPVVGQSGTCITDPLLAMDEGYFAYSHLERIGSCSRGVSKQRIIGVGEVLDRGDEVPSLFMTNVWTPPNPNTVYHCSAVYNNEFYYVLCAVSTVGDPILNSTYWSGSLMMTRLAVKPKSNGGGYNQHQLALRSIEKGRYDKVMPYGPSGIKQGDTLYFPAVGFLVRTEFKYNDSNCPITKCQYSKPENCRLSMGIRPNSHYILRSGLLKYNLSDGENPKVVFIEISDQRLSIGSPSKIYDSLGQPVFYQASFSWDTMIKFGDVLTVNPLVVNWRNNTVISRPGQSQCPRFNTCPEICWEGVYNDAFLIDRINWISAGVFLDSNQTAENPVFTVFKDNEILYRAQLASEDTNAQKTITNCFLLKNKIWCISLVEIYDTGDNVIRPKLFAVKIPEQCT | Nipah Virus G Protein |
| 27 | PAENKKVRFENTTSDKGKIPSKVIKSYYGTMDIKKINEGLLDSKILSAFNTVIALLGSIVIIVMNIMIIQNYTRSTDNQAVIKDALQGIQQQIKGLADKIGTEIGPKVSLIDTSSTITIPANIGLLGSKISQSTASINENVNEKCKFTLPPLKIHECNISCPNPLPFREYRPQTEGVSNLVGLPNNICLQKTSNQILKPKLISYTLPVVGQSGTCITDPLLAMDEGYFAYSHLERIGSCSRGVSKQRIIGVGEVLDRGDEVPSLFMTNVWTPPNPNTVYHCSAVYNNEFYYVLCAVSTVGDPILNSTYWSGSLMMTRLAVKPKSNGGGYNQHQLALRSIEKGRYDKVMPYGPSGIKQGDTLYFPAVGFLVRTEFKYNDSNCPITKCQYSKPENCRLSMGIRPNSHYILRSGLLKYNLSDGENPKVVFIEISDQRLSIGSPSKIYDSLGQPVFYQASFSWDTMIKFGDVLTVNPLVVNWRNNTVISRPGQSQCPRFNTCPEICWEGVY | Nipah Virus G Protein (No Met) |
| | NDAFLIDRINWISAGVFLDSNQTAENPVFTVFKDNEILYRAQLASEDTNAQKTITNCFLLKNKIWCISLVEIYDTGDNVIRPKLFAVKIPEQCT | |
| 28 | MLSQLQKNYLDNSNQQGDKMNNPDKKLSVNFNPLELDKGQKDLNKSYYVKNKNYNVSNLLNESLHDIKFCIYCIFSLLIIITIINIITISIVITRLKVHEENNGMESPNLQSIQDSLSSLTNMINTEITPRIGILVTATSVTLSSSINYVGTKTNQLVNELKDYITKSCGFKVPELKLHECNISCADPKISKSAMYSTNAYAELAGPPKIFCKSVSKDPDFRLKQIDYVIPVQQDRSICMNNPLLDISDGFFTYIHYEGINSCKKSDSFKVLLSHGEIVDRGDYRPSLYLLSSHYHPYSMQVINCVPVTCNQSSFVFCHISNNTKTLDNSDYSSDEYYITYFNGIDRPKTKKIPINNMTADNRYIHFTFSGGGGVCLGEEFIIPVTTVINTDVFTHDYCESFNCSVQTGKSLKEICSESLRSPTNSSRYNLNGIMIISQNNMTDFKIQLNGITYNKLSFGSPGRLSKTLGQVLYYQSSMSWDTYLKAGFVEKWKPFTPNWMNNTVISRPNQGNCPRYHKCPEICYGGTYNDIAPLDLGKDMYVSVILDSDQLAENPEITVFNSTTILYKERVSKDELNTRSTTTSCFLFLDEPWCISVLETNRFNGKSIRPEIYSYKIPKYC | Cedar Virus G Protein |
| 29 | LSQLQKNYLDNSNQQGDKMNNPDKKLSVNFNPLELDKGQKDLNKSYYVKNKNYNVSNLLNESLHDIKFCIYCIFSLLIIITIINIITISIVITRLKVHEENNGMESPNLQSIQDSLSSLTNMINTEITPRIGILVTATSVTLSSSINYVGTKTNQLVNELKDYITKSCGFKVPELKLHECNISCADPKISKSAMYSTNAYAELAGPPKIFCKSVSKDPDFRLKQIDYVIPVQQDRSICMNNPLLDISDGFFTYIHYEGINSCKKSDSFKVLLSHGEIVDRGDYRPSLYLLSSHYHPYSMQVINCVPVTCNQSSFVFCHISNNTKTLDNSDYSSDEYYITYFNGIDRPKTKKIPINNMTADNRYIHFTFSGGGGVCLGEEFIIPVTTVINTDVFTHDYCESFNCSVQTGKSLKEICSESLRSPTNSSRYNLNGIMIISQNNMTDFKIQLNGITYNKLSFGSPGRLSKTLGQVLYYQSSMSWDTYLKAGFVEKWKPFTPNWMNNTVISRPNQGNCPRYHKCPEICYGGTYNDIAPLDLGKDMYVSVILDSDQLAENPEITVFNSTTILYKERVSKDELNTRSTTTSCFLFLDEPWCISVLETNRFNGKSIRPEIYSYKIPKYC | Cedar Virus G Protein (No Met) |
| 30 | MPQKTVEFINMNSPLERGVSTLSDKKTLNQSKITKQGYFGLGSHSERNWKKQKNQNDHYMTVSTMILEILVVLGIMFNLIVLTMVYYQNDNINQRMAELTSNITVLNLNLNQLTNKIQREIIPRITLIDTATTITIPSAITYILATLTTRISELLPSINQKCEFKTPTLVLNDCRINCTPPLNPSDGVKMSSLATNLVAHGPSPCRNFSSVPTIYYYRIPGLYNRTALDERCILNPRLTISSTKFAYVHSEYDKNCTRGFKYYELMTFGEILEGPEKEPRMFSRSFYSPTNAVNYHSCTPIVTVNEGYFLCLECTSSDPLYKANLSNSTFHLVILRHNKDEKIVSMPSFNLSTDQEYVQIIPAEGGGTAESGNLYFPCIGRLLHKRVTHPLCKKSNCSRTDDESCLKSYYNQGSPQHQVVNCLIRIRNAQRDNPTWDVITVDLTNTYPGSRSRIFGSFSKPMLYQSSVSWHTLLQVAEITDLDKYQLDWLDTPYISRPGGSECPFGNYCPTVCWEGTYNDVYSLTPNNDLFVTVYLKSEQVAENPYFAIFSRDQILKEFPLDAWISSARTTTISCFMFNNEIWCIAALEITRLNDDIIRPIYYSFWLPTDCRTPYPHTGKMTRVPLRSTYNY | Bat Paramyxovirus G Protein |
| 31 | PQKTVEFINMNSPLERGVSTLSDKKTLNQSKITKQGYFGLGSHSERNWKKQKNQNDHYMTVSTMILEILVVLGIMFNLIVLTMVYYQNDNINQRMAELTSNITVLNLNLNQLTNKIQREIIPRITLIDTATTITIPSAITYILATLTTRISELLPSINQKCEFKTPTLVLNDCRINCTPPLNPSDGVKMSSLATNLVAHGPSPCRNFSSVPTIYYYRIPGLYNRTALDERCILNPRLTISSTKFAYVHSEYDKNCTRGFKYYELMTFGEILEGPEKEPRMFSRSFYSPTNAVNYHSCTPIVTVNEGYFLCLECTSSDPLYKANLSNSTFHLVILRHNKDEKIVSMPSFNLSTDQEYVQIIPAEGGGTAESGNLYFPCIGRLLHKRVTHPLCKKSNCSRTDDESCLKSYYNQGSPQHQVVNCLIRIRNAQRDNPTWDVITVDLTNTYPGSRSRIFGSFSKPMLYQSSVSWHTLLQVAEITDLDKYQLDWLDTPYISRPGGSECPFGNYCPTVCWEGTYNDVYSLTPNNDLFVTVYLKSEQVAENPYFAIFSRDQILKEFPLDAWISSARTTTISCFMFNNEIWCIAALEITRLNDDIIRPIYYSFWLPTDCRTPYPHTGKMTRVPLRSTYNY | Bat Paramyxovirus G Protein (No Met) |
| 32 | MATNRDNTITSAEVSQEDKVKKYYGVETAEKVADSISGNKVFILMNTLLILTGAIITITLNITNLTAAKSQQNMLKIIQDDVNAKLEMFVNLDQLVKGEIKPKVSLINTAVSVSIPGQISNLQTKFLQKYVYLEESITKQCTCNPLSGIFPTSGPTYPPTDKPDDDTTDDDKVDTTIKPIEYPKPDGCNRTGDHFTMEPGA | Mojiang virus, Tongguan 1 G Protein |
| | NFYTVPNLGPASSNSDECYTNPSFSIGSSIYMFSQEIRKTDCTAGEILSIQIVLGRIVDKGQQGPQASPLLVWAVPNPKIINSCAVAAGDEMGWVLCSVTLTAASGEPIPHMFDGFWLYKLEPDTEVVSYRITGYAYLLDKQYDSVFIGKGGGIQKGNDLYFQMYGLSRNRQSFKALCEHGSCLGTGGGGYQVLCDRAVMSFGSEESLITNAYLKVNDLASGKPVIIGQTFPPSDSYKGSNGRMYTIGDKYGLYLAPSSWNRYLRFGITPDISVRSTTWLKSQDPIMKILSTCTNTDRDMCPEICNTRGYQDIFPLSEDSEYYTYIGITPNNGGTKNFVAVRDSDGHIASIDILQNYYSITSATISCFMYKDEIWCIAITEGKKQKDNPQRIYAHSYKIRQMCYNMKSATVTVGNAKNITIRRY | |
| 33 | ATNRDNTITSAEVSQEDKVKKYYGVETAEKVADSISGNKVFILMNTLLILTGAIITITLNITNLTAAKSQQNMLKIIQDDVNAKLEMFVNLDQLVKGEIKPKVSLINTAVSVSIPGQISNLQTKFLQKYVYLEESITKQCTCNPLSGIFPTSGPTYPPTDKPDDDTTDDDKVDTTIKPIEYPKPDGCNRTGDHFTMEPGANFYTVPNLGPASSNSDECYTNPSFSIGSSIYMFSQEIRKTDCTAGEILSIQIVLGRIVDKGQQGPQASPLLVWAVPNPKIINSCAVAAGDEMGWVLCSVTLTAASGEPIPHMFDGFWLYKLEPDTEVVSYRITGYAYLLDKQYDSVFIGKGGGIQKGNDLYFQMYGLSRNRQSFKALCEHGSCLGTGGGGYQVLCDRAVMSFGSEESLITNAYLKVNDLASGKPVIIGQTFPPSDSYKGSNGRMYTIGDKYGLYLAPSSWNRYLRFGITPDISVRSTTWLKSQDPIMKILSTCTNTDRDMCPEICNTRGYQDIFPLSEDSEYYTYIGITPNNGGTKNFVAVRDSDGHIASIDILQNYYSITSATISCFMYKDEIWCIAITEGKKQKDNPQRIYAHSYKIRQMCYNMKSATVTVGNAKNITIRRY | Mojiang virus, Tongguan 1 G (No Met) |
| 34 | MATQEVRLKCLLCGIIVLVLSLEGLGILHYEKLSKIGLVKGITRKYKIKSNPLTKDIVIKMIPNVSNVSKCTGTVMENYKSRLTGILSPIKGAIELYNNNTHDLVGDVKLAGVVMAGIAIGIATAAQITAGVALYEAMKNADNINKLKSSIESTNEAVVKLQETAEKTVYVLTALQDYINTNLVPTIDQISCKQTELALDLALSKYLSDLLFVFGPNLQDPVSNSMTIQAISQAFGGNYETLLRTLGYATEDFDDLLESDSIAGQIVYVDLSSYYIIVRVYFPILTEIQQAYVQELLPVSFNNDNSEWISIVPNFVLIRNTLISNIEVKYCLITKKSVICNQDYATPMTASVREC LTGSTDKCPRELVVSSHVPRFALSGGVLFANCISVTCQCQTTGRAISQSGEQTLLMIDNTTCTTVVLGNIIISLGKYLGSINYNSESIAVGPPVYTDKVDISSQISSMNQSLQQSKDYIKEAQKILDTVNPSLISMLSMIILYVLSIAALCIGLITFISFVIVEKKRGNYSRLDDRQVRPVSNGDLYYIGT | Hendra virus F Protein |
| 35 | ILHYEKLSKIGLVKGITRKYKIKSNPLTKDIVIKMIPNVSNVSKCTGTVMENYKSRLTGILSPIKGAIELYNNNTHDLVGDVKLAGVVMAGIAIGIATAAQITAGVALYEAMKNADNINKLKSSIESTNEAVVKLQETAEKTVYVLTALQDYINTNLVPTIDQISCKQTELALDLALSKYLSDLLFVFGPNLQDPVSNSMTIQAISQAFGGNYETLLRTLGYATEDFDDLLESDSIAGQIVYVDLSSYYIIVRVYFPILTEIQQAYVQELLPVSFNNDNSEWISIVPNFVLIRNTLISNIEVKYCLITKKSVICNQDYATPMTASVRECLTGSTDKCPRELVVSSHVPRFALSG GVLFANCISVTCQCQTTGRAISQSGEQTLLMIDNTTCTTVVLGNIIISLGKYLGSINYNSESIAVGPPVYTDKVDISSQISSMNQSLQQSKDYIKEAQKILDTVNPSLISMLSMIILYVLSIAALCIGLITFISFVIVEKKRGNYSRLDDRQVRPVSNGDLYYIGT | Hendra virus F Protein, Without signal sequence |
| 36 | MVVILDKRCYCNLLILILMISECSVGILHYEKLSKIGLVKGVTRKYKIKSNPLTKDIVIKMIPNVSNMSQCTGSVMENYKTRLNGILTPIKGALEIYKNNTHDLVGDVRLAGVIMAGVAIGIATAAQITAGVALYEAMKNADNINKLKSSIESTNEAVVKLQETAEKTVYVLTALQDYINTNLVPTIDKISCKQTELSLDLALSKYLSDLLFVFGPNLQDPVSNSMTIQAISQAFGGNYETLLRTLGYATEDFDDLLESDSITGQIIYVDLSSYYIIVRVYFPILTEIQQAYIQELLPVSFNNDNSEWISIVPNFILVRNTLISNIEIGFCLITKRSVICNQDYATPMTNNMREC LTGSTEKCPRELVVSSHVPRFALSNGVLFANCISVTCQCQTTGRAISQSGEQTLLMIDNTTCPTAVLGNVIISLGKYLGSVNYNSEGIAIGPPVFTDKVDISSQISSMNQSLQQSKDYIKEAQRLLDTVNPSLISMLSMIILYVLSIASLCIGLITFISFIIVEKKRNTYSRLEDRRVRPTSSGDLYYIGT | Nipah virus F Protein |
| 37 | ILHYEKLSKIGLVKGVTRKYKIKSNPLTKDIVIKMIPNVSNMSQCTGSVMENYKTRLNGILTPIKGALEIYKNNTHDLVGDVRLAGVIMAGVAIGIATAAQITAGVALYEAMKNADNINKLKSSIESTNEAVVKLQETAEKTVYVLTALQDYINTNLVPTIDKISCKQTELSLDLALSKYLSDLLFVFGPNLQDPVSNSMTIQAISQAFGGNYETLLRTLGYATEDFDDLLESDSITGQIIYVDLSSYYIIVRVYFPILTEIQQAYIQELLPVSFNNDNSEWISIVPNFILVRNTLISNIEIGFCLITKRSVICNQDYATPMTNNMRECLTGSTEKCPRELVVSSHVPRFALSNGVLFANCISVTCQCQTTGRAISQSGEQTLLMIDNTTCPTAVLGNVIISLGKYLGSVNYNSEGIAIGPPVFTDKVDISSQISSMNQSLQQSKDYIKEAQRLLDTVNPSLISMLSMIILYVLSIASLCIGLITFISFIIVEKKRNTYSRLEDRRVRPTSSGDLYYIGT | Nipah virus F Protein, without signal sequence |
| 38 | MSNKRTTVLIIISYTLFYLNNAAIVGFDFDKLNKIGVVQGRVLNYKIKGDPMTKDLVLKFIPNIVNITECVREPLSRYNETVRRLLLPIHNMLGLYLNNTNAKMTGLMIAGVIMGGIAIGIATAAQITAGFALYEAKKNTENIQKLTDSIMKTQDSIDKLTDSVGTSILILNKLQTYINNQLVPNLELLSCRQNKIEFDLMLTKYLVDLMTVIGPNINNPVNKDMTIQSLSLLFDGNYDIMMSELGYTPQDFLDLIESKSITGQIIYVDMENLYVVIRTYLPTLIEVPDAQIYEFNKITMSSNGGEYLSTIPNFILIRGNYMSNIDVATCYMTKASVICNQDYSLPMSQNLRSCYQGETEYCPVEAVIASHSPRFALTNGVIFANCINTICRCQDNGKTITQNINQFVSMIDNSTCNDVMVDKFTIKVGKYMGRKDINNINIQIGPQIIIDKVDLSNEINKMNQSLKDSIFYLREAKRILDSVNISLISPSVQLFLIIISVLSFIILLIIIVYLYCKSKHSYKYNKFIDDPDYYNDYKRERINGKASKSNNIYYVGD | Cedar Virus F Protein |
| 39 | TVLIIISYTLFYLNNAAIVGFDFDKLNKIGVVQGRVLNYKIKGDPMTKDLVLKFIPNIVNITECVREPLSRYNETVRRLLLPIHNMLGLYLNNTNAKMTGLMIAGVIMGGIAIGIATAAQITAGFALYEAKKNTENIQKLTDSIMKTQDSIDKLTDSVGTSILILNKLQTYINNQLVPNLELLSCRQNKIEFDLMLTKYLVDLMTVIGPNINNPVNKDMTIQSLSLLFDGNYDIMMSELGYTPQDFLDLIESKSITGQIIYVDMENLYVVIRTYLPTLIEVPDAQIYEFNKITMSSNGGEYLSTIPNFILIRGNYMSNIDVATCYMTKASVICNQDYSLPMSQNLRSCYQGET EYCPVEAVIASHSPRFALTNGVIFANCINTICRCQDNGKTITQNINQFVSMIDNSTCNDVMVDKFTIKVGKYMGRKDINNINIQIGPQIIIDKVDLSNEINKMNQSLKDSIFYLREAKRILDSVNISLISPSVQLFLIIISVLSFIILLIIIVYLYCKSKHSYKYNKFIDDPDYYNDYKRERINGKASKSNNIYYVGD | Cedar Virus F Protein, without signal sequence |
| 40 | MALNKNMFSSLFLGYLLVYATTVQSSIHYDSLSKVGVIKGLTYNYKIKGSPSTKLMVVKLIPNIDSVKNCTQKQYDEYKNLVRKALEPVKMAIDTMLNNVKSGNNKYRFAGAIMAGVALGVATAATVTAGIALHRSNENAQAIANMKSAIQNTNEAVKQLQLANKQTLAVIDTIRGEINNNIIPVINQLSCDTIGLSVGIRLTQYYSEIITAFGPALQNPVNTRITIQAISSVFNGNFDELLKIMGYTSGDLYEILHSELIRGNIIDVDVDAGYIALEIEFPNLTLVPNAVVQELMPISYNIDGDEWVTLVPRFVLTRTTLLSNIDTSRCTITDSSVICDNDYALPMSHELIGCLQGDTSKCAREKVVSSYVPKFALSDGLVYANCLNTICRCMDTDTPISQSLGATVSLLDNKRCSVYQVGDVLISVGSYLGDGEYNADNVELGPPIVIDKIDIGNQLAGINQTLQEAEDYIEKSEEFLKGVNPSIITLGSMVVLYIFMILIAIVSVIALVLSIKLTVKGNVVRQQFTYTQHVPSMENINYVSH | Mojiang virus, Tongguan 1 F Protein |
| 41 | IHYDSLSKVGVIKGLTYNYKIKGSPSTKLMVVKLIPNIDSVKNCTQKQYDEYKNLVRKALEPVKMAIDTMLNNVKSGNNKYRFAGAIMAGVALGVATAATVTAGIALHRSNENAQAIANMKSAIQNTNEAVKQLQLANKQTLAVIDTIRGEINNNIIPVINQLSCDTIGLSVGIRLTQYYSEIITAFGPALQNPVNTRITIQAISSVFNGNFDELLKIMGYTSGDLYEILHSELIRGNIIDVDVDAGYIALEIEFPNLTLVPNAVVQELMPISYNIDGDEWVTLVPRFVLTRTTLLSNIDTSRCTITDSSVICDNDYALPMSHELIGCLQGDTSKCAREKVVSSYVPKFALSD GLVYANCLNTICRCMDTDTPISQSLGATVSLLDNKRCSVYQVGDVLISVGSYLGDGEYNADNVELGPPIVIDKIDIGNQLAGINQTLQEAEDYIEKSEEFLKGVNPSIITLGSMVVLYIFMILIAIVSVIALVLSIKLTVKGNVVRQQFTYTQHVPSMENINYVSH | Mojiang virus, Tongguan 1 F Protein, without signal sequence |
| 42 | MKKKTDNPTISKRGHNHSRGIKSRALLRETDNYSNGLIVENLVRNCHHPSKNNLNYTKTQKRDSTIPYRVEERKGHYPKIKHLIDKSYKHIKRGKRRNGHNGNTTTTTT T T TT TT KTQMSEGAIHYETLSKIGLIKGITREYKVKGTPSSK | Bat Paramyxovirus F Protein |
| | DIVIKLIPNVTGLNKCTNISMENYKEQLDKILIPINNIIELYANSTKSAPGNARFAGVIIAGVALGVAAAAQITAGIALHEARQNAERINLLKDSISATNNAVAELQEATGGIVNVITGMQDYINTNLVPQIDKLQCSQIKTALDISLSQYYSEILTVFGPNLQNPVTTSMSIQAISQSFGGNIDLLLNLLGYTANDLLDLLESKSITGQITYINLEHYFMVIRVYYPIMTTISNAYVQELIKISFNVDGSEWVSLVPSYILIRNSYLSNIDISECLITKNSVICRHDFAMPMSYTLKECLTGDTEKCPREAVVTSYVPRFAISGGVIYANCLSTTCQCYQTGKVIAQDGSQTLMMIDNQTCSIVRIEEILISTGKYLGSQEYNTMHVSVGNPVFTDKLDITSQISNINQSIEQSKFYLDKSKAILDKINLNLIGSVPISILFIIAILSLILSIITFVIVMIIVRRYNKYTPLINSDPSSRRSTIQDVYIIPNPGEHSIRSAARSIDRDRD | |
| 43 | SRALLRETDNYSNGLIVENLVRNCHHPSKNNLNYTKTQKRDSTIPYRVEERKGHYPKIKHLIDKSYKHIKRGKRRNGHNGNIITIILLLILILKTQMSEGAIHYETLSKIGLIKGITREYKVKGTPSSKDIVIKLIPNVTGLNKCTNISMENYKEQLDKILIPINNIIELYANSTKSAPGNARFAGVIIAGVALGVAAAAQITAGIALHEARQNAERINLLKDSISATNNAVAELQEATGGIVNVITGMQDYINTNLVPQIDKLQCSQIKTALDISLSQYYSEILTVFGPNLQNPVTTSMSIQAISQSFGGNIDLLLNLLGYTANDLLDLLESKSITGQITYINLEHYFMVIRVYYPIMTTISNAYVQELIKISFNVDGSEWVSLVPSYILIRNSYLSNIDISECLITKNSVICRHDFAMPMSYTLKECLTGDTEKCPREAVVTSYVPRFAISGGVIYANCLSTTCQCYQTGKVIAQDGSQTLMMIDNQTCSIVRIEEILISTGKYLGSQEYNTMHVSVGNPVFTDKLDITSQISNINQSIEQSKFYLDKSKAILDKINLNLIGSVPISILFIIAILSLILSIITFVIVMIIVRRYNKYTPLINSDPSSRRSTIQDVYIIPNPGEHSIRSAARSIDRDRD | Bat Paramyxovirus F Protein, without signal sequence |
| 44 | MGPAENKKVR FENTTSDKGK IPSKVIKSYY GTMDIKKINE GLLDSKILSA FNTVIALLGS IVIIVMNIMI IQNYTRSTDN QAVIKDALQG IQQQIKGLAD KIGTEIGPKV SLIDTSSTIT IPANIGLLGS KISQSTASIN ENVNEKCKFT LPPLKIHECN ISCPNPLPFR EYRPQTEGVS NLVGLPNNIC LQKTSNQILK PKLISYTLPV VGQSGTCITD PLLAMDEGYF AYSHLERIGS CSRGVSKQRI IGVGEVLDRG DEVPSLFMTN VWTPPNPNTV YHCSAVYNNE FYYVLCAVST VGDPILNSTY WSGSLMMTRL AVKPKSNGGG YNQHQLALRS IEKGRYDKVM PYGPSGIKQG DTLYFPAVGF LVRTEFKYND SNCPITKCQY SKPENCRLSM GIRPNSHYIL RSGLLKYNLS DGENPKVVFI EISDQRLSIG SPSKIYDSLG QPVFYQASFS WDTMIKFGDV LTVNPLVVNW RNNTVISRPG QSQCPRFNTC PEICWEGVYN DAFLIDRINW ISAGVFLDSN QTAENPVFTV FKDNEILYRA QLASEDTNAQ KTITNCFLLK NKIWCISLVE IYDTGDNVIR PKLFAVKIPE QC | NiVG protein attachment glycoprotein (602 aa) |
| 45 | FNTVIALLGS IVIIVMNIMI IQNYTRSTDN QAVIKDALQG IQQQIKGLAD KIGTEIGPKV SLIDTSSTIT IPANIGLLGS KISQSTASIN ENVNEKCKFT LPPLKIHECN ISCPNPLPFR EYRPQTEGVS NLVGLPNNIC LQKTSNQILK PKLISYTLPV VGQSGTCITD PLLAMDEGYF AYSHLERIGS CSRGVSKQRI IGVGEVLDRG DEVPSLFMTN VWTPPNPNTV YHCSAVYNNE FYYVLCAVST VGDPILNSTY WSGSLMMTRL AVKPKSNGGG YNQHQLALRS IEKGRYDKVM PYGPSGIKQG DTLYFPAVGF LVRTEFKYND SNCPITKCQY SKPENCRLSM GIRPNSHYIL RSGLLKYNLS DGENPKVVFI EISDQRLSIG SPSKIYDSLG QPVFYQASFS WDTMIKFGDV LTVNPLVVNW RNNTVISRPG QSQCPRFNTC PEICWEGVYN DAFLIDRINW ISAGVFLDSN QTAENPVFTV FKDNEILYRA QLASEDTNAQ KTITNCFLLK NKIWCISLVE IYDTGDNVIR PKLFAVKIPE QC | NivG protein attachment glycoprotein Without cytoplasmic tail Uniprot Q9IH62 |
| 46 | MGKVR FENTTSDKGK IPSKVIKSYY GTMDIKKINE GLLDSKILSA FNTVIALLGS IVIIVMNIMI IQNYTRSTDN QAVIKDALQG IQQQIKGLAD KIGTEIGPKV SLIDTSSTIT IPANIGLLGS KISQSTASIN ENVNEKCKFT LPPLKIHECN ISCPNPLPFR EYRPQTEGVS NLVGLPNNIC LQKTSNQILK PKLISYTLPV VGQSGTCITD PLLAMDEGYF AYSHLERIGS CSRGVSKQRI IGVGEVLDRG DEVPSLFMTN VWTPPNPNTV YHCSAVYNNE FYYVLCAVST VGDPILNSTY WSGSLMMTRL AVKPKSNGGG YNQHQLALRS IEKGRYDKVM PYGPSGIKQG | NiVG protein attachment glycoprotein Truncated Δ5 |
| | DTLYFPAVGF LVRTEFKYND SNCPITKCQY SKPENCRLSM GIRPNSHYIL RSGLLKYNLS DGENPKVVFI EISDQRLSIG SPSKIYDSLG QPVFYQASFS WDTMIKFGDV LTVNPLVVNW RNNTVISRPG QSQCPRFNTC PEICWEGVYN DAFLIDRINW ISAGVFLDSN QTAENPVFTV FKDNEILYRA QLASEDTNAQ KTITNCFLLK NKIWCISLVE IYDTGDNVIR PKLFAVKIPE QC | |
| 47 | MGNTTSDKGK IPSKVIKSYY GTMDIKKINE GLLDSKILSA FNTVIALLGS IVIIVMNIMI IQNYTRSTDN QAVIKDALQG IQQQIKGLAD KIGTEIGPKV SLIDTSSTIT IPANIGLLGS KISQSTASIN ENVNEKCKFT LPPLKIHECN ISCPNPLPFR EYRPQTEGVS NLVGLPNNIC LQKTSNQILK PKLISYTLPV VGQSGTCITD PLLAMDEGYF AYSHLERIGS CSRGVSKQRI IGVGEVLDRG DEVPSLFMTN VWTPPNPNTV YHCSAVYNNE FYYVLCAVST VGDPILNSTY WSGSLMMTRL AVKPKSNGGG YNQHQLALRS IEKGRYDKVM PYGPSGIKQG DTLYFPAVGF LVRTEFKYND SNCPITKCQY SKPENCRLSM GIRPNSHYIL RSGLLKYNLS DGENPKVVFI EISDQRLSIG SPSKIYDSLG QPVFYQASFS WDTMIKFGDV LTVNPLVVNW RNNTVISRPG QSQCPRFNTC PEICWEGVYN DAFLIDRINW ISAGVFLDSN QTAENPVFTV FKDNEILYRA QLASEDTNAQ KTITNCFLLK NKIWCISLVE IYDTGDNVIR PKLFAVKIPE QC | NiVG protein attachment glycoprotein Truncated Δ10 |
| 48 | MGKGK IPSKVIKSYY GTMDIKKINE GLLDSKILSA FNTVIALLGS IVIIVMNIMI IQNYTRSTDN QAVIKDALQG IQQQIKGLAD KIGTEIGPKV SLIDTSSTIT PANIGLLGS KISQSTASIN ENVNEKCKFT LPPLKIHECN ISCPNPLPFR EYRPQTEGVS NLVGLPNNIC LQKTSNQILK PKLISYTLPV VGQSGTCITD PLLAMDEGYF AYSHLERIGS CSRGVSKQRI IGVGEVLDRG DEVPSLFMTN VWTPPNPNTV YHCSAVYNNE FYYVLCAVST VGDPILNSTY WSGSLMMTRL AVKPKSNGGG YNQHQLALRS IEKGRYDKVM PYGPSGIKQG DTLYFPAVGF LVRTEFKYND SNCPITKCQY SKPENCRLSM GIRPNSHYIL RSGLLKYNLS DGENPKVVFI EISDQRLSIG SPSKIYDSLG QPVFYQASFS WDTMIKFGDV LTVNPLVVNW RNNTVISRPG QSQCPRFNTC PEICWEGVYN DAFLIDRINW ISAGVFLDSN QTAENPVFTV FKDNEILYRA QLASEDTNAQ KTITNCFLLK NKIWCISLVE IYDTGDNVIR PKLFAVKIPE QC | NiVG protein attachment glycoprotein Truncated Δ15 |
| 49 | MGSKVIKSYY GTMDIKKINE GLLDSKILSA FNTVIALLGS IVIIVMNIMI IQNYTRSTDN QAVIKDALQG IQQQIKGLAD KIGTEIGPKV SLIDTSSTIT IPANIGLLGS KISQSTASIN ENVNEKCKFT LPPLKIHECN ISCPNPLPFR EYRPQTEGVS NLVGLPNNIC LQKTSNQILK PKLISYTLPV VGQSGTCITD PLLAMDEGYF AYSHLERIGS CSRGVSKQRI IGVGEVLDRG DEVPSLFMTN VWTPPNPNTV YHCSAVYNNE FYYVLCAVST VGDPILNSTY WSGSLMMTRL AVKPKSNGGG YNQHQLALRS IEKGRYDKVM PYGPSGIKQG DTLYFPAVGF LVRTEFKYND SNCPITKCQY SKPENCRLSM GIRPNSHYIL RSGLLKYNLS DGENPKVVFI EISDQRLSIG SPSKIYDSLG QPVFYQASFS WDTMIKFGDV LTVNPLVVNW RNNTVISRPG QSQCPRFNTC PEICWEGVYN DAFLIDRINW ISAGVFLDSN QTAENPVFTV FKDNEILYRA QLASEDTNAQ KTITNCFLLK NKIWCISLVE IYDTGDNVIR PKLFAVKIPE QC | NiVG protein attachment glycoprotein Truncated Δ20 |
| 50 | MGSYY GTMDIKKINE GLLDSKILSA FNTVIALLGS IVIIVMNIMI IQNYTRSTDN QAVIKDALQG IQQQIKGLAD KIGTEIGPKV SLIDTSSTIT IPANIGLLGS KISQSTASIN ENVNEKCKFT LPPLKIHECN ISCPNPLPFR EYRPQTEGVS NLVGLPNNIC LQKTSNQILK PKLISYTLPV VGQSGTCITD PLLAMDEGYF AYSHLERIGS CSRGVSKQRI IGVGEVLDRG DEVPSLFMTN VWTPPNPNTV YHCSAVYNNE FYYVLCAVST VGDPILNSTY WSGSLMMTRL AVKPKSNGGG YNQHQLALRS IEKGRYDKVM PYGPSGIKQG DTLYFPAVGF LVRTEFKYND SNCPITKCQY SKPENCRLSM GIRPNSHYIL RSGLLKYNLS DGENPKVVFI EISDQRLSIG SPSKIYDSLG QPVFYQASFS | NiVG protein attachment glycoprotein Truncated Δ25 |
| | WDTMIKFGDV LTVNPLVVNW RNNTVISRPG QSQCPRFNTC PEICWEGVYN DAFLIDRINW ISAGVFLDSN QTAENPVFTV FKDNEILYRA QLASEDTNAQ KTITNCFLLK NKIWCISLVE IYDTGDNVIR PKLFAVKIPE QC | |
| 51 | MGTMDIKKINE GLLDSKILSA FNTVIALLGS IVIIVMNIMI IQNYTRSTDN QAVIKDALQG IQQQIKGLAD KIGTEIGPKV SLIDTSSTIT IPANIGLLGS KISQSTASIN ENVNEKCKFT LPPLKIHECN ISCPNPLPFR EYRPQTEGVS NLVGLPNNIC LQKTSNQILK PKLISYTLPV VGQSGTCITD PLLAMDEGYF AYSHLERIGS CSRGVSKQRI IGVGEVLDRG DEVPSLFMTN VWTPPNPNTV YHCSAVYNNE FYYVLCAVST VGDPILNSTY WSGSLMMTRL AVKPKSNGGG YNQHQLALRS IEKGRYDKVM PYGPSGIKQG DTLYFPAVGF LVRTEFKYND SNCPITKCQY SKPENCRLSM GIRPNSHYIL RSGLLKYNLS DGENPKVVFI EISDQRLSIG SPSKIYDSLG QPVFYQASFS WDTMIKFGDV LTVNPLVVNW RNNTVISRPG QSQCPRFNTC PEICWEGVYN DAFLIDRINW ISAGVFLDSN QTAENPVFTV FKDNEILYRA QLASEDTNAQ KTITNCFLLK NKIWCISLVE IYDTGDNVIR PKLFAVKIPE QC | NiVG protein attachment glycoprotein Truncated Δ30 |
| 52 | MKVR FENTTSDKGK IPSKVIKSYY GTMDIKKINE GLLDSKILSA FNTVIALLGS IVIIVMNIMI IQNYTRSTDN QAVIKDALQG IQQQIKGLAD KIGTEIGPKV SLIDTSSTIT IPANIGLLGS KISQSTASIN ENVNEKCKFT LPPLKIHECN ISCPNPLPFR EYRPQTEGVS NLVGLPNNIC LQKTSNQILK PKLISYTLPV VGQSGTCITD PLLAMDEGYF AYSHLERIGS CSRGVSKQRI IGVGEVLDRG DEVPSLFMTN VWTPPNPNTV YHCSAVYNNE FYYVLCAVST VGDPILNSTY WSGSLMMTRL AVKPKSNGGG YNQHQLALRS IEKGRYDKVM PYGPSGIKQG DTLYFPAVGF LVRTEFKYND SNCPITKCQY SKPENCRLSM GIRPNSHYIL RSGLLKYNLS DGENPKVVFI EISDQRLSIG SPSKIYDSLG QPVFYQASFS WDTMIKFGDV LTVNPLVVNW RNNTVISRPG QSQCPRFNTC PEICWEGVYN DAFLIDRINW ISAGVFLDSN QTAENPVFTV FKDNEILYRA QLASEDTNAQ KTITNCFLLK NKIWCISLVE IYDTGDNVIR PKLFAVKIPE QCT | NiVG protein attachment glycoprotein Truncated Δ5 |
| 53 | MNTTSDKGK IPSKVIKSYY GTMDIKKINE GLLDSKILSA FNTVIALLGS IVIIVMNIMI IQNYTRSTDN QAVIKDALQG IQQQIKGLAD KIGTEIGPKV SLIDTSSTIT IPANIGLLGS KISQSTASIN ENVNEKCKFT LPPLKIHECN ISCPNPLPFR EYRPQTEGVS NLVGLPNNIC LQKTSNQILK PKLISYTLPV VGQSGTCITD PLLAMDEGYF AYSHLERIGS CSRGVSKQRI IGVGEVLDRG DEVPSLFMTN VWTPPNPNTV YHCSAVYNNE FYYVLCAVST VGDPILNSTY WSGSLMMTRL AVKPKSNGGG YNQHQLALRS IEKGRYDKVM PYGPSGIKQG DTLYFPAVGF LVRTEFKYND SNCPITKCQY SKPENCRLSM GIRPNSHYIL RSGLLKYNLS DGENPKVVFI EISDQRLSIG SPSKIYDSLG QPVFYQASFS WDTMIKFGDV LTVNPLVVNW RNNTVISRPG QSQCPRFNTC PEICWEGVYN DAFLIDRINW ISAGVFLDSN QTAENPVFTV FKDNEILYRA QLASEDTNAQ KTITNCFLLK NKIWCISLVE IYDTGDNVIR PKLFAVKIPE QCT | NiVG protein attachment glycoprotein Truncated Δ10 |
| 54 | MKGK IPSKVIKSYY GTMDIKKINE GLLDSKILSA FNTVIALLGS IVIIVMNIMI IQNYTRSTDN QAVIKDALQG IQQQIKGLAD KIGTEIGPKV SLIDTSSTIT IPANIGLLGS KISQSTASIN ENVNEKCKFT LPPLKIHECN ISCPNPLPFR EYRPQTEGVS NLVGLPNNIC LQKTSNQILK PKLISYTLPV VGQSGTCITD PLLAMDEGYF AYSHLERIGS CSRGVSKQRI IGVGEVLDRG DEVPSLFMTN VWTPPNPNTV YHCSAVYNNE FYYVLCAVST VGDPILNSTY WSGSLMMTRL AVKPKSNGGG YNQHQLALRS IEKGRYDKVM PYGPSGIKQG DTLYFPAVGF LVRTEFKYND SNCPITKCQY SKPENCRLSM GIRPNSHYIL RSGLLKYNLS DGENPKVVFI EISDQRLSIG SPSKIYDSLG QPVFYQASFS WDTMIKFGDV LTVNPLVVNW RNNTVISRPG QSQCPRFNTC PEICWEGVYN DAFLIDRINW ISAGVFLDSN QTAENPVFTV | NiVG protein attachment glycoprotein Truncated Δ15 |
| | FKDNEILYRA QLASEDTNAQ KTITNCFLLK NKIWCISLVE IYDTGDNVIR PKLFAVKIPE QCT | |
| 55 | MSKVIKSYY GTMDIKKINE GLLDSKILSA FNTVIALLGS IVIIVMNIMI IQNYTRSTDN QAVIKDALQG IQQQIKGLAD KIGTEIGPKV SLIDTSSTIT IPANIGLLGS KISQSTASIN ENVNEKCKFT LPPLKIHECN ISCPNPLPFR EYRPQTEGVS NLVGLPNNIC LQKTSNQILK PKLISYTLPV VGQSGTCITD PLLAMDEGYF AYSHLERIGS CSRGVSKQRI IGVGEVLDRG DEVPSLFMTN VWTPPNPNTV YHCSAVYNNE FYYVLCAVST VGDPILNSTY WSGSLMMTRL AVKPKSNGG YNQHQLALRS IEKGRYDKVM PYGPSGIKQG DTLYFPAVGF LVRTEFKYND SNCPITKCQY SKPENCRLSM GIRPNSHYIL RSGLLKYNLS DGENPKVVFI EISDQRLSIG SPSKIYDSLG QPVFYQASFS WDTMIKFGDV LTVNPLVVNW RNNTVISRPG QSQCPRFNTC PEICWEGVYN DAFLIDRINW ISAGVFLDSN QTAENPVFTV FKDNEILYRA QLASEDTNAQ KTITNCFLLK NKIWCISLVE IYDTGDNVIR PKLFAVKIPE QCT | NiVG protein attachment glycoprotein Truncated ΔA20 |
| 56 | MSYY GTMDIKKINE GLLDSKILSA FNTVIALLGS IVIIVMNIMI IQNYTRSTDN QAVIKDALQG IQQQIKGLAD KIGTEIGPKV SLIDTSSTIT IPANIGLLGS KISQSTASIN ENVNEKCKFT LPPLKIHECN ISCPNPLPFR EYRPQTEGVS NLVGLPNNIC LQKTSNQILK PKLISYTLPV VGQSGTCITD PLLAMDEGYF AYSHLERIGS CSRGVSKQRI IGVGEVLDRG DEVPSLFMTN VWTPPNPNTV YHCSAVYNNE FYYVLCAVST VGDPILNSTY WSGSLMMTRL AVKPKSNGGG YNQHQLALRS IEKGRYDKVM PYGPSGIKQG DTLYFPAVGF LVRTEFKYND SNCPITKCQY SKPENCRLSM GIRPNSHYIL RSGLLKYNLS DGENPKVVFI EISDQRLSIG SPSKIYDSLG QPVFYQASFS WDTMIKFGDV LTVNPLVVNW RNNTVISRPG QSQCPRFNTC PEICWEGVYN DAFLIDRINW ISAGVFLDSN QTAENPVFTV FKDNEILYRA QLASEDTNAQ KTITNCFLLK NKIWCISLVE IYDTGDNVIR PKLFAVKIPE QCT | NiVG protein attachment glycoprotein Truncated Δ25 |
| 57 | MTMDIKKINE GLLDSKILSA FNTVIALLGS IVIIVMNIMI IQNYTRSTDN QAVIKDALQG IQQQIKGLAD KIGTEIGPKV SLIDTSSTIT IPANIGLLGS KISQSTASIN ENVNEKCKFT LPPLKIHECN ISCPNPLPFR EYRPQTEGVS NLVGLPNNIC LQKTSNQILK PKLISYTLPV VGQSGTCITD PLLAMDEGYF AYSHLERIGS CSRGVSKQRI IGVGEVLDRG DEVPSLFMTN VWTPPNPNTV YHCSAVYNNE FYYVLCAVST VGDPILNSTY WSGSLMMTRL AVKPKSNGGG YNQHQLALRS IEKGRYDKVM PYGPSGIKQG DTLYFPAVGF LVRTEFKYND SNCPITKCQY SKPENCRLSM GIRPNSHYIL RSGLLKYNLS DGENPKVVFI EISDQRLSIG SPSKIYDSLG QPVFYQASFS WDTMIKFGDV LTVNPLVVNW RNNTVISRPG QSQCPRFNTC PEICWEGVYN DAFLIDRINW ISAGVFLDSN QTAENPVFTV FKDNEILYRA QLASEDTNAQ KTITNCFLLK NKIWCISLVE IYDTGDNVIR PKLFAVKIPE QCT | NiVG protein attachment glycoprotein Truncated Δ30 |
| 58 | KVR FENTTSDKGK IPSKVIKSYY GTMDIKKINE GLLDSKILSA FNTVIALLGS IVIIVMNIMI IQNYTRSTDN QAVIKDALQG IQQQIKGLAD KIGTEIGPKV SLIDTSSTIT IPANIGLLGS KISQSTASIN ENVNEKCKFT LPPLKIHECN ISCPNPLPFR EYRPQTEGVS NLVGLPNNIC LQKTSNQILK PKLISYTLPV VGQSGTCITD PLLAMDEGYF AYSHLERIGS CSRGVSKQRI IGVGEVLDRG DEVPSLFMTN VWTPPNPNTV YHCSAVYNNE FYYVLCAVST VGDPILNSTY WSGSLMMTRL AVKPKSNGGG YNQHQLALRS IEKGRYDKVM PYGPSGIKQG DTLYFPAVGF LVRTEFKYND SNCPITKCQY SKPENCRLSM GIRPNSHYIL RSGLLKYNLS DGENPKVVFI EISDQRLSIG SPSKIYDSLG QPVFYQASFS WDTMIKFGDV LTVNPLVVNW RNNTVISRPG QSQCPRFNTC PEICWEGVYN DAFLIDRINW ISAGVFLDSN QTAENPVFTV FKDNEILYRA QLASEDTNAQ KTITNCFLLK NKIWCISLVE IYDTGDNVIR PKLFAVKIPE QC | NiVG protein attachment glycoprotein Truncated Δ5 Without N-terminal methionine |
| 59 | NTTSDKGK IPSKVIKSYY GTMDIKKINE GLLDSKILSA FNTVIALLGS IVIIVMNIMI IQNYTRSTDN QAVIKDALQG IQQQIKGLAD KIGTEIGPKV SLIDTSSTIT IPANIGLLGS KISQSTASIN ENVNEKCKFT LPPLKIHECN ISCPNPLPFR EYRPQTEGVS NLVGLPNNIC LQKTSNQILK PKLISYTLPV VGQSGTCITD PLLAMDEGYF AYSHLERIGS CSRGVSKQRI IGVGEVLDRG DEVPSLFMTN VWTPPNPNTV YHCSAVYNNE FYYVLCAVST VGDPILNSTY WSGSLMMTRL AVKPKSNGGG YNQHQLALRS IEKGRYDKVM PYGPSGIKQG DTLYFPAVGF LVRTEFKYND SNCPITKCQY SKPENCRLSM GIRPNSHYIL RSGLLKYNLS DGENPKVVFI EISDQRLSIG SPSKIYDSLG QPVFYQASFS WDTMIKFGDV LTVNPLVVNW RNNTVISRPG QSQCPRFNTC PEICWEGVYN DAFLIDRINW ISAGVFLDSN QTAENPVFTV FKDNEILYRA QLASEDTNAQ KTITNCFLLK NKIWCISLVE IYDTGDNVIR PKLFAVKIPE QC | NiVG protein attachment glycoprotein Truncated Δ10 Without N-terminal methionine |
| 60 | KGK IPSKVIKSYY GTMDIKKINE GLLDSKILSA FNTVIALLGS IVIIVMNIMI IQNYTRSTDN QAVIKDALQG IQQQIKGLAD KIGTEIGPKV SLIDTSSTIT PANIGLLGS KISQSTASIN ENVNEKCKFT LPPLKIHECN ISCPNPLPFR EYRPQTEGVS NLVGLPNNIC LQKTSNQILK PKLISYTLPV VGQSGTCITD PLLAMDEGYF AYSHLERIGS CSRGVSKQRI IGVGEVLDRG DEVPSLFMTN VWTPPNPNTV YHCSAVYNNE FYYVLCAVST VGDPILNSTY WSGSLMMTRL AVKPKSNGGG YNQHQLALRS EKGRYDKVM PYGPSGIKQG DTLYFPAVGF LVRTEFKYND SNCPITKCQY SKPENCRLSM GIRPNSHYIL RSGLLKYNLS DGENPKVVFI EISDQRLSIG SPSKIYDSLG QPVFYQASFS WDTMIKFGDV LTVNPLVVNW RNNTVISRPG QSQCPRFNTC PEICWEGVYN DAFLIDRINW ISAGVFLDSN QTAENPVFTV FKDNEILYRA QLASEDTNAQ KTITNCFLLK NKIWCISLVE IYDTGDNVIR PKLFAVKIPE QC | NiVG protein attachment glycoprotein Truncated Δ15 Without N-terminal methionine |
| 61 | SKVIKSYY GTMDIKKINE GLLDSKILSA FNTVIALLGS IVIIVMNIMI IQNYTRSTDN QAVIKDALQG IQQQIKGLAD KIGTEIGPKV SLIDTSSTIT IPANIGLLGS KISQSTASIN ENVNEKCKFT LPPLKIHECN ISCPNPLPFR EYRPQTEGVS NLVGLPNNIC LQKTSNQILK PKLISYTLPV VGQSGTCITD PLLAMDEGYF AYSHLERIGS CSRGVSKQRI IGVGEVLDRG DEVPSLFMTN VWTPPNPNTV YHCSAVYNNE FYYVLCAVST VGDPILNSTY WSGSLMMTRL AVKPKSNGGG YNQHQLALRS EKGRYDKVM PYGPSGIKQG DTLYFPAVGF LVRTEFKYND SNCPITKCQY SKPENCRLSM GIRPNSHYIL RSGLLKYNLS DGENPKVVFI EISDQRLSIG SPSKIYDSLG QPVFYQASFS WDTMIKFGDV LTVNPLVVNW RNNTVISRPG QSQCPRFNTC PEICWEGVYN DAFLIDRINW ISAGVFLDSN QTAENPVFTV FKDNEILYRA QLASEDTNAQ KTITNCFLLK NKIWCISLVE IYDTGDNVIR PKLFAVKIPE QC | NiVG protein attachment glycoprotein Truncated Δ20 Without N-terminal methionine |
| 62 | SYY GTMDIKKINE GLLDSKILSA FNTVIALLGS IVIIVMNIMI IQNYTRSTDN QAVIKDALQG IQQQIKGLAD KIGTEIGPKV SLIDTSSTIT IPANIGLLGS ISQSTASIN ENVNEKCKFT LPPLKIHECN ISCPNPLPFR EYRPQTEGVS NLVGLPNNIC LQKTSNQILK PKLISYTLPV VGQSGTCITD PLLAMDEGYF AYSHLERIGS CSRGVSKQRI IGVGEVLDRG DEVPSLFMTN VWTPPNPNTV YHCSAVYNNE FYYVLCAVST VGDPILNSTY WSGSLMMTRL AVKPKSNGGG YNQHQLALRS EKGRYDKVM PYGPSGIKQG DTLYFPAVGF LVRTEFKYND SNCPITKCQY SKPENCRLSM GIRPNSHYIL RSGLLKYNLS DGENPKVVFI EISDQRLSIG SPSKIYDSLG QPVFYQASFS WDTMIKFGDV LTVNPLVVNW RNNTVISRPG QSQCPRFNTC PEICWEGVYN DAFLIDRINW ISAGVFLDSN QTAENPVFTV FKDNEILYRA QLASEDTNAQ KTITNCFLLK NKIWCISLVE IYDTGDNVIR PKLFAVKIPE QC | NiVG protein attachment glycoprotein Truncated Δ25 Without N-terminal methionine |
| 63 | TMDIKKINE GLLDSKILSA FNTVIALLGS IVIIVMNIMI IQNYTRSTDN QAVIKDALQG IQQQIKGLAD KIGTEIGPKV SLIDTSSTIT IPANIGLLGS KISQSTASIN ENVNEKCKFT LPPLKIHECN ISCPNPLPFR EYRPQTEGVS NLVGLPNNIC LQKTSNQILK PKLISYTLPV VGQSGTCITD PLLAMDEGYF AYSHLERIGS CSRGVSKQRI IGVGEVLDRG DEVPSLFMTN VWTPPNPNTV YHCSAVYNNE FYYVLCAVST VGDPILNSTY WSGSLMMTRL AVKPKSNGGG YNQHQLALRS IEKGRYDKVM YGPSGIKQG DTLYFPAVGF LVRTEFKYND SNCPITKCQY SKPENCRLSM GIRPNSHYIL RSGLLKYNLS DGENPKVVFI EISDQRLSIG SPSKIYDSLG QPVFYQASFS WDTMIKFGDV LTVNPLVVNW RNNTVISRPG QSQCPRFNTC PEICWEGVYN DAFLIDRINW ISAGVFLDSN QTAENPVFTV FKDNEILYRA QLASEDTNAQ KTITNCFLLK NKIWCISLVE IYDTGDNVIR PKLFAVKIPE QC | NiVG protein attachment glycoprotein Truncated Δ30 Without N-terminal methionine |
| 64 | MKKINEGLLDSKILSA FNTVIALLGS IVIIVMNIMI IQNYTRSTDN QAVIKDALQG IQQQIKGLAD KIGTEIGPKV SLIDTSSTIT IPANIGLLGS KISQSTASIN ENVNEKCKFT LPPLKIHECN ISCPNPLPFR EYRPQTEGVS NLVGLPNNIC LQKTSNQILK PKLISYTLPV VGQSGTCITD PLLAMDEGYF AYSHLERIGS CSRGVSKQRI IGVGEVLDRG DEVPSLFMTN VWTPPNPNTV YHCSAVYNNE FYYVLCAVST VGDPILNSTY WSGSLMMTRL AVKPKSNGGG YNQHQLALRS IEKGRYDKVM PYGPSGIKQG DTLYFPAVGF LVRTEFKYND SNCPITKCQY SKPENCRLSM GIRPNSHYIL RSGLLKYNLS DGENPKVVFI EISDQRLSIG SPSKIYDSLG QPVFYQASFS WDTMIKFGDV LTVNPLVVNW RNNTVISRPG QSQCPRFNTC PEICWEGVYN DAFLIDRINW ISAGVFLDSN QTAENPVFTV FKDNEILYRA QLASEDTNAQ KTITNCFLLK NKIWCISLVE IYDTGDNVIR PKLFAVKIPE QCT | NiVG protein attachment glycoprotein Truncated (Gc Δ 34) |
| 65 | KKINEGLLDSKILSA FNTVIALLGS IVIIVMNIMI IQNYTRSTDN QAVIKDALQG IQQQIKGLAD KIGTEIGPKV SLIDTSSTIT IPANIGLLGS KISQSTASIN ENVNEKCKFT LPPLKIHECN ISCPNPLPFR EYRPQTEGVS NLVGLPNNIC LQKTSNQILK PKLISYTLPV VGQSGTCITD PLLAMDEGYF AYSHLERIGS CSRGVSKQRI IGVGEVLDRG DEVPSLFMTN VWTPPNPNTV YHCSAVYNNE FYYVLCAVST VGDPILNSTY WSGSLMMTRL AVKPKSNGGG YNQHQLALRS IEKGRYDKVM PYGPSGIKQG DTLYFPAVGF LVRTEFKYND SNCPITKCQY SKPENCRLSM GIRPNSHYIL RSGLLKYNLS DGENPKVVFI EISDQRLSIG SPSKIYDSLG QPVFYQASFS WDTMIKFGDV LTVNPLVVNW RNNTVISRPG QSQCPRFNTC PEICWEGVYN DAFLIDRINW ISAGVFLDSN QTAENPVFTV FKDNEILYRA QLASEDTNAQ KTITNCFLLK NKIWCISLVE IYDTGDNVIR PKLFAVKIPE QCT | NiVG protein attachment glycoprotein Truncated (Gc Δ 34) Without N-terminal methionine |
| 66 | MMADSKLVSL NNNLSGKIKD QGKVIKNYYG TMDIKKINDG LLDSKILGAF NTVIALLGSI IIIVMNIMII QNYTRTTDNQ ALIKESLQSV QQQIKALTDK IGTEIGPKVS LIDTSSTITI PANIGLLGSK ISQSTSSINE NVNDKCKFTL PPLKIHECNI SCPNPLPFRE YRPISQGVSD LVGLPNQICL QKTTSTILKP RLISYTLPIN TREGVCITDP LLAVDNGFFA YSHLEKIGSC TRGIAKQRII GVGEVLDRGD KVPSMFMTNV WTPPNPSTIH HCSSTYHEDF YYTLCAVSHV GDPILNSTSW TESLSLIRLA VRPKSDSGDY NQKYIAITKV ERGKYDKVMP YGPSGIKQGD TLYFPAVGFL PRTEFQYNDS NCPIIHCKYS KAENCRLSMG VNSKSHYILR SGLLKYNLSL GGDIILQFIE IADNRLTIGS PSKIYNSLGQ PVFYQASYSW DTMIKLGDVD TVDPLRVQWR NNSVISRPGQ SQCPRFNVCP EVCWEGTYND AFLIDRLNWV SAGVYLNSNQ TAENPVFAVF | Hendra virus G protein Uniprot 089343 |
| | KDNEILYQVP LAEDDTNAQK TITDCFLLEN VIWCISLVEI YDTGDSVIRP KLFAVKIPAQ CSES | |
| 67 | MADSKLVSL NNNLSGKIKD QGKVIKNYYG TMDIKKINDG LLDSKILGAF NTVIALLGSI IIIVMNIMII QNYTRTTDNQ ALIKESLQSV QQQIKALTDK IGTEIGPKVS LIDTSSTITI PANIGLLGSK ISQSTSSINE NVNDKCKFTL PPLKIHECNI SCPNPLPFRE YRPISQGVSD LVGLPNQICL QKTTSTILKP RLISYTLPIN TREGVCITDP LLAVDNGFFA YSHLEKIGSC TRGIAKQRII GVGEVLDRGD KVPSMFMTNV WTPPNPSTIH HCSSTYHEDF YYTLCAVSHV GDPILNSTSW TESLSLIRLA VRPKSDSGDY NQKYIAITKV ERGKYDKVMP YGPSGIKQGD TLYFPAVGFL PRTEFQYNDS NCPIIHCKYS KAENCRLSMG VNSKSHYILR SGLLKYNLSL GGDIILQFIE IADNRLTIGS PSKIYNSLGQ PVFYQASYSW DTMIKLGDVD TVDPLRVQWR NNSVISRPGQ SQCPRFNVCP EVCWEGTYND AFLIDRLNWV SAGVYLNSNQ TAENPVFAVF KDNEILYQVP LAEDDTNAQK TITDCFLLEN VIWCISLVEI YDTGDSVIRP KLFAVKIPAQ CSES | Hendra virus G protein Uniprot 089343 Without N-terminal methionine |
| 68 | FNTVIALLGSI IIIVMNIMII QNYTRTTDNQ ALIKESLQSV QQQIKALTDK IGTEIGPKVS LIDTSSTITI PANIGLLGSK ISQSTSSINE NVNDKCKFTL PPLKIHECNI SCPNPLPFRE YRPISQGVSD LVGLPNQICL QKTTSTILKP RLISYTLPIN TREGVCITDP LLAVDNGFFA YSHLEKIGSC TRGIAKQRII GVGEVLDRGD KVPSMFMTNV WTPPNPSTIH HCSSTYHEDF YYTLCAVSHV GDPILNSTSW TESLSLIRLA VRPKSDSGDY NQKYIAITKV ERGKYDKVMP YGPSGIKQGD TLYFPAVGFL PRTEFQYNDS NCPIIHCKYS KAENCRLSMG VNSKSHYILR SGLLKYNLSL GGDIILQFIE IADNRLTIGS PSKIYNSLGQ PVFYQASYSW DTMIKLGDVD TVDPLRVQWR NNSVISRPGQ SQCPRFNVCP EVCWEGTYND AFLIDRLNWV SAGVYLNSNQ TAENPVFAVF KDNEILYQVP LAEDDTNAQK TITDCFLLEN VIWCISLVEI YDTGDSVIRP KLFAVKIPAQ CSES | Hendra virus G protein Uniprot 089343 Without cytoplasmic tail |
| 69 | MKKINEGLLDSKILSA FNTVIALLGS IVIIVMNIMI IQNYTRSTDN QAVIKDALQG IQQQIKGLAD KIGTEIGPKV SLIDTSSTIT IPANIGLLGS KISQSTASIN ENVNEKCKFT LPPLKIHECN ISCPNPLPFR EYRPQTEGVS NLVGLPNNIC LQKTSNQILK PKLISYTLPV VGQSGTCITD PLLAMDEGYF AYSHLERIGS CSRGVSKQRI IGVGEVLDRG DEVPSLFMTN VWTPPNPNTV YHCSAVYNNE FYYVLCAVST VGDPILNSTY WSGSLMMTRL AVKPKSNGGG YNQHQLALRS IEKGRYDKVM PYGPSGIKQG DTLYFPAVGF LVRTEFKYND SNCPITKCQY SKPENCRLSM GIRPNSHYIL RSGLLKYNLS DGENPKVVFI EISDQRLSIG SPSKIYDSLG QPVFYQASFS WDTMIKFGDV LTVNPLVVNW RNNTVISRPG QSQCPRFNTC PAICAEGVYN DAFLIDRINW ISAGVFLDSN ATAANPVFTV FKDNEILYRA QLASEDTNAQ KTITNCFLLK NKIWCISLVE IYDTGDNVIR PKLFAVKIPE QCT | NiVG protein attachment glycoprotein Truncated and mutated (E501 A, W504A, Q530A, E533A) NiV G protein (Gc Δ 34) |
| 70 | KKINEGLLDSKILSA FNTVIALLGS IVIIVMNIMI IQNYTRSTDN QAVIKDALQG IQQQIKGLAD KIGTEIGPKV SLIDTSSTIT IPANIGLLGS KISQSTASIN ENVNEKCKFT LPPLKIHECN ISCPNPLPFR EYRPQTEGVS NLVGLPNNIC LQKTSNQILK PKLISYTLPV VGQSGTCITD PLLAMDEGYF AYSHLERIGS CSRGVSKQRI IGVGEVLDRG DEVPSLFMTN VWTPPNPNTV YHCSAVYNNE FYYVLCAVST VGDPILNSTY WSGSLMMTRL AVKPKSNGGG YNQHQLALRS IEKGRYDKVM PYGPSGIKQG DTLYFPAVGF LVRTEFKYND SNCPITKCQY SKPENCRLSM GIRPNSHYIL RSGLLKYNLS | NiVG protein attachment glycoprotein Truncated and mutated (E501 A, W504A, Q530A, E533A) NiV G protein (Gc Δ 34) |
| | DGENPKVVFI EISDQRLSIG SPSKIYDSLG QPVFYQASFS WDTMIKFGDV LTVNPLVVNW RNNTVISRPG QSQCPRFNTC PAICAEGVYN DAFLIDRINW ISAGVFLDSN ATAANPVFTV FKDNEILYRA QLASEDTNAQ KTITNCFLLK NKIWCISLVE IYDTGDNVIR PKLFAVKIPE QCT | Without N-terminal methionine |
| 71 | MVVILDKRCY CNLLILILMI SECSVG | signal sequence |
| 72 | ILHYEKLSKIGLVKGVTRKYKIKSNPLTKDIVIKMIPNVSNMSQCTGSVMENYKTRLNGILTPIKGALEIYKNNTHDLVGDVR | Nipah virus NiV-F F2 (aa 27-109) |
| 73 | LAGVIMAGVAIGIATAAQITAGVALYEAMKNADNINKLKSSIESTNEAVVKLQETAEKTVYVLTALQDYINTNLVPTIDKISCKQTELSLDLALSKYLSDLLFVFGPNLQDPVSNSMTIQAISQAFGGNYETLLRTLGYATEDFDDLLESDSITGQIIYVDLSSYYIIVRVYFPILTEIQQAYIQELLPVSFNNDNSEWISIVPNFILVRNTLISNIEIGFCLITKRSVICNQDYATPMTNNMRECLTGSTEKCPRELVVSSHVPRFALSNGVLFANCISVTCQCQTTGRAISQSGEQTLLMIDNTTCPTAVLGNVIISLGKYLGSVNYNSEGIAIGPPVFTDKVDISSQISSMNQSLQQSKDYIKEAQRLLDTVNPSLISMLSMIILYVLSIASLCIGLITFISFIIVEKKRNTYSRLEDRRVRPTSSGDLYYIGT | Nipah virus NiV F F1 (aa 110-546) |
| 74 | ILHY EKLSKIGLVK GVTRKYKIKS NPLTKDIVIK MIPNVSNMSQ CTGSVMENYK TRLNGILTPI KGALEIYKNQ THDLVGDVRL AGVIMAGVAI GIATAAQITA VALYEAMKN ADNINKLKSS IESTNEAVVK LQETAEKTVY VLTALQDYIN TNLVPTIDKI SCKQTELSLD LALSKYLSDL LFVFGPNLQD PVSNSMTIQA ISQAFGGNYE TLLRTLGYAT EDFDDLLESD SITGQIIYVD LSSYYIIVRV YFPILTEIQQ AYIQELLPVS FNNDNSEWIS IVPNFILVRN TLISNIEIGF CLITKRSVIC NQDYATPMTN NMRECLTGST EKCPRELVVS SHVPRFALSN GVLFANCISV TCQCQTTGRA ISQSGEQTLL MIDNTTCPTA VLGNVIISLG KYLGSVNYNS EGIAIGPPVF TDKVDISSQI SSMNQSLQQS KDYIKEAQRL LDTVNPSLIS MLSMIILYVL SIASLCIGLI TFISFIIVEK KRNTGT | Nipah virus NiV-F F0 T234 truncation (aa 525-544) AND mutation on N-linked glycosylation site |
| 75 | VVILDKRCY CNLLILILMI SECSVGILHY EKLSKIGLVK GVTRKYKIKS NPLTKDIVIK MIPNVSNMSQ CTGSVMENYK TRLNGILTPI KGALEIYKNN THDLVGDVRL AGVIMAGVAI GIATAAQITA GVALYEAMKN ADNINKLKSS IESTNEAVVK LQETAEKTVY VLTALQDYIN TNLVPTIDKI SCKQTELSLD LALSKYLSDL LFVFGPNLQD PVSNSMTIQA ISQAFGGNYE TLLRTLGYAT EDFDDLLESD SITGQIIYVD LSSYYIIVRV YFPILTEIQQ AYIQELLPVS FNNDNSEWIS IVPNFILVRN TLISNIEIGF CLITKRSVIC NQDYATPMTN NMRECLTGST EKCPRELVVS SHVPRFALSN GVLFANCISV TCQCQTTGRA ISQSGEQTLL MIDNTTCPTA VLGNVIISLG KYLGSVNYNS EGIAIGPPVF TDKVDISSQI SSMNQSLQQS KDYIKEAQRL LDTVNPSLIS MLSMIILYVL SIASLCIGLI TFISFIIVEK KRNT | Truncated NiV fusion glycoprotein (FcDelta22) at cytoplasmic tail (with signal sequence) without initiator met |
| 76 | ILHY EKLSKIGLVK GVTRKYKIKS NPLTKDIVIK MIPNVSNMSQ CTGSVMENYK TRLNGILTPI KGALEIYKNN THDLVGDVRL AGVIMAGVAI GIATAAQITA GVALYEAMKN ADNINKLKSS IESTNEAVVK LQETAEKTVY VLTALQDYIN TNLVPTIDKI SCKQTELSLD LALSKYLSDL LFVFGPNLQD PVSNSMTIQA ISQAFGGNYE TLLRTLGYAT EDFDDLLESD SITGQIIYVD LSSYYIIVRV YFPILTEIQQ AYIQELLPVS FNNDNSEWIS IVPNFILVRN TLISNIEIGF CLITKRSVIC NQDYATPMTN NMRECLTGST EKCPRELVVS SHVPRFALSN GVLFANCISV TCQCQTTGRA ISQSGEQTLL MIDNTTCPTA VLGNVIISLG KYLGSVNYNS EGIAIGPPVF TDKVDISSQI SSMNQSLQQS KDYIKEAQRL LDTVNPSLIS MLSMIILYVL SIASLCIGLI TFISFIIVEK KRNTGT | Nipah virus NiV-F F0 T234 truncation (aa 525-544) |
| 77 | MVVILDKRCY CNLLILILMI SECSVGILHY EKLSKIGLVK GVTRKYKIKS NPLTKDIVIK MIPNVSNMSQ CTGSVMENYK TRLNGILTPI KGALEIYKNN THDLVGDVRL AGVIMAGVAI GIATAAQITA GVALYEAMKN ADNINKLKSS IESTNEAVVK LQETAEKTVY | Nipah virus NiV-F F0 T234 truncation (aa 525-544) (with signal sequence) |
| | VLTALQDYIN TNLVPTIDKI SCKQTELSLD LALSKYLSDL LFVFGPNLQD PVSNSMTIQA ISQAFGGNYE TLLRTLGYAT EDFDDLLESD SITGQIIYVD LSSYYIIVRV YFPILTEIQQ AYIQELLPVS FNNDNSEWIS IVPNFILVRN TLISNIEIGF CLITKRSVIC NQDYATPMTN NMRECLTGST EKCPRELVVS SHVPRFALSN GVLFANCISV TCQCQTTGRA ISQSGEQTLL MIDNTTCPTA VLGNVIISLG KYLGSVNYNS EGIAIGPPVF TDKVDISSQI SSMNQSLQQS KDYIKEAQRL LDTVNPSLIS MLSMIILYVL SIASLCIGLI TFISFIIVEK KRNTGT | |
| 78 | MVVILDKRCY CNLLILILMI SECSVGILHY EKLSKIGLVK GVTRKYKIKS NPLTKDIVIK MIPNVSNMSQ CTGSVMENYK TRLNGILTPI KGALEIYKNQ THDLVGDVRL AGVIMAGVAI GIATAAQITA GVALYEAMKN ADNINKLKSS IESTNEAVVK LQETAEKTVY VLTALQDYIN TNLVPTIDKI SCKQTELSLD LALSKYLSDL LFVFGPNLQD PVSNSMTIQA ISQAFGGNYE TLLRTLGYAT EDFDDLLESD SITGQIIYVD LSSYYIIVRV YFPILTEIQQ AYIQELLPVS FNNDNSEWIS IVPNFILVRN TLISNIEIGF CLITKRSVIC NQDYATPMTN NMRECLTGST EKCPRELVVS SHVPRFALSN GVLFANCISV TCQCQTTGRA ISQSGEQTLL MIDNTTCPTA VLGNVIISLG KYLGSVNYNS EGIAIGPPVF TDKVDISSQI SSMNQSLQQS KDYIKEAQRL LDTVNPSLIS MLSMIILYVL SIASLCIGLI TFISFIIVEK KRNTGT | Nipah virus NiV-F F0 T234 truncation (aa 525-544) AND mutation on N-linked glycosylation site (with signal sequence) |
| 79 | MVVILDKRCY CNLLILILMI SECSVGILHY EKLSKIGLVK GVTRKYKIKS NPLTKDIVIK MIPNVSNMSQ CTGSVMENYK TRLNGILTPI KGALEIYKNN THDLVGDVRL AGVIMAGVAI GIATAAQITA GVALYEAMKN ADNINKLKSS IESTNEAVVK LQETAEKTVY VLTALQDYIN TNLVPTIDKI SCKQTELSLD LALSKYLSDL LFVFGPNLQD PVSNSMTIQA ISQAFGGNYE TLLRTLGYAT EDFDDLLESD SITGQIIYVD LSSYYIIVRV YFPILTEIQQ AYIQELLPVS FNNDNSEWIS IVPNFILVRN TLISNIEIGF CLITKRSVIC NQDYATPMTN NMRECLTGST EKCPRELVVS SHVPRFALSN GVLFANCISV TCQCQTTGRA ISQSGEQTLL MIDNTTCPTA VLGNVIISLG KYLGSVNYNS EGIAIGPPVF TDKVDISSQI SSMNQSLQQS KDYIKEAQRL LDTVNPSLIS MLSMIILYVL SIASLCIGLI TFISFIIVEK KRNT | Truncated NiV fusion glycoprotein (FcDelta22) at cytoplasmic tail (with signal sequence) |
| 80 | ILHY EKLSKIGLVK GVTRKYKIKS NPLTKDIVIK MIPNVSNMSQ CTGSVMENYK TRLNGILTPI KGALEIYKNN THDLVGDVRL AGVIMAGVAI GIATAAQITA GVALYEAMKN ADNINKLKSS IESTNEAVVK LQETAEKTVY VLTALQDYIN TNLVPTIDKI SCKQTELSLD LALSKYLSDL LFVFGPNLQD PVSNSMTIQA ISQAFGGNYE TLLRTLGYAT EDFDDLLESD SITGQIIYVD LSSYYIIVRV YFPILTEIQQ AYIQELLPVS FNNDNSEWIS IVPNFILVRN TLISNIEIGF CLITKRSVIC NQDYATPMTN NMRECLTGST EKCPRELVVS SHVPRFALSN GVLFANCISV TCQCQTTGRA ISQSGEQTLL MIDNTTCPTA VLGNVIISLG KYLGSVNYNS EGIAIGPPVF TDKVDISSQI SSMNQSLQQS KDYIKEAQRL LDTVNPSLIS MLSMIILYVL SIASLCIGLI TFISFIIVEK KRNT | Truncated mature NiV fusion glycoprotein (FcDelta22) at cytoplasmic tail |
| 81 | KKRXKR | Chelsky NLS Consensus |
| 82 | KR[PAATKKAGQA]KKKK | Bipartite NLS Consensus |
| 83 | PAAKRVKLD | c-Myc NLS |
| 84 | PKKKRKVEDP | SV40 Large T NLS |
| 85 | LEDLDNLIL | NES |
| 86 | RRVPQRKEVSRCRKCRK | NLS |
| 87 | LXXXLXXLXL | NES |
| 88 | QIPRDRLSNIGVIVDEGKSLKIAGSHESRYIVLSLVPGVDFENGCGTAQVIQYKSLLNRLLIPLRDALDLQEALITVTNDTTQNAGAPQSRFFGAVIGTIALGVATSAQITAGIALAEAREAKRDIALIKESMTKTHKSIELLQNAVGEQILALKTLQDFVNDEIKPAISELGCETAALRLGIKLTQHYSELLTAFGSNFGTIGEKSLTLQALSSLYSANITEIMTTIKTGQSNIYDVIYTEQIKGTVIDVDLERYMVTLSVKIPILSEVPGVLIHKASSISYNIDGEEWYVTVPSHILSRASFLGGADITDCVESRLTYICPRDPAQLIPDSQQKCILGDTTRCPVTKVVDSLIPKFAFVNGGVVANCIASTCTCGTGRRPISQDRSKGVVFLTHDNCGLIGVNGVELYANRRGHDATWGVQNLTVGPAIAIRPIDISLNLADATNFLQDSKAELEKARKILSEVGRWYNSRETVITIIVVMVVILVVIIVIIIVLYRLRRSMLMGNPDDRIPRDTYTLEPKIRHMYTNGGFDAMAEKR | Sendai F protein |
| 89 | MDGDRGKRDSYWSTSPSGSTTKPASGWERSSKADTWLLILSFTQWALSIATVIICIIISARQGYSMKEYSMTVEALNMSSREVKESLTSLIRQEVIARAVNIQSSVQTGIPVLLNKNSRDVIQMIDKSCSRQELTQHCESTIAVHHADGIAPLEPHSFWRCPVGEPYLSSDPEISLLPGPSLLSGSTTISGCVRLPSLSIGEAIYAYSSNLITQGCADIGKSYQVLQLGYILNSDMFPDLNPVVSHTYDINDNRKSCSVVATGTRGYQLCSMPTVDERTDYSSDGIEDLVLDVLDLKGRTKSHRYRNSEVDLDHPFSALYPSVGNGIATEGSLIFLGYGGLTTPLQGDTKCRTQGCQQVSQDTCNEALKITWLGGKQVVSVIIQVNDYLSERPKIRVTTIPITQNYLGAEGRLLKLGDRVYIYTRSSGWHSQLQIGVLDVSHPLTINWTPHEALSRPGNKECNWYNKCPKECISGVYTDAYPLSPDAANVATVTLYANTSRVNPTIMYSNTTNIINMLRIKDVQLEAAYTTTSCITHFGKGYCFHIIEINQKSLNTLQPMLFKTSIPKLCKAES | Sendai HN protein |
| 90 | MWSELKIRSNDGGEGPEDANDPRGKGVQHIHIQPSLPVGQRVRMDGDRGKRDSYWSTSPSGSTTKPASGWERSSKADTWLLILSFTQWALSIATVIICIIISARQGYSMKEYSMTVEALNMSSREVKESLTSLIRQEVIARAVNIQSSVQTGIPVLLNKNSRDVIQMIDKSCSRQELTQHCESTIAVHHADGIAPLEPHSFWRCPVGEPYLSSDPEISLLPGPSLLSGSTTISGCVRLPSLSIGEAIYAYSSNLITQGCADIGKSYQVLQLGYISLNSDMFPDLNPVVSHTYDINDNRKSCSVVATGTRGYQLCSMPTVDERTDYSSDGIEDLVLDVLDLKGRTKSHRYRNSEVDLDHPFSALYPSVGNGIATEGSLIFLGYGGLTTPLQGDTKCRTQGCQQVSQDTCNEALKITWLGGKQVVSVIIQVNDYLSERPKIRVTTIPITQNYLGAEGRLLKLGDRVYIYTRSSGWHSQLQIGVLDVSHPLTINWTPHEALSRPGNKECNWYNKCPKECISGVYTDAYPLSPDAANVATVTLYANTSRVNPTIMYSNTTNIINMLRIKDVQLEAAYTTTSCITHFGKGYCFHIIEINQKSLNTLQPMLFKTSIPKLCKAES | Sendai HN protein (modified CTD) |
| 91 | LAGVIMAGVAIGIATAAQITAGVALYEAMKNADNINKLKSSIESTNEAVVKLQETAEKTVYVLTALQDYINTNLVPTIDKISCKQTELSLDLALSKYLSDLLFVFGPNLQDPVSNSMTIQAISQAFGGNYETLLRTLGYATEDFDDLLESDSITGQIIYVDLSSYYIIVRVYFPILTEIQQAYIQELLPVSFNNDNSEWISIVPNFILVRNTLISNIEIGFCLITKRSVICNQDYATPMTNNMRECLTGSTEKCPRELVVSSHVPRFALSNGVLFANCISVTCQCQTTGRAISQSGEQTLLMIDNTTCPTAVLGNVIISLGKYLGSVNYNSEGIAIGPPVFTDKVDISSQISSMNQSLQQSKDYIKEAQRLLDTVNPSLISMLSMIILYVLSIASLCIGLITFISFIIVEKKRNTGT | Nipah virus NiV F F1 (aa 110-546) truncation (aa 525-544) |

1. A method of treating a coronavirus infection in a subject, the method comprising administering to a subject known or suspected of having a coronavirus infection a composition comprising an agent for delivery of a Chromosome 19 Open Reading Frame 66 (C19orf66) protein to the subject or an agent for delivery of a regulatory factor that increases expression of the gene encoding C19orf66 in a cell in the subject.

2. A method of reducing the likelihood of a coronavirus infection in a subject, the method comprising administering to a subject known or suspected of being exposed to a coronavirus a composition comprising an agent for delivery of a Chromosome 19 Open Reading Frame 66 (C19orf66) protein to the subject or an agent for delivery of a regulatory factor that increases expression of the gene encoding C19orf66 in a cell in the subject.

3. The method of claim 1 or claim 2, wherein the subject is administered an agent for delivery of a C19orf66 protein to the subject, wherein the agent is a nucleotide sequence encoding the C19orf66 protein.

4. The method of claim 1 or claim 2, wherein the subject is administered an agent for delivery of a regulatory factor that increases expression of the gene encoding C 19orf66, wherein the agent is a nucleotide sequence encoding the regulatory factor.

5. The method of claim 3 or claim 4, wherein the nucleotide sequence is operably linked to a promoter to control expression.

6. The method of claim 5, wherein the promoter is an inducible promoter.

7. The method of claim 3 or claim 4, wherein expression of the nucleotide sequence is controlled by an inducible expression system.

8. The method of claim 7, wherein the inducible expression system comprises a first nucleic acid sequence comprising the nucleotide sequence operably linked to a drug response element and a second nucleic acid sequence comprising a drug-controlled transactivator operably linked to a promoter.

9. The method of claim 8, wherein the drug response element is a tetracycline response element or a modified form thereof, optionally wherein the modified form is Tet-On 3G, and the drug-controlled transactivator is a reverse Tet transactivator (rtTA).

10. The method of any of claims 1-9, further comprising administering to the subject an effective amount of a drug for inducing expression of the nucleotide sequence by the inducible expression system, optionally wherein the drug is doxycycline.

11. The method of any of claims 5, and 8-10, wherein:
(i) the promoter is a constitutive promoter, optionally wherein the promoter is a human Ubiquitin C (UbC) promoter, a human elongation factor 1α (EF1α) promoter, an SV40 promoter, a Cytomegalovirus (CMV) promoter, or a PGK-1 promoter;
(ii) the promoter control expression in the lung;
(iii) the promoter is a human surfactant A promoter , a human surfactant B promoter, a human surfactant C promoter, a human surfactant D promoter, human ROBO4 promoter, or a human CDH1 gene; or
the promoter is the human surfactant B promoter set forth in SEQ ID NO: 10.

12. The method of claim 1 or claim 2, wherein the subject is administered an agent for delivery of a C19orf66 protein to the subject, wherein the agent is the C19orf66 protein, optionally wherein the C19orf66 protein is a recombinant protein.

13. The method of claim 12, wherein the C19orf66 protein is linked to a cell penetrating peptide, optionally via a peptide linker.

14. The method of claim 13, wherein the cell penetrating peptide is selected from the group consisting of:
TAT (SEQ ID NO: 13)
Penetratin (SEQ ID NO: 14)
Transporant (SEQ ID NO: 15)
Pept 1 (SEQ ID NO: 16)
Pept 2 (SEQ ID NO: 17)
Transportan (SEQ ID NO: 18)
IgV (SEQ ID NO: 19 ).

15. The method of any of claims 1-14, wherein the administration of the agent (i) inhibits or prevents viral replication of the coronavirus in the subject, (ii) inhibits or prevents ribosomal frameshifting in the subject, and/or (iii) inhibits or prevents viral RNA processing in the subject.

16. The method of any of claims 1-15, wherein the C19orf66 protein is or comprises the sequence of amino acids set forth in SEQ ID NO: 1, 3, 5 or 7, or a sequence of amino acids that has at least 90%, at least 92%, at least 95%, or at least 98% sequence identity to the sequence of amino acids set forth in SEQ ID NO:1, 3, 5, or 7.

17. The method of any of claims 1-16, wherein the C19orf66 protein is encoded by the nucleotide sequence set forth in SEQ ID NO: 2, 4, 6 or 8 or a nucleotide sequence that has at least 90%, at least 92%, at least 95%, or at least 98% sequence identity to SEQ ID NO:2, 4, 6, or 8.

18. The method of any of claims 1-17, wherein the C19orf66 protein further comprises a nuclear localization signal.

19. The method of any of claims 1-18, wherein the C19orf66 protein further comprises a nuclear export signal.

20. The method of any of claims 1, 2, 4-11, or 15-19, wherein nucleotide sequence encodes a regulatory factor that increases expression of the gene encoding C19orf66 and the regulatory factor controls targeted transcriptional activation of the gene encoding C19orf66.

21. The method of any of claims 1, 2, 4-11 and 15-20, wherein the regulatory factor is a fusion protein comprising a site-specific binding domain specific for the C19orf66 gene, and a transcriptional activator.

22. The method of any of claims 1-21, wherein the agent is comprised in a vehicle that is a lipid particle or a non-lipid particle.

23. The method of claim 22, wherein the lipid particle is a viral particle, a virus-like particle, a nanoparticle, a vesicle, an exosome, a dendrisome, an enucleated cell, a microvesicle, a membrane vesicle, an extracellular membrane vesicle, a plasma membrane vesicle, a giant plasma membrane vesicle, an apoptotic body, a mitoparticle, a micelle, a liposome, a pyrenocyte, a lysosome, another membrane enclosed vesicle, or a cell derived particle.

24. The method of claim 22 or claim 23, wherein the viral particle is a viral vector that is a lentiviral vector.

25. The method of claim 22, wherein the non-lipid particle is a non-lipid nanoparticle, a polymeric nanoparticle, a nanocapsule, a nanorod, a nanosphere, a nanogel, a dendrimer, or other synthetic or inorganic particle.


SEQUENCE LISTING (91 sequences)

SEQ ID NO: 1 - protein, 291 aa, Artificial Sequence
  SHFL, Variant 1
MSQEGVELEKSVRRLREKFHGKVSSKKAGALMRKFGSDHTGVGRSIVYGVKQKDGQELSNDLDAQDPPEDMKQDRDIQAVATSLLPLTEANLRMFQRAQDDLIPAVDRQFACSSCDHVWWRRVPQRKEVSRCRKCRKRYEPVPADKMWGLAEFHCPKCRHNFRGWAQMGSPSPCYGCGFPVYPTRILPPRWDRDPDRRSTHTHSCSAADCYNRREPHVPGTSCAHPKSRKQNHLPKVLHPSNPHISSGSTVATCLSQGGLLEDLDNLILEDLKEEEEEEEEVEDEEGGPRE

SEQ ID NO: 2 - DNA, 1928 bp, Artificial Sequence
  SHFL - Variant 1
gaggcaccgccccctgccctgcgcggctgctggaccgacgggcgcacccaggtaggggggcggctgagccgcgcagtgcggaccctcgcggggaactgcgccgccgccaccatgtctcaggaaggtgtggagctggagaagagcgtccggcgcctccgggagaagtttcatgggaaggtatcctccaagaaggcgggggctctgatgaggaaattcggcagcgaccacacgggagtggggcgctccatcgtgtacggggtaaagcaaaaagatggccaagaactaagtaacgatctggatgcccaggatccaccagaagatatgaagcaggaccgggacattcaggcagtggcgacctccctcctgccactgacagaagccaacctacgcatgtttcaacgtgcccaggacgaccttatccctgctgtggaccggcagtttgcctgctcctcctgcgaccacgtctggtggcgccgcgtgccccagcggaaggaggtatcccggtgccggaaatgccggaagcgctacgagccagtgccagctgacaagatgtggggcctggctgagttccactgcccgaagtgtcggcacaacttccggggctgggcacagatggggtccccgtccccctgctacgggtgcggcttccccgtgtatccaacacggatcctccccccgcgctgggaccgggacccggatcgccgcagcacccacactcactcctgctcagctgccgactgctacaaccggcgagagccccacgtgcctgggacatcctgtgctcaccccaagagccggaagcagaaccacctgcccaaagtgctccaccccagcaaccctcacattagcagtggctccactgtggccacctgcttgagccagggtggcctcctggaagacctggacaacctcatcctggaggacctgaaggaggaggaggaggaagaggaggaggtggaggacgaggagggcgggcccagggagtgacccctgccaggtgcagatacaaaccagacacggtctgtggctactttgtgttattataagatatgagctcaaaccgagatatgaatgaccttggggagccatctgaggccaagatattgacgggggggattcctgggtcccattttcagcgcccagggtcacagatccacagtgggaagttctgtgggacacattggcactgagccacaaagaaggtgtggccagaacaacttgggctcctgctgaccaatgtcctctagggcctaggggacagaggaacacagagtcacagcttcaggggccgaatgagcatggcggccttcctgagagaatatgccccaccacgaaactcagcccagtagacaccatcctggtagcggcttcggtagtggccgccgtggtgccacacaccgttgaggttggagtgggcacaggcatggtaccaccagcctccccgctggtacagggcacagttacctgaggggagagagagagtccatgtcctctcaccagaataaaagcctctacctgcacctcacagtgcaaggcttttgccaggcatcccctggcccctcccattcttattgaatacaagccctgatcttccatctcctcagcaaaaaaataggagccctggccccccaactttcttcagagtaatagccttaattccttccctatctccttaccaaagtacaagtcacatctttcccaccttttctgcaaactaggagtctaccgttcattcctttatcaaagaaaagtatctacttcctttctagaataagagtactagctctcaccctctgccctttacttgaacaggagtcttgattctttttttgcctcatcagagaaggaatctggactccccatccccccaccaggataaaagtcctgacctttgttctcttgacggaataaaagcttgcttatcctta

SEQ ID NO: 3 - protein, 240 aa, Artificial Sequence
  SHFL - Variant 2
MSQHQQACGICRQEDPPEDMKQDRDIQAVATSLLPLTEANLRMFQRAQDDLIPAVDRQFACSSCDHVWWRRVPQRKEVSRCRKCRKRYEPVPADKMWGLAEFHCPKCRHNFRGWAQMGSPSPCYGCGFPVYPTRILPPRWDRDPDRRSTHTHSCSAADCYNRREPHVPGTSCAHPKSRKQNHLPKVLHPSNPHISSGSTVATCLSQGGLLEDLDNLILEDLKEEEEEEEEVEDEEGGPRE

SEQ ID NO: 4 - DNA, 2302 bp, Artificial Sequence
  SHFL- Variant 2
gacatgtgaccgactgcagaggccgcgccgcctcccccgtccgaggtctgcgcgctccgccgcagggtgcagacccggggcgcccgcctgggtttggggcgcaagagcagaggcggagccagggcggagccagcgcgccgggtcccccctgaatcgaaagcgaaacaggggccggggaggaagggcggagccggccccgaggcaccgccccctgccctgcgcggctgctggaccgacgggcgcacccaggtaggggggcggctgagccgcgcagtgcggaccctcgcggggaactgcgccgccgccaccatgtctcaggaaggtgtggagctggagaagagcgtccggcgcctccgggagaagtttcatgggaaggtatcctccaagaaggcgggggctctgatgaggaaattcggcagcgaccacacgggagtggggcgctccatcgtgtacggggtaaagcaaaaagatggccaagaactaagtaacgatctggatgcccagacaaagaaactgaggccgaagtcacttgccagcaagcaggggcagctgcgatttgaaccctggccacctggagtcagagcctgggctctaacatgcctccgagcacaacgtggcctcaaaggggcgatatgtcacaacatcaacaagcatgtggcatttgcaggcaggaagatccaccagaagatatgaagcaggaccgggacattcaggcagtggcgacctccctcctgccactgacagaagccaacctacgcatgtttcaacgtgcccaggacgaccttatccctgctgtggaccggcagtttgcctgctcctcctgcgaccacgtctggtggcgccgcgtgccccagcggaaggaggtatcccggtgccggaaatgccggaagcgctacgagccagtgccagctgacaagatgtggggcctggctgagttccactgcccgaagtgtcggcacaacttccggggctgggcacagatggggtccccgtccccctgctacgggtgcggcttccccgtgtatccaacacggatcctccccccgcgctgggaccgggacccggatcgccgcagcacccacactcactcctgctcagctgccgactgctacaaccggcgagagccccacgtgcctgggacatcctgtgctcaccccaagagccggaagcagaaccacctgcccaaagtgctccaccccagcaaccctcacattagcagtggctccactgtggccacctgcttgagccagggtggcctcctggaagacctggacaacctcatcctggaggacctgaaggaggaggaggaggaagaggaggaggtggaggacgaggagggcgggcccagggagtgacccctgccaggtgcagatacaaaccagacacggtctgtggctactttgtgttattataagatatgagctcaaaccgagatatgaatgaccttggggagccatctgaggccaagatattgacgggggggattcctgggtcccattttcagcgcccagggtcacagatccacagtgggaagttctgtgggacacattggcactgagccacaaagaaggtgtggccagaacaacttgggctcctgctgaccaatgtcctctagggcctaggggacagaggaacacagagtcacagcttcaggggccgaatgagcatggcggccttcctgagagaatatgccccaccacgaaactcagcccagtagacaccatcctggtagcggcttcggtagtggccgccgtggtgccacacaccgttgaggttggagtgggcacaggcatggtaccaccagcctccccgctggtacagggcacagttacctgaggggagagagagagtccatgtcctctcaccagaataaaagcctctacctgcacctcacagtgcaaggcttttgccaggcatcccctggcccctcccattcttattgaatacaagccctgatcttccatctcctcagcaaaaaaataggagccctggccccccaactttcttcagagtaatagccttaattccttccctatctccttaccaaagtacaagtcacatctttcccaccttttctgcaaactaggagtctaccgttcattcctttatcaaagaaaagtatctacttcctttctagaataagagtactagctctcaccctctgccctttacttgaacaggagtcttgattctttttttgcctcatcagagaaggaatctggactccccatccccccaccaggataaaagtcctgacctttgttctcttgacggaataaaagcttgcttatccttatactta

SEQ ID NO: 5 - protein, 290 aa, Artificial Sequence
  SHFL, deltaM - Variant 1
SQEGVELEKSVRRLREKFHGKVSSKKAGALMRKFGSDHTGVGRSIVYGVKQKDGQELSNDLDAQDPPEDMKQDRDIQAVATSLLPLTEANLRMFQRAQDDLIPAVDRQFACSSCDHVWWRRVPQRKEVSRCRKCRKRYEPVPADKMWGLAEFHCPKCRHNFRGWAQMGSPSPCYGCGFPVYPTRILPPRWDRDPDRRSTHTHSCSAADCYNRREPHVPGTSCAHPKSRKQNHLPKVLHPSNPHISSGSTVATCLSQGGLLEDLDNLILEDLKEEEEEEEEVEDEEGGPRE

SEQ ID NO: 6 - DNA, 1814 bp, Artificial Sequence
  SHFL, deltaM - Variant 1
tctcaggaaggtgtggagctggagaagagcgtccggcgcctccgggagaagtttcatgggaaggtatcctccaagaaggcgggggctctgatgaggaaattcggcagcgaccacacgggagtggggcgctccatcgtgtacggggtaaagcaaaaagatggccaagaactaagtaacgatctggatgcccaggatccaccagaagatatgaagcaggaccgggacattcaggcagtggcgacctccctcctgccactgacagaagccaacctacgcatgtttcaacgtgcccaggacgaccttatccctgctgtggaccggcagtttgcctgctcctcctgcgaccacgtctggtggcgccgcgtgccccagcggaaggaggtatcccggtgccggaaatgccggaagcgctacgagccagtgccagctgacaagatgtggggcctggctgagttccactgcccgaagtgtcggcacaacttccggggctgggcacagatggggtccccgtccccctgctacgggtgcggcttccccgtgtatccaacacggatcctccccccgcgctgggaccgggacccggatcgccgcagcacccacactcactcctgctcagctgccgactgctacaaccggcgagagccccacgtgcctgggacatcctgtgctcaccccaagagccggaagcagaaccacctgcccaaagtgctccaccccagcaaccctcacattagcagtggctccactgtggccacctgcttgagccagggtggcctcctggaagacctggacaacctcatcctggaggacctgaaggaggaggaggaggaagaggaggaggtggaggacgaggagggcgggcccagggagtgacccctgccaggtgcagatacaaaccagacacggtctgtggctactttgtgttattataagatatgagctcaaaccgagatatgaatgaccttggggagccatctgaggccaagatattgacgggggggattcctgggtcccattttcagcgcccagggtcacagatccacagtgggaagttctgtgggacacattggcactgagccacaaagaaggtgtggccagaacaacttgggctcctgctgaccaatgtcctctagggcctaggggacagaggaacacagagtcacagcttcaggggccgaatgagcatggcggccttcctgagagaatatgccccaccacgaaactcagcccagtagacaccatcctggtagcggcttcggtagtggccgccgtggtgccacacaccgttgaggttggagtgggcacaggcatggtaccaccagcctccccgctggtacagggcacagttacctgaggggagagagagagtccatgtcctctcaccagaataaaagcctctacctgcacctcacagtgcaaggcttttgccaggcatcccctggcccctcccattcttattgaatacaagccctgatcttccatctcctcagcaaaaaaataggagccctggccccccaactttcttcagagtaatagccttaattccttccctatctccttaccaaagtacaagtcacatctttcccaccttttctgcaaactaggagtctaccgttcattcctttatcaaagaaaagtatctacttcctttctagaataagagtactagctctcaccctctgccctttacttgaacaggagtcttgattctttttttgcctcatcagagaaggaatctggactccccatccccccaccaggataaaagtcctgacctttgttctcttgacggaataaaagcttgcttatcctta

SEQ ID NO: 7 - protein, 251 aa, Artificial Sequence
  SHFL, deltaM - Variant 2
PPSTTWPQRGDMSQHQQACGICRQEDPPEDMKQDRDIQAVATSLLPLTEANLRMFQRAQDDLIPAVDRQFACSSCDHVWWRRVPQRKEVSRCRKCRKRYEPVPADKMWGLAEFHCPKCRHNFRGWAQMGSPSPCYGCGFPVYPTRILPPRWDRDPDRRSTHTHSCSAADCYNRREPHVPGTSCAHPKSRKQNHLPKVLHPSNPHISSGSTVATCLSQGGLLEDLDNLILEDLKEEEEEEEEVEDEEGGPRE

SEQ ID NO: 8 - DNA, 2296 bp, Artificial Sequence
  SHFL, deltaM - Variant 2
tgaccgactgcagaggccgcgccgcctcccccgtccgaggtctgcgcgctccgccgcagggtgcagacccggggcgcccgcctgggtttggggcgcaagagcagaggcggagccagggcggagccagcgcgccgggtcccccctgaatcgaaagcgaaacaggggccggggaggaagggcggagccggccccgaggcaccgccccctgccctgcgcggctgctggaccgacgggcgcacccaggtaggggggcggctgagccgcgcagtgcggaccctcgcggggaactgcgccgccgccaccatgtctcaggaaggtgtggagctggagaagagcgtccggcgcctccgggagaagtttcatgggaaggtatcctccaagaaggcgggggctctgatgaggaaattcggcagcgaccacacgggagtggggcgctccatcgtgtacggggtaaagcaaaaagatggccaagaactaagtaacgatctggatgcccagacaaagaaactgaggccgaagtcacttgccagcaagcaggggcagctgcgatttgaaccctggccacctggagtcagagcctgggctctaacatgcctccgagcacaacgtggcctcaaaggggcgatatgtcacaacatcaacaagcatgtggcatttgcaggcaggaagatccaccagaagatatgaagcaggaccgggacattcaggcagtggcgacctccctcctgccactgacagaagccaacctacgcatgtttcaacgtgcccaggacgaccttatccctgctgtggaccggcagtttgcctgctcctcctgcgaccacgtctggtggcgccgcgtgccccagcggaaggaggtatcccggtgccggaaatgccggaagcgctacgagccagtgccagctgacaagatgtggggcctggctgagttccactgcccgaagtgtcggcacaacttccggggctgggcacagatggggtccccgtccccctgctacgggtgcggcttccccgtgtatccaacacggatcctccccccgcgctgggaccgggacccggatcgccgcagcacccacactcactcctgctcagctgccgactgctacaaccggcgagagccccacgtgcctgggacatcctgtgctcaccccaagagccggaagcagaaccacctgcccaaagtgctccaccccagcaaccctcacattagcagtggctccactgtggccacctgcttgagccagggtggcctcctggaagacctggacaacctcatcctggaggacctgaaggaggaggaggaggaagaggaggaggtggaggacgaggagggcgggcccagggagtgacccctgccaggtgcagatacaaaccagacacggtctgtggctactttgtgttattataagatatgagctcaaaccgagatatgaatgaccttggggagccatctgaggccaagatattgacgggggggattcctgggtcccattttcagcgcccagggtcacagatccacagtgggaagttctgtgggacacattggcactgagccacaaagaaggtgtggccagaacaacttgggctcctgctgaccaatgtcctctagggcctaggggacagaggaacacagagtcacagcttcaggggccgaatgagcatggcggccttcctgagagaatatgccccaccacgaaactcagcccagtagacaccatcctggtagcggcttcggtagtggccgccgtggtgccacacaccgttgaggttggagtgggcacaggcatggtaccaccagcctccccgctggtacagggcacagttacctgaggggagagagagagtccatgtcctctcaccagaataaaagcctctacctgcacctcacagtgcaaggcttttgccaggcatcccctggcccctcccattcttattgaatacaagccctgatcttccatctcctcagcaaaaaaataggagccctggccccccaactttcttcagagtaatagccttaattccttccctatctccttaccaaagtacaagtcacatctttcccaccttttctgcaaactaggagtctaccgttcattcctttatcaaagaaaagtatctacttcctttctagaataagagtactagctctcaccctctgccctttacttgaacaggagtcttgattctttttttgcctcatcagagaaggaatctggactccccatccccccaccaggataaaagtcctgacctttgttctcttgacggaataaaagcttgcttatccttatactta

SEQ ID NO: 9 - protein, 10 aa, Artificial Sequence
  NES Consensus
  VARIANT 2, 3, 4, 6, 7, 9: Xaa is any amino acid
LXXXLXXLXL

SEQ ID NO: 10 - DNA, 635 bp, Artificial Sequence
  Promoter region, SB gene
gtatagggctgtctgggagccactccagggccacagaaatcttgtctctgactcagggtattttgttttctgttttgtgtaaatgctcttctgactaatgcaaaccatgtgtccatagaaccagaagatttttccaggggaaaaggtaaggaggtggtgagagtgtcctgggtctgcccttccagggcttgccctgggttaagagccaggcaggaagctctcaagagcattgctcaagagtagagggggcctgggaggcccagggaggggatgggaggggaacacccaggctgcccccaaccagatgccctccaccctcctcaacctccctcccacggcctggagaggtgggaccaggtatggaggcttgagagcccctggttggaggaagccacaagtccaggaacatgggagtctgggcagggggcaaaggaggcaggaacaggccatcagccaggacaggtggtaaggcaggcaggagtgttcctgctgggaaaaggtgggatcaagcacctggagggctcttcagagcaaagacaaacactgaggtcgctgccactcctacagagcccccacgccccgcccagctataaggggccatgcaccaagcagggtacccaggctgcagaggtgcc

SEQ ID NO: 11 - protein, 17 aa, Artificial Sequence
  SHFL AA 121-137
RRVPQRKEVSRCRKCRK

SEQ ID NO: 12 - protein, 9 aa, Artificial Sequence
  SHFL AA 261-269
LEDLDNLIL

SEQ ID NO: 13 - protein, 13 aa, Artificial Sequence
  TAT
GRKKRRQRRRPPQ

SEQ ID NO: 14 - protein, 16 aa, Artificial Sequence
  Penetratin
RQIKIWFQNRRMKWKK

SEQ ID NO: 15 - protein, 27 aa, Artificial Sequence
  Transporant
GWTLNSAGYLLGKINLKALAALAKKIL

SEQ ID NO: 16 - protein, 12 aa, Artificial Sequence
  Pept 1
PLILLRLLRGQF

SEQ ID NO: 17 - protein, 12 aa, Artificial Sequence
  Pept 2
PLIYLRLLRGQF

SEQ ID NO: 18 - protein, 27 aa, Artificial Sequence
  Transportan
GWTLNSAGYLLGKINLKALAALAKKIL

SEQ ID NO: 19 - protein, 23 aa, Artificial Sequence
  IgV
MGLGLHLLVLAAALQGAKKKRKV

SEQ ID NO: 20 - protein, 5 aa, Artificial Sequence
  Peptide Linker
GGGGS

SEQ ID NO: 21 - protein, 6 aa, Artificial Sequence
  Peptide Linker
GGGGGS

SEQ ID NO: 22 - protein, 5 aa, Artificial Sequence
  Peptide Linker
  REPEAT (1)...(5): Occuring 1 to 6 times
GGGGS

SEQ ID NO: 23 - protein, 5 aa, Artificial Sequence
  Peptde Linker
  REPEAT (1)...(5): Occuring 1 to 10 times
GGGGS

SEQ ID NO: 24 - protein, 604 aa, Artificial Sequence
  Hendra Virus G Protein
MMADSKLVSLNNNLSGKIKDQGKVIKNYYGTMDIKKINDGLLDSKILGAFNTVIALLGSIIIIVMNIMIIQNYTRTTDNQALIKESLQSVQQQIKALTDKIGTEIGPKVSLIDTSSTITIPANIGLLGSKISQSTSSINENVNDKCKFTLPPLKIHECNISCPNPLPFREYRPISQGVSDLVGLPNQICLQKTTSTILKPRLISYTLPINTREGVCITDPLLAVDNGFFAYSHLEKIGSCTRGIAKQRIIGVGEVLDRGDKVPSMFMTNVWTPPNPSTIHHCSSTYHEDFYYTLCAVSHVGDPILNSTSWTESLSLIRLAVRPKSDSGDYNQKYIAITKVERGKYDKVMPYGPSGIKQGDTLYFPAVGFLPRTEFQYNDSNCPIIHCKYSKAENCRLSMGVNSKSHYILRSGLLKYNLSLGGDIILQFIEIADNRLTIGSPSKIYNSLGQPVFYQASYSWDTMIKLGDVDTVDPLRVQWRNNSVISRPGQSQCPRFNVCPEVCWEGTYNDAFLIDRLNWVSAGVYLNSNQTAENPVFAVFKDNEILYQVPLAEDDTNAQKTITDCFLLENVIWCISLVEIYDTGDSVIRPKLFAVKIPAQCSES

SEQ ID NO: 25 - protein, 603 aa, Artificial Sequence
  Hendra Virus G Protein (No Met)
MADSKLVSLNNNLSGKIKDQGKVIKNYYGTMDIKKINDGLLDSKILGAFNTVIALLGSIIIIVMNIMIIQNYTRTTDNQALIKESLQSVQQQIKALTDKIGTEIGPKVSLIDTSSTITIPANIGLLGSKISQSTSSINENVNDKCKFTLPPLKIHECNISCPNPLPFREYRPISQGVSDLVGLPNQICLQKTTSTILKPRLISYTLPINTREGVCITDPLLAVDNGFFAYSHLEKIGSCTRGIAKQRIIGVGEVLDRGDKVPSMFMTNVWTPPNPSTIHHCSSTYHEDFYYTLCAVSHVGDPILNSTSWTESLSLIRLAVRPKSDSGDYNQKYIAITKVERGKYDKVMPYGPSGIKQGDTLYFPAVGFLPRTEFQYNDSNCPIIHCKYSKAENCRLSMGVNSKSHYILRSGLLKYNLSLGGDIILQFIEIADNRLTIGSPSKIYNSLGQPVFYQASYSWDTMIKLGDVDTVDPLRVQWRNNSVISRPGQSQCPRFNVCPEVCWEGTYNDAFLIDRLNWVSAGVYLNSNQTAENPVFAVFKDNEILYQVPLAEDDTNAQKTITDCFLLENVIWCISLVEIYDTGDSVIRPKLFAVKIPAQCSES

SEQ ID NO: 26 - protein, 602 aa, Artificial Sequence
  Nipah Virus G Protein
MPAENKKVRFENTTSDKGKIPSKVIKSYYGTMDIKKINEGLLDSKILSAFNTVIALLGSIVIIVMNIMIIQNYTRSTDNQAVIKDALQGIQQQIKGLADKIGTEIGPKVSLIDTSSTITIPANIGLLGSKISQSTASINENVNEKCKFTLPPLKIHECNISCPNPLPFREYRPQTEGVSNLVGLPNNICLQKTSNQILKPKLISYTLPVVGQSGTCITDPLLAMDEGYFAYSHLERIGSCSRGVSKQRIIGVGEVLDRGDEVPSLFMTNVWTPPNPNTVYHCSAVYNNEFYYVLCAVSTVGDPILNSTYWSGSLMMTRLAVKPKSNGGGYNQHQLALRSIEKGRYDKVMPYGPSGIKQGDTLYFPAVGFLVRTEFKYNDSNCPITKCQYSKPENCRLSMGIRPNSHYILRSGLLKYNLSDGENPKVVFIEISDQRLSIGSPSKIYDSLGQPVFYQASFSWDTMIKFGDVLTVNPLVVNWRNNTVISRPGQSQCPRFNTCPEICWEGVYNDAFLIDRINWISAGVFLDSNQTAENPVFTVFKDNEILYRAQLASEDTNAQKTITNCFLLKNKIWCISLVEIYDTGDNVIRPKLFAVKIPEQCT

SEQ ID NO: 27 - protein, 601 aa, Artificial Sequence
  Nipah Virus G Protein (No Met)
PAENKKVRFENTTSDKGKIPSKVIKSYYGTMDIKKINEGLLDSKILSAFNTVIALLGSIVIIVMNIMIIQNYTRSTDNQAVIKDALQGIQQQIKGLADKIGTEIGPKVSLIDTSSTITIPANIGLLGSKISQSTASINENVNEKCKFTLPPLKIHECNISCPNPLPFREYRPQTEGVSNLVGLPNNICLQKTSNQILKPKLISYTLPVVGQSGTCITDPLLAMDEGYFAYSHLERIGSCSRGVSKQRIIGVGEVLDRGDEVPSLFMTNVWTPPNPNTVYHCSAVYNNEFYYVLCAVSTVGDPILNSTYWSGSLMMTRLAVKPKSNGGGYNQHQLALRSIEKGRYDKVMPYGPSGIKQGDTLYFPAVGFLVRTEFKYNDSNCPITKCQYSKPENCRLSMGIRPNSHYILRSGLLKYNLSDGENPKVVFIEISDQRLSIGSPSKIYDSLGQPVFYQASFSWDTMIKFGDVLTVNPLVVNWRNNTVISRPGQSQCPRFNTCPEICWEGVYNDAFLIDRINWISAGVFLDSNQTAENPVFTVFKDNEILYRAQLASEDTNAQKTITNCFLLKNKIWCISLVEIYDTGDNVIRPKLFAVKIPEQCT

SEQ ID NO: 28 - protein, 622 aa, Artificial Sequence
  Cedar Virus G Protein
MLSQLQKNYLDNSNQQGDKMNNPDKKLSVNFNPLELDKGQKDLNKSYYVKNKNYNVSNLLNESLHDIKFCIYCIFSLLIIITIINIITISIVITRLKVHEENNGMESPNLQSIQDSLSSLTNMINTEITPRIGILVTATSVTLSSSINYVGTKTNQLVNELKDYITKSCGFKVPELKLHECNISCADPKISKSAMYSTNAYAELAGPPKIFCKSVSKDPDFRLKQIDYVIPVQQDRSICMNNPLLDISDGFFTYIHYEGINSCKKSDSFKVLLSHGEIVDRGDYRPSLYLLSSHYHPYSMQVINCVPVTCNQSSFVFCHISNNTKTLDNSDYSSDEYYITYFNGIDRPKTKKIPINNMTADNRYIHFTFSGGGGVCLGEEFIIPVTTVINTDVFTHDYCESFNCSVQTGKSLKEICSESLRSPTNSSRYNLNGIMIISQNNMTDFKIQLNGITYNKLSFGSPGRLSKTLGQVLYYQSSMSWDTYLKAGFVEKWKPFTPNWMNNTVISRPNQGNCPRYHKCPEICYGGTYNDIAPLDLGKDMYVSVILDSDQLAENPEITVFNSTTILYKERVSKDELNTRSTTTSCFLFLDEPWCISVLETNRFNGKSIRPEIYSYKIPKYC

SEQ ID NO: 29 - protein, 621 aa, Artificial Sequence
  Cedar Virus G Protein (No Met)
LSQLQKNYLDNSNQQGDKMNNPDKKLSVNFNPLELDKGQKDLNKSYYVKNKNYNVSNLLNESLHDIKFCIYCIFSLLIIITIINIITISIVITRLKVHEENNGMESPNLQSIQDSLSSLTNMINTEITPRIGILVTATSVTLSSSINYVGTKTNQLVNELKDYITKSCGFKVPELKLHECNISCADPKISKSAMYSTNAYAELAGPPKIFCKSVSKDPDFRLKQIDYVIPVQQDRSICMNNPLLDISDGFFTYIHYEGINSCKKSDSFKVLLSHGEIVDRGDYRPSLYLLSSHYHPYSMQVINCVPVTCNQSSFVFCHISNNTKTLDNSDYSSDEYYITYFNGIDRPKTKKIPINNMTADNRYIHFTFSGGGGVCLGEEFIIPVTTVINTDVFTHDYCESFNCSVQTGKSLKEICSESLRSPTNSSRYNLNGIMIISQNNMTDFKIQLNGITYNKLSFGSPGRLSKTLGQVLYYQSSMSWDTYLKAGFVEKWKPFTPNWMNNTVISRPNQGNCPRYHKCPEICYGGTYNDIAPLDLGKDMYVSVILDSDQLAENPEITVFNSTTILYKERVSKDELNTRSTTTSCFLFLDEPWCISVLETNRFNGKSIRPEIYSYKIPKYC

SEQ ID NO: 30 - protein, 632 aa, Artificial Sequence
  Bat Paramyxovirus G Protein
MPQKTVEFINMNSPLERGVSTLSDKKTLNQSKITKQGYFGLGSHSERNWKKQKNQNDHYMTVSTMILEILVVLGIMFNLIVLTMVYYQNDNINQRMAELTSNITVLNLNLNQLTNKIQREIIPRITLIDTATTITIPSAITYILATLTTRISELLPSINQKCEFKTPTLVLNDCRINCTPPLNPSDGVKMSSLATNLVAHGPSPCRNFSSVPTIYYYRIPGLYNRTALDERCILNPRLTISSTKFAYVHSEYDKNCTRGFKYYELMTFGEILEGPEKEPRMFSRSFYSPTNAVNYHSCTPIVTVNEGYFLCLECTSSDPLYKANLSNSTFHLVILRHNKDEKIVSMPSFNLSTDQEYVQIIPAEGGGTAESGNLYFPCIGRLLHKRVTHPLCKKSNCSRTDDESCLKSYYNQGSPQHQVVNCLIRIRNAQRDNPTWDVITVDLTNTYPGSRSRIFGSFSKPMLYQSSVSWHTLLQVAEITDLDKYQLDWLDTPYISRPGGSECPFGNYCPTVCWEGTYNDVYSLTPNNDLFVTVYLKSEQVAENPYFAIFSRDQILKEFPLDAWISSARTTTISCFMFNNEIWCIAALEITRLNDDIIRPIYYSFWLPTDCRTPYPHTGKMTRVPLRSTYNY

SEQ ID NO: 31 - protein, 631 aa, Artificial Sequence
  Bat Paramyxovirus G Protein (No Met)
PQKTVEFINMNSPLERGVSTLSDKKTLNQSKITKQGYFGLGSHSERNWKKQKNQNDHYMTVSTMILEILVVLGIMFNLIVLTMVYYQNDNINQRMAELTSNITVLNLNLNQLTNKIQREIIPRITLIDTATTITIPSAITYILATLTTRISELLPSINQKCEFKTPTLVLNDCRINCTPPLNPSDGVKMSSLATNLVAHGPSPCRNFSSVPTIYYYRIPGLYNRTALDERCILNPRLTISSTKFAYVHSEYDKNCTRGFKYYELMTFGEILEGPEKEPRMFSRSFYSPTNAVNYHSCTPIVTVNEGYFLCLECTSSDPLYKANLSNSTFHLVILRHNKDEKIVSMPSFNLSTDQEYVQIIPAEGGGTAESGNLYFPCIGRLLHKRVTHPLCKKSNCSRTDDESCLKSYYNQGSPQHQVVNCLIRIRNAQRDNPTWDVITVDLTNTYPGSRSRIFGSFSKPMLYQSSVSWHTLLQVAEITDLDKYQLDWLDTPYISRPGGSECPFGNYCPTVCWEGTYNDVYSLTPNNDLFVTVYLKSEQVAENPYFAIFSRDQILKEFPLDAWISSARTTTISCFMFNNEIWCIAALEITRLNDDIIRPIYYSFWLPTDCRTPYPHTGKMTRVPLRSTYNY

SEQ ID NO: 32 - protein, 625 aa, Artificial Sequence
  Mojiang virus, Tongguan 1 G Protein
MATNRDNTITSAEVSQEDKVKKYYGVETAEKVADSISGNKVFILMNTLLILTGAIITITLNITNLTAAKSQQNMLKIIQDDVNAKLEMFVNLDQLVKGEIKPKVSLINTAVSVSIPGQISNLQTKFLQKYVYLEESITKQCTCNPLSGIFPTSGPTYPPTDKPDDDTTDDDKVDTTIKPIEYPKPDGCNRTGDHFTMEPGANFYTVPNLGPASSNSDECYTNPSFSIGSSIYMFSQEIRKTDCTAGEILSIQIVLGRIVDKGQQGPQASPLLVWAVPNPKIINSCAVAAGDEMGWVLCSVTLTAASGEPIPHMFDGFWLYKLEPDTEVVSYRITGYAYLLDKQYDSVFIGKGGGIQKGNDLYFQMYGLSRNRQSFKALCEHGSCLGTGGGGYQVLCDRAVMSFGSEESLITNAYLKVNDLASGKPVIIGQTFPPSDSYKGSNGRMYTIGDKYGLYLAPSSWNRYLRFGITPDISVRSTTWLKSQDPIMKILSTCTNTDRDMCPEICNTRGYQDIFPLSEDSEYYTYIGITPNNGGTKNFVAVRDSDGHIASIDILQNYYSITSATISCFMYKDEIWCIAITEGKKQKDNPQRIYAHSYKIRQMCYNMKSATVTVGNAKNITIRRY

SEQ ID NO: 33 - protein, 624 aa, Artificial Sequence
  Mojiang virus, Tongguan 1 G (No Met)
ATNRDNTITSAEVSQEDKVKKYYGVETAEKVADSISGNKVFILMNTLLILTGAIITITLNITNLTAAKSQQNMLKIIQDDVNAKLEMFVNLDQLVKGEIKPKVSLINTAVSVSIPGQISNLQTKFLQKYVYLEESITKQCTCNPLSGIFPTSGPTYPPTDKPDDDTTDDDKVDTTIKPIEYPKPDGCNRTGDHFTMEPGANFYTVPNLGPASSNSDECYTNPSFSIGSSIYMFSQEIRKTDCTAGEILSIQIVLGRIVDKGQQGPQASPLLVWAVPNPKIINSCAVAAGDEMGWVLCSVTLTAASGEPIPHMFDGFWLYKLEPDTEVVSYRITGYAYLLDKQYDSVFIGKGGGIQKGNDLYFQMYGLSRNRQSFKALCEHGSCLGTGGGGYQVLCDRAVMSFGSEESLITNAYLKVNDLASGKPVIIGQTFPPSDSYKGSNGRMYTIGDKYGLYLAPSSWNRYLRFGITPDISVRSTTWLKSQDPIMKILSTCTNTDRDMCPEICNTRGYQDIFPLSEDSEYYTYIGITPNNGGTKNFVAVRDSDGHIASIDILQNYYSITSATISCFMYKDEIWCIAITEGKKQKDNPQRIYAHSYKIRQMCYNMKSATVTVGNAKNITIRRY

SEQ ID NO: 34 - protein, 546 aa, Artificial Sequence
  Hendra virus F Protein
MATQEVRLKCLLCGIIVLVLSLEGLGILHYEKLSKIGLVKGITRKYKIKSNPLTKDIVIKMIPNVSNVSKCTGTVMENYKSRLTGILSPIKGAIELYNNNTHDLVGDVKLAGVVMAGIAIGIATAAQITAGVALYEAMKNADNINKLKSSIESTNEAVVKLQETAEKTVYVLTALQDYINTNLVPTIDQISCKQTELALDLALSKYLSDLLFVFGPNLQDPVSNSMTIQAISQAFGGNYETLLRTLGYATEDFDDLLESDSIAGQIVYVDLSSYYIIVRVYFPILTEIQQAYVQELLPVSFNNDNSEWISIVPNFVLIRNTLISNIEVKYCLITKKSVICNQDYATPMTASVRECLTGSTDKCPRELVVSSHVPRFALSGGVLFANCISVTCQCQTTGRAISQSGEQTLLMIDNTTCTTVVLGNIIISLGKYLGSINYNSESIAVGPPVYTDKVDISSQISSMNQSLQQSKDYIKEAQKILDTVNPSLISMLSMIILYVLSIAALCIGLITFISFVIVEKKRGNYSRLDDRQVRPVSNGDLYYIGT

SEQ ID NO: 35 - protein, 520 aa, Artificial Sequence
  Hendra virus F Protein, Without signal sequence
ILHYEKLSKIGLVKGITRKYKIKSNPLTKDIVIKMIPNVSNVSKCTGTVMENYKSRLTGILSPIKGAIELYNNNTHDLVGDVKLAGVVMAGIAIGIATAAQITAGVALYEAMKNADNINKLKSSIESTNEAVVKLQETAEKTVYVLTALQDYINTNLVPTIDQISCKQTELALDLALSKYLSDLLFVFGPNLQDPVSNSMTIQAISQAFGGNYETLLRTLGYATEDFDDLLESDSIAGQIVYVDLSSYYIIVRVYFPILTEIQQAYVQELLPVSFNNDNSEWISIVPNFVLIRNTLISNIEVKYCLITKKSVICNQDYATPMTASVRECLTGSTDKCPRELVVSSHVPRFALSGGVLFANCISVTCQCQTTGRAISQSGEQTLLMIDNTTCTTVVLGNIIISLGKYLGSINYNSESIAVGPPVYTDKVDISSQISSMNQSLQQSKDYIKEAQKILDTVNPSLISMLSMIILYVLSIAALCIGLITFISFVIVEKKRGNYSRLDDRQVRPVSNGDLYYIGT

SEQ ID NO: 36 - protein, 546 aa, Artificial Sequence
  Nipah virus F Protein
MVVILDKRCYCNLLILILMISECSVGILHYEKLSKIGLVKGVTRKYKIKSNPLTKDIVIKMIPNVSNMSQCTGSVMENYKTRLNGILTPIKGALEIYKNNTHDLVGDVRLAGVIMAGVAIGIATAAQITAGVALYEAMKNADNINKLKSSIESTNEAVVKLQETAEKTVYVLTALQDYINTNLVPTIDKISCKQTELSLDLALSKYLSDLLFVFGPNLQDPVSNSMTIQAISQAFGGNYETLLRTLGYATEDFDDLLESDSITGQIIYVDLSSYYIIVRVYFPILTEIQQAYIQELLPVSFNNDNSEWISIVPNFILVRNTLISNIEIGFCLITKRSVICNQDYATPMTNNMRECLTGSTEKCPRELVVSSHVPRFALSNGVLFANCISVTCQCQTTGRAISQSGEQTLLMIDNTTCPTAVLGNVIISLGKYLGSVNYNSEGIAIGPPVFTDKVDISSQISSMNQSLQQSKDYIKEAQRLLDTVNPSLISMLSMIILYVLSIASLCIGLITFISFIIVEKKRNTYSRLEDRRVRPTSSGDLYYIGT

SEQ ID NO: 37 - protein, 520 aa, Artificial Sequence
  Nipah virus F Protein, without signal sequence
ILHYEKLSKIGLVKGVTRKYKIKSNPLTKDIVIKMIPNVSNMSQCTGSVMENYKTRLNGILTPIKGALEIYKNNTHDLVGDVRLAGVIMAGVAIGIATAAQITAGVALYEAMKNADNINKLKSSIESTNEAVVKLQETAEKTVYVLTALQDYINTNLVPTIDKISCKQTELSLDLALSKYLSDLLFVFGPNLQDPVSNSMTIQAISQAFGGNYETLLRTLGYATEDFDDLLESDSITGQIIYVDLSSYYIIVRVYFPILTEIQQAYIQELLPVSFNNDNSEWISIVPNFILVRNTLISNIEIGFCLITKRSVICNQDYATPMTNNMRECLTGSTEKCPRELVVSSHVPRFALSNGVLFANCISVTCQCQTTGRAISQSGEQTLLMIDNTTCPTAVLGNVIISLGKYLGSVNYNSEGIAIGPPVFTDKVDISSQISSMNQSLQQSKDYIKEAQRLLDTVNPSLISMLSMIILYVLSIASLCIGLITFISFIIVEKKRNTYSRLEDRRVRPTSSGDLYYIGT

SEQ ID NO: 38 - protein, 557 aa, Artificial Sequence
  Cedar Virus F Protein
MSNKRTTVLIIISYTLFYLNNAAIVGFDFDKLNKIGVVQGRVLNYKIKGDPMTKDLVLKFIPNIVNITECVREPLSRYNETVRRLLLPIHNMLGLYLNNTNAKMTGLMIAGVIMGGIAIGIATAAQITAGFALYEAKKNTENIQKLTDSIMKTQDSIDKLTDSVGTSILILNKLQTYINNQLVPNLELLSCRQNKIEFDLMLTKYLVDLMTVIGPNINNPVNKDMTIQSLSLLFDGNYDIMMSELGYTPQDFLDLIESKSITGQIIYVDMENLYVVIRTYLPTLIEVPDAQIYEFNKITMSSNGGEYLSTIPNFILIRGNYMSNIDVATCYMTKASVICNQDYSLPMSQNLRSCYQGETEYCPVEAVIASHSPRFALTNGVIFANCINTICRCQDNGKTITQNINQFVSMIDNSTCNDVMVDKFTIKVGKYMGRKDINNINIQIGPQIIIDKVDLSNEINKMNQSLKDSIFYLREAKRILDSVNISLISPSVQLFLIIISVLSFIILLIIIVYLYCKSKHSYKYNKFIDDPDYYNDYKRERINGKASKSNNIYYVGD

SEQ ID NO: 39 - protein, 551 aa, Artificial Sequence
  Cedar Virus F Protein, without signal sequence
TVLIIISYTLFYLNNAAIVGFDFDKLNKIGVVQGRVLNYKIKGDPMTKDLVLKFIPNIVNITECVREPLSRYNETVRRLLLPIHNMLGLYLNNTNAKMTGLMIAGVIMGGIAIGIATAAQITAGFALYEAKKNTENIQKLTDSIMKTQDSIDKLTDSVGTSILILNKLQTYINNQLVPNLELLSCRQNKIEFDLMLTKYLVDLMTVIGPNINNPVNKDMTIQSLSLLFDGNYDIMMSELGYTPQDFLDLIESKSITGQIIYVDMENLYVVIRTYLPTLIEVPDAQIYEFNKITMSSNGGEYLSTIPNFILIRGNYMSNIDVATCYMTKASVICNQDYSLPMSQNLRSCYQGETEYCPVEAVIASHSPRFALTNGVIFANCINTICRCQDNGKTITQNINQFVSMIDNSTCNDVMVDKFTIKVGKYMGRKDINNINIQIGPQIIIDKVDLSNEINKMNQSLKDSIFYLREAKRILDSVNISLISPSVQLFLIIISVLSFIILLIIIVYLYCKSKHSYKYNKFIDDPDYYNDYKRERINGKASKSNNIYYVGD

SEQ ID NO: 40 - protein, 545 aa, Artificial Sequence
  Mojiang virus, Tongguan 1 F Protein
MALNKNMFSSLFLGYLLVYATTVQSSIHYDSLSKVGVIKGLTYNYKIKGSPSTKLMVVKLIPNIDSVKNCTQKQYDEYKNLVRKALEPVKMAIDTMLNNVKSGNNKYRFAGAIMAGVALGVATAATVTAGIALHRSNENAQAIANMKSAIQNTNEAVKQLQLANKQTLAVIDTIRGEINNNIIPVINQLSCDTIGLSVGIRLTQYYSEIITAFGPALQNPVNTRITIQAISSVFNGNFDELLKIMGYTSGDLYEILHSELIRGNIIDVDVDAGYIALEIEFPNLTLVPNAVVQELMPISYNIDGDEWVTLVPRFVLTRTTLLSNIDTSRCTITDSSVICDNDYALPMSHELIGCLQGDTSKCAREKVVSSYVPKFALSDGLVYANCLNTICRCMDTDTPISQSLGATVSLLDNKRCSVYQVGDVLISVGSYLGDGEYNADNVELGPPIVIDKIDIGNQLAGINQTLQEAEDYIEKSEEFLKGVNPSIITLGSMVVLYIFMILIAIVSVIALVLSIKLTVKGNVVRQQFTYTQHVPSMENINYVSH

SEQ ID NO: 41 - protein, 519 aa, Artificial Sequence
  Mojiang virus, Tongguan 1 F Protein, without signal sequence
IHYDSLSKVGVIKGLTYNYKIKGSPSTKLMVVKLIPNIDSVKNCTQKQYDEYKNLVRKALEPVKMAIDTMLNNVKSGNNKYRFAGAIMAGVALGVATAATVTAGIALHRSNENAQAIANMKSAIQNTNEAVKQLQLANKQTLAVIDTIRGEINNNIIPVINQLSCDTIGLSVGIRLTQYYSEIITAFGPALQNPVNTRITIQAISSVFNGNFDELLKIMGYTSGDLYEILHSELIRGNIIDVDVDAGYIALEIEFPNLTLVPNAVVQELMPISYNIDGDEWVTLVPRFVLTRTTLLSNIDTSRCTITDSSVICDNDYALPMSHELIGCLQGDTSKCAREKVVSSYVPKFALSDGLVYANCLNTICRCMDTDTPISQSLGATVSLLDNKRCSVYQVGDVLISVGSYLGDGEYNADNVELGPPIVIDKIDIGNQLAGINQTLQEAEDYIEKSEEFLKGVNPSIITLGSMVVLYIFMILIAIVSVIALVLSIKLTVKGNVVRQQFTYTQHVPSMENINYVSH

SEQ ID NO: 42 - protein, 662 aa, Artificial Sequence
  Bat Paramyxovirus F Protein
MKKKTDNPTISKRGHNHSRGIKSRALLRETDNYSNGLIVENLVRNCHHPSKNNLNYTKTQKRDSTIPYRVEERKGHYPKIKHLIDKSYKHIKRGKRRNGHNGNIITIILLLILILKTQMSEGAIHYETLSKIGLIKGITREYKVKGTPSSKDIVIKLIPNVTGLNKCTNISMENYKEQLDKILIPINNIIELYANSTKSAPGNARFAGVIIAGVALGVAAAAQITAGIALHEARQNAERINLLKDSISATNNAVAELQEATGGIVNVITGMQDYINTNLVPQIDKLQCSQIKTALDISLSQYYSEILTVFGPNLQNPVTTSMSIQAISQSFGGNIDLLLNLLGYTANDLLDLLESKSITGQITYINLEHYFMVIRVYYPIMTTISNAYVQELIKISFNVDGSEWVSLVPSYILIRNSYLSNIDISECLITKNSVICRHDFAMPMSYTLKECLTGDTEKCPREAVVTSYVPRFAISGGVIYANCLSTTCQCYQTGKVIAQDGSQTLMMIDNQTCSIVRIEEILISTGKYLGSQEYNTMHVSVGNPVFTDKLDITSQISNINQSIEQSKFYLDKSKAILDKINLNLIGSVPISILFIIAILSLILSIITFVIVMIIVRRYNKYTPLINSDPSSRRSTIQDVYIIPNPGEHSIRSAARSIDRDRD

SEQ ID NO: 43 - protein, 640 aa, Artificial Sequence
  Bat Paramyxovirus F Protein, without signal sequence
SRALLRETDNYSNGLIVENLVRNCHHPSKNNLNYTKTQKRDSTIPYRVEERKGHYPKIKHLIDKSYKHIKRGKRRNGHNGNIITIILLLILILKTQMSEGAIHYETLSKIGLIKGITREYKVKGTPSSKDIVIKLIPNVTGLNKCTNISMENYKEQLDKILIPINNIIELYANSTKSAPGNARFAGVIIAGVALGVAAAAQITAGIALHEARQNAERINLLKDSISATNNAVAELQEATGGIVNVITGMQDYINTNLVPQIDKLQCSQIKTALDISLSQYYSEILTVFGPNLQNPVTTSMSIQAISQSFGGNIDLLLNLLGYTANDLLDLLESKSITGQITYINLEHYFMVIRVYYPIMTTISNAYVQELIKISFNVDGSEWVSLVPSYILIRNSYLSNIDISECLITKNSVICRHDFAMPMSYTLKECLTGDTEKCPREAVVTSYVPRFAISGGVIYANCLSTTCQCYQTGKVIAQDGSQTLMMIDNQTCSIVRIEEILISTGKYLGSQEYNTMHVSVGNPVFTDKLDITSQISNINQSIEQSKFYLDKSKAILDKINLNLIGSVPISILFIIAILSLILSIITFVIVMIIVRRYNKYTPLINSDPSSRRSTIQDVYIIPNPGEHSIRSAARSIDRDRD

SEQ ID NO: 44 - protein, 602 aa, Artificial Sequence
  NiVG protein attachment glycoprotein (602 aa)
MGPAENKKVRFENTTSDKGKIPSKVIKSYYGTMDIKKINEGLLDSKILSAFNTVIALLGSIVIIVMNIMIIQNYTRSTDNQAVIKDALQGIQQQIKGLADKIGTEIGPKVSLIDTSSTITIPANIGLLGSKISQSTASINENVNEKCKFTLPPLKIHECNISCPNPLPFREYRPQTEGVSNLVGLPNNICLQKTSNQILKPKLISYTLPVVGQSGTCITDPLLAMDEGYFAYSHLERIGSCSRGVSKQRIIGVGEVLDRGDEVPSLFMTNVWTPPNPNTVYHCSAVYNNEFYYVLCAVSTVGDPILNSTYWSGSLMMTRLAVKPKSNGGGYNQHQLALRSIEKGRYDKVMPYGPSGIKQGDTLYFPAVGFLVRTEFKYNDSNCPITKCQYSKPENCRLSMGIRPNSHYILRSGLLKYNLSDGENPKVVFIEISDQRLSIGSPSKIYDSLGQPVFYQASFSWDTMIKFGDVLTVNPLVVNWRNNTVISRPGQSQCPRFNTCPEICWEGVYNDAFLIDRINWISAGVFLDSNQTAENPVFTVFKDNEILYRAQLASEDTNAQKTITNCFLLKNKIWCISLVEIYDTGDNVIRPKLFAVKIPEQC

SEQ ID NO: 45 - protein, 552 aa, Artificial Sequence
  NivG protein attachment glycoprotein, Without cytoplasmic tail, Uniprot Q9IH62
FNTVIALLGSIVIIVMNIMIIQNYTRSTDNQAVIKDALQGIQQQIKGLADKIGTEIGPKVSLIDTSSTITIPANIGLLGSKISQSTASINENVNEKCKFTLPPLKIHECNISCPNPLPFREYRPQTEGVSNLVGLPNNICLQKTSNQILKPKLISYTLPVVGQSGTCITDPLLAMDEGYFAYSHLERIGSCSRGVSKQRIIGVGEVLDRGDEVPSLFMTNVWTPPNPNTVYHCSAVYNNEFYYVLCAVSTVGDPILNSTYWSGSLMMTRLAVKPKSNGGGYNQHQLALRSIEKGRYDKVMPYGPSGIKQGDTLYFPAVGFLVRTEFKYNDSNCPITKCQYSKPENCRLSMGIRPNSHYILRSGLLKYNLSDGENPKVVFIEISDQRLSIGSPSKIYDSLGQPVFYQASFSWDTMIKFGDVLTVNPLVVNWRNNTVISRPGQSQCPRFNTCPEICWEGVYNDAFLIDRINWISAGVFLDSNQTAENPVFTVFKDNEILYRAQLASEDTNAQKTITNCFLLKNKIWCISLVEIYDTGDNVIRPKLFAVKIPEQC

SEQ ID NO: 46 - protein, 597 aa, Artificial Sequence
  NiVG protein attachment glycoprotein Truncated ?5
MGKVRFENTTSDKGKIPSKVIKSYYGTMDIKKINEGLLDSKILSAFNTVIALLGSIVIIVMNIMIIQNYTRSTDNQAVIKDALQGIQQQIKGLADKIGTEIGPKVSLIDTSSTITIPANIGLLGSKISQSTASINENVNEKCKFTLPPLKIHECNISCPNPLPFREYRPQTEGVSNLVGLPNNICLQKTSNQILKPKLISYTLPVVGQSGTCITDPLLAMDEGYFAYSHLERIGSCSRGVSKQRIIGVGEVLDRGDEVPSLFMTNVWTPPNPNTVYHCSAVYNNEFYYVLCAVSTVGDPILNSTYWSGSLMMTRLAVKPKSNGGGYNQHQLALRSIEKGRYDKVMPYGPSGIKQGDTLYFPAVGFLVRTEFKYNDSNCPITKCQYSKPENCRLSMGIRPNSHYILRSGLLKYNLSDGENPKVVFIEISDQRLSIGSPSKIYDSLGQPVFYQASFSWDTMIKFGDVLTVNPLVVNWRNNTVISRPGQSQCPRFNTCPEICWEGVYNDAFLIDRINWISAGVFLDSNQTAENPVFTVFKDNEILYRAQLASEDTNAQKTITNCFLLKNKIWCISLVEIYDTGDNVIRPKLFAVKIPEQC

SEQ ID NO: 47 - protein, 592 aa, Artificial Sequence
  NiVG protein attachment glycoprotein Truncated ?10
MGNTTSDKGKIPSKVIKSYYGTMDIKKINEGLLDSKILSAFNTVIALLGSIVIIVMNIMIIQNYTRSTDNQAVIKDALQGIQQQIKGLADKIGTEIGPKVSLIDTSSTITIPANIGLLGSKISQSTASINENVNEKCKFTLPPLKIHECNISCPNPLPFREYRPQTEGVSNLVGLPNNICLQKTSNQILKPKLISYTLPVVGQSGTCITDPLLAMDEGYFAYSHLERIGSCSRGVSKQRIIGVGEVLDRGDEVPSLFMTNVWTPPNPNTVYHCSAVYNNEFYYVLCAVSTVGDPILNSTYWSGSLMMTRLAVKPKSNGGGYNQHQLALRSIEKGRYDKVMPYGPSGIKQGDTLYFPAVGFLVRTEFKYNDSNCPITKCQYSKPENCRLSMGIRPNSHYILRSGLLKYNLSDGENPKVVFIEISDQRLSIGSPSKIYDSLGQPVFYQASFSWDTMIKFGDVLTVNPLVVNWRNNTVISRPGQSQCPRFNTCPEICWEGVYNDAFLIDRINWISAGVFLDSNQTAENPVFTVFKDNEILYRAQLASEDTNAQKTITNCFLLKNKIWCISLVEIYDTGDNVIRPKLFAVKIPEQC

SEQ ID NO: 48 - protein, 587 aa, Artificial Sequence
  NiVG protein attachment glycoprotein Truncated ?15
MGKGKIPSKVIKSYYGTMDIKKINEGLLDSKILSAFNTVIALLGSIVIIVMNIMIIQNYTRSTDNQAVIKDALQGIQQQIKGLADKIGTEIGPKVSLIDTSSTITIPANIGLLGSKISQSTASINENVNEKCKFTLPPLKIHECNISCPNPLPFREYRPQTEGVSNLVGLPNNICLQKTSNQILKPKLISYTLPVVGQSGTCITDPLLAMDEGYFAYSHLERIGSCSRGVSKQRIIGVGEVLDRGDEVPSLFMTNVWTPPNPNTVYHCSAVYNNEFYYVLCAVSTVGDPILNSTYWSGSLMMTRLAVKPKSNGGGYNQHQLALRSIEKGRYDKVMPYGPSGIKQGDTLYFPAVGFLVRTEFKYNDSNCPITKCQYSKPENCRLSMGIRPNSHYILRSGLLKYNLSDGENPKVVFIEISDQRLSIGSPSKIYDSLGQPVFYQASFSWDTMIKFGDVLTVNPLVVNWRNNTVISRPGQSQCPRFNTCPEICWEGVYNDAFLIDRINWISAGVFLDSNQTAENPVFTVFKDNEILYRAQLASEDTNAQKTITNCFLLKNKIWCISLVEIYDTGDNVIRPKLFAVKIPEQC

SEQ ID NO: 49 - protein, 582 aa, Artificial Sequence
  NiVG protein attachment glycoprotein Truncated ?20
MGSKVIKSYYGTMDIKKINEGLLDSKILSAFNTVIALLGSIVIIVMNIMIIQNYTRSTDNQAVIKDALQGIQQQIKGLADKIGTEIGPKVSLIDTSSTITIPANIGLLGSKISQSTASINENVNEKCKFTLPPLKIHECNISCPNPLPFREYRPQTEGVSNLVGLPNNICLQKTSNQILKPKLISYTLPVVGQSGTCITDPLLAMDEGYFAYSHLERIGSCSRGVSKQRIIGVGEVLDRGDEVPSLFMTNVWTPPNPNTVYHCSAVYNNEFYYVLCAVSTVGDPILNSTYWSGSLMMTRLAVKPKSNGGGYNQHQLALRSIEKGRYDKVMPYGPSGIKQGDTLYFPAVGFLVRTEFKYNDSNCPITKCQYSKPENCRLSMGIRPNSHYILRSGLLKYNLSDGENPKVVFIEISDQRLSIGSPSKIYDSLGQPVFYQASFSWDTMIKFGDVLTVNPLVVNWRNNTVISRPGQSQCPRFNTCPEICWEGVYNDAFLIDRINWISAGVFLDSNQTAENPVFTVFKDNEILYRAQLASEDTNAQKTITNCFLLKNKIWCISLVEIYDTGDNVIRPKLFAVKIPEQC

SEQ ID NO: 50 - protein, 577 aa, Artificial Sequence
  NiVG protein attachment glycoprotein Truncated ?25
MGSYYGTMDIKKINEGLLDSKILSAFNTVIALLGSIVIIVMNIMIIQNYTRSTDNQAVIKDALQGIQQQIKGLADKIGTEIGPKVSLIDTSSTITIPANIGLLGSKISQSTASINENVNEKCKFTLPPLKIHECNISCPNPLPFREYRPQTEGVSNLVGLPNNICLQKTSNQILKPKLISYTLPVVGQSGTCITDPLLAMDEGYFAYSHLERIGSCSRGVSKQRIIGVGEVLDRGDEVPSLFMTNVWTPPNPNTVYHCSAVYNNEFYYVLCAVSTVGDPILNSTYWSGSLMMTRLAVKPKSNGGGYNQHQLALRSIEKGRYDKVMPYGPSGIKQGDTLYFPAVGFLVRTEFKYNDSNCPITKCQYSKPENCRLSMGIRPNSHYILRSGLLKYNLSDGENPKVVFIEISDQRLSIGSPSKIYDSLGQPVFYQASFSWDTMIKFGDVLTVNPLVVNWRNNTVISRPGQSQCPRFNTCPEICWEGVYNDAFLIDRINWISAGVFLDSNQTAENPVFTVFKDNEILYRAQLASEDTNAQKTITNCFLLKNKIWCISLVEIYDTGDNVIRPKLFAVKIPEQC

SEQ ID NO: 51 - protein, 573 aa, Artificial Sequence
  NiVG protein attachment glycoprotein Truncated ?30
MGTMDIKKINEGLLDSKILSAFNTVIALLGSIVIIVMNIMIIQNYTRSTDNQAVIKDALQGIQQQIKGLADKIGTEIGPKVSLIDTSSTITIPANIGLLGSKISQSTASINENVNEKCKFTLPPLKIHECNISCPNPLPFREYRPQTEGVSNLVGLPNNICLQKTSNQILKPKLISYTLPVVGQSGTCITDPLLAMDEGYFAYSHLERIGSCSRGVSKQRIIGVGEVLDRGDEVPSLFMTNVWTPPNPNTVYHCSAVYNNEFYYVLCAVSTVGDPILNSTYWSGSLMMTRLAVKPKSNGGGYNQHQLALRSIEKGRYDKVMPYGPSGIKQGDTLYFPAVGFLVRTEFKYNDSNCPITKCQYSKPENCRLSMGIRPNSHYILRSGLLKYNLSDGENPKVVFIEISDQRLSIGSPSKIYDSLGQPVFYQASFSWDTMIKFGDVLTVNPLVVNWRNNTVISRPGQSQCPRFNTCPEICWEGVYNDAFLIDRINWISAGVFLDSNQTAENPVFTVFKDNEILYRAQLASEDTNAQKTITNCFLLKNKIWCISLVEIYDTGDNVIRPKLFAVKIPEQC

SEQ ID NO: 52 - protein, 597 aa, Artificial Sequence
  NiVG protein attachment glycoprotein Truncated ?5
MKVRFENTTSDKGKIPSKVIKSYYGTMDIKKINEGLLDSKILSAFNTVIALLGSIVIIVMNIMIIQNYTRSTDNQAVIKDALQGIQQQIKGLADKIGTEIGPKVSLIDTSSTITIPANIGLLGSKISQSTASINENVNEKCKFTLPPLKIHECNISCPNPLPFREYRPQTEGVSNLVGLPNNICLQKTSNQILKPKLISYTLPVVGQSGTCITDPLLAMDEGYFAYSHLERIGSCSRGVSKQRIIGVGEVLDRGDEVPSLFMTNVWTPPNPNTVYHCSAVYNNEFYYVLCAVSTVGDPILNSTYWSGSLMMTRLAVKPKSNGGGYNQHQLALRSIEKGRYDKVMPYGPSGIKQGDTLYFPAVGFLVRTEFKYNDSNCPITKCQYSKPENCRLSMGIRPNSHYILRSGLLKYNLSDGENPKVVFIEISDQRLSIGSPSKIYDSLGQPVFYQASFSWDTMIKFGDVLTVNPLVVNWRNNTVISRPGQSQCPRFNTCPEICWEGVYNDAFLIDRINWISAGVFLDSNQTAENPVFTVFKDNEILYRAQLASEDTNAQKTITNCFLLKNKIWCISLVEIYDTGDNVIRPKLFAVKIPEQCT

SEQ ID NO: 53 - protein, 592 aa, Artificial Sequence
  NiVG protein attachment glycoprotein Truncated ?10
MNTTSDKGKIPSKVIKSYYGTMDIKKINEGLLDSKILSAFNTVIALLGSIVIIVMNIMIIQNYTRSTDNQAVIKDALQGIQQQIKGLADKIGTEIGPKVSLIDTSSTITIPANIGLLGSKISQSTASINENVNEKCKFTLPPLKIHECNISCPNPLPFREYRPQTEGVSNLVGLPNNICLQKTSNQILKPKLISYTLPVVGQSGTCITDPLLAMDEGYFAYSHLERIGSCSRGVSKQRIIGVGEVLDRGDEVPSLFMTNVWTPPNPNTVYHCSAVYNNEFYYVLCAVSTVGDPILNSTYWSGSLMMTRLAVKPKSNGGGYNQHQLALRSIEKGRYDKVMPYGPSGIKQGDTLYFPAVGFLVRTEFKYNDSNCPITKCQYSKPENCRLSMGIRPNSHYILRSGLLKYNLSDGENPKVVFIEISDQRLSIGSPSKIYDSLGQPVFYQASFSWDTMIKFGDVLTVNPLVVNWRNNTVISRPGQSQCPRFNTCPEICWEGVYNDAFLIDRINWISAGVFLDSNQTAENPVFTVFKDNEILYRAQLASEDTNAQKTITNCFLLKNKIWCISLVEIYDTGDNVIRPKLFAVKIPEQCT

SEQ ID NO: 54 - protein, 587 aa, Artificial Sequence
  NiVG protein attachment glycoprotein Truncated ?15
MKGKIPSKVIKSYYGTMDIKKINEGLLDSKILSAFNTVIALLGSIVIIVMNIMIIQNYTRSTDNQAVIKDALQGIQQQIKGLADKIGTEIGPKVSLIDTSSTITIPANIGLLGSKISQSTASINENVNEKCKFTLPPLKIHECNISCPNPLPFREYRPQTEGVSNLVGLPNNICLQKTSNQILKPKLISYTLPVVGQSGTCITDPLLAMDEGYFAYSHLERIGSCSRGVSKQRIIGVGEVLDRGDEVPSLFMTNVWTPPNPNTVYHCSAVYNNEFYYVLCAVSTVGDPILNSTYWSGSLMMTRLAVKPKSNGGGYNQHQLALRSIEKGRYDKVMPYGPSGIKQGDTLYFPAVGFLVRTEFKYNDSNCPITKCQYSKPENCRLSMGIRPNSHYILRSGLLKYNLSDGENPKVVFIEISDQRLSIGSPSKIYDSLGQPVFYQASFSWDTMIKFGDVLTVNPLVVNWRNNTVISRPGQSQCPRFNTCPEICWEGVYNDAFLIDRINWISAGVFLDSNQTAENPVFTVFKDNEILYRAQLASEDTNAQKTITNCFLLKNKIWCISLVEIYDTGDNVIRPKLFAVKIPEQCT

SEQ ID NO: 55 - protein, 582 aa, Artificial Sequence
  NiVG protein attachment glycoprotein Truncated ?20
MSKVIKSYYGTMDIKKINEGLLDSKILSAFNTVIALLGSIVIIVMNIMIIQNYTRSTDNQAVIKDALQGIQQQIKGLADKIGTEIGPKVSLIDTSSTITIPANIGLLGSKISQSTASINENVNEKCKFTLPPLKIHECNISCPNPLPFREYRPQTEGVSNLVGLPNNICLQKTSNQILKPKLISYTLPVVGQSGTCITDPLLAMDEGYFAYSHLERIGSCSRGVSKQRIIGVGEVLDRGDEVPSLFMTNVWTPPNPNTVYHCSAVYNNEFYYVLCAVSTVGDPILNSTYWSGSLMMTRLAVKPKSNGGGYNQHQLALRSIEKGRYDKVMPYGPSGIKQGDTLYFPAVGFLVRTEFKYNDSNCPITKCQYSKPENCRLSMGIRPNSHYILRSGLLKYNLSDGENPKVVFIEISDQRLSIGSPSKIYDSLGQPVFYQASFSWDTMIKFGDVLTVNPLVVNWRNNTVISRPGQSQCPRFNTCPEICWEGVYNDAFLIDRINWISAGVFLDSNQTAENPVFTVFKDNEILYRAQLASEDTNAQKTITNCFLLKNKIWCISLVEIYDTGDNVIRPKLFAVKIPEQCT

SEQ ID NO: 56 - protein, 577 aa, Artificial Sequence
  NiVG protein attachment glycoprotein Truncated ?25
MSYYGTMDIKKINEGLLDSKILSAFNTVIALLGSIVIIVMNIMIIQNYTRSTDNQAVIKDALQGIQQQIKGLADKIGTEIGPKVSLIDTSSTITIPANIGLLGSKISQSTASINENVNEKCKFTLPPLKIHECNISCPNPLPFREYRPQTEGVSNLVGLPNNICLQKTSNQILKPKLISYTLPVVGQSGTCITDPLLAMDEGYFAYSHLERIGSCSRGVSKQRIIGVGEVLDRGDEVPSLFMTNVWTPPNPNTVYHCSAVYNNEFYYVLCAVSTVGDPILNSTYWSGSLMMTRLAVKPKSNGGGYNQHQLALRSIEKGRYDKVMPYGPSGIKQGDTLYFPAVGFLVRTEFKYNDSNCPITKCQYSKPENCRLSMGIRPNSHYILRSGLLKYNLSDGENPKVVFIEISDQRLSIGSPSKIYDSLGQPVFYQASFSWDTMIKFGDVLTVNPLVVNWRNNTVISRPGQSQCPRFNTCPEICWEGVYNDAFLIDRINWISAGVFLDSNQTAENPVFTVFKDNEILYRAQLASEDTNAQKTITNCFLLKNKIWCISLVEIYDTGDNVIRPKLFAVKIPEQCT

SEQ ID NO: 57 - protein, 573 aa, Artificial Sequence
  NiVG protein attachment glycoprotein Truncated ?30
MTMDIKKINEGLLDSKILSAFNTVIALLGSIVIIVMNIMIIQNYTRSTDNQAVIKDALQGIQQQIKGLADKIGTEIGPKVSLIDTSSTITIPANIGLLGSKISQSTASINENVNEKCKFTLPPLKIHECNISCPNPLPFREYRPQTEGVSNLVGLPNNICLQKTSNQILKPKLISYTLPVVGQSGTCITDPLLAMDEGYFAYSHLERIGSCSRGVSKQRIIGVGEVLDRGDEVPSLFMTNVWTPPNPNTVYHCSAVYNNEFYYVLCAVSTVGDPILNSTYWSGSLMMTRLAVKPKSNGGGYNQHQLALRSIEKGRYDKVMPYGPSGIKQGDTLYFPAVGFLVRTEFKYNDSNCPITKCQYSKPENCRLSMGIRPNSHYILRSGLLKYNLSDGENPKVVFIEISDQRLSIGSPSKIYDSLGQPVFYQASFSWDTMIKFGDVLTVNPLVVNWRNNTVISRPGQSQCPRFNTCPEICWEGVYNDAFLIDRINWISAGVFLDSNQTAENPVFTVFKDNEILYRAQLASEDTNAQKTITNCFLLKNKIWCISLVEIYDTGDNVIRPKLFAVKIPEQCT

SEQ ID NO: 58 - protein, 595 aa, Artificial Sequence
  NiVG protein attachment glycoprotein Truncated ?5 Without N-terminal methionine
KVRFENTTSDKGKIPSKVIKSYYGTMDIKKINEGLLDSKILSAFNTVIALLGSIVIIVMNIMIIQNYTRSTDNQAVIKDALQGIQQQIKGLADKIGTEIGPKVSLIDTSSTITIPANIGLLGSKISQSTASINENVNEKCKFTLPPLKIHECNISCPNPLPFREYRPQTEGVSNLVGLPNNICLQKTSNQILKPKLISYTLPVVGQSGTCITDPLLAMDEGYFAYSHLERIGSCSRGVSKQRIIGVGEVLDRGDEVPSLFMTNVWTPPNPNTVYHCSAVYNNEFYYVLCAVSTVGDPILNSTYWSGSLMMTRLAVKPKSNGGGYNQHQLALRSIEKGRYDKVMPYGPSGIKQGDTLYFPAVGFLVRTEFKYNDSNCPITKCQYSKPENCRLSMGIRPNSHYILRSGLLKYNLSDGENPKVVFIEISDQRLSIGSPSKIYDSLGQPVFYQASFSWDTMIKFGDVLTVNPLVVNWRNNTVISRPGQSQCPRFNTCPEICWEGVYNDAFLIDRINWISAGVFLDSNQTAENPVFTVFKDNEILYRAQLASEDTNAQKTITNCFLLKNKIWCISLVEIYDTGDNVIRPKLFAVKIPEQC

SEQ ID NO: 59 - protein, 590 aa, Artificial Sequence
  NiVG protein attachment glycoprotein Truncated ?10 Without N-terminal methionine
NTTSDKGKIPSKVIKSYYGTMDIKKINEGLLDSKILSAFNTVIALLGSIVIIVMNIMIIQNYTRSTDNQAVIKDALQGIQQQIKGLADKIGTEIGPKVSLIDTSSTITIPANIGLLGSKISQSTASINENVNEKCKFTLPPLKIHECNISCPNPLPFREYRPQTEGVSNLVGLPNNICLQKTSNQILKPKLISYTLPVVGQSGTCITDPLLAMDEGYFAYSHLERIGSCSRGVSKQRIIGVGEVLDRGDEVPSLFMTNVWTPPNPNTVYHCSAVYNNEFYYVLCAVSTVGDPILNSTYWSGSLMMTRLAVKPKSNGGGYNQHQLALRSIEKGRYDKVMPYGPSGIKQGDTLYFPAVGFLVRTEFKYNDSNCPITKCQYSKPENCRLSMGIRPNSHYILRSGLLKYNLSDGENPKVVFIEISDQRLSIGSPSKIYDSLGQPVFYQASFSWDTMIKFGDVLTVNPLVVNWRNNTVISRPGQSQCPRFNTCPEICWEGVYNDAFLIDRINWISAGVFLDSNQTAENPVFTVFKDNEILYRAQLASEDTNAQKTITNCFLLKNKIWCISLVEIYDTGDNVIRPKLFAVKIPEQC

SEQ ID NO: 60 - protein, 585 aa, Artificial Sequence
  NiVG protein attachment glycoprotein Truncated ?15 Without N-terminal methionine
KGKIPSKVIKSYYGTMDIKKINEGLLDSKILSAFNTVIALLGSIVIIVMNIMIIQNYTRSTDNQAVIKDALQGIQQQIKGLADKIGTEIGPKVSLIDTSSTITIPANIGLLGSKISQSTASINENVNEKCKFTLPPLKIHECNISCPNPLPFREYRPQTEGVSNLVGLPNNICLQKTSNQILKPKLISYTLPVVGQSGTCITDPLLAMDEGYFAYSHLERIGSCSRGVSKQRIIGVGEVLDRGDEVPSLFMTNVWTPPNPNTVYHCSAVYNNEFYYVLCAVSTVGDPILNSTYWSGSLMMTRLAVKPKSNGGGYNQHQLALRSIEKGRYDKVMPYGPSGIKQGDTLYFPAVGFLVRTEFKYNDSNCPITKCQYSKPENCRLSMGIRPNSHYILRSGLLKYNLSDGENPKVVFIEISDQRLSIGSPSKIYDSLGQPVFYQASFSWDTMIKFGDVLTVNPLVVNWRNNTVISRPGQSQCPRFNTCPEICWEGVYNDAFLIDRINWISAGVFLDSNQTAENPVFTVFKDNEILYRAQLASEDTNAQKTITNCFLLKNKIWCISLVEIYDTGDNVIRPKLFAVKIPEQC

SEQ ID NO: 61 - protein, 580 aa, Artificial Sequence
  NiVG protein attachment glycoprotein Truncated ?20 Without N-terminal methionine
SKVIKSYYGTMDIKKINEGLLDSKILSAFNTVIALLGSIVIIVMNIMIIQNYTRSTDNQAVIKDALQGIQQQIKGLADKIGTEIGPKVSLIDTSSTITIPANIGLLGSKISQSTASINENVNEKCKFTLPPLKIHECNISCPNPLPFREYRPQTEGVSNLVGLPNNICLQKTSNQILKPKLISYTLPVVGQSGTCITDPLLAMDEGYFAYSHLERIGSCSRGVSKQRIIGVGEVLDRGDEVPSLFMTNVWTPPNPNTVYHCSAVYNNEFYYVLCAVSTVGDPILNSTYWSGSLMMTRLAVKPKSNGGGYNQHQLALRSIEKGRYDKVMPYGPSGIKQGDTLYFPAVGFLVRTEFKYNDSNCPITKCQYSKPENCRLSMGIRPNSHYILRSGLLKYNLSDGENPKVVFIEISDQRLSIGSPSKIYDSLGQPVFYQASFSWDTMIKFGDVLTVNPLVVNWRNNTVISRPGQSQCPRFNTCPEICWEGVYNDAFLIDRINWISAGVFLDSNQTAENPVFTVFKDNEILYRAQLASEDTNAQKTITNCFLLKNKIWCISLVEIYDTGDNVIRPKLFAVKIPEQC

SEQ ID NO: 62 - protein, 575 aa, Artificial Sequence
  NiVG protein attachment glycoprotein Truncated ?25 Without N-terminal methionine
SYYGTMDIKKINEGLLDSKILSAFNTVIALLGSIVIIVMNIMIIQNYTRSTDNQAVIKDALQGIQQQIKGLADKIGTEIGPKVSLIDTSSTITIPANIGLLGSKISQSTASINENVNEKCKFTLPPLKIHECNISCPNPLPFREYRPQTEGVSNLVGLPNNICLQKTSNQILKPKLISYTLPVVGQSGTCITDPLLAMDEGYFAYSHLERIGSCSRGVSKQRIIGVGEVLDRGDEVPSLFMTNVWTPPNPNTVYHCSAVYNNEFYYVLCAVSTVGDPILNSTYWSGSLMMTRLAVKPKSNGGGYNQHQLALRSIEKGRYDKVMPYGPSGIKQGDTLYFPAVGFLVRTEFKYNDSNCPITKCQYSKPENCRLSMGIRPNSHYILRSGLLKYNLSDGENPKVVFIEISDQRLSIGSPSKIYDSLGQPVFYQASFSWDTMIKFGDVLTVNPLVVNWRNNTVISRPGQSQCPRFNTCPEICWEGVYNDAFLIDRINWISAGVFLDSNQTAENPVFTVFKDNEILYRAQLASEDTNAQKTITNCFLLKNKIWCISLVEIYDTGDNVIRPKLFAVKIPEQC

SEQ ID NO: 63 - protein, 571 aa, Artificial Sequence
  NiVG protein attachment glycoprotein Truncated ?30 Without N-terminal methionine
TMDIKKINEGLLDSKILSAFNTVIALLGSIVIIVMNIMIIQNYTRSTDNQAVIKDALQGIQQQIKGLADKIGTEIGPKVSLIDTSSTITIPANIGLLGSKISQSTASINENVNEKCKFTLPPLKIHECNISCPNPLPFREYRPQTEGVSNLVGLPNNICLQKTSNQILKPKLISYTLPVVGQSGTCITDPLLAMDEGYFAYSHLERIGSCSRGVSKQRIIGVGEVLDRGDEVPSLFMTNVWTPPNPNTVYHCSAVYNNEFYYVLCAVSTVGDPILNSTYWSGSLMMTRLAVKPKSNGGGYNQHQLALRSIEKGRYDKVMPYGPSGIKQGDTLYFPAVGFLVRTEFKYNDSNCPITKCQYSKPENCRLSMGIRPNSHYILRSGLLKYNLSDGENPKVVFIEISDQRLSIGSPSKIYDSLGQPVFYQASFSWDTMIKFGDVLTVNPLVVNWRNNTVISRPGQSQCPRFNTCPEICWEGVYNDAFLIDRINWISAGVFLDSNQTAENPVFTVFKDNEILYRAQLASEDTNAQKTITNCFLLKNKIWCISLVEIYDTGDNVIRPKLFAVKIPEQC

SEQ ID NO: 64 - protein, 569 aa, Artifical Sequence
  NiVG protein attachment glycoprotein Truncated (Gc ? 34)
MKKINEGLLDSKILSAFNTVIALLGSIVIIVMNIMIIQNYTRSTDNQAVIKDALQGIQQQIKGLADKIGTEIGPKVSLIDTSSTITIPANIGLLGSKISQSTASINENVNEKCKFTLPPLKIHECNISCPNPLPFREYRPQTEGVSNLVGLPNNICLQKTSNQILKPKLISYTLPVVGQSGTCITDPLLAMDEGYFAYSHLERIGSCSRGVSKQRIIGVGEVLDRGDEVPSLFMTNVWTPPNPNTVYHCSAVYNNEFYYVLCAVSTVGDPILNSTYWSGSLMMTRLAVKPKSNGGGYNQHQLALRSIEKGRYDKVMPYGPSGIKQGDTLYFPAVGFLVRTEFKYNDSNCPITKCQYSKPENCRLSMGIRPNSHYILRSGLLKYNLSDGENPKVVFIEISDQRLSIGSPSKIYDSLGQPVFYQASFSWDTMIKFGDVLTVNPLVVNWRNNTVISRPGQSQCPRFNTCPEICWEGVYNDAFLIDRINWISAGVFLDSNQTAENPVFTVFKDNEILYRAQLASEDTNAQKTITNCFLLKNKIWCISLVEIYDTGDNVIRPKLFAVKIPEQCT

SEQ ID NO: 65 - protein, 568 aa, Artificial Sequence
  NiVG protein attachment glycoprotein Truncated (Gc ? 34) Without N-terminal methionine
KKINEGLLDSKILSAFNTVIALLGSIVIIVMNIMIIQNYTRSTDNQAVIKDALQGIQQQIKGLADKIGTEIGPKVSLIDTSSTITIPANIGLLGSKISQSTASINENVNEKCKFTLPPLKIHECNISCPNPLPFREYRPQTEGVSNLVGLPNNICLQKTSNQILKPKLISYTLPVVGQSGTCITDPLLAMDEGYFAYSHLERIGSCSRGVSKQRIIGVGEVLDRGDEVPSLFMTNVWTPPNPNTVYHCSAVYNNEFYYVLCAVSTVGDPILNSTYWSGSLMMTRLAVKPKSNGGGYNQHQLALRSIEKGRYDKVMPYGPSGIKQGDTLYFPAVGFLVRTEFKYNDSNCPITKCQYSKPENCRLSMGIRPNSHYILRSGLLKYNLSDGENPKVVFIEISDQRLSIGSPSKIYDSLGQPVFYQASFSWDTMIKFGDVLTVNPLVVNWRNNTVISRPGQSQCPRFNTCPEICWEGVYNDAFLIDRINWISAGVFLDSNQTAENPVFTVFKDNEILYRAQLASEDTNAQKTITNCFLLKNKIWCISLVEIYDTGDNVIRPKLFAVKIPEQCT

SEQ ID NO: 66 - protein, 604 aa, Artificial Sequence; identical to SEQ ID NO: 24
  Hendra virus G protein Uniprot O89343

SEQ ID NO: 67 - protein, 603 aa, Artificial Sequence; identical to SEQ ID NO: 25
  Hendra virus G protein Uniprot O89343 Without N-terminal methionine

SEQ ID NO: 68 - protein, 555 aa, Artificial Sequence
  Hendra virus G protein Uniprot O89343, Without cytoplasmic tail
FNTVIALLGSIIIIVMNIMIIQNYTRTTDNQALIKESLQSVQQQIKALTDKIGTEIGPKVSLIDTSSTITIPANIGLLGSKISQSTSSINENVNDKCKFTLPPLKIHECNISCPNPLPFREYRPISQGVSDLVGLPNQICLQKTTSTILKPRLISYTLPINTREGVCITDPLLAVDNGFFAYSHLEKIGSCTRGIAKQRIIGVGEVLDRGDKVPSMFMTNVWTPPNPSTIHHCSSTYHEDFYYTLCAVSHVGDPILNSTSWTESLSLIRLAVRPKSDSGDYNQKYIAITKVERGKYDKVMPYGPSGIKQGDTLYFPAVGFLPRTEFQYNDSNCPIIHCKYSKAENCRLSMGVNSKSHYILRSGLLKYNLSLGGDIILQFIEIADNRLTIGSPSKIYNSLGQPVFYQASYSWDTMIKLGDVDTVDPLRVQWRNNSVISRPGQSQCPRFNVCPEVCWEGTYNDAFLIDRLNWVSAGVYLNSNQTAENPVFAVFKDNEILYQVPLAEDDTNAQKTITDCFLLENVIWCISLVEIYDTGDSVIRPKLFAVKIPAQCSES

SEQ ID NO: 69 - protein, 569 aa, Artificial Sequence
  NiVG protein attachment glycoprotein Truncated and mutated (E501 A, W504A, Q530A, E533A) NiV G protein (Gc ? 34)
MKKINEGLLDSKILSAFNTVIALLGSIVIIVMNIMIIQNYTRSTDNQAVIKDALQGIQQQIKGLADKIGTEIGPKVSLIDTSSTITIPANIGLLGSKISQSTASINENVNEKCKFTLPPLKIHECNISCPNPLPFREYRPQTEGVSNLVGLPNNICLQKTSNQILKPKLISYTLPVVGQSGTCITDPLLAMDEGYFAYSHLERIGSCSRGVSKQRIIGVGEVLDRGDEVPSLFMTNVWTPPNPNTVYHCSAVYNNEFYYVLCAVSTVGDPILNSTYWSGSLMMTRLAVKPKSNGGGYNQHQLALRSIEKGRYDKVMPYGPSGIKQGDTLYFPAVGFLVRTEFKYNDSNCPITKCQYSKPENCRLSMGIRPNSHYILRSGLLKYNLSDGENPKVVFIEISDQRLSIGSPSKIYDSLGQPVFYQASFSWDTMIKFGDVLTVNPLVVNWRNNTVISRPGQSQCPRFNTCPAICAEGVYNDAFLIDRINWISAGVFLDSNATAANPVFTVFKDNEILYRAQLASEDTNAQKTITNCFLLKNKIWCISLVEIYDTGDNVIRPKLFAVKIPEQCT

SEQ ID NO: 70 - protein, 568 aa, Artificial Sequence
  NiVG protein attachment glycoprotein Truncated and mutated (E501 A, W504A, Q530A, E533A) NiV G protein (Gc ? 34) Without N-terminal methionine
KKINEGLLDSKILSAFNTVIALLGSIVIIVMNIMIIQNYTRSTDNQAVIKDALQGIQQQIKGLADKIGTEIGPKVSLIDTSSTITIPANIGLLGSKISQSTASINENVNEKCKFTLPPLKIHECNISCPNPLPFREYRPQTEGVSNLVGLPNNICLQKTSNQILKPKLISYTLPVVGQSGTCITDPLLAMDEGYFAYSHLERIGSCSRGVSKQRIIGVGEVLDRGDEVPSLFMTNVWTPPNPNTVYHCSAVYNNEFYYVLCAVSTVGDPILNSTYWSGSLMMTRLAVKPKSNGGGYNQHQLALRSIEKGRYDKVMPYGPSGIKQGDTLYFPAVGFLVRTEFKYNDSNCPITKCQYSKPENCRLSMGIRPNSHYILRSGLLKYNLSDGENPKVVFIEISDQRLSIGSPSKIYDSLGQPVFYQASFSWDTMIKFGDVLTVNPLVVNWRNNTVISRPGQSQCPRFNTCPAICAEGVYNDAFLIDRINWISAGVFLDSNATAANPVFTVFKDNEILYRAQLASEDTNAQKTITNCFLLKNKIWCISLVEIYDTGDNVIRPKLFAVKIPEQCT

SEQ ID NO: 71 - protein, 26 aa, Artificial Sequence
  signal sequence
MVVILDKRCYCNLLILILMISECSVG

SEQ ID NO: 72 - protein, 83 aa, Artificial Sequence
  Nipah virus NiV-F F2 (aa 27-109)
ILHYEKLSKIGLVKGVTRKYKIKSNPLTKDIVIKMIPNVSNMSQCTGSVMENYKTRLNGILTPIKGALEIYKNNTHDLVGDVR

SEQ ID NO: 73 - protein, 437 aa, Artificial Sequence
  Nipah virus NiV F F1 (aa 110-546)
LAGVIMAGVAIGIATAAQITAGVALYEAMKNADNINKLKSSIESTNEAVVKLQETAEKTVYVLTALQDYINTNLVPTIDKISCKQTELSLDLALSKYLSDLLFVFGPNLQDPVSNSMTIQAISQAFGGNYETLLRTLGYATEDFDDLLESDSITGQIIYVDLSSYYIIVRVYFPILTEIQQAYIQELLPVSFNNDNSEWISIVPNFILVRNTLISNIEIGFCLITKRSVICNQDYATPMTNNMRECLTGSTEKCPRELVVSSHVPRFALSNGVLFANCISVTCQCQTTGRAISQSGEQTLLMIDNTTCPTAVLGNVIISLGKYLGSVNYNSEGIAIGPPVFTDKVDISSQISSMNQSLQQSKDYIKEAQRLLDTVNPSLISMLSMIILYVLSIASLCIGLITFISFIIVEKKRNTYSRLEDRRVRPTSSGDLYYIGT

SEQ ID NO: 74 - protein, 500 aa, Artificial Sequence
  Nipah virus NiV-F F0 T234 truncation (aa 525-544) AND mutation on N-linked glycosylation site
ILHYEKLSKIGLVKGVTRKYKIKSNPLTKDIVIKMIPNVSNMSQCTGSVMENYKTRLNGILTPIKGALEIYKNQTHDLVGDVRLAGVIMAGVAIGIATAAQITAGVALYEAMKNADNINKLKSSIESTNEAVVKLQETAEKTVYVLTALQDYINTNLVPTIDKISCKQTELSLDLALSKYLSDLLFVFGPNLQDPVSNSMTIQAISQAFGGNYETLLRTLGYATEDFDDLLESDSITGQIIYVDLSSYYIIVRVYFPILTEIQQAYIQELLPVSFNNDNSEWISIVPNFILVRNTLISNIEIGFCLITKRSVICNQDYATPMTNNMRECLTGSTEKCPRELVVSSHVPRFALSNGVLFANCISVTCQCQTTGRAISQSGEQTLLMIDNTTCPTAVLGNVIISLGKYLGSVNYNSEGIAIGPPVFTDKVDISSQISSMNQSLQQSKDYIKEAQRLLDTVNPSLISMLSMIILYVLSIASLCIGLITFISFIIVEKKRNTGT

SEQ ID NO: 75 - protein, 524 aa, Artificial Sequence
  Truncated NiV fusion glycoprotein (FcDelta22) at cytoplasmic tail (with signal sequence)
MVVILDKRCYCNLLILILMISECSVGILHYEKLSKIGLVKGVTRKYKIKSNPLTKDIVIKMIPNVSNMSQCTGSVMENYKTRLNGILTPIKGALEIYKNNTHDLVGDVRLAGVIMAGVAIGIATAAQITAGVALYEAMKNADNINKLKSSIESTNEAVVKLQETAEKTVYVLTALQDYINTNLVPTIDKISCKQTELSLDLALSKYLSDLLFVFGPNLQDPVSNSMTIQAISQAFGGNYETLLRTLGYATEDFDDLLESDSITGQIIYVDLSSYYIIVRVYFPILTEIQQAYIQELLPVSFNNDNSEWISIVPNFILVRNTLISNIEIGFCLITKRSVICNQDYATPMTNNMRECLTGSTEKCPRELVVSSHVPRFALSNGVLFANCISVTCQCQTTGRAISQSGEQTLLMIDNTTCPTAVLGNVIISLGKYLGSVNYNSEGIAIGPPVFTDKVDISSQISSMNQSLQQSKDYIKEAQRLLDTVNPSLISMLSMIILYVLSIASLCIGLITFISFIIVEKKRNT

SEQ ID NO: 76 - protein, 500 aa, Artificial Sequence
  Nipah virus NiV-F F0 T234 truncation (aa 525-544)
ILHYEKLSKIGLVKGVTRKYKIKSNPLTKDIVIKMIPNVSNMSQCTGSVMENYKTRLNGILTPIKGALEIYKNNTHDLVGDVRLAGVIMAGVAIGIATAAQITAGVALYEAMKNADNINKLKSSIESTNEAVVKLQETAEKTVYVLTALQDYINTNLVPTIDKISCKQTELSLDLALSKYLSDLLFVFGPNLQDPVSNSMTIQAISQAFGGNYETLLRTLGYATEDFDDLLESDSITGQIIYVDLSSYYIIVRVYFPILTEIQQAYIQELLPVSFNNDNSEWISIVPNFILVRNTLISNIEIGFCLITKRSVICNQDYATPMTNNMRECLTGSTEKCPRELVVSSHVPRFALSNGVLFANCISVTCQCQTTGRAISQSGEQTLLMIDNTTCPTAVLGNVIISLGKYLGSVNYNSEGIAIGPPVFTDKVDISSQISSMNQSLQQSKDYIKEAQRLLDTVNPSLISMLSMIILYVLSIASLCIGLITFISFIIVEKKRNTGT

SEQ ID NO: 77 - protein, 526 aa, Artificial Sequence
  Nipah virus NiV-F F0 T234 truncation (aa 525-544) (with signal sequence)
MVVILDKRCYCNLLILILMISECSVGILHYEKLSKIGLVKGVTRKYKIKSNPLTKDIVIKMIPNVSNMSQCTGSVMENYKTRLNGILTPIKGALEIYKNNTHDLVGDVRLAGVIMAGVAIGIATAAQITAGVALYEAMKNADNINKLKSSIESTNEAVVKLQETAEKTVYVLTALQDYINTNLVPTIDKISCKQTELSLDLALSKYLSDLLFVFGPNLQDPVSNSMTIQAISQAFGGNYETLLRTLGYATEDFDDLLESDSITGQIIYVDLSSYYIIVRVYFPILTEIQQAYIQELLPVSFNNDNSEWISIVPNFILVRNTLISNIEIGFCLITKRSVICNQDYATPMTNNMRECLTGSTEKCPRELVVSSHVPRFALSNGVLFANCISVTCQCQTTGRAISQSGEQTLLMIDNTTCPTAVLGNVIISLGKYLGSVNYNSEGIAIGPPVFTDKVDISSQISSMNQSLQQSKDYIKEAQRLLDTVNPSLISMLSMIILYVLSIASLCIGLITFISFIIVEKKRNTGT

SEQ ID NO: 78 - protein, 526 aa, Artificial Sequence
  Nipah virus NiV-F F0 T234 truncation (aa 525-544) AND mutation on N-linked glycosylation site (with signal sequence)
MVVILDKRCYCNLLILILMISECSVGILHYEKLSKIGLVKGVTRKYKIKSNPLTKDIVIKMIPNVSNMSQCTGSVMENYKTRLNGILTPIKGALEIYKNQTHDLVGDVRLAGVIMAGVAIGIATAAQITAGVALYEAMKNADNINKLKSSIESTNEAVVKLQETAEKTVYVLTALQDYINTNLVPTIDKISCKQTELSLDLALSKYLSDLLFVFGPNLQDPVSNSMTIQAISQAFGGNYETLLRTLGYATEDFDDLLESDSITGQIIYVDLSSYYIIVRVYFPILTEIQQAYIQELLPVSFNNDNSEWISIVPNFILVRNTLISNIEIGFCLITKRSVICNQDYATPMTNNMRECLTGSTEKCPRELVVSSHVPRFALSNGVLFANCISVTCQCQTTGRAISQSGEQTLLMIDNTTCPTAVLGNVIISLGKYLGSVNYNSEGIAIGPPVFTDKVDISSQISSMNQSLQQSKDYIKEAQRLLDTVNPSLISMLSMIILYVLSIASLCIGLITFISFIIVEKKRNTGT

SEQ ID NO: 79 - protein, 524 aa, Artificial Sequence; identical to SEQ ID NO: 75
  Truncated NiV fusion glycoprotein (FcDelta22) at cytoplasmic tail (with signal sequence)

SEQ ID NO: 80 - protein, 498 aa, Artificial Sequence
  Truncated mature NiV fusion glycoprotein (FcDelta22) at cytoplasmic tail
ILHYEKLSKIGLVKGVTRKYKIKSNPLTKDIVIKMIPNVSNMSQCTGSVMENYKTRLNGILTPIKGALEIYKNNTHDLVGDVRLAGVIMAGVAIGIATAAQITAGVALYEAMKNADNINKLKSSIESTNEAVVKLQETAEKTVYVLTALQDYINTNLVPTIDKISCKQTELSLDLALSKYLSDLLFVFGPNLQDPVSNSMTIQAISQAFGGNYETLLRTLGYATEDFDDLLESDSITGQIIYVDLSSYYIIVRVYFPILTEIQQAYIQELLPVSFNNDNSEWISIVPNFILVRNTLISNIEIGFCLITKRSVICNQDYATPMTNNMRECLTGSTEKCPRELVVSSHVPRFALSNGVLFANCISVTCQCQTTGRAISQSGEQTLLMIDNTTCPTAVLGNVIISLGKYLGSVNYNSEGIAIGPPVFTDKVDISSQISSMNQSLQQSKDYIKEAQRLLDTVNPSLISMLSMIILYVLSIASLCIGLITFISFIIVEKKRNT

SEQ ID NO: 81 - protein, 6 aa, Artificial Sequence
  Chelsky NLS Consensus
  VARIANT 4: Xaa is any amino acid
KKRXKR

SEQ ID NO: 82 - protein, 16 aa, Artificial Sequence
  Bipartite NLS Consensus
KRPAATKKAGQAKKKK

SEQ ID NO: 83 - protein, 9 aa, Artificial Sequence
  c-Myc NLS
PAAKRVKLD

SEQ ID NO: 84 - protein, 10 aa, Artificial Sequence
  SV40 Large T NLS
PKKKRKVEDP

SEQ ID NO: 85 - protein, 9 aa, Artificial Sequence
  NES
LEDLDNLIL

SEQ ID NO: 86 - protein, 17 aa, Artificial Sequence
  NLS
RRVPQRKEVSRCRKCRK

SEQ ID NO: 87 - protein, 10 aa, Artificial Sequence
  NES
  VARIANT 2, 3, 4, 6, 7, 9: Xaa is any amino acid
LXXXLXXLXL

SEQ ID NO: 88 - protein, 540 aa, Artificial Sequence
  Sendai F protein
QIPRDRLSNIGVIVDEGKSLKIAGSHESRYIVLSLVPGVDFENGCGTAQVIQYKSLLNRLLIPLRDALDLQEALITVTNDTTQNAGAPQSRFFGAVIGTIALGVATSAQITAGIALAEAREAKRDIALIKESMTKTHKSIELLQNAVGEQILALKTLQDFVNDEIKPAISELGCETAALRLGIKLTQHYSELLTAFGSNFGTIGEKSLTLQALSSLYSANITEIMTTIKTGQSNIYDVIYTEQIKGTVIDVDLERYMVTLSVKIPILSEVPGVLIHKASSISYNIDGEEWYVTVPSHILSRASFLGGADITDCVESRLTYICPRDPAQLIPDSQQKCILGDTTRCPVTKVVDSLIPKFAFVNGGVVANCIASTCTCGTGRRPISQDRSKGVVFLTHDNCGLIGVNGVELYANRRGHDATWGVQNLTVGPAIAIRPIDISLNLADATNFLQDSKAELEKARKILSEVGRWYNSRETVITIIVVMVVILVVIIVIIIVLYRLRRSMLMGNPDDRIPRDTYTLEPKIRHMYTNGGFDAMAEKR

SEQ ID NO: 89 - protein, 575 aa, Artificial Sequence
  Sendai HN protein
MDGDRGKRDSYWSTSPSGSTTKPASGWERSSKADTWLLILSFTQWALSIATVIICIIISARQGYSMKEYSMTVEALNMSSREVKESLTSLIRQEVIARAVNIQSSVQTGIPVLLNKNSRDVIQMIDKSCSRQELTQHCESTIAVHHADGIAPLEPHSFWRCPVGEPYLSSDPEISLLPGPSLLSGSTTISGCVRLPSLSIGEAIYAYSSNLITQGCADIGKSYQVLQLGYISLNSDMFPDLNPVVSHTYDINDNRKSCSVVATGTRGYQLCSMPTVDERTDYSSDGIEDLVLDVLDLKGRTKSHRYRNSEVDLDHPFSALYPSVGNGIATEGSLIFLGYGGLTTPLQGDTKCRTQGCQQVSQDTCNEALKITWLGGKQVVSVIIQVNDYLSERPKIRVTTIPITQNYLGAEGRLLKLGDRVYIYTRSSGWHSQLQIGVLDVSHPLTINWTPHEALSRPGNKECNWYNKCPKECISGVYTDAYPLSPDAANVATVTLYANTSRVNPTIMYSNTTNIINMLRIKDVQLEAAYTTTSCITHFGKGYCFHIIEINQKSLNTLQPMLFKTSIPKLCKAES

SEQ ID NO: 90 - protein, 618 aa, Artificial Sequence
  Sendai HN protein (modified CTD)
MWSELKIRSNDGGEGPEDANDPRGKGVQHIHIQPSLPVGQRVRMDGDRGKRDSYWSTSPSGSTTKPASGWERSSKADTWLLILSFTQWALSIATVIICIIISARQGYSMKEYSMTVEALNMSSREVKESLTSLIRQEVIARAVNIQSSVQTGIPVLLNKNSRDVIQMIDKSCSRQELTQHCESTIAVHHADGIAPLEPHSFWRCPVGEPYLSSDPEISLLPGPSLLSGSTTISGCVRLPSLSIGEAIYAYSSNLITQGCADIGKSYQVLQLGYISLNSDMFPDLNPVVSHTYDINDNRKSCSVVATGTRGYQLCSMPTVDERTDYSSDGIEDLVLDVLDLKGRTKSHRYRNSEVDLDHPFSALYPSVGNGIATEGSLIFLGYGGLTTPLQGDTKCRTQGCQQVSQDTCNEALKITWLGGKQVVSVIIQVNDYLSERPKIRVTTIPITQNYLGAEGRLLKLGDRVYIYTRSSGWHSQLQIGVLDVSHPLTINWTPHEALSRPGNKECNWYNKCPKECISGVYTDAYPLSPDAANVATVTLYANTSRVNPTIMYSNTTNIINMLRIKDVQLEAAYTTTSCITHFGKGYCFHIIEINQKSLNTLQPMLFKTSIPKLCKAES

SEQ ID NO: 91 - protein, 417 aa, Artificial Sequence
  Nipah virus NiV F F1 (aa 110-546) truncation (aa 525-544)
LAGVIMAGVAIGIATAAQITAGVALYEAMKNADNINKLKSSIESTNEAVVKLQETAEKTVYVLTALQDYINTNLVPTIDKISCKQTELSLDLALSKYLSDLLFVFGPNLQDPVSNSMTIQAISQAFGGNYETLLRTLGYATEDFDDLLESDSITGQIIYVDLSSYYIIVRVYFPILTEIQQAYIQELLPVSFNNDNSEWISIVPNFILVRNTLISNIEIGFCLITKRSVICNQDYATPMTNNMRECLTGSTEKCPRELVVSSHVPRFALSNGVLFANCISVTCQCQTTGRAISQSGEQTLLMIDNTTCPTAVLGNVIISLGKYLGSVNYNSEGIAIGPPVFTDKVDISSQISSMNQSLQQSKDYIKEAQRLLDTVNPSLISMLSMIILYVLSIASLCIGLITFISFIIVEKKRNTGT